(19) United States
(12) Patent Application Publication
(10) Pub. No.: US 2008/0207573 A1
(43) Pub. Date: Aug. 28, 2008

(54) COMPOUNDS FOR TREATING VIRAL INFECTIONS

(75) Inventors: Kraig M. Yager, Murray, UT (US); Esther Arranz Plaza, Madrid (ES); Dange Vijay Kumar, Salt Lake City, UT (US); In Chul Kim, Sandy, UT (US)

Correspondence Address: MYRIAD GENETICS INC.;INTELLECUTAL PROPERTY DEPARTMENT, 320 WAKARA WAY, SALT LAKE CITY, UT, 84108, US

(73) Assignee: Myriad Genetics, Incorporated, Salt Lake City, UT (US)

(21) Appl. No.: 12/116,864

(22) Filed: May 7, 2008

Related U.S. Application Data
(63) Continuation of application No. 11/873,342, filed on Oct. 16, 2007.
(60) Provisional application No. 60/852,141, filed on Oct. 16, 2006.
(60) Provisional application No. 60/877,584, filed on Dec. 27, 2006.

(51) Int. Cl. A61K 31/56; C07J 53/00

(52) U.S. Cl. 514/171; 552/510; 514/182

(57) ABSTRACT

The invention relates to compounds, pharmaceutical compositions and methods useful for treating viral infection.

### CROSS REFERENCES TO RELATED U.S. APPLICATIONS

This application is a continuation of Ser. No. 11/873,342, filed Oct. 16, 2007, which claims benefit of U.S. Provisional Patent Application No. 60/852,141, filed Oct. 16, 2006, and U.S. Provisional Patent Application No. 60/877,584, filed Dec. 27, 2006, which are hereby incorporated by reference in their entirety.

### FIELD OF THE INVENTION

The present invention relates generally to methods, compounds, and pharmaceutical compositions for treating (and delaying the onset of) viral infection, and particularly HIV infection and AIDS, and for treating cancer.

### BACKGROUND OF THE INVENTION

Viral infection of humans is a major health problem, and viral infection of domesticated animals is a major economic concern. Combating viral infection has proven to be highly effective in some cases like smallpox where the disease was essentially eradicated with the advent of smallpox vaccination. Although smallpox was essentially eradicated by about 1980, there is considerable justified fear of the emergence of a new epidemic of smallpox since there are existing stockpiles of the virus and bioterrorism has moved beyond the realm of possibility to reality. Other viral infections have been much more difficult to fight. Hepatitis B and C, human immunodeficiency virus (HIV), herpes simplex viruses, and influenza are just a few prominent members of a list of viruses that pose significant health threats worldwide. Additionally, emerging viral infections continue to threaten the world with human epidemics, as is illustrated by the recent outbreak of severe acute respiratory syndrome (SARS) which has now been associated with coronavirus infection. Treatments currently available for many viral infections are often associated with adverse side effects. In addition, antiviral therapeutics directed towards specific viral gene products frequently have the effect of driving the selection of viruses resistant to such therapeutics, and viral strains resistant to current methods of treatment are an increasing problem. Accordingly, there is a clear and ever-present need for new antiviral treatments.

A number of patent publications and articles disclose compounds that are betulinic acid derivatives useful for treating HIV infection, including, for example, WO 96/39033; Sun et al., J. Med. Chem., 41:4648-4657 (1998); U.S. Pat. No. 7,026,305; and WO 2006/053255.

### BRIEF SUMMARY OF THE INVENTION

The present invention generally relates to compounds useful for treating viral infections, particularly HIV infection. Specifically, the present invention provides compounds of Formulae I′-IV:

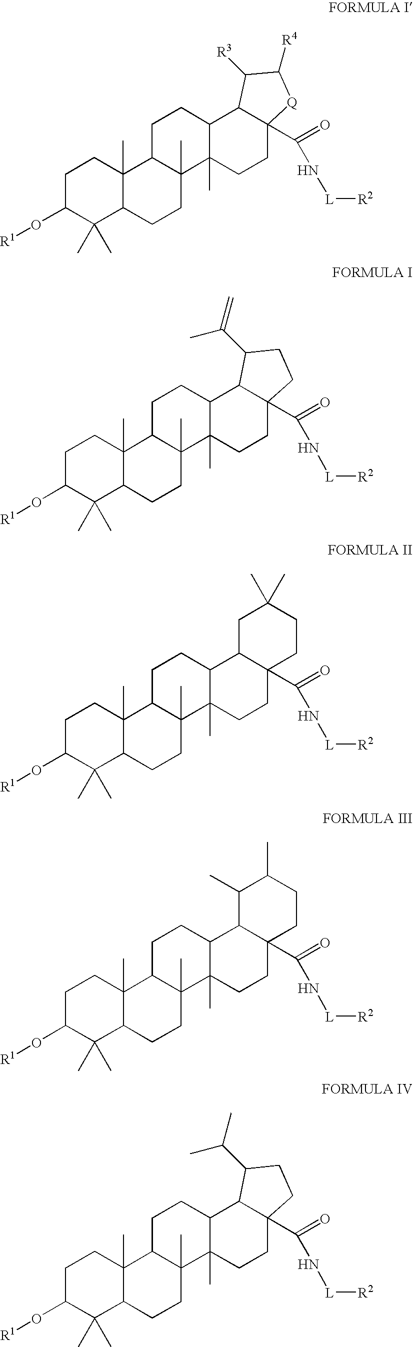

and pharmaceutically acceptable salts and stereoisomers thereof,
wherein Q, R¹, R², R³, R⁴ and L are as defined herein below.

Some of the compounds in the invention have chiral centers, and the invention therefore encompasses all stereoisomers, enantiomers, diastereomers, and mixtures thereof.

The compounds of the present invention are effective HIV inhibitors, and are useful in inhibiting HIV infection and transmission. Thus, in a related aspect, the present invention also provides a method for treating viral infection, particularly HIV infection and AIDS, by administering to a patient in need of such treatment a therapeutically effective amount of a compound of the present invention.

Also provided in the present invention is a pharmaceutical composition having one or more compounds of the present invention and one or more pharmaceutically acceptable excipients. A method for treating viral infection, particularly HIV infection and AIDS, by administering to a patient in need of the treatment the pharmaceutical composition is also encompassed.

In addition, the present invention further provides methods for inhibiting, or reducing the likelihood of, HIV transmission, or delaying the onset of the symptoms associated with HIV infection, or delaying the onset of AIDS, comprising administering an effective amount of a compound of the present invention, preferably in a pharmaceutical composition or medicament to an individual having an HIV infection, or at risk of HIV infection, or at risk of developing symptoms of HIV infection or AIDS.

The compounds of the present invention are also effective in treating cancer. Thus, in a related aspect, the present invention also provides a method for treating a patient for cancer, by administering to the patient in need of such treatment a therapeutically effective amount of a compound of the present invention.

The compounds of the present invention for use in the instant invention can be provided as a pharmaceutical composition with one or more salts, carriers, or excipients.

The compounds of the present invention can be used in combination therapies. Thus, combination therapy methods are also provided for treating HIV infection, inhibiting, or reducing the likelihood of, HIV transmission, or delaying the onset of the symptoms associated with HIV infection, or delaying the onset of AIDS. Such methods comprise administering to a patient in need thereof a compound of the present invention, and together or separately, at least one other anti-HIV compound. For the convenience of combination therapy, the compound of the present invention is administered together in the same formulation with such other anti-HIV compound. Thus, the present invention also provides a pharmaceutical composition or medicament for the combination therapy, comprising an effective amount of a first compound according to the present invention and an effective amount of at least one other anti-HIV compound, which is different from the first compound. Examples of antiviral compounds include, but are not limited to, protease inhibitors, nucleoside reverse transcriptase inhibitors, non-nucleoside reverse transcriptase inhibitors, integrase inhibitors, fusion inhibitors, immunomodulators, and vaccines.

The foregoing and other advantages and features of the invention, and the manner in which they are accomplished, will become more readily apparent upon consideration of the following detailed description of the invention taken in conjunction with the accompanying examples, which illustrate preferred and exemplary embodiments.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

Other features and advantages of the invention will be apparent from the following detailed description, and from the claims.

### DETAILED DESCRIPTION OF THE INVENTION

The invention provides compounds of Formula I′, including compounds of the present invention, which are useful for treating viral infections and symptoms thereof. Compounds of Formula I′ and the present invention include:

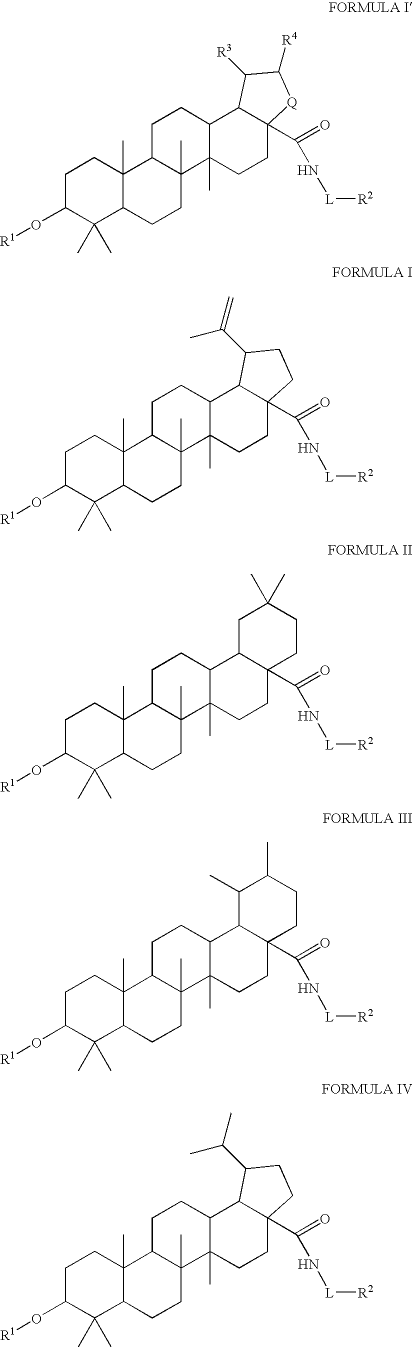

and pharmaceutically acceptable salts and stereoisomers thereof,
wherein

### Q is (CH₂)₁₋₂;

L is a bond or an alkyl group having from 1 to 10 carbons, or a C₁₋₁₀ alkynyl or alkenyl group, wherein one or more of the carbons of the alkyl, alkynyl or alkenyl group of L can be replaced with —O—, —S—, —N—, —C(═O)—, —NC(═O)—, —C(═O)N—, —SO₂, —NSO₂, —SO₂N—, cycloalkyl, and —NC(═O)N—; L can be substituted with one or more substituents chosen from hydroxyl, halo, alkyl, alkoxy, haloalkyl, haloalkoxy, —N(C₁₋₃ alkyl)₂, —NH(C₁₋₃ alkyl), —C(═O)OH, —C(═O)O(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂;
R¹ is chosen from hydro, —C(═O)—(CH₂)ₘ—CH₃, —C(═O)—(CH₂)ₘ—C(CH₃)₂—COOH;
R² is chosen from cycloalkyl, aryl, heterocycle, and heteroaryl, optionally substituted with one or more substituents chosen from hydro, hydroxyl, halo, alkyl, alkoxy, alkylthio, arylthio, thiocarbonyl, O-carboxy, C-carboxy, O-carbamyl, O-thiocarbamyl, N-carbamyl, N-thiocarbamyl, ester, haloalkyl, haloalkoxy, cycloalkyl, aryl, heteroaryl, heterocycle, —C(═O)OH, —CH(CH₃)C(═O)OH; —CH₂C(═O)OH, —C(CH₃)₂C(═O)OH, —C(CH₃)(CH₂CH₃)C(═O)OH, —CH(CH₂CH₃)C(═O)OH, —CH═C(CH₃)C(═O)OH, —C(CH₂CH₃)₂C(═O)OH, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂;
m is an integer chosen from 0-10; and
R³ and R⁴ are independently selected from —H, —CH₃, —(CH₃)₂, —CH(CH₃)₂, and —C(═CH₂)CH₃.

In some embodiments of the present invention, R¹ is —C(═O)—(CH₂)ₘ—CH₃ and m is an integer chosen from 0-10. In some embodiments of the present invention, R¹ is —C(═O)—(CH₂)ₘ—C(CH₃)₂—COOH and m is an integer chosen from 0-10. In specific embodiments of the present invention, L is an alkyl group having 0, 1, 2, 3, 4, or 5 carbons that can be saturated or partially saturated; and can be replaced and/or have substituents as defined for L above.

In some embodiments, L can have one or more substituents chosen from halo, alkyl, haloalkyl, —C(═O)OH, —C(═O)O(C₁₋₃ alkyl), —C(═O)NH₂, —C(═O)NH(C₁₋₃ alkyl), —C(═O)N(C₁₋₃ alkyl)₂, —CHF₂, —CF₃, and —CN. In some embodiments, L can have one or more substituents chosen from hydroxyl, alkoxy, haloalkoxy, —S(═O)₂(C₁₋₃alkyl), —S(═O)₂NH₂, —S(═O)₂N(C₁₋₃ alkyl)₂, —S(═O)₂NH(C₁₋₃ alkyl), OCF₃, —OCHF₂, and —SCF₃. In certain embodiments, L can have one or more substituents chosen from —N(C₁₋₃ alkyl)₂, —NH(C₁₋₃ alkyl), —NH₂, and —NO₂.

In some embodiments, L is methyl optionally substituted by one or more methyl, cyclopropyl, or cyclobutyl groups.

In some embodiments, R² is a phenyl group substituted with one or more substituents chosen from halo, alkyl, C-carboxy, haloalkyl, —C(═O)OH, —CH(CH₃)C(═O)OH; —CH₂C(═O)OH, —C(CH₃)₂C(═O)OH, —C(CH₃)(CH₂CH₃)C(═O)OH, —CH(CH₂CH₃)C(═O)OH, —CH═C(CH₃)C(═O)OH, —C(CH₂CH₃)₂C(═O)OH, —C(═O)NH₂, —C(═O)NH(C₁₋₃ alkyl), —C(═O)N(C₁₋₃ alkyl)₂, —CHF₂, —CF₃, and —CN.

In some embodiments, R² is a phenyl group substituted with one or more substituents chosen from hydroxyl, alkoxy, alkylthio, arylthio, thiocarbonyl, O-carboxy, O-carbamyl, O-thiocarbamyl, ester, haloalkoxy, —S(═O)₂(C₁₋₃alkyl), —S(═O)₂NH₂, —S(═O)₂N(C₁₋₃ alkyl)₂, —S(═O)₂NH(C₁₋₃ alkyl), —OCF₃, —OCHF₂, and —SCF₃.

In some embodiments, R² is a phenyl group substituted with one or more substituents chosen from N-carbamyl, N-thiocarbamyl, —N(C₁₋₃ alkyl)₂, —NH(C₁₋₃ alkyl), —NH₂, and —NO₂. In certain embodiments, R² is a phenyl group substituted with one or more substituents chosen from cycloalkyl, aryl, heteroaryl, and heterocycle.

In some embodiments, R² is chosen from pyridine, pyrimidine, pyrazine, pyridazine, or triazine, each optionally substituted with one or more substituents chosen from hydro, hydroxyl, halo, alkyl, alkoxy, alkylthio, arylthio, thiocarbonyl, O-carboxy, C-carboxy, O-carbamyl, O-thiocarbamyl, N-carbamyl, N-thiocarbamyl, ester, haloalkyl, haloalkoxy, cycloalkyl, aryl, heteroaryl, heterocycle, —C(═O)OH, —CH(CH₃)C(═O)OH; —CH₂C(═O)OH, —C(CH₃)₂C(═O)OH, —C(CH₃)(CH₂CH₃)C(═O)OH, —CH(CH₂CH₃)C(═O)OH, —CH═C(CH₃)C(═O)OH, —C(CH₂CH₃)₂C(═O)OH, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂.

In some embodiments, R² is a pyridine ring optionally substituted with one or more substituents chosen from hydro, hydroxyl, halo, alkyl, alkoxy, alkylthio, arylthio, thiocarbonyl, O-carboxy, C-carboxy, O-carbamyl, O-thiocarbamyl, N-carbamyl, N-thiocarbamyl, ester, haloalkyl, haloalkoxy, cycloalkyl, aryl, heteroaryl, heterocycle, —C(═O)OH, —CH(CH₃)C(═O)OH; —CH₂C(═O)OH, —C(CH₃)₂C(═O)OH, —C(CH₃)(CH₂CH₃)C(═O)OH, —CH(CH₂CH₃)C(═O)OH, —CH═C(CH₃)C(═O)OH, —C(CH₂CH₃)₂C(═O)OH, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂.

In some embodiments, R² is chosen from unsubstituted pyridine, pyrimidine, pyrazine, pyridazine, or triazine. In certain embodiments, R² is an unsubstituted pyridine.

In one embodiment, the invention provides compounds of Formula I(a)-IV(a)

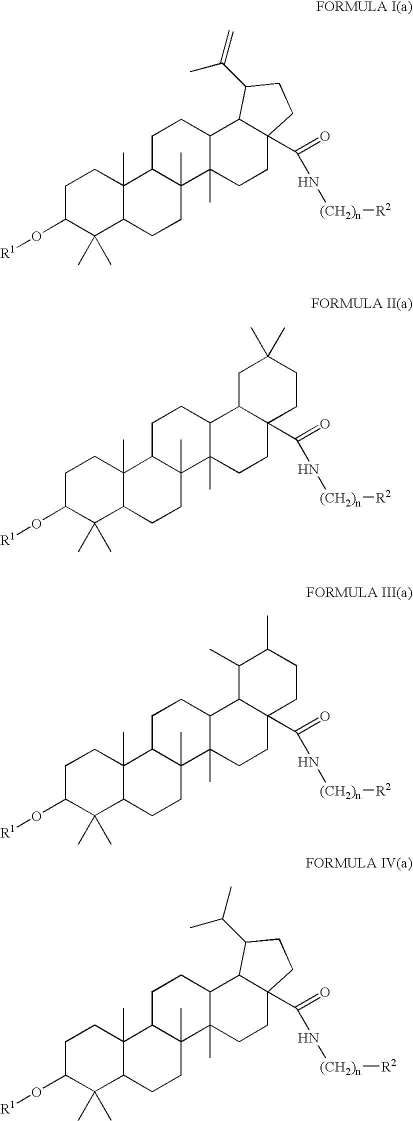

where R¹ is —C(═O)—CH₂—C(CH₃)₂—COOH;
R² is chosen from a cycloalkyl, aryl, heterocycle, and heteroaryl ring optionally substituted with one or more substituents chosen from hydro, hydroxyl, halo, alkyl, alkoxy, alkylthio, arylthio, thiocarbonyl, O-carboxy, C-carboxy, O-carbamyl, O-thiocarbamyl, N-carbamyl, N-thiocarbamyl, ester, haloalkyl, haloalkoxy, cycloalkyl, aryl, heteroaryl, heterocycle, —C(═O)OH, —CH(CH₃)C(═O)OH; —CH₂C(═O)OH, —C(CH₃)₂C(═O)OH, —C(CH₃)(CH₂CH₃)C(═O)OH, —CH(CH₂CH₃)C(═O)OH, —CH═C(CH₃)C(═O)OH, —C(CH₂CH₃)₂C(═O)OH, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —C(═O)NH(C₁₋₃ alkyl)NHC(═O)(C₁₋₃ alkyl), —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂;
n is an integer chosen from 0, 1, 2, and 3; and pharmaceutically acceptable salts thereof.

In one embodiment, the invention provides compounds of Formulae I(a)-IV(a) and pharmaceutical compositions comprising the compound and one or more pharmaceutically acceptable excipients, wherein R¹ is —C(═O)—CH₂—C(CH₃)₂—COOH; R² is a phenyl group optionally substituted with one or more substituents chosen from hydroxyl, halo, alkyl, alkoxy, alkylthio, arylthio, thiocarbonyl, O-carboxy, C-carboxy, O-carbamyl, O-thiocarbamyl, N-carbamyl, N-thiocarbamyl, ester, haloalkyl, haloalkoxy, cycloalkyl, aryl, heteroaryl, heterocycle, —C(═O)OH, —CH(CH₃)C(═O)OH; —CH₂C(═O)OH, —C(CH₃)₂C(═O)OH, —C(CH₃)(CH₂CH₃)C(═O)OH, —CH(CH₂CH₃)C(═O)OH, —CH═C(CH₃)C(═O)OH, —C(CH₂CH₃)₂C(═O)OH, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂; and n is an integer chosen from 0, 1, 2, and 3.

In certain embodiments, the invention provides compounds of Formulae I(a)-IV(a) and pharmaceutical compositions comprising the compound and one or more pharmaceutically acceptable excipients, wherein R¹ is —C(═O)—CH₂—C(CH₃)₂—COOH; R² is a pyridine ring optionally substituted with one or more substituents chosen from hydroxyl, halo, alkyl, alkoxy, alkylthio, arylthio, thiocarbonyl, O-carboxy, C-carboxy, O-carbamyl, O-thiocarbamyl, N-carbamyl, N-thiocarbamyl, ester, haloalkyl, haloalkoxy, cycloalkyl, aryl, heteroaryl, heterocycle, —C(═O)OH, —CH(CH₃)C(═O)OH; —CH₂C(═O)OH, —C(CH₃)₂C(═O)OH, —C(CH₃)(CH₂CH₃)C(═O)OH, —CH(CH₂CH₃)C(═O)OH, —CH═C(CH₃)C(═O)OH, —C(CH₂CH₃)₂C(═O)OH, —N(C₁₋₃ alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —CHF₂, —OCF₃, —OCHF₂, —SCF₃, —CF₃, —CN, —NH₂, and —NO₂; and n is an integer chosen from 0, 1, 2, and 3.

In specific embodiments, the invention provides compounds of Formulae I(a)-IV(a) and pharmaceutical compositions comprising the compound and one or more pharmaceutically acceptable excipients, wherein R¹ is —C(═O)—CH₂—C(CH₃)₂—COOH; R² is chosen from unsubstituted pyridine, pyrimidine, pyrazine, pyridazine, and triazine; and n is an integer chosen from 0, 1, 2, and 3. In one embodiment, R¹ is —C(═O)—CH₂—C(CH₃)₂—COOH; R² is unsubstituted pyridine; and n is an integer chosen from 0, 1, 2, and 3.

In one embodiment, the stereochemistry of the core betulin moiety is preserved. For example, a compound of the invention may have the stereochemistry according to Formula I(b):

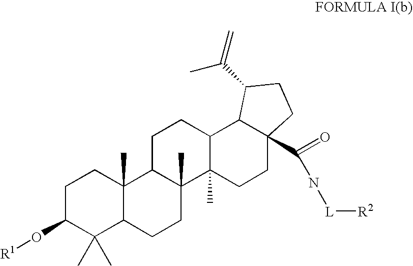

wherein L, R¹, and R² are as defined for Formula I above.

In one embodiment, the present invention provides compounds of Formula V

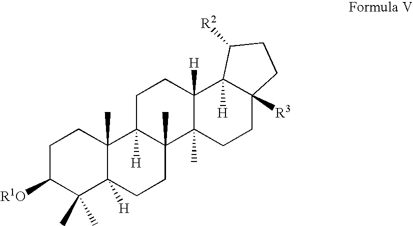

and pharmaceutically acceptable salts and stereoisomers thereof,
wherein

R¹ is R¹¹—C(O)— wherein R¹¹ is C₁₋₂₀ (preferably C₁₋₁₀, more preferably C₁₋₆) alkyl, C₁₋₂₀ (preferably C₁₋₁₀, more preferably C₁₋₆) alkenyl, or C₁₋₂₀ (preferably C₁₋₁₀, more preferably C₁₋₆) alkynyl, each being optionally substituted with one or more substituents independently chosen from the group of:
halo (e.g., F, Cl, Br, I); C₁₋₆ alkyl; —CN; hydroxyl; aryl; heteroaryl; cycloalkyl; heterocycle;
—C(O)R¹² where R¹² is —OH, C₁₋₆ alkoxy, C₁₋₆ alkenyloxy, C₁₋₆ alkynyloxy, C₃₋₆ cycloalkoxy or heterocycle;
—C(O)—N(R¹³)(R¹⁴) where R¹³ and R¹⁴ are independently H, C₁₋₆ alkyl, aryl, heteroaryl, C₃₋₆ cycloalkyl, —P(O)(OH)₂, (C₁₋₆ alkyl)phosphono, or —SO₃R¹⁵ where R¹⁵ is H, C₁₋₆ alkyl or aryl, or R¹³ and R¹⁴ together with the nitrogen atom they are linked to form a 3 to 6-membered heterocycle;
—N(R¹³)(R¹⁴) where R¹³ and R¹⁴ are independently H, C₁₋₆ alkyl, aryl, heteroaryl, C₃₋₆ cycloalkyl, or R¹³ and R¹⁴ together with the nitrogen atom they are linked to form a 3 to 6-membered heterocycle;
—SO₃R¹⁵, where R¹⁵ is C₁₋₆ alkyl, aryl or heteroaryl;
—NHSO₃R¹⁶, where R¹⁶ is C₁₋₆ alkyl, aryl, or heteroaryl; and
—P(O)(OR¹⁷)₂ where R¹⁷ is H or C₁₋₆ alkyl;
wherein optionally two substituents (e.g., one alkyl and one hydroxyl) at one carbon atom of R¹¹ may, together with the one carbon atom they are attached to, form a 3 to 6-membered cycloalkyl or heterocycle.

R² is isopropenyl or isopropyl, optionally substituted with one or two substituents independently selected from hydroxyl, halo, amino, and pyrrolidinyl, piperidinyl, and preferably R² is isopropenyl, isopropyl, 1′-hydroxyisopropyl, 2′-hydroxyisopryl, 1′,2′-dihydroxyisopropyl, and 1′-pyrrolidinyl-2′-hydroxyisopropyl;

R³ is represented by

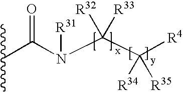

wherein

R³¹ is H or methyl or ethyl, preferably H or methyl;

R³², R³³, R³⁴ and R³⁵ are independently H, methyl, ethyl, and either R³² and R³³
or R³⁴ and R³⁵ can be taken together with the carbon they are attached to
form a cyclopropyl or cyclobutyl or cyclopentyl (preferably cyclopropyl), and wherein at least one of R³², R³³, R³⁴ and R³⁵, when present, is not H;

x and y are independently an integer of 0 or 1, at least one of x and y is not 0; and

R⁴ is an aryl, heteroaryl, arylalkyl (preferably benzyl, phenylethyl) or heteroarylalkyl (preferably heteroarylmethyl or heteroarylethyl), each being optionally substituted with 1, 2, 3 or 4 or 5 or 6 (preferably 1-3) substituents each being independently chosen from:
(1) halo (e.g., F, Cl, Br, I);
(2) hydroxyl;
(3) C₁₋₁₀ alkyl (preferably C₁₋₆ alkyl) or C₃₋₆ cycloalkyl, optionally substituted with 1, 2 or 3 moieties independently chosen from: hydroxyl; halo (preferably F. e.g., monofluoro, difluoro, or trifluoro); C₁₋₆ alkoxy; C₁₋₆ haloalkoxy; C₃₋₁₀ cycloalkyl; heterocycle; aryl; heteroaryl; —C(O)R^{4a} where R^{4a} is —OH, C₁₋₆ alkoxy, C₁₋₆ alkenyloxy, C₁₋₆ alkynyloxy, C₃₋₆ cycloalkoxy or heterocycle; —C(O)—N(R^{4c})(R^{4d}) where R^{4c} and R^{4d} are independently H, C₁₋₆ alkyl, aryl, heteroaryl, C₃₋₆ cycloalkyl, or —SO₃R^{4e} where R^{4e} is H, C₁₋₆ alkyl or aryl, or R^{4c} and R^{4d} together with the nitrogen atom they are linked to form a 3 to 6-membered heterocycle; —N(R^{4c})(R^{4d}) where R^{4c} and R^{4d} are independently H, C₁₋₆ alkyl, C₁₋₆ hydroxyalkyl, aryl, heteroaryl, C₃₋₆ cycloalkyl, or —SO₃R^{4e} where R^{4e} is H, C₁₋₆ alkyl or aryl, or R^{4c} and R^{4d} together with the nitrogen atom they are linked to form a 3 to 6-membered heterocycle; —SO₃R^{4f}, where R^{4f} is C₁₋₆ alkyl, aryl or heteroaryl; —NHSO₃R^{4g}, where R^{4g} is C₁₋₆ alkyl, aryl, or heteroaryl; —N(R^{4b})—C(O)R^{4h} where R^{4b} is H or methyl or ethyl, R^{4h} is C₁₋₆ alkyl; and —N(R^{4b})—C(O)—N(R^{4c})(R^{4d}) where R^{4b} is H or methyl or ethyl, R^{4c} and R^{4d} are independently H, C₁₋₆ alkyl, aryl, heteroaryl, C₃₋₆ cycloalkyl, or —SO₃R^{4e} where R^{4e} is H, C₁₋₆ alkyl or aryl, or R^{4c} and R^{4d} together with the nitrogen atom they are linked to form a 3 to 6-membered heterocycle;
(4) —CO₂R⁴ⁱ or —O(C═O)R⁴ⁱ wherein R⁴ⁱ is H or C₁₋₆ alkyl, preferably methyl or ethyl;
(5) —N(R^{4c})(R^{4d}) or —SO₂N(R^{4c})(R^{4d}), wherein R^{4c} and R^{4d} are independently H, OH(R^{4c} and R^{4d} are not both OH), C₁₋₆ hydroxyalkyl, or C₁₋₆ alkyl (preferably C₁₋₃ alkyl), aryl or heteroaryl, or R^{4c} and R^{4d} taken together with the nitrogen they are attached to form a 3, 4, 5 or 6-membered heterocycle;
(6) —SO₃R^{4e}, where R^{4e} is C₁₋₆ alkyl, aryl or heteroaryl;
(7) —NHSO₃R^{4f}, where R^{4f} is C₁₋₆ alkyl, aryl, or heteroaryl;
(8) —N(R^{4b})C(═O)R^{4h}, —N(R^{4b})C(═O)N(R^{4c})(R^{4d}), or —OC(═O)N(R^{4c})(R^{4d}), where R^{4b} is H or methyl or ethyl; R^{4h}, R^{4c} and R^{4d} are independently H, OH(R^{4c} and R^{4d} are not both OH), C₁₋₁₀ alkyl, C₂₋₁₀ alkenyl, C₂₋₁₀ alkynyl, C₁₋₁₀ alkoxy, C₁₋₁₀ alkylthiol, C₂₋₁₀ alkenyloxy, C₂₋₁₀ alkynyloxy, C₁₋₁₀ haloalkyl, C₂₋₆ hydroxyalkyl, C₁₋₆ alkyl-O—C₁₋₆ alkyl-, cycloalkyl, heterocycle, aryl, heteroaryl, or R^{4c} and R^{4d} together with the nitrogen atom to which they are both linked form a 3, 4, 5 or 6-membered heterocycle (e.g., piperidinyl, pyrrolidinyl, and morpholinyl);
(9) C₁₋₆ alkoxy optionally substituted with 1, 2 or 3 substituents each being independently chosen from the group consisting of:
   hydroxyl;
   halo (e.g., F, Cl, Br, I);
   —CO₂R⁴ⁱ where R⁴ⁱ is H or C₁₋₆ alkyl (preferably methyl);
   heterocycle

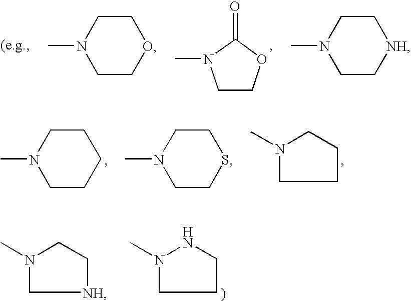

optionally substituted with 1, 2, or 3 substituents each being independently halo (e.g., F, Cl, Br, I), C₁₋₆ alkyl, or C₁₋₃ haloalkyl;
heteroaryl (e.g., imidazolyl) optionally substituted with 1, 2, or 3 substituents each being independent halo (e.g., F, Cl, Br, I), hydroxyl, C₁₋₆ alkyl (preferably methyl), C₁₋₆ alkoxy, carboxyl, C₁₋₃ alkoxycarbonyl, C₁₋₃ hydroxyalkyl, C₁₋₃ haloalkyl, or —N(R^{4c})(R^{4d}) or —SO₂N(R^{4c})(R^{4d}), wherein R^{4c} and R^{4d} are independently H, OH (R^{4c} and R^{4d} are not both OH), C₁₋₃ alkyl, C₁₋₆ hydroxyalkyl, or C₁₋₆ alkyl (preferably C₁₋₃ alkyl), or R^{ae} and R^{af} taken together with the nitrogen they are attached to form a 3, 4, 5 or 6-membered heterocycle; and
—N(R^{4c})(R⁴) where R^{4c} and R^{4d} are independently H, hydroxyl, C₁₋₆ alkyl, C₁₋₆ hydroxyalkyl, or —N(R^{4m})(R⁴ⁿ) where R^{4m} and R⁴ⁿ are independently H or C₁₋₃ alkyl, or R^{4c} and R^{4d} can be taken together with the nitrogen they are attached to form a 3, 4, 5 or 6-membered heterocycle, and/or R^{4m} and R⁴ⁿ can be taken together with the nitrogen they are attached to form a 3, 4, 5 or 6-membered heterocycle; and
(10) —CON(R^{4p})(R^{4q}) wherein R^{4p} and R^{4q} are independently H, or C₁₋₁₀ alkyl that is optionally substituted with 1, 2, or 3 substituents each being independently
   hydroxyl;
   halo;
   —N(R^{4r})(R^{4t}) where R^{4r} and R^{4t} are independently H, C₁₋₃ alkyl, hydroxyl, or C₁₋₃ hydroxylalkyl;
   heterocycle

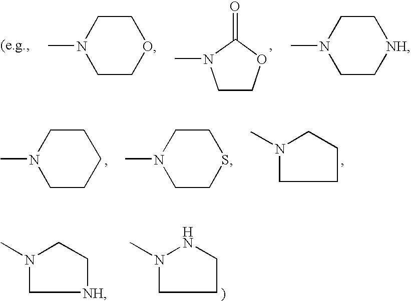

optionally substituted with 1, 2, or 3 substituents each being independently halo (e.g., F, Cl, Br, I), C₁₋₆ alkyl, or C₁₋₃ haloalkyl;
C₁₋₁₀ alkoxy, C₁₋₁₀ alkylthiol, C₂₋₁₀ alkenyloxy, C₂₋₁₀ alkynyloxy; and
aryl or heteroaryl, optionally substituted with 1, 2, or 3 substituents each being independently halo (e.g., F, Cl, Br, I), hydroxyl, C₁₋₆ alkyl (preferably methyl), C₁₋₃ haloalkyl, carboxyl, C₁₋₃ alkyoxycarbonyl, —N(R^{4c})(R^{4d}) or —SO₂N(R^{4c})(R^{4d}), wherein R^{4c} and R^{4d} are independently H, OH(R^{4c} and R^{4d} are not both OH), C₁₋₃ alkyl, C₁₋₆ hydroxyalkyl, or C₁₋₆ alkyl (preferably C₁₋₃ alkyl), or R^{4c} and R^{4d} taken together with the nitrogen they are attached to form a 3, 4, 5 or 6-membered heterocycle; and
(11) cycloalkyl, heterocycle, aryl or heteroaryl, optionally substituted with 1, 2, or 3 substituents each being independently halo (e.g., F, Cl, Br, I); hydroxyl; C₁₋₆ alkyl (preferably methyl); C₁₋₃ haloalkyl; —CO₂R⁴ⁱ or —O(C═O)R⁴ⁱ wherein R⁴ⁱ is H or C₁₋₃ alkyl; —N(R^{4c})(R^{4d}) or —SO₂N(R^{4c})(R^{4d}) wherein R^{4c} and R^{4d} are independently H, OH(R^{4c} and R^{4d} are not both OH), C₁₋₃ alkyl, C₁₋₆ hydroxyalkyl, or C₁₋₆ alkyl (preferably C₁₋₃ alkyl), or R^{4c} and R^{4d} taken together with the nitrogen they are attached to form a 3, 4, 5 or 6-membered heterocycle.

In some embodiments of the compounds of Formula V, R¹ carboxyalkanoyl having 3-10 carbon atoms, and optionally substituted with 1, 2, 3, 4, 5, 6 halo atoms (e.g., F). In some embodiments of the compounds of Formula V, R¹ carboxyhaloalkanoyl having 3-10 carbon atoms.

In some embodiments of the compounds of Formula V, R¹ is chosen from succinyl, glutaryl, 3′-methylglutaryl, 3′-methylsuccinyl, 3′3′-dimethylsuccinyl, 3′3′-dimethylglutaryl, 3′-methyl-3′-ethylsuccinyl, 3′-methyl-3′-ethylglutaryl, and C₁₋₆ alkyl ester thereof, optionally substituted with 1, 2, 3, 4, 5, 6 halo atoms (e.g., F).

In some embodiments of the compounds of Formula V, R¹ is —C(═O)—(CH₂)ₘ—C(CH₃)₂—(CH₂)ₙ—COOH or —C(═O)—(CH₂)ₘ—C(CH₃)₂—(CH₂)ₙ—C(O)—C₁₋₆ alkyl, wherein m and n are independently an integer of 0, 1, 2 or 3, and more preferably R¹ is —C(═O)—CH₂—C(CH₃)₂COOH (3′,3′-dimethylsuccinyl) or —C(═O)—CH₂—C(CH₃)₂CH₂COOH (3′,3′-dimethylglutaryl), —C(═O)—CH₂—C(CH₃)₂C(O)—C₁₋₆ alkyl or —C(═O)—CH₂—C(CH₃)₂CH₂C(O)—C₁₋₆ alkyl, each being optionally substituted with 1, 2, 3, 4, 5, 6 halo atoms (e.g., F).

In some embodiments of the compounds of Formula V, R¹ is —C(═O)—CH₂—C(R¹⁸)(R¹⁹)COOH, —C(═O)—CH₂—C(R¹⁸)(R¹⁹)CH₂COOH, —C(═O)—CH₂—C(R¹⁸)(R¹⁹)C(O)—C₁₋₆ alkyl or —C(═O)—CH₂—C(R¹⁸)(R¹⁹)CH₂C(O)—C₁₋₆ alkyl, wherein R¹⁸ and R¹⁹ are independently trifluoromethyl, trifluoroethyl, methyl, ethyl, or R¹⁸ and R¹⁹ together with the carbon atom they are attached to form a 3, 4 or 5-membered cycloalkyl or heterocycle having an O or S atom. In specific embodiments, R¹⁸ and R¹⁹ are not both methyl.

In some embodiments of the compounds of Formula V, R¹ is —C(═O)—CH₂—C(CF₃)₂COOH or —C(═O)—CH₂—C(CF₃)₂CH₂COOH.

In some embodiments of the compounds of Formula V, R¹ is

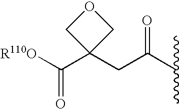

wherein R¹¹⁰ is H or C₁₋₆ alkyl.

In some specific embodiments, the compound is not one of Compounds 81, 105, 117 and 121 below.

In some specific embodiments of the compounds of Formula V, R¹ is not —C(═O)—(CH₂)ₘ—C(CH₃)₂—(CH₂)ₙ—COOH wherein m and n are independently an integer of 0, 1, 2 or 3.

In certain embodiments, R² is isopropenyl or isopropyl, preferably isopropenyl.

In some embodiments, R⁴ is a heteroaryl or heteroarylmethyl or heteroarylethyl having at least one nitrogen and optionally substituted with 1, 2 or 3 substituents independently chosen from halo (e.g., F, Cl, Br, I); C₁₋₆ alkyl; C₁₋₆ haloalkyl; hydroxyl; amino or C₁₋₃ alkylamino; C₁₋₆ alkoxy optionally substituted with 1-3 halo (e.g., F, Cl, Br, I); carboxyl; C₁₋₆ alkoxycarbonyl. Preferably R⁴ is chosen from pyridine, pyrimidine, pyrazine, pyridazine, and triazine, optionally substituted with 1, 2 or 3 above substituents. In some specific embodiments, R⁴ is unsubstituted pyridine.

In some specific forms of any one of the above embodiments, R⁴ is not p-methoxyphenyl or 2-pyridinyl.

In some embodiments, R⁴ is a fused heteroaryl. By “fused heteroaryl,” it is meant a heteroaryl group fused to another heteroaryl group or an aryl group.

In another aspect, the present invention provides compounds of Formula V

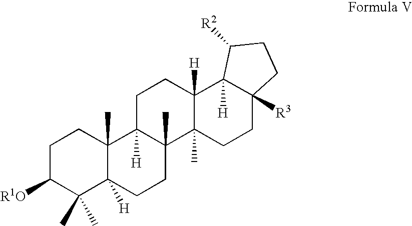

and pharmaceutically acceptable salts and stereoisomers thereof, wherein

R¹ is R¹¹—C(O)— wherein R¹¹ is C₁₋₂₀ (preferably C₁₋₁₀, more preferably C₁₋₆) alkyl, C₁₋₂₀ (preferably C₁₋₁₀, more preferably C₁₋₆) alkenyl, or C₁₋₂₀ (preferably C₁₋₁₀, more preferably C₁₋₆) alkynyl, each being optionally substituted with one or more substituents independently chosen from the group of:
halo (e.g., F, Cl, Br, I); C₁₋₆ alkyl; —CN; hydroxyl; aryl; heteroaryl; cycloalkyl; heterocycle;
—C(O)R¹² where R¹² is —OH, C₁₋₆ alkoxy, C₁₋₆ alkenyloxy, C₁₋₆ alkynyloxy, C₃₋₆ cycloalkoxy or heterocycle;
—C(O)—N(R¹³)(R¹⁴) where R¹³ and R¹⁴ are independently H, C₁₋₆ alkyl, aryl, heteroaryl, C₃₋₆ cycloalkyl, —P(O)(OH)₂, (C₁₋₆ alkyl)phosphono, or —SO₃R¹⁵ where R¹⁵ is H, C₁₋₆ alkyl or aryl, or R¹³ and R¹⁴ together with the nitrogen atom they are linked to form a 3 to 6-membered heterocycle;
—N(R¹³)(R¹⁴) where R¹³ and R¹⁴ are independently H, C₁₋₆ alkyl, aryl, heteroaryl, C₃₋₆ cycloalkyl, or R¹³ and R¹⁴ together with the nitrogen atom they are linked to form a 3 to 6-membered heterocycle;
—SO₃R¹⁵, where R¹⁵ is C₁₋₆ alkyl, aryl or heteroaryl;
—NHSO₃R¹⁶, where R¹⁶ is C₁₋₆ alkyl, aryl, or heteroaryl; and
—P(O)(OR¹⁷)₂ where R¹⁷ is H or C₁₋₆ alkyl;
wherein optionally two substituents (e.g., one alkyl and one hydroxyl) at one carbon atom of R¹¹ may, together with the one carbon atom they are attached to, form a 3 to 6-membered cycloalkyl or heterocycle.

R² is isopropenyl or isopropyl, optionally substituted with one or two substituents independently selected from hydroxyl, halo, amino, and pyrrolidinyl, piperidinyl, and preferably R² is isopropenyl, isopropyl, 1′-hydroxyisopropyl, 2′-hydroxyisopryl, 1′,2′-dihydroxyisopropyl, and 1′-pyrrolidinyl-2′-hydroxyisopropyl;

R³ is represented by

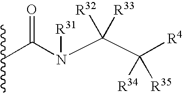

wherein

R³¹ is H or methyl or ethyl, preferably H or methyl;

R³², R³³, R³⁴ and R³⁵ are independently H, methyl, ethyl, and either R³² and R³³
or R³⁴ and R³⁵ can be taken together with the carbon they are attached to
form a cyclopropyl or cyclobutyl (preferably cyclopropyl), and wherein
all of R³², R³³, R³⁴ and R³⁵ is not H; and

R⁴ is an aryl, heteroaryl, arylalkyl (preferably benzyl, phenylethyl) or heteroarylalkyl (preferably heteroarylmethyl or heteroarylethyl), each being optionally substituted with 1, 2, 3 or 4 or 5 or 6 (preferably 1-3) substituents each being independently chosen from:
(1) halo (e.g., F, Cl, Br, I);
(2) hydroxyl;
(3) C₁₋₁₀ alkyl (preferably C₁₋₆ alkyl) or C₃₋₆ cycloalkyl, optionally substituted with 1, 2 or 3 moieties independently chosen from: hydroxyl; halo (preferably F. e.g., monofluoro, difluoro, or trifluoro); C₁₋₆ alkoxy; C₁₋₆ haloalkoxy; C₃₋₁₀ cycloalkyl; heterocycle; aryl; heteroaryl; —C(O)R^{4a} where R^{4a} is —OH, C₁₋₆ alkoxy, C₁₋₆ alkenyloxy, C₁₋₆ alkynyloxy, C₃₋₆ cycloalkoxy or heterocycle; —C(O)—N(R^{4c})(R^{4d}) where R^{4c} and R^{4d} are independently H, C₁₋₆ alkyl, aryl, heteroaryl, C₃₋₆ cycloalkyl, or —SO₃R^{4e} where R^{4e} is H, C₁₋₆ alkyl or aryl, or R^{4c} and R^{4d} together with the nitrogen atom they are linked to form a 3 to 6-membered heterocycle; —N(R^{4c})(R^{4d}) where R^{4c} and R^{4d} are independently H, C₁₋₆ alkyl, C₁₋₆ hydroxyalkyl, aryl, heteroaryl, C₃₋₆ cycloalkyl, or —SO₃R^{4e} where R^{4e} is H, C₁₋₆ alkyl or aryl, or R^{4c} and R^{4d} together with the nitrogen atom they are linked to form a 3 to 6-membered heterocycle; —SO₃R^{4f}, where R^{4f} is C₁₋₆ alkyl, aryl or heteroaryl; —NHSO₃R^{4g}, where R^{4g} is C₁₋₆ alkyl, aryl, or heteroaryl; —N(R^{4b})—C(O)R^{4h} where R^{4b} is H or methyl or ethyl, R^{4h} is C₁₋₆ alkyl; and —N(R^{4b})—C(O)—N(R^{4c})(R^{4d}) where R^{4b} is H or methyl or ethyl, R^{4c} and R^{4d} are independently H, C₁₋₆ alkyl, aryl, heteroaryl, C₃₋₆ cycloalkyl, or —SO₃R^{4e} where R^{4e} is H, C₁₋₆ alkyl or aryl, or R^{4c} and R^{4d} together with the nitrogen atom they are linked to form a 3 to 6-membered heterocycle;
(4) —CO₂R⁴¹ or —O(C═O)R⁴¹ wherein R⁴¹ is H or C₁₋₆ alkyl, preferably methyl or ethyl;
(5) —N(R^{4c})(R^{4d}) or —SO₂N(R^{4c})(R^{4d}), wherein R^{4c} and R^{4d} are independently H, OH(R^{4c} and R^{4d} are not both OH), C₁₋₆ hydroxyalkyl, or C₁₋₆ alkyl (preferably C₁₋₃ alkyl), aryl or heteroaryl, or R^{4c} and R^{4d} taken together with the nitrogen they are attached to form a 3, 4, 5 or 6-membered heterocycle;
(6) —SO₃R^{4e}, where R^{4e} is C₁₋₆ alkyl, aryl or heteroaryl;
(7) —NHSO₃R^{4f}, where R^{4f} is C₁₋₆ alkyl, aryl, or heteroaryl;
(8) —N(R^{4b})C(═O)R^{4h}, —N(R^{4b})C(═O)N(R^{4c})(R^{4d}), or —OC(═O)N(R^{4c})(R^{4d}), where R^{4b} is H or methyl or ethyl; R^{4h}, R^{4c} and R^{4d} are independently H, OH(R^{4c} and R^{4d} are not both OH), C₁₋₁₀ alkyl, C₂₋₁₀ alkenyl, C₂₋₁₀ alkynyl, C₁₋₁₀ alkoxy, C₁₋₁₀ alkylthiol, C₂₋₁₀ alkenyloxy, C₂₋₁₀ alkynyloxy, C₁₋₁₀ haloalkyl, C₂₋₆ hydroxyalkyl, C₁₋₆ alkyl-O—C₁₋₆ alkyl-, cycloalkyl, heterocycle, aryl, heteroaryl, or R^{4c} and R^{4d} together with the nitrogen atom to which they are both linked form a 3, 4, 5 or 6-membered heterocycle (e.g., piperidinyl, pyrrolidinyl, and morpholinyl);
(9) C₁₋₆ alkoxy optionally substituted with 1, 2 or 3 substituents each being independently chosen from the group consisting of:
   hydroxyl;
   halo (e.g., F, Cl, Br, I);
   —CO₂R⁴¹ where R⁴¹ is H or C₁₋₆ alkyl (preferably methyl);
   heterocycle

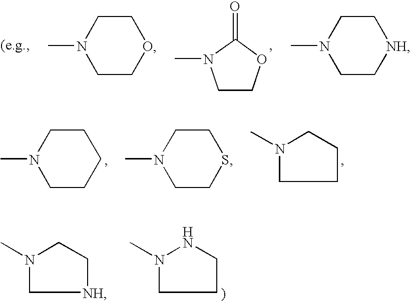

optionally substituted with 1, 2, or 3 substituents each being independently halo (e.g., F, Cl, Br, I), C₁₋₆ alkyl, or C₁₋₃ haloalkyl;
heteroaryl (e.g., imidazolyl) optionally substituted with 1, 2, or 3 substituents each being independent halo (e.g., F, Cl, Br, I), hydroxyl, C₁₋₆ alkyl (preferably methyl), C₁₋₆ alkoxy, carboxyl, C₁₋₃ alkoxycarbonyl, C₁₋₃ hydroxyalkyl, C₁₋₃ haloalkyl, or —N(R^{4c})(R^{4d}) or —SO₂N(R^{4c})(R^{4d}), wherein R^{4c} and R^{4d} are independently H, OH (R^{4c} and R^{4d} are not both OH), C₁₋₃ alkyl, C₁₋₆ hydroxyalkyl, or C₁₋₆ alkyl (preferably C₁₋₃ alkyl), or R^{ae} and R^{af} taken together with the nitrogen they are attached to form a 3, 4, 5 or 6-membered heterocycle; and —N(R^{4c})(R^{4d}) where R^{4c} and R^{4d} are independently H, hydroxyl, C₁₋₆ alkyl, C₁₋₆ hydroxyalkyl, or —N(R^{4m})(R⁴ⁿ) where R^{4m} and R⁴ⁿ are independently H or C₁₋₃ alkyl, or R^{4c} and R^{4d} can be taken together with the nitrogen they are attached to form a 3, 4, 5 or 6-membered heterocycle, and/or R^{4m} and R⁴ⁿ can be taken together with the nitrogen they are attached to form a 3, 4, 5 or 6-membered heterocycle; and
(10) —CON(R^{4p})(R^{4q}) wherein R^{4p} and R^{4q} are independently H, or C₁₋₁₀ alkyl that is optionally substituted with 1, 2, or 3 substituents each being independently
   hydroxyl;
   halo;
   —N(R^{4r})(R^{4t}) where R^{4r} and R^{4t} are independently H, C₁₋₃ alkyl, hydroxyl, or C₁₋₃ hydroxylalkyl;
   heterocycle

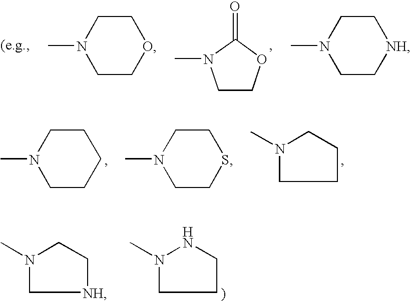

optionally substituted with 1, 2, or 3 substituents each being independently halo (e.g., F, Cl, Br, I), C₁₋₆ alkyl, or C₁₋₃ haloalkyl;
C₁₋₁₀ alkoxy, C₁₋₁₀ alkylthiol, C₂₋₁₀ alkenyloxy, C₂₋₁₀ alkynyloxy; and
aryl or heteroaryl, optionally substituted with 1, 2, or 3 substituents each being independently halo (e.g., F, Cl, Br, I), hydroxyl, C₁₋₆ alkyl (preferably methyl), C₁₋₃ haloalkyl, carboxyl, C₁₋₃ alkyoxycarbonyl, —N(R^{4c})(R^{4d}) or —SO₂N(R^{4c})(R^{4d}), wherein R^{4c} and R^{4d} are independently H, OH(R^{4c} and R^{4d} are not both OH), C₁₋₃ alkyl, C₁₋₆ hydroxyalkyl, or C₁₋₆ alkyl (preferably C₁₋₃ alkyl), or R^{4c} and R^{4d} taken together with the nitrogen they are attached to form a 3, 4, 5 or 6-membered heterocycle; and
(11) cycloalkyl, heterocycle, aryl or heteroaryl, optionally substituted with 1, 2, or 3 substituents each being independently halo (e.g., F, Cl, Br, I); hydroxyl; C₁₋₆ alkyl (preferably methyl); C₁₋₃ haloalkyl; —CO₂R⁴¹ or —O(C═O)R⁴¹ wherein R⁴¹ is H or C₁₋₃ alkyl; —N(R^{4c})(R^{4d}) or —SO₂N(R^{4c})(R^{4d}) wherein R^{4c} and R^{4d} are independently H, OH(R^{4c} and R^{4d} are not both OH), C₁₋₃ alkyl, C₁₋₆ hydroxyalkyl, or C₁₋₆ alkyl (preferably C₁₋₃ alkyl), or R^{4c} and R^{4d} taken together with the nitrogen they are attached to form a 3, 4, 5 or 6-membered heterocycle.

In some embodiments of the compounds of Formula V, R¹ carboxyalkanoyl having 3-10 carbon atoms, and optionally substituted with 1, 2, 3, 4, 5, 6 halo atoms (e.g., F). In some embodiments of the compounds of Formula V, R¹ carboxyhaloalkanoyl having 3-10 carbon atoms.

In some embodiments of the compounds of Formula V, R¹ is chosen from succinyl, glutaryl, 3′-methylglutaryl, 3′-methylsuccinyl, 3′3′-dimethylsuccinyl, 3′3′-dimethylglutaryl, 3′-methyl-3′-ethylsuccinyl, 3′-methyl-3′-ethylglutaryl, and C₁₋₆ alkyl ester thereof, optionally substituted with 1, 2, 3, 4, 5, 6 halo atoms (e.g., F).

In some embodiments of the compounds of Formula V, R¹ is —C(═O)—(CH₂)ₘ—C(CH₃)₂—(CH₂)ₙ—COOH or —C(═O)—(CH₂)ₘ—C(CH₃)₂—(CH₂)ₙ—C(O)—C₁₋₆ alkyl, wherein m and n are independently an integer of 0, 1, 2 or 3, and more preferably R¹ is —C(═O)—CH₂—C(CH₃)₂COOH (3′,3′-dimethylsuccinyl) or —C(═O)—CH₂—C(CH₃)₂CH₂COOH (3′,3′-dimethylglutaryl), —C(═O)—CH₂—C(CH₃)₂C(O)—C₁₋₆ alkyl or —C(═O)—CH₂—C(CH₃)₂CH₂C(O)—C₁₋₆ alkyl, each being optionally substituted with 1, 2, 3, 4, 5, 6 halo atoms (e.g., F).

In some embodiments of the compounds of Formula V, R¹ is —C(═O)—CH₂—C(R¹⁸)(R¹⁹)COOH, —C(═O)—CH₂—C(R¹⁸)(R¹⁹)CH₂COOH, —C(═O)—CH₂—C(R¹⁸)(R¹⁹)C(O)—C₁₋₆ alkyl or —C(═O)—CH₂—C(R¹⁸)(R¹⁹)CH₂C(O)—C₁₋₆ alkyl, wherein R¹⁸ and R¹⁹ are independently trifluoromethyl, trifluoroethyl, methyl, ethyl, or R¹⁸ and R¹⁹ together with the carbon atom they are attached to form a 3, 4 or 5-membered cycloalkyl or heterocycle having an O or S atom. In specific embodiments, R¹⁸ and R¹⁹ are not both methyl.

In some embodiments of the compounds of Formula V, R¹ is —C(═O)—CH₂—C(CF₃)₂COOH or —C(═O)—CH₂—C(CF₃)₂CH₂COOH.

In some embodiments of the compounds of Formula V, R¹ is

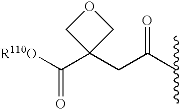

wherein R¹¹⁰ is H or C₁₋₆ alkyl.

In some specific embodiments, the compound is not one of Compounds 81, 105, 117 and 121 below.

In some specific embodiments of the compounds of Formula V, R¹ is not —C(═O)—(CH₂)ₘ—C(CH₃)₂—(CH₂)ₙ—COOH wherein m and n are independently an integer of 0, 1, 2 or 3.

In certain embodiments, R² is isopropenyl or isopropyl, preferably isopropenyl.

In some embodiments, R⁴ is a heteroaryl or heteroarylmethyl or heteroarylethyl having at least one nitrogen and optionally substituted with 1, 2 or 3 substituents independently chosen from halo (e.g., F, Cl, Br, I); C₁₋₆ alkyl; C₁₋₆ haloalkyl; hydroxyl; amino or C₁₋₃ alkylamino; C₁₋₆ alkoxy optionally substituted with 1-3 halo (e.g., F, Cl, Br, I); carboxyl; C₁₋₆ alkoxycarbonyl. Preferably R⁴ is chosen from pyridine, pyrimidine, pyrazine, pyridazine, and triazine, optionally substituted with 1, 2 or 3 above substituents. In some specific embodiments, R⁴ is unsubstituted pyridine.

In another aspect, the present invention provides compounds of Formula V

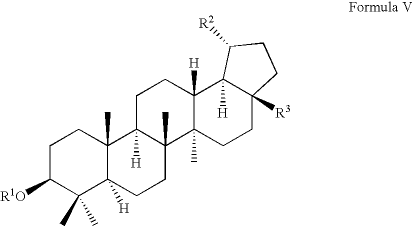

and pharmaceutically acceptable salts and stereoisomers thereof,
wherein

R¹ is R¹¹—C(O)— wherein R¹¹ is C₁₋₂₀ (preferably C₁₋₁₀, more preferably C₁₋₆) alkyl, C₁₋₂₀ (preferably C₁₋₁₀, more preferably C₁₋₆) alkenyl, or C₁₋₂₀ (preferably C₁₋₁₀, more preferably C₁₋₆) alkynyl, each being optionally substituted with one or more substituents independently chosen from the group of:
halo (e.g., F, Cl, Br, I); C₁₋₆ alkyl; —CN; hydroxyl; aryl; heteroaryl; cycloalkyl; heterocycle;
—C(O)R¹² where R¹² is —OH, C₁₋₆ alkoxy, C₁₋₆ alkenyloxy, C₁₋₆ alkynyloxy, C₃₋₆ cycloalkoxy or heterocycle;
—C(O)—N(R¹³)(R¹⁴) where R¹³ and R¹⁴ are independently H, C₁₋₆ alkyl, aryl, heteroaryl, C₃₋₆ cycloalkyl, —P(O)(OH)₂, (C₁₋₆ alkyl)phosphono, or —SO₃R¹⁵ where R¹⁵ is H, C₁₋₆ alkyl or aryl, or R¹³ and R¹⁴ together with the nitrogen atom they are linked to form a 3 to 6-membered heterocycle;
—N(R¹³)(R¹⁴) where R¹³ and R¹⁴ are independently H, C₁₋₆ alkyl, aryl, heteroaryl, C₃₋₆ cycloalkyl, or R¹³ and R¹⁴ together with the nitrogen atom they are linked to form a 3 to 6-membered heterocycle;
—SO₃R¹⁵, where R¹⁵ is C₁₋₆ alkyl, aryl or heteroaryl;
—NHSO₃R¹⁶, where R¹⁶ is C₁₋₆ alkyl, aryl, or heteroaryl; and
—P(O)(OR¹⁷)₂ where R¹⁷ is H or C₁₋₆ alkyl;
wherein optionally two substituents (e.g., one alkyl and one hydroxyl) at one carbon atom of R¹¹ may, together with the one carbon atom they are attached to, form a 3 to 6-membered cycloalkyl or heterocycle.

R² is isopropenyl or isopropyl, optionally substituted with one or two substituents independently selected from hydroxyl, halo, amino, and pyrrolidinyl, piperidinyl, and preferably R² is isopropenyl, isopropyl, 1′-hydroxyisopropyl, 2′-hydroxyisopryl, 1′,2′-dihydroxyisopropyl, and 1′-pyrrolidinyl-2′-hydroxyisopropyl;

R³ is represented by

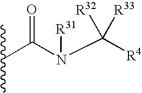

wherein

R³¹ is H or methyl or ethyl, preferably H or methyl;

R³² and R³³ are independently H, methyl or ethyl, or R³² and R³³ together with
the carbon they are attached to form a cyclopropyl or cyclobutyl or cyclopentyl (preferably cyclopropyl), wherein at least one of R³² and R³³ is not H; and

R⁴ is an aryl, heteroaryl, arylalkyl (preferably benzyl, phenylethyl) or heteroarylalkyl (preferably heteroarylmethyl or heteroarylethyl), each being optionally substituted with 1, 2, 3 or 4 or 5 or 6 (preferably 1-3) substituents each being independently chosen from:
(1) halo (e.g., F, Cl, Br, I);
(2) hydroxyl;
(3) C₁₋₁₀ alkyl (preferably C₁₋₆ alkyl) or C₃₋₆ cycloalkyl, optionally substituted with 1, 2 or 3 moieties independently chosen from: hydroxyl; halo (preferably F. e.g., monofluoro, difluoro, or trifluoro); C₁₋₆ alkoxy; C₁₋₆ haloalkoxy; C₃₋₁₀ cycloalkyl; heterocycle; aryl; heteroaryl; —C(O)R^{4a} where R^{4a} is —OH, C₁₋₆ alkoxy, C₁₋₆ alkenyloxy, C₁₋₆ alkynyloxy, C₃₋₆ cycloalkoxy or heterocycle; —C(O)—N(R^{4c})(R^{4d}) where R^{4c} and R^{4d} are independently H, C₁₋₆ alkyl, aryl, heteroaryl, C₃₋₆ cycloalkyl, or —SO₃R^{4e} where R^{4e} is H, C₁₋₆ alkyl or aryl, or R^{4c} and R^{4d} together with the nitrogen atom they are linked to form a 3 to 6-membered heterocycle; —N(R^{4c})(R^{4d}) where R^{4c} and R^{4d} are independently H, C₁₋₆ alkyl, C₁₋₆ hydroxyalkyl, aryl, heteroaryl, C₃₋₆ cycloalkyl, or —SO₃R^{4e} where R^{4e} is H, C₁₋₆ alkyl or aryl, or R^{4c} and R^{4d} together with the nitrogen atom they are linked to form a 3 to 6-membered heterocycle; —SO₃R⁴, where R^{4f} is C₁₋₆ alkyl, aryl or heteroaryl; —NHSO₃R^{4g}, where R^{4g} is C₁₋₆ alkyl, aryl, or heteroaryl; —N(R^{4b})—C(O)R^{4h} where R^{4b} is H or methyl or ethyl, R^{4h} is C₁₋₆ alkyl; and —N(R^{4b})—C(O)—N(R^{4c})(R^{4d}) where R^{4b} is H or methyl or ethyl, R^{4c} and R^{4d} are independently H, C₁₋₆ alkyl, aryl, heteroaryl, C₃₋₆ cycloalkyl, or —SO₃R^{4e} where R^{4e} is H, C₁₋₆ alkyl or aryl, or R^{4c} and R^{4d} together with the nitrogen atom they are linked to form a 3 to 6-membered heterocycle;
(4) —CO₂R⁴ⁱ or —O(C═O)R⁴ⁱ wherein R⁴ⁱ is H or C₁₋₆ alkyl, preferably methyl or ethyl;
(5) —N(R^{4c})(R^{4d}) or —SO₂N(R^{4c})(R^{4d}), wherein R^{4c} and R^{4d} are independently H, OH(R^{4c} and R^{4d} are not both OH), C₁₋₆ hydroxyalkyl, or C₁₋₆ alkyl (preferably C₁₋₃ alkyl), aryl or heteroaryl, or R^{4c} and R^{4d} taken together with the nitrogen they are attached to form a 3, 4, 5 or 6-membered heterocycle;
(6) —SO₃R^{4e}, where R^{4e} is C₁₋₆ alkyl, aryl or heteroaryl;
(7) —NHSO₃R^{4f}, where R^{4f} is C₁₋₆ alkyl, aryl, or heteroaryl;
(8) —N(R^{4b})C(═O)R^{4h}, —N(R^{4b})C(═O)N(R^{4c})(R^{4d}), or —OC(═O)N(R^{4c})(R^{4d}), where R^{4b} is H or methyl or ethyl; R^{4h}, R^{4c} and R^{4d} are independently H, OH(R^{4c} and R^{4d} are not both OH), C₁₋₁₀ alkyl, C₂₋₁₀ alkenyl, C₂₋₁₀ alkynyl, C₁₋₁₀ alkoxy, C₁₋₁₀ alkylthiol, C₂₋₁₀ alkenyloxy, C₂₋₁₀ alkynyloxy, C₁₋₁₀ haloalkyl, C₂₋₆ hydroxyalkyl, C₁₋₆ alkyl-O—C₁₋₆ alkyl-, cycloalkyl, heterocycle, aryl, heteroaryl, or R^{4c} and R^{4d} together with the nitrogen atom to which they are both linked form a 3, 4, 5 or 6-membered heterocycle (e.g., piperidinyl, pyrrolidinyl, and morpholinyl);
(9) C₁₋₆ alkoxy optionally substituted with 1, 2 or 3 substituents each being independently chosen from the group consisting of:
   hydroxyl;
   halo (e.g., F, Cl, Br, I);
   —CO₂R⁴ⁱ where R⁴ⁱ is H or C₁₋₆ alkyl (preferably methyl);
   heterocycle

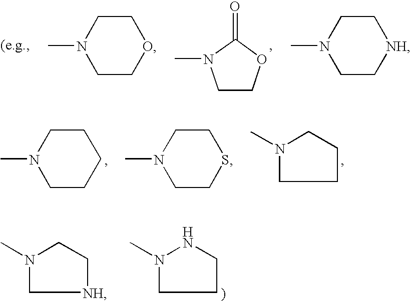

optionally substituted with 1, 2, or 3 substituents each being independently halo (e.g., F, Cl, Br, I), C₁₋₆ alkyl, or C₁₋₃ haloalkyl;
heteroaryl (e.g., imidazolyl) optionally substituted with 1, 2, or 3 substituents each being independent halo (e.g., F, Cl, Br, I), hydroxyl, C₁₋₆ alkyl (preferably methyl), C₁₋₆ alkoxy, carboxyl, C₁₋₃ alkoxycarbonyl, C₁₋₃ hydroxyalkyl, C₁₋₃ haloalkyl, or —N(R^{4c})(R^{4d}) or —SO₂N(R^{4c})(R^{4d}), wherein R^{4c} and R^{4d} are independently H, OH (R^{4c} and R^{4d} are not both OH), C₁₋₃ alkyl, C₁₋₆ hydroxyalkyl, or C₁₋₆ alkyl (preferably C₁₋₃ alkyl), or R^{ae} and R^{af} taken together with the nitrogen they are attached to form a 3, 4, 5 or 6-membered heterocycle; and
—N(R^{4c})(R^{4d}) where R^{4c} and R^{4d} are independently H, hydroxyl, C₁₋₆ alkyl, C₁₋₆ hydroxyalkyl, or —N(R^{4m})(R⁴) where R^{4m} and R⁴ⁿ are independently H or C₁₋₃ alkyl, or R^{4c} and R^{4d} can be taken together with the nitrogen they are attached to form a 3, 4, 5 or 6-membered heterocycle, and/or R^{4m} and R⁴ⁿ can be taken together with the nitrogen they are attached to form a 3, 4, 5 or 6-membered heterocycle; and
(10) —CON(R^{4p})(R^{4q}) wherein R^{4p} and R^{4q} are independently H, or C₁₀ alkyl that is optionally substituted with 1, 2, or 3 substituents each being independently
   hydroxyl;
   halo;
   —N(R^{4r})(R^{4t}) where R^{4r} and R^{4t} are independently H, C₁₋₃ alkyl, hydroxyl, or C₁₋₃ hydroxylalkyl;
   heterocycle

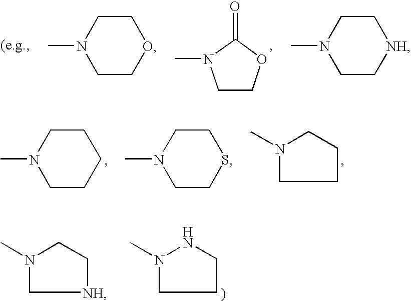

optionally substituted with 1, 2, or 3 substituents each being independently halo (e.g., F, Cl, Br, I), C₁₋₆ alkyl, or C₁₋₃ haloalkyl;
C₁₋₁₀ alkoxy, C₁₋₁₀ alkylthiol, C₂₋₁₀ alkenyloxy, C₂₋₁₀ alkynyloxy; and
aryl or heteroaryl, optionally substituted with 1, 2, or 3 substituents each being independently halo (e.g., F, Cl, Br, I), hydroxyl, C₁₋₆ alkyl (preferably methyl), C₁₋₃ haloalkyl, carboxyl, C₁₋₃ alkyoxycarbonyl, —N(R^{4c})(R^{4d}) or —SO₂N(R^{4c})(R^{4d}), wherein R^{4c} and R^{4d} are independently H, OH(R^{4c} and R^{4d} are not both OH), C₁₋₃ alkyl, C₁₋₆ hydroxyalkyl, or C₁₋₆ alkyl (preferably C₁₋₃ alkyl), or R^{4c} and R^{4d} taken together with the nitrogen they are attached to form a 3, 4, 5 or 6-membered heterocycle; and
(11) cycloalkyl, heterocycle, aryl or heteroaryl, optionally substituted with 1, 2, or 3 substituents each being independently halo (e.g., F, Cl, Br, I); hydroxyl; C₁₋₆ alkyl (preferably methyl); C₁₋₃ haloalkyl; —CO₂R⁴¹ or —O(C═O)R⁴¹ wherein R⁴ⁱ is H or C₁₋₃ alkyl; —N(R^{4c})(R^{4d}) or —SO₂N(R^{4c})(R^{4d}) wherein R^{4c} and R^{4d} are independently H, OH(R^{4c} and R^{4d} are not both OH), C₁₋₃ alkyl, C₁₋₆ hydroxyalkyl, or C₁₋₆ alkyl (preferably C₁₋₃ alkyl), or R^{4c} and R^{4d} taken together with the nitrogen they are attached to form a 3, 4, 5 or 6-membered heterocycle.

In some embodiments of the compounds of Formula V, R¹ is carboxyalkanoyl having 3-10 carbon atoms, and optionally substituted with 1, 2, 3, 4, 5, 6 halo atoms (e.g., F). In some embodiments of the compounds of Formula V, R¹ carboxyhaloalkanoyl having 3-10 carbon atoms.

In some embodiments of the compounds of Formula V, R¹ is chosen from succinyl, glutaryl, 3′-methylglutaryl, 3′-methylsuccinyl, 3′3′-dimethylsuccinyl, 3′3′-dimethylglutaryl, 3′-methyl-3′-ethylsuccinyl, 3′-methyl-3′-ethylglutaryl, and C₁₋₆ alkyl ester thereof, optionally substituted with 1, 2, 3, 4, 5, 6 halo atoms (e.g., F).

In some embodiments of the compounds of Formula V, R¹ is —C(═O)—(CH₂)ₘ—C(CH₃)₂—(CH₂)ₙ—COOH or —C(═O)—(CH₂)ₘ—C(CH₃)₂—(CH₂)ₙ—C(O)—C₁₋₆ alkyl, wherein m and n are independently an integer of 0, 1, 2 or 3, and more preferably R¹ is —C(═O)—CH₂—C(CH₃)₂COOH (3′,3′-dimethylsuccinyl) or —C(═O)—CH₂—C(CH₃)₂CH₂COOH (3′,3′-dimethylglutaryl), —C(═O)—CH₂—C(CH₃)₂C(O)—C₁₋₆ alkyl or —C(═O)—CH₂—C(CH₃)₂CH₂C(O)—C₁₋₆ alkyl, each being optionally substituted with 1, 2, 3, 4, 5, 6 halo atoms (e.g., F).

In some embodiments of the compounds of Formula V, R¹ is —C(═O)—CH₂—C(R¹¹⁸)(R¹¹⁹)COOH, —C(═O)—CH₂—C(R¹¹⁸)(R¹¹⁹)CH₂COOH, —C(═O)—CH₂—C(R¹¹⁸)(R¹¹⁹)C(O)—C₁₋₆ alkyl or —C(═O)—CH₂—C(R¹¹⁸)(R¹¹⁹)CH₂C(O)—C₁₋₆ alkyl, wherein R¹¹⁸ and R¹¹⁹ are independently trifluoromethyl, trifluoroethyl, methyl, ethyl, or R¹¹⁸ and R¹¹⁹ together with the carbon atom they are attached to form a 3, 4 or 5-membered cycloalkyl or heterocycle having an O or S atom. In specific embodiments, R¹¹⁸ and R¹¹⁹ are not both methyl.

In some embodiments of the compounds of Formula V, R¹ is —C(═O)—CH₂—C(CF₃)₂COOH or —C(═O)—CH₂—C(CF₃)₂CH₂COOH.

In some embodiments of the compounds of Formula V, R¹ is

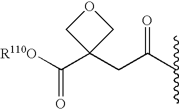

wherein R¹¹⁰ is H or C₁₋₆ alkyl.

In some specific embodiments, the compound is not one of Compounds 81, 105, 117 and 121 below.

In some specific embodiments of the compounds of Formula V, R¹ is not —C(═O)—(CH₂)ₘ—C(CH₃)₂—(CH₂)ₙ—COOH wherein m and n are independently an integer of 0, 1, 2 or 3.

In certain embodiments, R² is isopropenyl or isopropyl, preferably isopropenyl.

In some embodiments, R⁴ is a heteroaryl or heteroarylmethyl or heteroarylethyl having at least one nitrogen and optionally substituted with 1, 2 or 3 substituents independently chosen from halo (e.g., F, Cl, Br, I); C₁₋₆ alkyl; C₁₋₆ haloalkyl; hydroxyl; amino or C₁₋₃ alkylamino; C₁₋₆ alkoxy optionally substituted with 1-3 halo (e.g., F, Cl, Br, I); carboxyl; C₁₋₆ alkoxycarbonyl. Preferably R⁴ is chosen from pyridine, pyrimidine, pyrazine, pyridazine, and triazine, optionally substituted with 1, 2 or 3 above substituents. In some specific embodiments, R⁴ is unsubstituted pyridine.

In some specific forms of any one of the above embodiments, R⁴ is not p-methoxyphenyl or 2-pyridinyl.

In yet another aspect, the present invention provides compounds of Formula VI

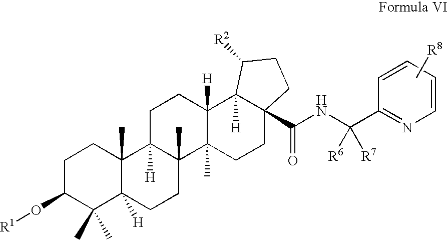

and pharmaceutically acceptable salts and stereoisomers thereof,
wherein

R¹ is R¹¹—C(O)— wherein R¹¹ is C₁₋₂₀ (preferably C₁₋₁₀, more preferably C₁₋₆) alkyl, C₁₋₂₀ (preferably C₁₋₁₀, more preferably C₁₋₆) alkenyl, or C₁₋₂₀ (preferably C₁₋₁₀, more preferably C₁₋₆) alkynyl, each being optionally substituted with one or more substituents independently chosen from the group of:
halo (e.g., F, Cl, Br, I); C₁₋₆ alkyl; —CN; hydroxyl; aryl; heteroaryl; cycloalkyl; heterocycle;
—C(O)R¹² where R¹² is —OH, C₁₋₆ alkoxy, C₁₋₆ alkenyloxy, C₁₋₆ alkynyloxy, C₃₋₆ cycloalkoxy or heterocycle;
—C(O)—N(R¹³)(R¹⁴) where R¹³ and R¹⁴ are independently H, C₁₋₆ alkyl, aryl, heteroaryl, C₃₋₆ cycloalkyl, —P(O)(OH)₂, (C₁₋₆ alkyl)phosphono, or —SO₃R¹⁵ where R¹⁵ is H, C₁₋₆ alkyl or aryl, or R¹³ and R¹⁴ together with the nitrogen atom they are linked to form a 3 to 6-membered heterocycle;
—N(R¹³)(R¹⁴) where R¹³ and R¹⁴ are independently H, C₁₋₆ alkyl, aryl, heteroaryl, C₃₋₆ cycloalkyl, or R¹³ and R¹⁴ together with the nitrogen atom they are linked to form a 3 to 6-membered heterocycle;
—SO₃R¹⁵, where R¹⁵ is C₁₋₆ alkyl, aryl or heteroaryl;
—NHSO₃R¹⁶, where R¹⁶ is C₁₋₆ alkyl, aryl, or heteroaryl; and
—P(O)(OR¹⁷)₂ where R¹⁷ is H or C₁₋₆ alkyl;
wherein optionally two substituents (e.g., one alkyl and one hydroxyl) at one carbon atom of R¹¹ may, together with the one carbon atom they are attached to, form a 3 to 6-membered cycloalkyl or heterocycle;

R² is isopropenyl or isopropyl, optionally substituted with one or two substituents independently selected from hydroxyl, halo, amino, and pyrrolidinyl, piperidinyl, and preferably R² is isopropenyl, isopropyl, 1′-hydroxyisopropyl, 2′-hydroxyisopryl, 1′,2′-dihydroxyisopropyl, and 1′-pyrrolidinyl-2′-hydroxyisopropyl;

R⁶ and R⁷ are independently H, methyl or ethyl, or R⁶ and R⁷ together with the carbon they are attached to form a cyclopropyl, and wherein at least one of R⁶ and R⁷ is not H; and

R⁸ is 1, 2, or 3 same or different substituents on the pyridine ring each independently being H or
(1) halo (e.g., F, Cl, Br, I);
(2) hydroxyl;
(3) C₁₋₁₀ alkyl (preferably C₁₋₆ alkyl) or C₃₋₆ cycloalkyl, optionally substituted with 1, 2 or 3 moieties independently chosen from: hydroxyl; halo (preferably F. e.g., monofluoro, difluoro, or trifluoro); C₁₋₆ alkoxy; C₁₋₆ haloalkoxy; C₃₋₁₀ cycloalkyl; heterocycle; aryl; heteroaryl; —C(O)R^{4a} where R^{4a} is —OH, C₁₋₆ alkoxy, C₁₋₆ alkenyloxy, C₁₋₆ alkynyloxy, C₃₋₆ cycloalkoxy or heterocycle; —C(O)—N(R^{4c})(R^{4d}) where R^{4c} and R^{4d} are independently H, C₁₋₆ alkyl, aryl, heteroaryl, C₃₋₆ cycloalkyl, or —SO₃R^{4e} where R^{4e} is H, C₁₋₆ alkyl or aryl, or R^{4c} and R^{4d} together with the nitrogen atom they are linked to form a 3 to 6-membered heterocycle; —N(R^{4c})(R^{4d}) where R^{4c} and R^{4d} are independently H, C₁₋₆ alkyl, C₁₋₆ hydroxyalkyl, aryl, heteroaryl, C₃₋₆ cycloalkyl, or —SO₃R^{4e} where R^{4e} is H, C₁₋₆ alkyl or aryl, or R^{4c} and R^{4d} together with the nitrogen atom they are linked to form a 3 to 6-membered heterocycle; —SO₃R^{4f}, where R^{4f} is C₁₋₆ alkyl, aryl or heteroaryl; —NHSO₃R^{4g}, where R^{4g} is C₁₋₆ alkyl, aryl, or heteroaryl; —N(R^{4b})—C(O)R^{4h} where R^{4b} is H or methyl or ethyl, R^{4h} is C₁₋₆ alkyl; and —N(R^{4b})—C(O)—N(R^{4c})(R^{4d}) where R^{4b} is H or methyl or ethyl, R^{4c} and R^{4d} are independently H, C₁₋₆ alkyl, aryl, heteroaryl, C₃₋₆ cycloalkyl, or —SO₃R^{4e} where R^{4e} is H, C₁₋₆ alkyl or aryl, or R^{4c} and R^{4d} together with the nitrogen atom they are linked to form a 3 to 6-membered heterocycle;
(4) —CO₂R⁴ⁱ or —O(C═O)R⁴ⁱ wherein R⁴ⁱ is H or C₁₋₆ alkyl, preferably methyl or ethyl;
(5) —N(R^{4c})(R^{4d}) or —SO₂N(R^{4c})(R^{4d}), wherein R^{4c} and R^{4d} are independently H, OH(R^{4c} and R^{4d} are not both OH), C₁₋₆ hydroxyalkyl, or C₁₋₆ alkyl (preferably C₁₋₃ alkyl), aryl or heteroaryl, or R^{4c} and R^{4d} taken together with the nitrogen they are attached to form a 3, 4, 5 or 6-membered heterocycle;
(6) —SO₃R^{4e}, where R^{4e} is C₁₋₆ alkyl, aryl or heteroaryl;
(7) —NHSO₃R^{4f}, where R^{4f} is C₁₋₆ alkyl, aryl, or heteroaryl;
(8) —N(R^{4b})C(═O)R^{4h}, —N(R^{4b})C(═O)N(R^{4c})(R^{4d}), or —OC(═O)N(R^{4c})(R^{4d}), where R^{4b} is H or methyl or ethyl; R^{4h}, R^{4c} and R^{4d} are independently H, OH(R^{4c} and R^{4d} are not both OH), C₁₋₁₀ alkyl, C₂₋₁₀ alkenyl, C₂₋₁₀ alkynyl, C₁₋₁₀ alkoxy, C₁₋₁₀ alkylthiol, C₂₋₁₀ alkenyloxy, C₂₋₁₀ alkynyloxy, C₁₋₁₀ haloalkyl, C₂₋₆ hydroxyalkyl, C₁₋₆ alkyl-O—C₁₋₆ alkyl-, cycloalkyl, heterocycle, aryl, heteroaryl, or R^{4c} and R^{4d} together with the nitrogen atom to which they are both linked form a 3, 4, 5 or 6-membered heterocycle (e.g., piperidinyl, pyrrolidinyl, and morpholinyl);
(9) C₁₋₆ alkoxy optionally substituted with 1, 2 or 3 substituents each being independently chosen from the group consisting of:
   hydroxyl;
   halo (e.g., F, Cl, Br, I);
   —CO₂R⁴ⁱ where R⁴ⁱ is H or C₁₋₆ alkyl (preferably methyl);
   heterocycle

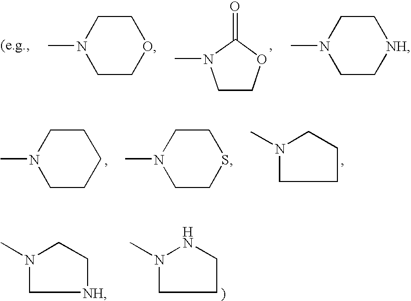

optionally substituted with 1, 2, or 3 substituents each being independently halo (e.g., F, Cl, Br, I), C₁₋₆ alkyl, or C₁₋₃ haloalkyl;
heteroaryl (e.g., imidazolyl) optionally substituted with 1, 2, or 3 substituents each being independent halo (e.g., F, Cl, Br, I), hydroxyl, C₁₋₆ alkyl (preferably methyl), C₁₋₆ alkoxy, carboxyl, C₁₋₃ alkoxycarbonyl, C₁₋₃ hydroxyalkyl, C₁₋₃ haloalkyl, or —N(R^{4c})(R^{4d}) or —SO₂N(R^{4c})(R^{4d}), wherein R^{4c} and R^{4d} are independently H, OH (R^{4c} and R^{4d} are not both OH), C₁₋₃ alkyl, C₁₋₆ hydroxyalkyl, or C₁₋₆ alkyl (preferably C₁₋₃ alkyl), or R^{ae} and R^{af} taken together with the nitrogen they are attached to form a 3, 4, 5 or 6-membered heterocycle; and
—N(R^{4c})(R^{4d}) where R^{4c} and R^{4d} are independently H, hydroxyl, C₁₋₆ alkyl, C₁₋₆ hydroxyalkyl, or —N(R^{4m})(R⁴ⁿ) where R^{4m} and R⁴ⁿ are independently H or C₁₋₃ alkyl, or R^{4c} and R^{4d} can be taken together with the nitrogen they are attached to form a 3, 4, 5 or 6-membered heterocycle, and/or R^{4m} and R⁴ⁿ can be taken together with the nitrogen they are attached to form a 3, 4, 5 or 6-membered heterocycle; and
(10) —CON(R^{4p})(R^{4q}) wherein R^{4p} and R^{4q} are independently H, or C₁₀ alkyl that is optionally substituted with 1, 2, or 3 substituents each being independently
   hydroxyl;
   halo;
   —N(R^{4r})(R^{4t}) where R^{4r} and R^{4t} are independently H, C₁₋₃ alkyl, hydroxyl, or C₁₋₃ hydroxylalkyl;
   heterocycle

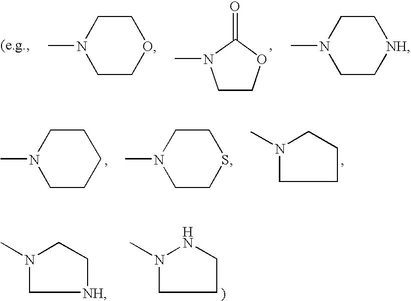

optionally substituted with 1, 2, or 3 substituents each being independently halo (e.g., F, Cl, Br, I), C₁₋₆ alkyl, or C₁₋₃ haloalkyl;
C₁₋₁₀ alkoxy, C₁₋₁₀ alkylthiol, C₂₋₁₀ alkenyloxy, C₂₋₁₀ alkynyloxy; and
aryl or heteroaryl, optionally substituted with 1, 2, or 3 substituents each being independently halo (e.g., F, Cl, Br, I), hydroxyl, C₁₋₆ alkyl (preferably methyl), C₁₋₃ haloalkyl, carboxyl, C₁₋₃ alkyoxycarbonyl, —N(R^{4c})(R^{4d}) or —SO₂N(R^{4c})(R^{4d}), wherein R^{4c} and R^{4d} are independently H, OH(R^{4c} and R^{4d} are not both OH), C₁₋₃ alkyl, C₁₋₆ hydroxyalkyl, or C₁₋₆ alkyl (preferably C₁₋₃ alkyl), or R^{4c} and R^{4d} taken together with the nitrogen they are attached to form a 3, 4, 5 or 6-membered heterocycle; and
(11) cycloalkyl, heterocycle, aryl or heteroaryl, optionally substituted with 1, 2, or 3 substituents each being independently halo (e.g., F, Cl, Br, I); hydroxyl; C₁₋₆ alkyl (preferably methyl); C₁₋₃ haloalkyl; —CO₂R⁴ⁱ or —O(C═O)R⁴¹ wherein R⁴ⁱ is H or C₁₋₃ alkyl; —N(R^{4c})(R^{4d}) or —SO₂N(R^{4c})(R^{4d}) wherein R^{4c} and R^{4d} are independently H, OH(R^{4c} and R^{4d} are not both OH), C₁₋₃ alkyl, C₁₋₆ hydroxyalkyl, or C₁₋₆ alkyl (preferably C₁₋₃ alkyl), or R^{4c} and R^{4d} taken together with the nitrogen they are attached to form a 3, 4, 5 or 6-membered heterocycle.

In some embodiments of the compounds of Formula VI, R¹ is carboxyalkanoyl having 3-10 carbon atoms, and optionally substituted with 1, 2, 3, 4, 5, 6 halo atoms (e.g., F). In some embodiments of the compounds of Formula VI, R¹ is carboxyhaloalkanoyl having 3-10 carbon atoms.

In some embodiments of the compounds of Formula VI, R¹ is chosen from succinyl, glutaryl, 3′-methylglutaryl, 3′-methylsuccinyl, 3′3′-dimethylsuccinyl, 3′3′-dimethylglutaryl, 3′-methyl-3′-ethylsuccinyl, 3′-methyl-3′-ethylglutaryl, and C₁₋₆ alkyl ester thereof, optionally substituted with 1, 2, 3, 4, 5, 6 halo atoms (e.g., F).

In some embodiments of the compounds of Formula VI, R¹ is —C(═O)—(CH₂)ₘ—C(CH₃)₂—(CH₂)ₙ—COOH or —C(═O)—(CH₂)ₘ—C(CH₃)₂—(CH₂)ₙ—C(O)—C₁₋₆ alkyl, wherein m and n are independently an integer of 0, 1, 2 or 3, and more preferably R¹ is —C(═O)—CH₂—C(CH₃)₂COOH (3′,3′-dimethylsuccinyl) or —C(═O)—CH₂—C(CH₃)₂CH₂COOH (3′,3′-dimethylglutaryl), —C(═O)—CH₂—C(CH₃)₂C(O)—C₁₋₆ alkyl or —C(═O)—CH₂—C(CH₃)₂CH₂C(O)—C₁₋₆ alkyl, each being optionally substituted with 1, 2, 3, 4, 5, 6 halo atoms (e.g., F).

In some embodiments of the compounds of Formula VI, R¹ is —C(═O)—CH₂—C(R¹¹⁸)(R¹¹⁹)COOH, —C(═O)—CH₂—C(R¹¹⁸)(R¹¹⁹)CH₂COOH, —C(═O)—CH₂—C(R¹¹⁸)(R¹¹⁹)C(O)—C₁₋₆ alkyl or —C(═O)—CH₂—C(R¹¹⁸)(R¹¹⁹)CH₂C(O)—C₁₋₆ alkyl, wherein R¹¹⁸ and R¹¹⁹ are independently trifluoromethyl, trifluoroethyl, methyl, ethyl, or R¹¹⁸ and R¹¹⁹ together with the carbon atom they are attached to form a 3, 4 or 5-membered cycloalkyl or heterocycle having an O or S atom. In specific embodiments, R¹¹⁸ and R¹¹⁹ are not both methyl.

In some embodiments of the compounds of Formula VI, R¹ is —C(═O)—CH₂—C(CF₃)₂COOH or —C(═O)—CH₂—C(CF₃)₂CH₂COOH.

In some embodiments of the compounds of Formula VI, R¹ is

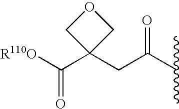

wherein R¹¹⁰ is H or C₁₋₆ alkyl.

In some specific embodiments, the compound is not one of Compounds 105 and 121 below.

In some specific embodiments of the compounds of Formula VI, R¹ is not —C(═O)—(CH₂)ₘ—C(CH₃)₂—(CH₂)ₙ—COOH wherein m and n are independently an integer of 0, 1, 2 or 3.

In certain embodiments, R² is isopropenyl or isopropyl, preferably isopropenyl.

In another aspect, the compound of Formula VI is according to Formula VII:

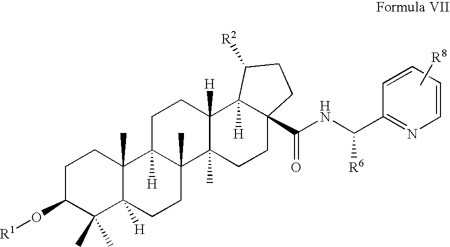

and pharmaceutically acceptable salts thereof, wherein R⁶ is methyl or ethyl, and R¹, R² and R⁸ are as defined above for Formula VI.

In yet another aspect, the compound of Formula VI has is according to Formula VIII:

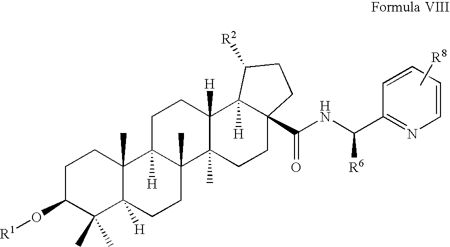

and pharmaceutically acceptable salts thereof, wherein R⁶ is methyl or ethyl, and R¹, R² and R⁸ as defined above for Formula VI.

In yet another aspect, the present invention provides a compound of Formula IX:

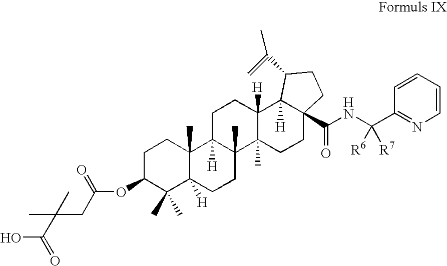

and pharmaceutically acceptable salts and stereoisomers thereof, wherein R⁶ and R⁷ are independently H, methyl or ethyl, or R⁶ and R⁷ together with the carbon they are attached to form a cyclopropyl or cyclobutyl or cyclopentyl, and wherein at least one of R⁶ and R⁷ is not H.

In yet another aspect, the present invention provides a compound of Formula X

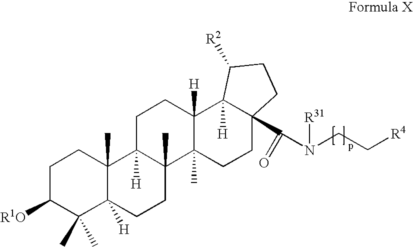

and pharmaceutically acceptable salts and stereoisomers thereof, wherein R¹, R², R³¹ and R⁴ are as defined for Formula V, and p is an integer of 2, 3 or 4.

In yet another aspect, the present invention provides a compound of Formula XI

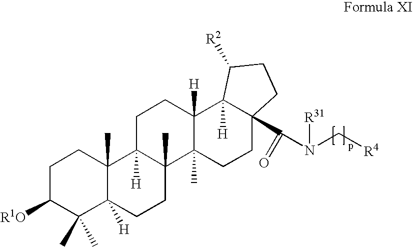

and pharmaceutically acceptable salts and stereoisomers thereof, wherein R¹, R², R³¹ and R⁴ are as defined for Formula V, and p is an integer of 0, 1, 2, 3, 4, 5, 6, 7, 8, 9 or 10.

In some specific embodiments of the compounds of Formula XI, R¹ is —C(═O)—CH₂—C(R¹⁸)(R¹⁹)COOH, —C(═O)—CH₂—C(R¹⁸)(R¹⁹)CH₂COOH, —C(═O)—CH₂—C(R¹⁸)(R¹⁹)C(O)—C₁₋₆ alkyl or —C(═O)—CH₂—C(R¹⁸)(R¹⁹)CH₂C(O)—C₁₋₆ alkyl, wherein R¹⁸ and R¹⁹ are independently trifluoromethyl, trifluoroethyl, methyl, ethyl, or R¹⁸ and R¹⁹ together with the carbon atom they are attached to form a 3, 4 or 5-membered cycloalkyl or heterocycle having an O or S atom, wherein R¹⁸ and R¹⁹ are not both methyl.

In some specific embodiments of the compounds of Formula XI, R¹ is —C(═O)—CH₂—C(CF₃)₂COOH or —C(═O)—CH₂—C(CF₃)₂CH₂COOH.

In some specific embodiments of the compounds of Formula XI, R¹ is

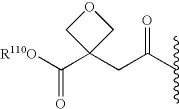

wherein R¹¹⁰ is H or C₁₋₆ alkyl.

In some specific embodiments of the compounds of Formula XI, R¹ is not —C(═O)—(CH₂)ₘ—C(CH₃)₂—(CH₂)ₙ—COOH wherein m and n are independently an integer of 0, 1, 2 or 3.

In some specific embodiments, the present invention provides compounds according to the above Formulae and various embodiments thereof and having and EC50 of less than about 1000 nM, 500 nM, 200 nM, and preferably 100 nM as determined in the PBMC Drug Susceptibility Assay described in Example 2.

Synthesis of compounds of Formula V as described herein are prepared by providing a compound according to Formula (100)

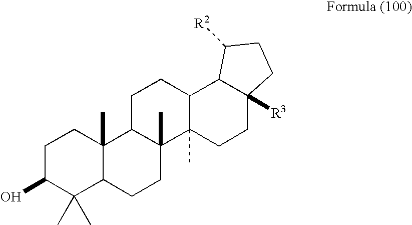

and converting the compound according to Formula (100) to a compound of Formula V.

The step of converting the compound according to Formula (100) to a compound of Formula V can be carried out by allowing a compound according to Formula (100) to react with an activated carbonyl compound, e.g., an anhydride such as 2,2-dimethyl succinic anhydride or by reaction with an acid chloride, e.g., 3-chlorocarbonyl-2,2-dimethyl-propionic acid methyl ester. Reaction with an anhydride occurs in pyridine solvent with an acylation catalyst such as 4-dimethylaminopyridine (DMAP) at a temperature of between 90° C. and 115° C. for between 12 and 24 hours. Reaction with an acid chloride takes place in methylene chloride and is conducted in the presence of an organic base such as triethylamine or diisopropylethylamine, an acylation catalyst such as DMAP and at ambient temperatures for between 12 and 24 hours.

The compound according to Formula (100) is provided by converting a compound according to Formula (110)

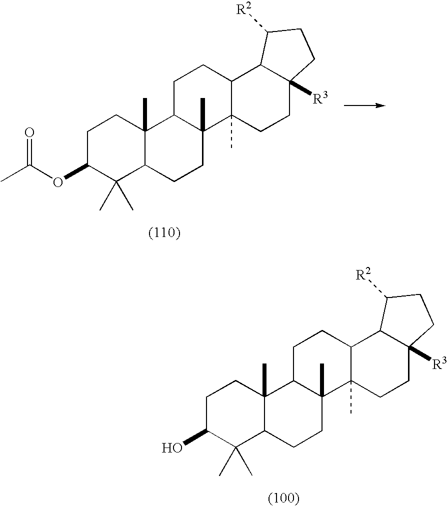

to the compound according to Formula (100). A compound of Formula (110) may be converted to a compound of Formula (100) by exposing a solution of the compound according to Formula (110) in a mixture of methanol and tetrahydrofuran to aqueous sodium hydroxide solution (usually between 2 M and 4 M, 5 equivalents of hydroxide ion). This occurs at ambient temperatures during from about 12 to 24 hours.

Alternatively, the compound according to Formula (100) may be provided by converting a compound according to Formula (140)

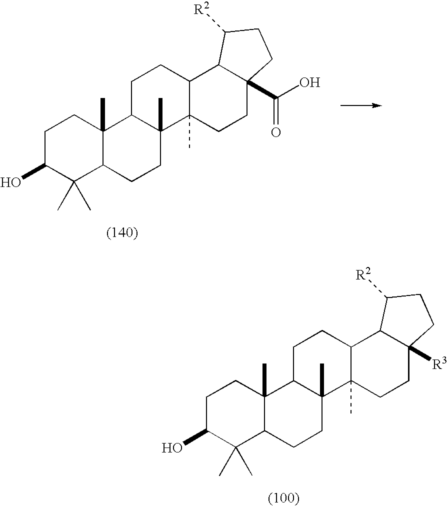

to the compound according to Formula (100). A solution of a compound according to Formula (140) (0.18 g, 0.394 mmol) in dry dimethylformamide (0.1 M in compound) is allowed to react with an activating diimide reagent, usually 1-ethyl-3-3(3-dimethylaminopropyl)carbodiimide hydrochloride (EDCl—HCl, 1.5 equivalents), 1-hydroxybenzotriazole [HOBt] or 1-hydroxy-7-azabenzotriazole [HOAt] (1 equivalent) and an organic base such as triethylamine or diisopropylamine (3 equivalents) at ambient temperatures for between 10 and 30 minutes. This mixture is then allowed to react with an appropriate amine compound (1.5 to 2 equivalents) for 18 to 24 hours at ambient temperatures. The compound of Formula (100) is usually purified by either silica gel chromatography or reversed phase HPLC.

The compound according to Formula (110) is provided by converting a compound according to Formula (120)

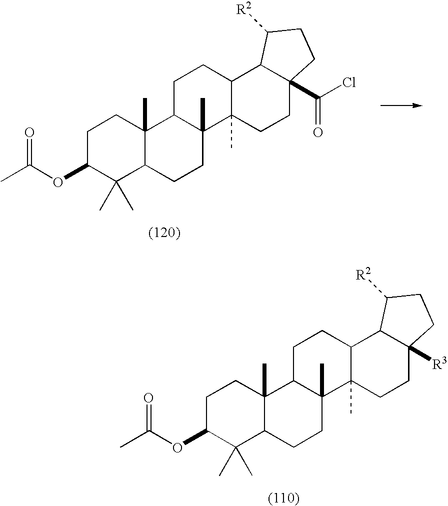

to the compound according to Formula (110). A solution of compound according to Formula (120) in dry dichloromethane (0.4 to 0.2 M in Formula 120) under an inert atmosphere of nitrogen is allowed to react with an appropriate amine compound (2 to 2.5 equivalents) and organic base such as triethylamine or diisopropylethylamine (3 to 5 equivalents). The mixture is allowed to react at ambient temperatures for 18 to 24 hours. The compound of Formula (110) is usually purified by either silica gel chromatography or reversed phase HPLC.

The compound according to Formula (120) is provided by converting a compound according to Formula (130)

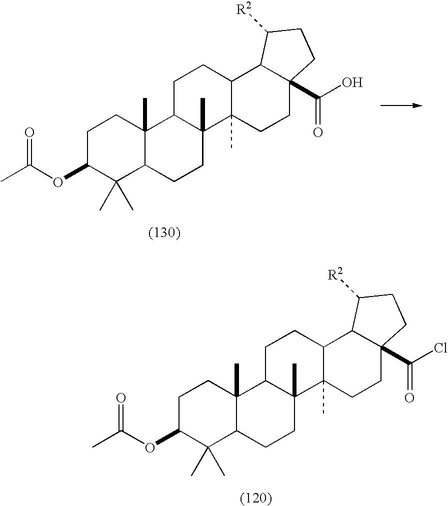

to the compound according to Formula (120). A compound of Formula (120) is prepared by exposing a compound of Formula (130) with an active chlorinating agent such as oxalyl chloride or thionyl chloride. In the former case, the reaction is conducted in a solvent (dichloromethane) with a catalyst (dimethylformamide) at room temperature for between 2 and 5 hours. In the latter case with thionyl chloride the reaction proceeds in thionyl chloride as solvent and with a catalyst (dimethylformamide) at 76° C. for between 3 and 6 hours.

The compound according to Formula (130) is provided by converting a compound according to Formula (140)

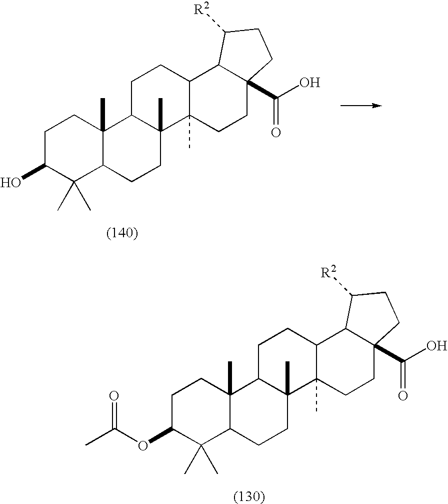

to the compound according to Formula (130). A compound according to Formula (130) is prepared by allowing a solution of compound Formula (140) in anhydrous pyridine (1 M in compound) to react with acetic anhydride (between 2.5 and 5 equivalents) and DMAP (1 equivalent) under an inert atmosphere of nitrogen at 115° C. for between 3 to 18 hours.

Another aspect of the present invention is directed to compounds according to Formula (100)

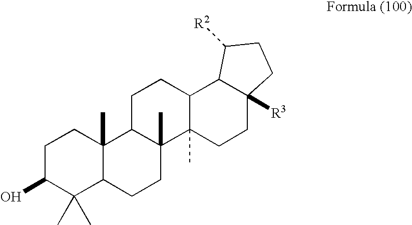

where

R² is isopropenyl or isopropyl, optionally substituted with one or two substituents independently selected from hydroxyl, halo, amino, pyrrolidinyl, and piperidinyl; and

R³ is represented by

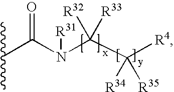

wherein
R³¹ is H or methyl or ethyl;
R³², R³³, R³⁴ and R³⁵ are independently H, methyl, ethyl, and either R³² and R³³ or R³⁴ and R³⁵ can be taken together with the carbon they are attached to form a cyclopropyl or cyclobutyl or cyclopentyl, and wherein at least one of R³², R³³, R³⁴ and R³⁵ when present, is not H;
x and y are independently an integer of 0 or 1, at least one of x and y is not 0; and

R⁴ is aryl, heteroaryl, arylalkyl, or heteroarylalkyl, each being optionally substituted with 1-6 substituents independently chosen from:
(1) halo;
(2) hydroxyl;
(3) C₁₋₁₀ alkyl or C₃₋₆ cycloalkyl, optionally substituted with 1-3 moieties independently chosen from: hydroxyl; halo; C₁₋₆ alkoxy; C₁₋₆ haloalkoxy; C₃₋₁₀ cycloalkyl; heterocycle; aryl; heteroaryl; —C(O)R^{4a}, wherein R^{4a} is —OH, C₁₋₆ alkoxy, C₁₋₆ alkenyloxy, C₁₋₆ alkynyloxy, C₃₋₆ cycloalkoxy or heterocycle; —C(O)—N(R^{4c})(R^{4d}), wherein R^{4c} and R^{4d} are independently H, C₁₋₆ alkyl, aryl, heteroaryl, C₃₋₆ cycloalkyl, or —SO₃R^{4e}, wherein R^{4e} is H, C₁₋₆ alkyl or aryl, or R^{4c} and R^{4d} together with the nitrogen atom they are linked to form a 3 to 6-membered heterocycle; —N(R^{4c})(R^{4d}), wherein R^{4c} and R^{4d} are independently H, C₁₋₆ alkyl, C₁₋₆ hydroxyalkyl, aryl, heteroaryl, C₃₋₆ cycloalkyl, or —SO₃R^{4e}, wherein R^{4e} is H, C₁₋₆ alkyl or aryl, or R^{4c} and R^{4d} together with the nitrogen atom they are linked to form a 3 to 6-membered heterocycle; —SO₃R^{4f}, wherein R^{4f} is C₁₋₆ alkyl, aryl or heteroaryl; —NHSO₃R^{4g}, wherein R^{4g} is C₁₋₆ alkyl, aryl, or heteroaryl; —N(R^{4b})—C(O)R^{4h}, wherein R^{4b} is H or methyl or ethyl, R^{4h} is C₁₋₆ alkyl; and —N(R^{4b})—C(O)—N(R^{4c})(R^{4d}), wherein R^{4b} is H or methyl or ethyl, R^{4c} and R^{4d} are independently H, C₁₋₆ alkyl, aryl, heteroaryl, C₃₋₆ cycloalkyl, or —SO₃R^{4e}, wherein R^{4e} is H, C₁₋₆ alkyl or aryl, or R^{4c} and R^{4d} together with the nitrogen atom they are linked to form a 3 to 6-membered heterocycle;
(4) —CO₂R⁴ⁱ or —O(C═O)R⁴ⁱ, wherein R⁴ⁱ is H or C₁₋₆ alkyl;
(5) —N(R^{4c})(R^{4d}) or —SO₂N(R^{4c})(R^{4d}), wherein R^{4c} and R^{4d} are independently H, OH(R^{4c} and R^{4d} are not both OH), C₁₋₆ hydroxyalkyl, or C₁₋₆ alkyl, aryl or heteroaryl, or R^{4c} and R^{4d} taken together with the nitrogen they are attached to form a 3 to 6-membered heterocycle;
(6) —SO₃R^{4e}, wherein R^{4e} is C₁₋₆ alkyl, aryl or heteroaryl;
(7) —NHSO₃R^{4f}, wherein R^{4f} is C₁₋₆ alkyl, aryl, or heteroaryl;
(8) —N(R^{4b})C(═O)R^{4h}, —N(R^{4b})C(═O)N(R^{4c})(R^{4d}), or —OC(═O)N(R^{4c})(R^{4d}), wherein R^{4b} is H or methyl or ethyl; R^{4h}, R^{4c} and R^{4d} are independently H, OH(R^{4c} and R^{4d} are not both OH), C₁₋₁₀ alkyl, C₂₋₁₀ alkenyl, C₂₋₁₀ alkynyl, C₁₋₁₀ alkoxy, C₁₋₁₀ alkylthiol, C₂₋₁₀ alkenyloxy, C₂₋₁₀ alkynyloxy, C₁₋₁₀ haloalkyl, C₂₋₆ hydroxyalkyl, C₁₋₆ alkyl-O—C₁₋₆ alkyl-, cycloalkyl, heterocycle, aryl, heteroaryl, or R^{4c} and R^{4d} together with the nitrogen atom to which they are both linked form a 3 to 6-membered heterocycle;
(9) C₁₋₆ alkoxy optionally substituted with 1-3 substituents each being independently chosen from the group consisting of: hydroxyl; halo; —CO₂R⁴ⁱ, wherein R⁴ⁱ is H or C₁₋₆ alkyl; heterocycle optionally substituted with 1-3 substituents each being independently halo, C₁₋₆ alkyl, or C₁₋₃ haloalkyl; heteroaryl optionally substituted with 1-3 substituents each being independent halo, hydroxyl, C₁₋₆ alkyl, C₁₋₆ alkoxy, carboxyl, C₁₋₃ alkoxycarbonyl, C₁₋₃ hydroxyalkyl, C₁₋₃ haloalkyl, or —N(R^{4c})(R^{4d}) or —SO₂N(R^{4c})(R^{4d}), wherein R^{4c} and R^{4d} are independently H, OH(R^{4c} and R^{4d} are not both OH), C₁₋₃ alkyl, C₁₋₆ hydroxyalkyl, or C₁₋₆ alkyl, or R^{ae} and R^{af} taken together with the nitrogen they are attached to form a 3 to 6-membered heterocycle; and —N(R^{4c})(R^{4d}), wherein R^{4c} and R^{4d} are independently H, hydroxyl, C₁₋₆ alkyl, C₁₋₆ hydroxyalkyl, or —N(R^{4m})(R⁴ⁿ), wherein R^{4m} and R⁴ⁿ are independently H or C₁₋₃ alkyl, or R^{4c} and R^{4d} can be taken together with the nitrogen they are attached to form a 3 to 6-membered heterocycle, and/or R^{4m} and R⁴ⁿ can be taken together with the nitrogen they are attached to form a 3 to 6-membered heterocycle;
(10) —CON(R^{4p})(R^{4q}), wherein R^{4p} and R^{4q} are independently H, or C₁₋₁₀ alkyl that is optionally substituted with 1-3 substituents each being independently hydroxyl; halo; —N(R^{4r})(R^{4t}), wherein R^{4r} and R^{4t} are independently H, C₁₋₃ alkyl, hydroxyl, or C₁₋₃ hydroxylalkyl; heterocycle optionally substituted with 1-3 substituents each being independently halo, C₁₋₆ alkyl, or C₁₋₃ haloalkyl; C₁₀ alkoxy, C₁₀ alkylthiol, C₂₋₁₀ alkenyloxy, C₂₋₁₀ alkynyloxy; and aryl or heteroaryl, optionally substituted with 1-3 substituents each being independently halo, hydroxyl, C₁₋₆ alkyl, C₁₋₃ haloalkyl, carboxyl, C₁₋₃ alkyoxycarbonyl, —N(R^{4c})(R^{4d}) or —SO₂N(R^{4c})(R^{4d}), wherein R^{4c} and R^{4d} are independently H, OH(R^{4c} and R^{4d} are not both OH), C₁₋₃ alkyl, C₁₋₆ hydroxyalkyl, or C₁₋₆ alkyl, or R^{4c} and R^{4d} taken together with the nitrogen they are attached to form a 3 to 6-membered heterocycle; and
(11) cycloalkyl, heterocycle, aryl, or heteroaryl, optionally substituted with 1-3 substituents each being independently halo; hydroxyl; C₁₋₆ alkyl; C₁₋₃ haloalkyl; —CO₂R⁴ⁱ or —O(C═O)R⁴, wherein R⁴ⁱ is H or C₁₋₃ alkyl; —N(R^{4c})(R^{4d}) or —SO₂N(R^{4c})(R^{4d}), wherein R^{4c} and R^{4d} are independently H, OH (R^{4c} and R^{4d} are not both OH), C₁₋₃ alkyl, C₁₋₆ hydroxyalkyl, or C₁₋₆ alkyl, or R^{4c} and R^{4d} taken together with the nitrogen they are attached to form a 3 to 6-membered heterocycle.

In some embodiments of the compounds according to Formula (100), R⁴ is not p-methoxyphenyl or 2-pyridinyl in the form of a racemic mixture.

Another aspect of the present invention is directed to compounds according to Formula (110)

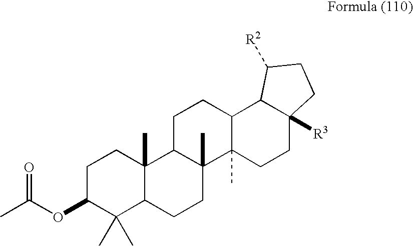

where

R² is isopropenyl or isopropyl, optionally substituted with one or two substituents independently selected from hydroxyl, halo, amino, pyrrolidinyl, and piperidinyl; and

R³ is represented by

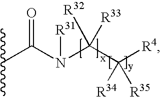

wherein
R³¹ is H or methyl or ethyl;
R³², R³³, R³⁴ and R³⁵ are independently H, methyl, ethyl, and either R³² and R³³ or R³⁴ and R³⁵ can be taken together with the carbon they are attached to form a cyclopropyl or cyclobutyl or cyclopentyl, and wherein at least one of R³², R³³, R³⁴ and R³⁵, when present, is not H;
x and y are independently an integer of 0 or 1, at least one of x and y is not 0; and

R⁴ is aryl, heteroaryl, arylalkyl, or heteroarylalkyl, each being optionally substituted with 1-6 substituents independently chosen from:
(1) halo;
(2) hydroxyl;
(3) C₁₋₁₀ alkyl or C₃₋₆ cycloalkyl, optionally substituted with 1-3 moieties independently chosen from: hydroxyl; halo; C₁₋₆ alkoxy; C₁₋₆ haloalkoxy; C₃₋₁₀ cycloalkyl; heterocycle; aryl; heteroaryl; —C(O)R^{4a}, wherein R^{4a} is —OH, C₁₋₆ alkoxy, C₁₋₆ alkenyloxy, C₁₋₆ alkynyloxy, C₃₋₆ cycloalkoxy or heterocycle; —C(O)—N(R^{4c})(R^{4d}), wherein R^{4c} and R^{4d} are independently H, C₁₋₆ alkyl, aryl, heteroaryl, C₃₋₆ cycloalkyl, or —SO₃R^{4e}, wherein R^{4e} is H, C₁₋₆ alkyl or aryl, or R^{4c} and R^{4d} together with the nitrogen atom they are linked to form a 3 to 6-membered heterocycle; —N(R^{4c})(R^{4d}), wherein R^{4c} and R^{4d} are independently H, C₁₋₆ alkyl, C₁₋₆ hydroxyalkyl, aryl, heteroaryl, C₃₋₆ cycloalkyl, or —SO₃R^{4e}, wherein R^{4e} is H, C₁₋₆ alkyl or aryl, or R^{4c} and R^{4d} together with the nitrogen atom they are linked to form a 3 to 6-membered heterocycle; —SO₃R⁴, wherein R^{4f} is C₁₋₆ alkyl, aryl or heteroaryl; —NHSO₃R^{4g}, wherein R^{4g} is C₁₋₆ alkyl, aryl, or heteroaryl; —N(R^{4b})—C(O)R^{4h}, wherein R^{4b} is H or methyl or ethyl, R^{4h} is C₁₋₆ alkyl; and —N(R^{4b})—C(O)—N(R^{4c})(R^{4d}), wherein R^{4b} is H or methyl or ethyl, R^{4c} and R^{4d} are independently H, C₁₋₆ alkyl, aryl, heteroaryl, C₃₋₆ cycloalkyl, or —SO₃R^{4e}, wherein R^{4e} is H, C₁₋₆ alkyl or aryl, or R^{4c} and R^{4d} together with the nitrogen atom they are linked to form a 3 to 6-membered heterocycle;
(4) —CO₂R⁴ⁱ or —O(C═O)R⁴ⁱ, wherein R⁴ⁱ is H or C₁₋₆ alkyl;
(5) —N(R^{4c})(R^{4d}) or —SO₂N(R^{4c})(R^{4d}), wherein R^{4c} and R^{4d} are independently H, OH(R^{4c} and R^{4d} are not both OH), C₁₋₆ hydroxyalkyl, or C₁₋₆ alkyl, aryl or heteroaryl, or R^{4c} and R^{4d} taken together with the nitrogen they are attached to form a 3 to 6-membered heterocycle;
(6) —SO₃R^{4e}, wherein R^{4e} is C₁₋₆ alkyl, aryl or heteroaryl;
(7) —NHSO₃R^{4f}, wherein R^{4f} is C₁₋₆ alkyl, aryl, or heteroaryl;
(8) —N(R^{4b})C(═O)R^{4h}, —N(R^{4b})C(═O)N(R^{4c})(R^{4d}), or —OC(═O)N(R^{4c})(R^{4d}), wherein R^{4b} is H or methyl or ethyl; R^{4h}, R^{4c} and R^{4d} are independently H, OH(R^{4c} and R^{4d} are not both OH), C₁₋₁₀ alkyl, C₂₋₁₀ alkenyl, C₂₋₁₀ alkynyl, C₁₋₁₀ alkoxy, C₁₋₁₀ alkylthiol, C₂₋₁₀ alkenyloxy, C₂₋₁₀ alkynyloxy, C₁₋₁₀ haloalkyl, C₂₋₆ hydroxyalkyl, C₁₋₆ alkyl-O—C₁₋₆ alkyl-, cycloalkyl, heterocycle, aryl, heteroaryl, or R^{4c} and R^{4d} together with the nitrogen atom to which they are both linked form a 3 to 6-membered heterocycle;
(9) C₁₋₆ alkoxy optionally substituted with 1-3 substituents each being independently chosen from the group consisting of: hydroxyl; halo; —CO₂R⁴ⁱ, wherein R⁴ⁱ is H or C₁₋₆ alkyl; heterocycle optionally substituted with 1-3 substituents each being independently halo, C₁₋₆ alkyl, or C₁₋₃ haloalkyl; heteroaryl optionally substituted with 1-3 substituents each being independent halo, hydroxyl, C₁₋₆ alkyl, C₁₋₆ alkoxy, carboxyl, C₁₋₃ alkoxycarbonyl, C₁₋₃ hydroxyalkyl, C₁₋₃ haloalkyl, or —N(R^{4c})(R^{d}) or —SO₂N(R^{4c})(R^{4d}), wherein R^{4c} and R^{4d} are independently H, OH(R^{4c} and R^{4d} are not both OH), C₁₋₃ alkyl, C₁₋₆ hydroxyalkyl, or C₁₋₆ alkyl, or R^{ae} and R^{af} taken together with the nitrogen they are attached to form a 3 to 6-membered heterocycle; and —N(R^{4c})(R^{4d}), wherein R^{4c} and R^{4d} are independently H, hydroxyl, C₁₋₆ alkyl, C₁₋₆ hydroxyalkyl, or —N(R^{4m})(R⁴ⁿ), wherein R^{4m} and R⁴ⁿ are independently H or C₁₋₃ alkyl, or R^{4c} and R^{4d} can be taken together with the nitrogen they are attached to form a 3 to 6-membered heterocycle, and/or R^{4m} and R⁴ⁿ can be taken together with the nitrogen they are attached to form a 3 to 6-membered heterocycle;
(10) —CON(R^{4p})(R^{4q}), wherein R^{4p} and R^{4q} are independently H, or C₁₋₁₀ alkyl that is optionally substituted with 1-3 substituents each being independently hydroxyl; halo; —N(R^{4r})(R^{4t}), wherein R^{4r} and R^{4t} are independently H, C₁₋₃ alkyl, hydroxyl, or C₁₋₃ hydroxylalkyl; heterocycle optionally substituted with 1-3 substituents each being independently halo, C₁₋₆ alkyl, or C₁₋₃ haloalkyl; C₁₋₁₀ alkoxy, C₁₋₁₀ alkylthiol, C₂₋₁₀ alkenyloxy, C₂₋₁₀ alkynyloxy; and aryl or heteroaryl, optionally substituted with 1-3 substituents each being independently halo, hydroxyl, C₁₋₆ alkyl, C₁₋₃ haloalkyl, carboxyl, C₁₋₃ alkyoxycarbonyl, —N(R^{4c})(R^{4d}) or —SO₂N(R^{4c})(R^{4d}), wherein R^{4c} and R^{4d} are independently H, OH(R^{4c} and R^{4d} are not both OH), C₁₋₃ alkyl, C₁₋₆ hydroxyalkyl, or C₁₋₆ alkyl, or R^{4c} and R^{4d} taken together with the nitrogen they are attached to form a 3 to 6-membered heterocycle; and
(11) cycloalkyl, heterocycle, aryl, or heteroaryl, optionally substituted with 1-3 substituents each being independently halo; hydroxyl; C₁₋₆ alkyl; C₁₋₃ haloalkyl; —CO₂R⁴ⁱ or —O(C═O)R⁴ⁱ, wherein R⁴ⁱ is H or C₁₋₃ alkyl; —N(R^{4c})(R^{4d}) or —SO₂N(R^{4c})(R^{4d}), wherein R^{4c} and R^{4d} are independently H, OH (R^{4c} and R^{4d} are not both OH), C₁₋₃ alkyl, C₁₋₆ hydroxyalkyl, or C₁₋₆ alkyl, or R^{4c} and R^{4d} taken together with the nitrogen they are attached to form a 3 to 6-membered heterocycle.

In some embodiments of the compounds according to Formula (110), R⁴ is not p-methoxyphenyl or 2-pyridinyl in the form of a racemic mixture.

Another aspect of the present invention is directed to a method of making a compound according to Formula (100). This method involves providing a compound according to Formula (110) and converting the compound according to Formula (110) to form a compound according to Formula (100).

Another aspect of the present invention is directed to a method of making a compound according to Formula (110). This method involves providing a compound according to Formula (120) and converting the compound according to Formula (120) to form a compound according to Formula (110).

In another aspect, compounds of the present invention have a general structure of Formula (300)

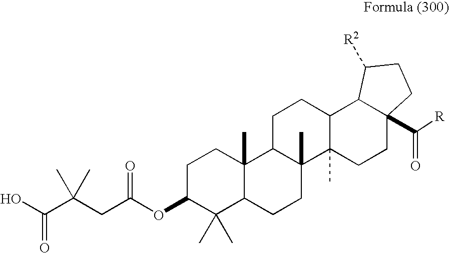

wherein

R² is isopropenyl or isopropyl, optionally substituted with one or two substituents independently selected from hydroxyl, halo, amino, pyrrolidinyl, and piperidinyl and

R is represented by

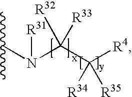

wherein
R³¹ is H or methyl or ethyl;
R³², R³³, R³⁴ and R³⁵ are independently H, methyl, ethyl, and either R³² and R³³ or R³⁴ and R³⁵ can be taken together with the carbon they are attached to form a cyclopropyl or cyclobutyl or cyclopentyl, and wherein at least one of R³², R³³, R³⁴ and R³⁵, when present, is not H;
x and y are independently an integer of 0 or 1, at least one of x and y is not 0; and

R⁴ is aryl, heteroaryl, arylalkyl, or heteroarylalkyl, each being optionally substituted with 1-6 substituents independently chosen from:
(1) halo;
(2) hydroxyl;
(3) C₁₋₁₀ alkyl or C₃₋₆ cycloalkyl, optionally substituted with 1-3 moieties independently chosen from: hydroxyl; halo; C₁₋₆ alkoxy; C₁₋₆ haloalkoxy; C₃₋₁₀ cycloalkyl; heterocycle; aryl; heteroaryl; —C(O)R^{4a}, wherein R^{4a} is —OH, C₁₋₆ alkoxy, C₁₋₆ alkenyloxy, C₁₋₆ alkynyloxy, C₃₋₆ cycloalkoxy or heterocycle; —C(O)—N(R^{4c})(R^{4d}), wherein R^{4c} and R^{4d} are independently H, C₁₋₆ alkyl, aryl, heteroaryl, C₃₋₆ cycloalkyl, or —SO₃R^{4e}, wherein R^{4e} is H, C₁₋₆ alkyl or aryl, or R^{4c} and R^{4d} together with the nitrogen atom they are linked to form a 3 to 6-membered heterocycle; —N(R^{4c})(R^{4d}), wherein R^{4c} and R^{4d} are independently H, C₁₋₆ alkyl, C₁₋₆ hydroxyalkyl, aryl, heteroaryl, C₃₋₆ cycloalkyl, or —SO₃R^{4e}, wherein R^{4e} is H, C₁₋₆ alkyl or aryl, or R^{4c} and R^{4d} together with the nitrogen atom they are linked to form a 3 to 6-membered heterocycle; —SO₃R^{4f}, wherein R^{4f} is C₁₋₆ alkyl, aryl or heteroaryl; —NHSO₃R^{4g}, wherein R^{4g} is C₁₋₆ alkyl, aryl, or heteroaryl; —N(R^{4b})—C(O)R^{4h}, wherein R^{4b} is H or methyl or ethyl, R^{4h} is C₁₋₆ alkyl; and —N(R^{4b})—C(O)—N(R^{4c})(R^{4d}), wherein R^{4b} is H or methyl or ethyl, R^{4c} and R^{4d} are independently H, C₁₋₆ alkyl, aryl, heteroaryl, C₃₋₆ cycloalkyl, or —SO₃R^{4e}, wherein R^{4e} is H, C₁₋₆ alkyl or aryl, or R^{4c} and R^{4d} together with the nitrogen atom they are linked to form a 3 to 6-membered heterocycle;
(4) —CO₂R⁴ⁱ or —O(C═O)R⁴ⁱ, wherein R⁴ⁱ is H or C₁₋₆ alkyl;
(5) —N(R^{4c})(R^{4d}) or —SO₂N(R^{4c})(R^{4d}), wherein R^{4c} and R^{4d} are independently H, OH(R^{4c} and R^{4d} are not both OH), C₁₋₆ hydroxyalkyl, or C₁₋₆ alkyl, aryl or heteroaryl, or R^{4c} and R^{4d} taken together with the nitrogen they are attached to form a 3 to 6-membered heterocycle;
(6) —SO₃R^{4e}, wherein R^{4e} is C₁₋₆ alkyl, aryl or heteroaryl;
(7) —NHSO₃R^{4f}, wherein R^{4f} is C₁₋₆ alkyl, aryl, or heteroaryl;
(8) —N(R^{4b})C(═O)R^{4h}, —N(R^{4b})C(═O)N(R^{4c})(R^{4d}), or —OC(═O)N(R^{4c})(R^{4d}), wherein R^{4b} is H or methyl or ethyl; R^{4h}, R^{4c} and R^{4d} are independently H, OH(R^{4c} and R^{4d} are not both OH), C₁₋₁₀ alkyl, C₂₋₁₀ alkenyl, C₂₋₁₀ alkynyl, C₁₋₁₀ alkoxy, C₁₋₁₀ alkylthiol, C₂₋₁₀ alkenyloxy, C₂₋₁₀ alkynyloxy, C₁₋₁₀ haloalkyl, C₂₋₆ hydroxyalkyl, C₁₋₆ alkyl-O—C₁₋₆ alkyl-, cycloalkyl, heterocycle, aryl, heteroaryl, or R^{4c} and R^{4d} together with the nitrogen atom to which they are both linked form a 3 to 6-membered heterocycle;
(9) C₁₋₆ alkoxy optionally substituted with 1-3 substituents each being independently chosen from the group consisting of: hydroxyl; halo; —CO₂R⁴ⁱ, wherein R⁴ⁱ is H or C₁₋₆ alkyl; heterocycle optionally substituted with 1-3 substituents each being independently halo, C₁₋₆ alkyl, or C₁₋₃ haloalkyl; heteroaryl optionally substituted with 1-3 substituents each being independent halo, hydroxyl, C₁₋₆ alkyl, C₁₋₆ alkoxy, carboxyl, C₁₋₃ alkoxycarbonyl, C₁₋₃ hydroxyalkyl, C₁₋₃ haloalkyl, or —N(R^{4c})(R^{4d}) or —SO₂N(R^{4c})(R^{4d}), wherein R^{4c} and R^{4d} are independently H, OH(R^{4c} and R^{4d} are not both OH), C₁₋₃ alkyl, C₁₋₆ hydroxyalkyl, or C₁₋₆ alkyl, or R^{ae} and R^{af} taken together with the nitrogen they are attached to form a 3 to 6-membered heterocycle; and —N(R^{4c})(R^{4d}), wherein R^{4c} and R^{4d} are independently H, hydroxyl, C₁₋₆ alkyl, C₁₋₆ hydroxyalkyl, or —N(R^{4m})(R⁴ⁿ), wherein R^{4m} and R⁴ⁿ are independently H or C₁₋₃ alkyl, or R^{4c} and R^{4d} can be taken together with the nitrogen they are attached to form a 3 to 6-membered heterocycle, and/or R^{4m} and R⁴ⁿ can be taken together with the nitrogen they are attached to form a 3 to 6-membered heterocycle;
(10) —CON(R^{4p})(R^{4q}), wherein R^{4p} and R^{4q} are independently H, or C₁₋₁₀ alkyl that is optionally substituted with 1-3 substituents each being independently hydroxyl; halo; —N(R^{4r})(R^{4t}), wherein R^{4r} and R^{4t} are independently H, C₁₋₃ alkyl, hydroxyl, or C₁₋₃ hydroxylalkyl; heterocycle optionally substituted with 1-3 substituents each being independently halo, C₁₋₆ alkyl, or C₁₋₃ haloalkyl; C₁₋₁₀ alkoxy, C₁₋₁₀ alkylthiol, C₂₋₁₀ alkenyloxy, C₂₋₁₀ alkynyloxy; and aryl or heteroaryl, optionally substituted with 1-3 substituents each being independently halo, hydroxyl, C₁₋₆ alkyl, C₁₋₃ haloalkyl, carboxyl, C₁₋₃ alkyoxycarbonyl, —N(R^{4c})(R^{4d}) or —SO₂N(R^{4c})(R^{4d}), wherein R^{4c} and R^{4d} are independently H, OH(R^{4c} and R^{4d} are not both OH), C₁₋₃ alkyl, C₁₋₆ hydroxyalkyl, or C₁₋₆ alkyl, or R^{4c} and R^{4d} taken together with the nitrogen they are attached to form a 3 to 6-membered heterocycle; and
(11) cycloalkyl, heterocycle, aryl, or heteroaryl, optionally substituted with 1-3 substituents each being independently halo; hydroxyl; C₁₋₆ alkyl; C₁₋₃ haloalkyl; —CO₂R⁴ⁱ or —O(C═O)R⁴ⁱ, wherein R⁴ⁱ is H or C₁₋₃ alkyl; —N(R^{4c})(R^{4d}) or —SO₂N(R^{4c})(R^{4d}), wherein R^{4c} and R^{4d} are independently H, OH (R^{4c} and R^{4d} are not both OH), C₁₋₃ alkyl, C₁₋₆ hydroxyalkyl, or C₁₋₆ alkyl, or R^{4c} and R^{4d} taken together with the nitrogen they are attached to form a 3 to 6-membered heterocycle.

In a particular embodiments of the compounds of Formula (300), R⁴ is not p-methoxyphenyl or 2-pyridinyl in the form of a racemic mixture.

Enantiomers of the compound according to Formula (300)

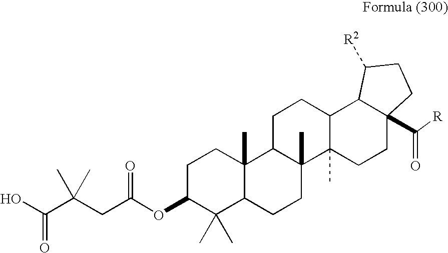

wherein

R² is isopropenyl or isopropyl, optionally substituted with one or two substituents independently selected from hydroxyl, halo, amino, pyrrolidinyl, and piperidinyl and

R is represented by

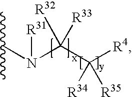

wherein
R³¹ is H or methyl or ethyl;
R³², R³³, R³⁴ and R³⁵ are independently H, methyl, ethyl, and either R³² and R³³ or R³⁴ and R³⁵ can be taken together with the carbon they are attached to form a cyclopropyl or cyclobutyl or cyclopentyl, and wherein at least one of R³², R³³, R³⁴ and R³⁵, when present, is not H;
x and y are independently an integer of 0 or 1, at least one of x and y is not 0; and

R⁴ is aryl, heteroaryl, arylalkyl, or heteroarylalkyl, each being optionally substituted with 1-6 substituents independently chosen from:
(1) halo;
(2) hydroxyl;
(3) C₁₋₁₀ alkyl or C₃₋₆ cycloalkyl, optionally substituted with 1-3 moieties independently chosen from: hydroxyl; halo; C₁₋₆ alkoxy; C₁₋₆ haloalkoxy; C₃₋₁₀ cycloalkyl; heterocycle; aryl; heteroaryl; —C(O)R^{4a}, wherein R^{4a} is —OH, C₁₋₆ alkoxy, C₁₋₆ alkenyloxy, C₁₋₆ alkynyloxy, C₃₋₆ cycloalkoxy or heterocycle; —C(O)—N(R^{4c})(R^{4d}), wherein R^{4c} and R^{4d} are independently H, C₁₋₆ alkyl, aryl, heteroaryl, C₃₋₆ cycloalkyl, or —SO₃R^{4e}, wherein R^{4e} is H, C₁₋₆ alkyl or aryl, or R^{4c} and R^{4d} together with the nitrogen atom they are linked to form a 3 to 6-membered heterocycle; —N(R^{4c})(R^{4d}), wherein R^{4c} and R^{4d} are independently H, C₁₋₆ alkyl, C₁₋₆ hydroxyalkyl, aryl, heteroaryl, C₃₋₆ cycloalkyl, or —SO₃R^{4e}, wherein R^{4e} is H, C₁₋₆ alkyl or aryl, or R^{4c} and R^{4d} together with the nitrogen atom they are linked to form a 3 to 6-membered heterocycle; —SO₃R⁴, wherein R^{4f} is C₁₋₆ alkyl, aryl or heteroaryl; —NHSO₃R^{4g}, wherein R^{4g} is C₁₋₆ alkyl, aryl, or heteroaryl; —N(R^{4b})—C(O)R^{4h}, wherein R^{4b} is H or methyl or ethyl, R^{4h} is C₁₋₆ alkyl; and —N(R^{4b})—C(O)—N(R^{4c})(R^{4d}), wherein R^{4b} is H or methyl or ethyl, R^{4c} and R^{4d} are independently H, C₁₋₆ alkyl, aryl, heteroaryl, C₃₋₆ cycloalkyl, or —SO₃R^{4e}, wherein R^{4e} is H, C₁₋₆ alkyl or aryl, or R^{4c} and R^{4d} together with the nitrogen atom they are linked to form a 3 to 6-membered heterocycle;
(4) —CO₂R⁴ⁱ or —O(C═O)R⁴ⁱ, wherein R⁴ⁱ is H or C₁₋₆ alkyl;
(5) —N(R^{4c})(R^{4d}) or —SO₂N(R^{4c})(R^{4d}), wherein R^{4c} and R^{4d} are independently H, OH(R^{4c} and R^{4d} are not both OH), C₁₋₆ hydroxyalkyl, or C₁₋₆ alkyl, aryl or heteroaryl, or R^{4c} and R^{4d} taken together with the nitrogen they are attached to form a 3 to 6-membered heterocycle;
(6) —SO₃R^{4e}, wherein R^{4e} is C₁₋₆ alkyl, aryl or heteroaryl;
(7) —NHSO₃R^{4f}, wherein R^{4f} is C₁₋₆ alkyl, aryl, or heteroaryl;
(8) —N(R^{4b})C(═O)R^{4h}, —N(R^{4b})C(═O)N(R^{4c})(R^{4d}), or —OC(═O)N(R^{4c})(R^{4d}), wherein R^{4b} is H or methyl or ethyl; R^{4h}, R^{4c} and R^{4d} are independently H, OH(R^{4c} and R^{4d} are not both OH), C₁₋₁₀ alkyl, C₂₋₁₀ alkenyl, C₂₋₁₀ alkynyl, C₁₋₁₀ alkoxy, C₁₋₁₀ alkylthiol, C₂₋₁₀ alkenyloxy, C₂₋₁₀ alkynyloxy, C₁₋₁₀ haloalkyl, C₂₋₆ hydroxyalkyl, C₁₋₆ alkyl-O—C₁₋₆ alkyl-, cycloalkyl, heterocycle, aryl, heteroaryl, or R^{4c} and R^{4d} together with the nitrogen atom to which they are both linked form a 3 to 6-membered heterocycle;
(9) C₁₋₆ alkoxy optionally substituted with 1-3 substituents each being independently chosen from the group consisting of: hydroxyl; halo; —CO₂R⁴ⁱ, wherein R⁴ⁱ is H or C₁₋₆ alkyl; heterocycle optionally substituted with 1-3 substituents each being independently halo, C₁₋₆ alkyl, or C₁₋₃ haloalkyl; heteroaryl optionally substituted with 1-3 substituents each being independent halo, hydroxyl, C₁₋₆ alkyl, C₁₋₆ alkoxy, carboxyl, C₁₋₃ alkoxycarbonyl, C₁₋₃ hydroxyalkyl, C₁₋₃ haloalkyl, or —N(R^{4c})(R^{4d}) or —SO₂N(R^{4c})(R^{4d}), wherein R^{4c} and R^{4d} are independently H, OH(R^{4c} and R^{4d} are not both OH), C₁₋₃ alkyl, C₁₋₆ hydroxyalkyl, or C₁₋₆ alkyl, or R^{ae} and R^{af} taken together with the nitrogen they are attached to form a 3 to 6-membered heterocycle; and —N(R^{4c})(R^{4d}), wherein R^{4c} and R^{4d} are independently H, hydroxyl, C₁₋₆ alkyl, C₁₋₆ hydroxyalkyl, or —N(R^{4m})(R⁴ⁿ), wherein R^{4m} and R⁴ⁿ are independently H or C₁₋₃ alkyl, or R^{4c} and R^{4d} can be taken together with the nitrogen they are attached to form a 3 to 6-membered heterocycle, and/or R^{4m} and R⁴ⁿ can be taken together with the nitrogen they are attached to form a 3 to 6-membered heterocycle;
(10) —CON(R^{4p})(R^{4q}), wherein R^{4p} and R^{4q} are independently H, or C₁₋₁₀ alkyl that is optionally substituted with 1-3 substituents each being independently hydroxyl; halo; —N(R^{4r})(R^{4t}), wherein R^{4r} and R^{4t} are independently H, C₁₋₃ alkyl, hydroxyl, or C₁₋₃ hydroxylalkyl; heterocycle optionally substituted with 1-3 substituents each being independently halo, C₁₋₆ alkyl, or C₁₋₃ haloalkyl; C₁₀ alkoxy, C₁₀ alkylthiol, C₂₋₁₀ alkenyloxy, C₂₋₁₀ alkynyloxy; and aryl or heteroaryl, optionally substituted with 1-3 substituents each being independently halo, hydroxyl, C₁₋₆ alkyl, C₁₋₃ haloalkyl, carboxyl, C₁₋₃ alkyoxycarbonyl, —N(R^{4c})(R^{4d}) or —SO₂N(R^{4c})(R^{4d}), wherein R^{4c} and R^{4d} are independently H, OH(R^{4c} and R^{4d} are not both OH), C₁₋₃ alkyl, C₁₋₆ hydroxyalkyl, or C₁₋₆ alkyl, or R^{4c} and R^{4d} taken together with the nitrogen they are attached to form a 3 to 6-membered heterocycle; and
(11) cycloalkyl, heterocycle, aryl, or heteroaryl, optionally substituted with 1-3 substituents each being independently halo; hydroxyl; C₁₋₆ alkyl; C₁₋₃ haloalkyl; —CO₂R⁴ⁱ or —O(C═O)R⁴ⁱ, wherein R⁴ⁱ is H or C₁₋₃ alkyl; —N(R^{4c})(R^{4d}) or —SO₂N(R^{4c})(R^{4d}), wherein R^{4c} and R^{4d} are independently H, OH (R^{4c} and R^{4d} are not both OH), C₁₋₃ alkyl, C₁₋₆ hydroxyalkyl, or C₁₋₆ alkyl, or R^{4c} and R^{4d} taken together with the nitrogen they are attached to form a 3 to 6-membered heterocycle, can be prepared by providing a compound according to Formula (310) and converting the compound according to Formula (310) to a compound according to Formula (300), as follows:

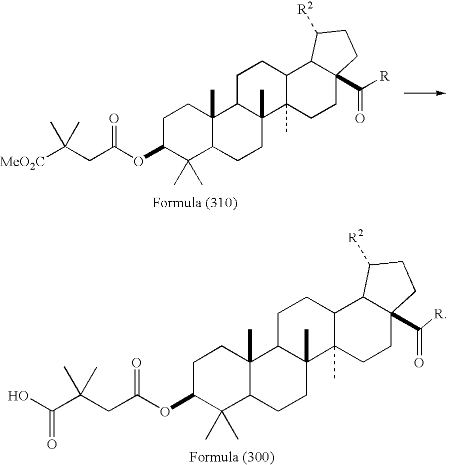

Converting a compound of Formula (310) to a compound of Formula (300) can be carried out by allowing a compound according to Formula (310) to react as a solution in THF and methanol (between 0.1 and 0.2 M in compound) with aqueous sodium hydroxide (between 5 and 6 equivalents) at ambient temperatures for between 3 and 8 hours. The product is obtained by adjusting the pH to between 4.5 and 5.5 with aqueous hydrochloric acid.

The compound according to Formula (310) is provided by converting a compound according to Formula (320) to the compound according to Formula (310), as follows:

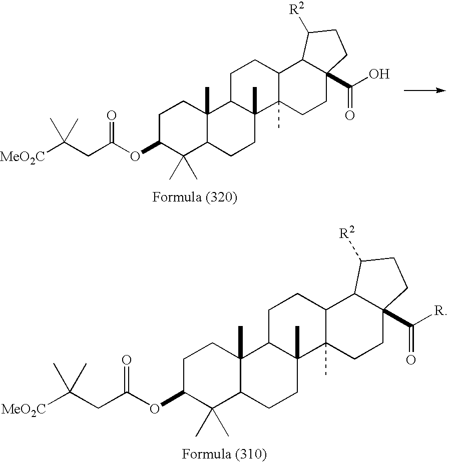

A compound of Formula (320) may be converted to a compound of Formula (310) by allowing an ice cold solution (ice-water bath, approximately 0° C.) of a compound of Formula (320) in dichloromethane (0.2 M in compound) to react with an active halogenating agent such as thionyl chloride (3.5 equivalents) and catalytic dimethylformamide (between 1 and 3 drops) for 30 minutes. The mixture is then heated at 39° C. for between 2 and 6 hours. The mixture is concentrated, dissolved in a halogenated solvent (one of higher boiling point than methylene chloride, for example chloroform), and evaporated to remove excess chlorinating agent. A solution of this material in dry methylene chloride (0.2 M in compound) maintained at 0° C. (ice-water bath) is allowed to react with an appropriate amine (between 1.2 and 1.5 equivalents) and an organic base such as triethylamine or diisopropylamine (3 equivalents) and then is allowed to warm to ambient temperatures and stir for between 18 and 24 hours.

The compound according to Formula (320) is provided by converting a compound according to Formula (330) to the compound according to Formula (320), as follows:

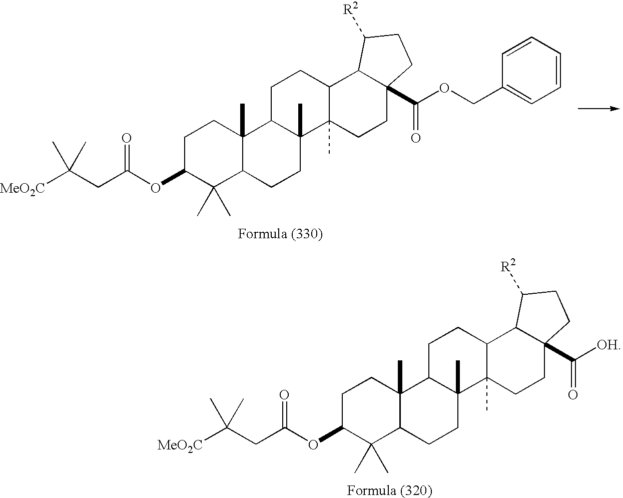

A compound of Formula (330) may be converted to a compound of Formula (320) by allowing a solution of a compound of Formula (330) in a mixture of tetrahydrofuran and methanol (0.2 M in compound) to react with palladium metal (10% by weight on activated carbon, 10 weight percent based on compound of Formula (330)) and ammonium formate (1.1 equivalents) at ambient temperatures for 2 hours.

The compound according to Formula (330) is provided by converting a compound according to Formula (340) to the compound according to Formula (330), as follows:

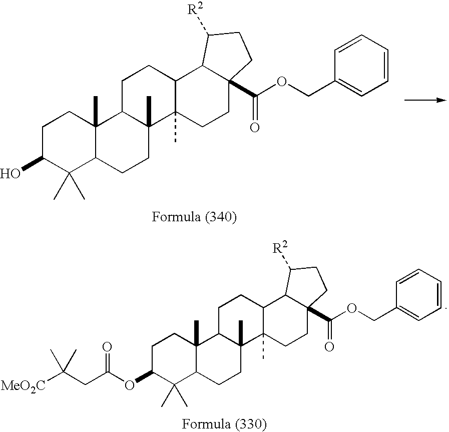

A compound of Formula (340) may be converted to a compound of Formula (330) by allowing a solution of a compound of Formula (340) in dry pyridine (0.2 M in compound) to react with an acylation catalyst such as 4-dimethylaminopyridine (1.2 equivalents) and a succinic anhydride such as 2,2-dimethylsuccinic anhydride (5 equivalents) at between 100° C. and 115° C. for 24 hours. After complete removal of pyridine, the material is suspended in ice-cold aqueous hydrochloric acid (1 M acid, 0.2 M in compound) and allowed to stir for between 2 and 3 hours. The intermediate is collected by filtration, dissolved in methanol and tetrahydrofuran (0.26 M in compound), cooled to ice-cold temperature, and treated with thionyl chloride (4 equivalents) during 2 hours. The resultant mixture is allowed to warm to ambient temperatures and stir for between 18 and 24 hours.

The compound according to Formula (340) is provided by converting a compound according to Formula (350) to the compound according to Formula (340), as follows:

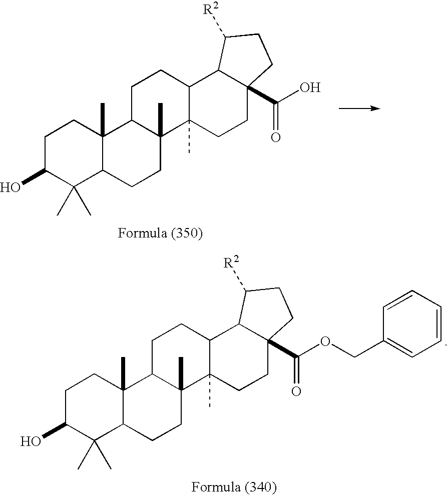

A compound of Formula (350) may be converted to a compound of Formula (340) by allowing a suspension of a compound of Formula (350) and anhydrous potassium carbonate (1.5 equivalents) in a dry acetone (0.1 M in compound) to react with benzylbromide (1.1 equivalents) at ambient temperatures for between 18 and 24 hours.

In an alternative embodiment, the compounds according to Formula (300) may be synthesized by providing a compound according to Formula (350) and converting the compound of Formula (350) to the compound according to Formula (300), as follows:

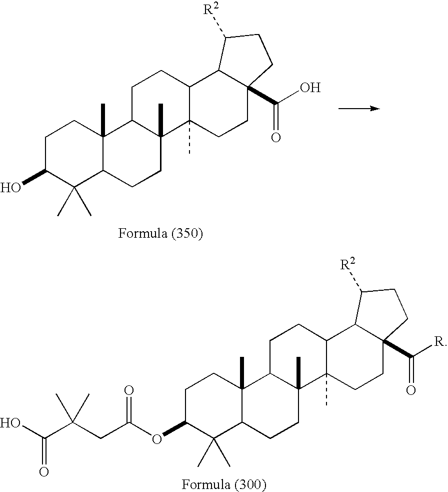

This process may be carried out by allowing a solution of a compound of Formula (350) in dichloromethane (0.02 M in compound) to react with an ester, such as benzyl or methyl, of 3-chlorocarbonyl-2,2-dimethyl-propionic acid (5 equivalents) in the presence of an organic base such as triethylamine or diisopropylamine (2 equivalents) for between 3 and 4 hours at 39° C.

Finally, compounds of Formula (300) can be prepared by preparing separately an appropriate amine compound according to the three processes described below, and then forming an amide bond with that amine followed by treatment with hydroxide ion as described above.

In a first process, compounds of Formula (300) where x is 1, y is 0, and one of R³² and R³³ is H and the other of R³² and R³³ is methyl, may be prepared by allowing a solution of heteroaryl methyl ketone in ethanol and water (between 2.0 and 2.5 M in compound) to react with hydroxylamine (1.5 equivalents) between 80° C. and 90° C. for between 5 and 15 minutes. The derived oxime is allowed to react with zinc metal (5 mass equivalents) at ambient temperatures for between 20 and 24 hours. The derived heteroaryl ethyl amine can be made optically pure by crystallization from ethanol and water of the derived D- or L-tartaric acid salts.

In a second process, compounds of Formula (300) where x is 1, y is 0, and R³² and R³³ are taken together with the carbon they are attached to form a cyclopropyl, may be prepared by allowing a solution of lithium hexamethyldisilazide (1 N solution in tetrahydrofuran, between 3.8 and 4 equivalents) under inert atmosphere, to react with a heteroaryl acetic ester compound for 10 minutes at ambient temperatures. After this time, tert butyl alcohol (3 equivalents) is added followed after 10 minutes by 1,2-dibromoethane (3 equivalents) and heating at 60° C. for between 16 and 18 hours. A solution of the concentrate in methanol (0.3 M in compound) is allowed to react with aqueous sodium hydroxide (6 equivalents) at ambient temperatures for between 4 and 6 hours and then acidified to pH<1 by addition of concentrated hydrochloric acid. A suspension of the derived acid product in toluene (0.3 M in compound) an organic base such as triethylamine or diisopropylamine (between 1 and 2 equivalents) and diphenylphosphorylazide (between 1 and 2 equivalents) is heated at 90° C. for between 18 and 24 hours and then concentrated to dryness.

A solution of the concentrate in methanol and aqueous sodium hydroxide (4 M hydroxide, 1:1 V/V, 0.3 M in compound) is heated at 70° C. for between 3 and 5 hours to provide the desired amine compound.

In a third process, compounds of Formula (300) where x is 1, y is 0, and R³² and R³³ are both methyl, may be prepared by allowing a solution of a hetero aryl nitrile in toluene (0.4 M in compound) to react with methyl magnesium bromide (2.5 equivalents) at 0° C. (ice bath) and then at 100° C. for between 18 and 24 hours.

A pharmaceutically acceptable salt of the compound of the present invention is exemplified by a salt with an inorganic acid such as hydrochloric acid, hydrobromic acid, sulfuric acid, phosphoric acid, nitric acid and the like, and a salt with an organic acid such as acetic acid, propionic acid, succinic acid, maleic acid, fumaric acid, benzoic acid, citric acid, malic acid, methanesulfonic acid, benzenesulfonic acid and the like. Their hydrates (1 hydrate, 2 hydrate, 3 hydrate, 1/2 hydrate, 3/2 hydrate, 1/4 hydrate, 4/5 hydrate, 1/5 hydrate, 3/4 hydrate, 1/3 hydrate, 5/3 hydrate, 5/4 hydrate etc.), solvates and the like are also encompassed in the compound of the present invention. In addition, N-oxide compounds are also encompassed in the compound of the present invention.

In addition, pharmaceutically acceptable salts include acid salt of inorganic bases, such as salts containing alkaline cations (e.g., Li+, Na+ or K+), alkaline earth cations (e.g., Mg++, Ca++ or Ba++), the ammonium cation, as well as acid salts of organic bases, including aliphatic and aromatic substituted ammonium, and quaternary ammonium cations, such as those arising from protonation of peralkylation of triethylamine, N,N-diethylamine, N,N-dicyclohexylamine, pyridine, N,N-dimethylaminopyridine (DMAP), 1,4-diazabiclo[2.2.2]octane (DABCO), 1,5-diazavicyclo[4.3.0]non-5-ene (DBN) and 1,8-diazabicyclo[5.4.0]undec-7-ene (DBU).

Additionally, the compounds of the present invention can contain asymmetric carbon atoms and can therefore exist in racemic and optically active forms. Thus, optical isomers or enantiomers, racemates, and diastereomers are also encompassed in the compounds of the present invention. The methods of present invention include the use of all such isomers and mixtures thereof. Methods of separation of enantiomeric and diastereomeric mixtures are well known to one skilled in the art. The present invention encompasses any isolated racemic or optically active form of compounds described in the present invention, or any mixture thereof, which possesses anti-viral activity.

In one embodiment of the invention, the stereochemistry of the compounds of the present invention is equivalent to that of the natural product from which the compound was derived (e.g., betulinic acid).

Unless specifically stated otherwise or indicated by a bond symbol (dash or double dash), the connecting point to a recited group will be on the right-most stated group. Thus, for example, a hydroxyalkyl group is connected to the main structure through the alkyl and the hydroxyl is a substituent on the alkyl.

As used herein, the term “alkyl” refers to a saturated aliphatic hydrocarbon including straight chain and branched chain groups. Preferably, the alkyl group has 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc. up to and including 20 carbon atoms). More preferably, it is a medium size alkyl having 1 to 10 carbon atoms. Even more preferably, it is a lower alkyl having 1 to 6 carbon atoms, and even more preferably 1 to 4 carbon atoms. The alkyl group may be substituted or unsubstituted. When substituted, the substituent group(s) is preferably one or more individually selected from cycloalkyl, aryl, heteroaryl, heteroalicyclic, hydroxy, alkoxy, aryloxy, mercapto, alkylthio, arylthio, cyano, halo, carbonyl, thiocarbonyl, O-carbamyl, N-carbamyl, O-thiocarbamyl, N-thiocarbamyl, C-amido, N-amido, C-carboxy, O-carboxy, cyanato, isocyanato, thiocyanato, isothiocyanato, nitro, silyl, and amino.

The term “alkenyl” as employed herein by itself or as part of another group means a straight or branched chain radical of 2-10 carbon atoms, unless the chain length is limited thereto, including at least one double bond between two of the carbon atoms in the chain. Typical alkenyl groups include ethenyl, 1-propenyl, 2-propenyl, 2-methyl-1-propenyl, 1-butenyl and 2-butenyl.

The term “alkynyl” is used herein to mean a straight or branched chain radical of 2-10 carbon atoms, unless the chain length is limited thereto, wherein there is at least one triple bond between two of the carbon atoms in the chain. Typical alkynyl groups include ethynyl, 1-propynyl, 1-methyl-2-propynyl, 2-propynyl, 1-butynyl and 2-butynyl.

The term “carbocycle” as employed herein refers to an all-carbon monocyclic or fused ring (i.e., rings which share an adjacent pair of carbon atoms) group including cycloalkyl and partially saturated carbocyclic groups. In partially saturated carbocyclic groups, one or more of the rings has an unsaturated bond between two carbon atoms, but does not have a completely conjugated pi-electron system.

Useful cycloalkyl groups are C₃₋₈ cycloalkyl. Typical cycloalkyl groups include cyclopropyl, cyclobutyl, cyclopentyl, cyclohexyl and cycloheptyl.

Useful partially saturated carbocyclic groups are cycloalkenyl groups, such as cyclopentenyl, cycloheptenyl and cyclooctenyl.

The term heterocycle is used herein to mean a saturated or partially saturated 3-7 membered monocyclic, or 7-10 membered bicyclic ring system, which consists of carbon atoms and from one to four heteroatoms independently selected from the group consisting of O, N, and S, wherein the nitrogen and sulfur heteroatoms can be optionally oxidized, the nitrogen can be optionally quaternized, and including any bicyclic group in which any of the above-defined heterocyclic rings is fused to a benzene ring, and wherein the heterocyclic ring can be substituted on carbon or on a nitrogen atom if the resulting compound is stable. One or more of the rings of a heterocycle does not have a completely conjugated pi-electron system.

Useful saturated or partially saturated heterocyclic groups include tetrahydrofuranyl, pyranyl, piperidinyl, piperazinyl, pyrrolidinyl, imidazolidinyl, imidazolinyl, indolinyl, isoindolinyl, quinuclidinyl, morpholinyl, isochromanyl, chromanyl, pyrazolidinyl, pyrazolinyl, tetronoyl and tetramoyl groups.

As used herein, “Aryl” refers to all-carbon monocyclic or fused-ring polycyclic (i.e., rings which share adjacent pairs of carbon atoms) groups having a completely conjugated pi-electron system. Examples, without limitation, of aryl groups are phenyl, naphthalenyl and anthracenyl.

The term “heteroaryl” as employed herein refers to groups having 5 to 14 ring atoms; 6, 10 or 14 π electrons shared in a cyclic array; and containing carbon atoms and 1, 2 or 3 oxygen, nitrogen or sulfur heteroactoms.

Useful heteroaryl groups include thienyl (thiophenyl), benzo[b]thienyl, naphtho[2,3-b]thienyl, thianthrenyl, furyl (furanyl), isobenzofuranyl, chromenyl, xanthenyl, phenoxanthiinyl, pyrrolyl, including without limitation 2H-pyrrolyl, imidazolyl, pyrazolyl, pyridyl (pyridinyl), including without limitation 2-pyridyl, 3-pyridyl, and 4-pyridyl, pyrazinyl, pyrimidinyl, pyridazinyl, indolizinyl, isoindolyl, 3H-indolyl, indolyl, indazolyl, purinyl, 4H-quinolizinyl, isoquinolyl, quinolyl, phthalzinyl, naphthyridinyl, quinozalinyl, cinnolinyl, pteridinyl, carbazolyl, β-carbolinyl, phenanthridinyl, acrindinyl, perimidinyl, phenanthrolinyl, phenazinyl, isothiazolyl, phenothiazinyl, isoxazolyl, furazanyl, phenoxazinyl, 1,4-dihydroquinoxaline-2,3-dione, 7-aminoisocoumarin, pyrido[1,2-a]pyrimidin-4-one, pyrazolo[1,5-a]pyrimidinyl, including without limitation pyrazolo[1,5-a]pyrimidin-3-yl, 1,2-benzoisoxazol-3-yl, benzimidazolyl, 2-oxindolyl and 2-oxobenzimidazolyl. Where the heteroaryl group contains a nitrogen atom in a ring, such nitrogen atom may be in the form of an N-oxide, e.g., a pyridyl N-oxide, pyrazinyl N-oxide and pyrimidinyl N-oxide.

As used herein, the term “halo” refers to chloro, fluoro, bromo, and iodo.

As used herein, the term “hydro” refers to a hydrogen atom (—H group).

As used herein, the term “hydroxy” refers to an —OH group.

As used herein, the term “alkoxy” refers to an —O—C₁₋₁₂ alkyl. Lower alkoxy refers to —O-lower alkyl groups.

As used herein, the term “cycloalkyloxy” refers to an —O-cycloalkyl group.

As used herein, the term “aryloxy” refers to both an —O-aryl group, as defined herein.

As used herein, the term “heteroaryloxy” refers to both an —O-heteroaryl group, as defined herein.

Useful acyloxy groups are any C₁₋₆ acyl (alkanoyl) attached to an oxy (—O—) group, e.g., formyloxy, acetoxy, propionoyloxy, butanoyloxy, pentanoyloxy and hexanoyloxy.

As used herein, the term “mercapto” group refers to an —SH group.

As used herein, the term “alkylthio” group refers to an —S-alkyl group, as defined herein.

As used herein, the term “arylthio” group refers to both an —S-aryl group, as defined herein.

The term “arylalkyl” is used herein to mean any of the above-mentioned C₁₋₁₀ alkyl groups substituted by any of the above-mentioned C₆₋₁₄ aryl groups. Preferably the arylalkyl group is benzyl, phenethyl or naphthylmethyl.

The term “heteroarylalkyl” is used herein to mean any of the above-mentioned C₁₋₁₀ alkyl groups substituted by any of the above-mentioned heteroaryl groups.

The term “arylalkenyl” is used herein to mean any of the above-mentioned C₂₋₁₀ alkenyl groups substituted by any of the above-mentioned C₆₋₁₄ aryl groups.

The term “heteroarylalkenyl” is used herein to mean any of the above-mentioned C₂₋₁₀ alkenyl groups substituted by any of the above-mentioned heteroaryl groups.

The term “arylalkynyl” is used herein to mean any of the above-mentioned C₂₋₁₀ alkynyl groups substituted by any of the above-mentioned C₆₋₁₄ aryl groups.

The term “heteroarylalkynyl” is used herein to mean any of the above-mentioned C₂₋₁₀ alkynyl groups substituted by any of the above-mentioned heteroaryl groups.

The term “aryloxy” is used herein to mean oxygen substituted by one of the above-mentioned C₆₋₁₄ aryl groups, which may be optionally substituted. Useful aryloxy groups include phenoxy and 4-methylphenoxy.

The term “heteroaryloxy” is used herein to mean oxygen substituted by one of the above-mentioned heteroaryl groups.

The term “arylalkoxy” is used herein to mean any of the above mentioned C₁₋₁₀ alkoxy groups substituted by any of the above-mentioned aryl groups, which may be optionally substituted. Useful arylalkoxy groups include benzyloxy and phenethyloxy.

“Heteroarylalkoxy” is used herein to mean any of the above mentioned C₁₋₁₀ alkoxy groups substituted by any of the above-mentioned heteroaryl groups, which may be optionally substituted.

Useful haloalkyl groups include C₁₋₁₀ alkyl groups substituted by one or more fluorine, chlorine, bromine or iodine atoms, e.g., fluoromethyl, difluoromethyl, trifluoromethyl, pentafluoroethyl, 1,1-difluoroethyl, chloromethyl, chlorofluoromethyl and trichloromethyl groups.

Useful acylamino (acylamido) groups are any C₁₋₆ acyl (alkanoyl) attached to an amino nitrogen, e.g., acetamido, chloroacetamido, propionamido, butanoylamido, pentanoylamido and hexanoylamido, as well as aryl-substituted C₁₋₆ acylamino groups, e.g., benzoylamido, and pentafluorobenzoylamido.

As used herein, the term “carbonyl” group refers to a —C(═O)R″ group, where R″ is selected from the group consisting of hydro, alkyl, cycloalkyl, aryl, heteroaryl (bonded through a ring carbon) and heterocyclic (bonded through a ring carbon), as defined herein.

As used herein, the term “aldehyde” group refers to a carbonyl group where R″ is hydro.

As used herein, the term “cycloketone” refer to a cycloalkyl group in which one of the carbon atoms which form the ring has a “═O” bonded to it; i.e. one of the ring carbon atoms is a —C(═O)-group.

As used herein, the term “thiocarbonyl” group refers to a —C(═S)R″ group, with R″ as defined herein.

As used herein, the term “O-carboxy” group refers to a R″C(═O)O-group, with R″ as defined herein.

As used herein, the term “C-carboxy” group refers to a —C(═O)OR″ groups with R″ as defined herein.

As used herein, the term “ester” is a C-carboxy group, as defined herein, wherein R″ is any of the listed groups other than hydro (e.g., methyl, ethyl, lower alkyl).

As used herein, the term “C-carboxy salt” refers to a —C(═O)O— M⁺ group wherein M⁺ is selected from the group consisting of lithium, sodium, magnesium, calcium, potassium, barium, iron, zinc and quaternary ammonium.

As used herein, the term “acetyl” group refers to a —C(═O)CH₃ group.

As used herein, the term “carboxyalkyl” refers to —(CH₂)ᵣC(═O)OR″ wherein r is 1-6 and R″ is as defined above.

As used herein, the term “carboxyalkyl salt” refers to a —(CH₂)ᵣC(═O)O⁻M⁺ wherein M⁺ is selected from the group consisting of lithium, sodium, potassium, calcium, magnesium, barium, iron, zinc and quaternary ammonium.

As used herein, the term “carboxylic acid” refers to a C-carboxy group in which R″ is hydro.

As used herein, the term “haloalkyl” refers to an alkyl group substituted with 1 to 6 halo groups, preferably haloalkyl is a —CX₃ group wherein X is a halo group. The halo groups can be independently selected.

As used herein, the term “trihalomethanesulfonyl” refers to a X₃ CS(═O)₂— group with X as defined above.

As used herein, the term “cyano” refers to a —C≡N group.

As used herein, the term “cyanato” refers to a —CNO group.

As used herein, the term “isocyanato” refers to a —NCO group.

As used herein, the term “thiocyanato” refers to a —CNS group.

As used herein, the term “isothiocyanato” refers to a —NCS group.

As used herein, the term “sulfinyl” refers to a —S(═O)R″ group, with R″ as defined herein.

As used herein, the term “sulfonyl” refers to a —S(═O)₂R″ group, with R″ as defined herein.

As used herein, the term “sulfonamido” refers to a —S(═O)₂NR¹⁷R¹⁸, with R¹⁷ and R¹⁸ as defined herein.

As used herein, the term “trihalomethanesulfonamido” refers to a X₃CS(═O)₂ NR¹⁷-group with X and R¹⁷ as defined herein.

As used herein, the term “O-carbamyl” refers to a —OC(═O)NR¹⁷R¹⁸ group with R¹⁷ and R¹⁸ as defined herein.

As used herein, the term “N-carbamyl” refers to a R¹⁸C(═O)NR¹⁷— group, with R¹⁷ and R¹⁸ as defined herein.

As used herein, the term “O-thiocarbamyl” refers to a —OC(═S)NR¹⁷R¹⁸ group with R¹⁷ and R¹⁸ as defined herein.

As used herein, the term “N-thiocarbamyl” refers to a R¹⁷OC(═S)NR¹⁸— group, with R¹⁷ and R¹⁸ as defined herein.

As used herein, the term “amino” refers to an —NR¹⁷R¹⁸ group, with R¹⁷ and R¹⁸ as defined herein.

As used herein, the term “C-amido” refers to a —C(═O)NR¹⁷R¹⁸ group with R¹⁷ and R¹⁸ as defined herein. An “N-amido” refers to a R¹⁷C(═O)NR¹⁸— group with R¹⁷ and R¹⁸ as defined herein.

As used herein, the term “nitro” refers to a —NO₂ group.

As used herein, the term “quaternary ammonium” refers to a —⁺NR¹⁷R¹⁸R¹⁹ group wherein R¹⁷, R¹⁸, and R¹⁹ are as defined herein.

R¹⁷, R¹⁸, and R¹⁹ are independently selected from the group consisting of hydro and unsubstituted lower alkyl.

As used herein, the term “methylenedioxy” refers to a —OCH₂O— group wherein the oxygen atoms are bonded to adjacent ring carbon atoms.

As used herein, the term “ethylenedioxy” refers to a —OCH₂CH₂O— group wherein the oxygen atoms are bonded to adjacent ring carbon atoms.

As used herein, the phrase “treating . . . with . . . a compound” means either administering the compound to cells or an animal, or administering to cells or an animal the compound or another agent to cause the presence or formation of the compound inside the cells or the animal. Preferably, the methods of the present invention comprise administering to cells in vitro or to a warm-blood animal, particularly mammal, more particularly a human a pharmaceutical composition comprising an effective amount of a compound according to the present invention.

The present invention provides methods for treating viral infection by administering to a patient (either a human or other animal) that is a carrier of a virus a pharmaceutical composition or medicament having a therapeutically effective amount of a compound of the present invention. For example, a carrier of a virus can be identified by conventional diagnostic techniques known in the art, as described above. The identified carrier can be administered with a compound of the present invention, preferably in a pharmaceutical composition having a pharmaceutically acceptable carrier.

In another aspect, the present invention provides methods for treating an active viral infection by administering to a patient (either a human or other animal) that exhibits characteristic symptoms of a viral infection a pharmaceutical composition or medicament having a therapeutically effective amount of a compound of the present invention. Alternatively, the presence of viral infection may be detected or determined directly by any appropriate method in the art. The infected individual so identified can be administered with a compound of the present invention, preferably in a pharmaceutical composition having a pharmaceutically acceptable carrier.

Consequently, the methods of the present invention may be generally useful in treating or preventing diseases or disorders associated with viral infection in animals, particularly humans. Such viral infection can be caused by viruses including, but not limited to, lentiviruses such as human immunodeficiency virus types 1 and 2 (HIV), human T-cell lymphotropic virus type 1 and 2 (HTLV-I and HTLV-II), SIV, EIAV (equine infectious anemia virus), BIV, FIV, CAEV, VMV, and MMLV (Moloney murine leukemia virus). Such viral infections can also be caused by hepatitis A virus, hepatitis B virus, hepatitis C virus, hepatitis D virus, hepatitis E virus, hepatitis G virus, human foamy virus, or by human herpes viruses (e.g., herpes simplex virus type-1, herpes simplex virus type-2, herpes simplex virus type-3 (also known as Varicella-zoster virus), herpes simplex virus type-4 (also known as Epstein Barr virus or EBV), herpes simplex virus type-5, herpes simplex virus type-7). Such viral infections can also be caused by influenza viruses (types A, B or C), human parainfluenza viruses, respiratory syncytial virus, smallpox virus (variola virus), monkeypox virus, vaccinia virus, human papilloma virus, human parechovirus 2, mumps virus, Measles virus, Rubella virus, Semliki Forest virus, West Nile virus, Colorado tick fever virus, foot-and-mouth disease virus, Ebola virus, Marburg virus, polyomavirus, TT virus, Lassa virus, lymphocytic choriomeningitis virus, vesicular stomatitis virus, rotavirus, varicella virus, parvovirus, cytomegalovirus, encephalitis viruses, adenovirus, echovirus, rhinoviruses, filoviruses, coxachievirus, coronavirus (such as SARS-associated coronavirus), Dengue viruses, yellow fever virus, hantaviruses, regional hemorrhagic fever viruses, molluscum virus, poliovirus, rabiesvirus, etc. In some embodiments, the methods are used in treating or preventing infections by enveloped viruses. In specific embodiments, as described below, particular viruses known to infect humans and cause disease are treated by the methods of the present invention.

The present invention provides methods for treating viral infection, particularly HIV infection, delaying the onset of HIV infection, treating AIDS, delay the onset of AIDS, by treating a patient (either a human or another animal) in need of the treatment, with a compound of the present invention.

As used herein, the term “HIV infection” generally encompasses infection of a host animal, particularly a human host, by the human immunodeficiency virus (HIV) family of retroviruses including, but not limited to, HIV-1, HIV-2, HIV I (also known as HTLV-III), HIV II (also known as LAV-1), HIV III (also known as LAV-2), and the like. “HIV” can be used herein to refer to any strains, forms, subtypes, clades and variations in the HIV family. Thus, treating HIV infection will encompass the treatment of a person who is a carrier of any of the HIV family of retroviruses or a person who is diagnosed of active AIDS, as well as the treatment or delay the onset of AIDS or AIDS-related conditions in such persons. A carrier of HIV may be identified by any methods known in the art. For example, a person can be identified as HIV carrier on the basis that the person is anti-HIV antibody positive, or is HIV-positive, or has symptoms of AIDS. That is, “treating HIV infection” should be understood as treating a patient who is at any one of the several stages of HIV infection progression, which, for example, include acute primary infection syndrome (which can be asymptomatic or associated with an influenza-like illness with fevers, malaise, diarrhea and neurologic symptoms such as headache), asymptomatic infection (which is the long latent period with a gradual decline in the number of circulating CD4 T-cells), and AIDS (which is defined by more serious AIDS-defining illnesses and/or a decline in the circulating CD4 T-cell count to below a level that is compatible with effective immune function).

As used herein, the term “delaying the onset of HIV infection” means treating an individual who (1) is at risk of infection by HIV, or (2) is suspected of infection by HIV or of exposure to HIV, or (3) has suspected past exposure to HIV, to delay the onset of acute primary infection syndrome by at least three months. As is known in the art, clinical findings typically associated with acute primary infection syndrome may include an influenza-like illness with fevers, malaise, nausea/vomiting/diarrhea, pharyngitis, lymphadenopathy, myalgias, and neurologic symptoms such as headache, encephalitis, etc. The individuals at risk may be people who perform any of following acts: contact with HIV-contaminated blood, blood transfusion, exchange of body fluids, “unsafe” sex with an infected person, accidental needle stick, injection of drug with contaminated needles or syringes, receiving a tattoo or acupuncture with contaminated instruments, or transmission of the virus from a mother to a baby during pregnancy, delivery or shortly thereafter. The term “delaying the onset of HIV infection” also encompasses treating a person who has not been diagnosed as having HIV infection but is believed to be at risk of infection by HIV, or has been exposed to HIV through contaminated blood, etc.

In addition, the term “delay the onset of AIDS” means delaying the onset of AIDS (which is characterized by more serious AIDS-defining illnesses and/or a decline in the circulating CD4 cell count to below a level that is compatible with effective immune function, i.e. below about 200/μl) and/or AIDS-related conditions, by treating an individual (1) at risk of infection by HIV, or suspected of being infected with HIV, or (2) having HIV infection but not AIDS, to delay the onset of AIDS by at least six months. Individuals at risk of HIV infection may be those who are suspected of past exposure, or considered to be at risk of present or future exposure, to HIV by, e.g., contact with HIV-contaminated blood, blood transfusion, transplantation, exchange of body fluids, “unsafe” sex with an infected person, accidental needle stick, receiving a tattoo or acupuncture with contaminated instruments, or transmission of the virus from a mother to a baby during pregnancy, delivery or shortly thereafter.

The term “treating AIDS” means treating a patient who exhibits more serious AIDS-defining illnesses and/or a decline in the circulating CD4 cell count to below a level that is compatible with effective immune function (typically below about 200 μl). The term “treating AIDS” also encompasses treating AIDS-related conditions, which means disorders and diseases incidental to or associated with AIDS or HIV infection such as AIDS-related complex (ARC), progressive generalized lymphadenopathy (PGL), anti-HIV antibody positive conditions, and HIV-positive conditions, AIDS-related neurological conditions (such as dementia or tropical paraparesis), Kaposi's sarcoma, thrombocytopenia purpurea and associated opportunistic infections such as Pneumocystis carinii pneumonia, Mycobacterial tuberculosis, esophageal candidiasis, toxoplasmosis of the brain, CMV retinitis, HIV-related encephalopathy, HIV-related wasting syndrome, etc.

For example, a carrier of HIV can be identified by conventional diagnostic techniques known in the art, and the identified carrier can be treated with a compound of the present invention, preferably in a pharmaceutical composition having a pharmaceutically acceptable carrier.

In one aspect, the present invention provides methods for combination therapy for treating viral infection, particularly HIV infection, delaying the onset of HIV infection, treating AIDS, delay the onset of AIDS, by treating a patient (either a human or another animal) in need of the treatment, with a compound of the present invention together with one or more other anti-HIV agents. Such other anti-HIV agents include those agents targeting a viral protein such as viral protease, reverse transcriptase, integrase, envelope protein (e.g., gp120 and gp41 for anti-fusion or homolog thereof). Thus, examples of such other antiviral compounds include, but are not limited to, protease inhibitors, nucleoside reverse transcriptase inhibitors, non-nucleoside reverse transcriptase inhibitors, integrase inhibitors, fusion inhibitors, and a combination thereof. In the combination therapy, the compound of the present invention can be administered separately from, or together with the one or more other anti-HIV agents.

HBV

As used herein, the term “HBV infection” generally encompasses infection of a human by any strain or serotype of hepatitis B virus, including acute hepatitis B infection and chronic hepatitis B infection. Thus, treating HBV infection means the treatment of a person who is a carrier of any strain or serotype of hepatitis B virus, or a person who is diagnosed with active hepatitis B, to reduce the HBV viral load in that person or to alleviate one or more symptoms associated with HBV infection and/or hepatitis B, including, e.g., nausea and vomiting, loss of appetite, fatigue, muscle and joint aches, elevated transaminase blood levels, increased prothrombin time, jaundice (yellow discoloration of the eyes and body) and dark urine. A carrier of HBV may be identified by any method known in the art. For example, a person can be identified as HBV carrier on the basis that the person is anti-HBV antibody positive (e.g., based on hepatitis B core antibody or hepatitis B surface antibody), or is HBV-positive (e.g., based on hepatitis B surface antigens (HBeAg or HbsAg) or HBV RNA or DNA) or has symptoms of hepatitis B infection or hepatitis B. Hence, “treating HBV infection” should be understood as treating a patient who is at any one of the several stages of HBV infection progression. In addition, the term “treating HBV infection” will also encompass treating individuals with a suspected HBV infection after suspected exposure to HBV by, e.g., contact with HBV-contaminated blood, blood transfusion, exchange of body fluids, “unsafe” sex with an infected person, accidental needle stick, receiving a tattoo or acupuncture with contaminated instruments, or transmission of the virus from a mother to a baby during pregnancy, delivery or shortly thereafter. The term “treating HBV infection” will also encompass treating a person who is free of HBV infection but is believed to be at risk of infection by HBV.

In yet another aspect, a method of treating HBV infection in a patient co-infected with HBV and HIV is provided by administering a therapeutically effective amount of a compound according to the present invention to such a patient. Particularly, HIV infection is associated with an approximate threefold increase in the development of persistent hepatitis B. The compounds according to the present invention are particularly suitable for patients co-infected with HIV and HBV. The presently marketed drug interferon alpha is not effective in treating HBV and HIV co-infection. Lamivudine and some other reverse transcriptase inhibitors are useful in treating such co-infections, but Lamivudine is particularly toxic and can cause hepatic injury which worsens hepatitis B. In addition, such reverse transcriptase inhibitors often must be used in cocktails. In contrast, the compounds according to the present invention are significantly less toxic, and are less likely to result in evolved viral resistance. Thus, in accordance with the present invention, a compound according to the present invention is administered alone, or in combination with another anti-HIV or anti-HBV drug, in a therapeutically effective amount to a mammal, particularly a human co-infected with both HBV and HIV. The method may include a step of identifying a patient co-infected with HBV and HIV by techniques commonly known in the art. For example, PCR tests can be used to detect HBV DNA or RNA and HIV RNA in blood samples obtained from a test subject. Alternatively, virus-specific antibodies or antigens may be also employed for the detection of HBV and HIV infection.

The term “preventing hepatitis B” as used herein means preventing in a patient who has an HBV infection, is suspected to have an HBV infection, or is at risk of contracting an HBV infection, from developing hepatitis B (which are characterized by more serious hepatitis-defining symptoms), cirrhosis, or hepatocellular carcinoma.

HCV

As used herein, the term “HCV infection” generally encompasses infection of a human by any types or subtypes of hepatitis C virus, including acute hepatitis C infection and chronic hepatitis C infection. Thus, treating HCV infection means the treatment of a person who is a carrier of any types or subtypes of hepatitis C virus, or a person who is diagnosed with active hepatitis C, to reduce the HCV viral load in that person or to alleviate one or more symptoms associated with HCV infection and/or hepatitis C. A carrier of HCV may be identified by any methods known in the art. For example, a person can be identified as HCV carrier on the basis that the person is anti-HCV antibody positive, or is HCV-positive (e.g., based on HCV RNA or DNA) or has symptoms of hepatitis C infection or hepatitis C (e.g., elevated serum transaminases). Hence, “treating HCV infection” should be understood as treating a patient who is at any one of the several stages of HCV infection progression. In addition, the term “treating HCV infection” will also encompass treating individuals with a suspected HCV infection after suspected past exposure to HCV by, e.g., contact with HCV-contaminated blood, blood transfusion, exchange of body fluids, “unsafe” sex with an infected person, accidental needle stick, receiving a tattoo or acupuncture with contaminated instruments, or transmission of the virus from a mother to a baby during pregnancy, delivery or shortly thereafter. The term “treating HCV infection” will also encompass treating a person who is free of HCV infection but is believed to be at risk of infection by HCV. The term of “preventing HCV” as used herein means preventing in a patient who has HCV infection or is suspected to have HCV infection or is at risk of HCV infection from developing hepatitis C (which is characterized by more serious hepatitis-defining symptoms), cirrhosis, or hepatocellular carcinoma.

Importantly, about one quarter of all HIV-infected persons in the United States, or an estimated 200,000 people, are infected with both HCV and HIV (See National Center for HIV, STD and TB Prevention report at http://www.cdc.gov/hiv/pubs/facts/HIV-HCV_Coinfection.htm and Thomas, D. L. Hepatology 36:S201-S209 (2002)). As the lives of HIV-infected persons have been prolonged by use of highly active antiretroviral therapy, liver disease has emerged as an important, and in some settings, the leading cause of morbidity and mortality. HIV infection appears to adversely affect all stages of HCV infection. Particularly, HIV infection is associated with a significant increase in the development of persistent hepatitis C, with higher titers of HCV, more rapid progression to HCV-related liver disease, and an increased risk for HCV-related cirrhosis (scarring) of the liver. In turn, HCV may affect the management of HIV infection, increasing the incidence of liver toxicity caused by antiretroviral medications (Thomas, D. L. Hepatology 36:S201-S209, (2002) and National Center for HIV, STD and TB Prevention report at http://www.cdc.gov/hiv/pubs/facts/HIV-HCV_Coinfection.htm).

In the United States, two different treatment regimens have been approved as therapy for chronic hepatitis C: monotherapy with alpha interferon and combination therapy with alpha interferon and ribavirin. Among HIV-negative persons with chronic hepatitis C, combination therapy consistently yields higher rates (30%-40%) of sustained response than monotherapy (10%-20%). Combination therapy is more effective against viral genotypes 2 and 3, and requires a shorter course of treatment; however, viral genotype 1 is the most common among U.S. patients. Combination therapy is associated with more side effects than monotherapy, but, in most situations, it is preferable. At present, interferon monotherapy is reserved for patients who have contraindications to the use of ribavirin. (See, http://www.cdc.gov/hiv/pubs/facts/HIV-HCV_Coinfection.htm)

Hence, in yet another aspect, a method of treating HCV infection in a patient co-infected with HCV and HIV is provided by administering a therapeutically effective amount of a compound according to the present invention to such a patient. The compounds according to the present invention are particularly suitable for patients co-infected with HIV and HCV. Particularly, the compounds are especially effective in inhibiting HCV infection and/or egress from host cells. Moreover, the compounds can also be effective in inhibiting HIV entry into and/or egress from host cells. In contrast to the combination therapy described above, the compounds according to the present invention can be significantly less toxic, and less likely to result in evolved viral resistance. Thus, in accordance with the present invention, a compound according to the present invention is administered alone, or in combination with another anti-HIV or anti-HCV drug, in a therapeutically effective amount to a mammal, particularly a human co-infected with both HCV and HIV. The method may include a step of identifying a patient co-infected with HCV and HIV by techniques commonly known in the art. For example, PCR tests can be used to detect HCV DNA or RNA and HIV RNA in blood samples obtained from a test subject. Alternatively, virus-specific antibodies or antigens may be also employed for the detection of HCV and HIV infection.

Herpesviruses

Herpesviruses are one of the most common human pathogens. Members of the herpesvirus family include herpes simplex virus type-1 (HSV-1), herpes simplex virus type-2 (HSV-2), Varicella-zoster virus (herpes simplex virus type-3 or HSV-3; also known as chicken pox), and Epstein-Barr virus (herpes simplex virus type-4 or HSV-4). HSV-1 commonly causes herpes labialis (also called oral herpes, cold sores, fever blisters), which are highly infectious open sores that crust over before healing. HSV-1 can also cause eye and brain infection. HSV-2 commonly causes genital herpes. HSV-1 can also cause genital herpes, though far less frequently than HSV-2. After an initial infectious cycle, HSV-1 and HSV-2 generally establish life-long latent infections in sensory neurons near the site of infection. These latent infections exist without showing any signs or symptoms of infection or disease, until some event reactivates the virus. Reactivation generally causes recurrent lesions close to, or in the same location as, the site of initial infection. Reactivation seems to occur during periods of emotional stress, or periods of reduced immune system function.

In addition to oral and genital herpes, HSV-1 and HSV-2 can cause other diseases. Examples of such diseases include herpes simplex encephalitis—a rare but potentially fatal herpetic infection of the brain; neonatal herpes, —a rare but potentially severe HSV infection in newborns (resulting from transmission of the virus from the mother to the baby during delivery); herpetic whitlow—an HSV infection of the finger (acquired either from transfer of the infection from another part of the body or from direct contact with another party having an HSV infection); and herpes keratitis—an HSV infection of the eye (one of the most common causes of blindness). Thus, herpes simplex virus infection of humans is a significant health problem.

Genital herpes is primarily treated with suppressive and episodic therapies. Suppressive therapy is used to treat outbreaks before they occur, while episodic therapy treats outbreaks when they occur. Treatment with valacyclovir HCl, acyclovir, and famciclovir, can be used in both suppressive and episodic therapies.

Currently there is no known cure for HSV-1 infection. The available antiviral therapies are not completely effective and there is a chance that the virus will become resistant to the treatment. Thus, there is a clear need for improved methods and compositions for treating HSV-1.

Epstein-Barr virus (herpes simplex virus-4), hereafter referred to as “EBV”, occurs worldwide. In fact, most people become infected with EBV during their lives. A large percentage of adults in the United States have been infected. Infants are susceptible to EBV as soon as maternal antibody protection present at birth disappears. Many children become infected with EBV, and these infections usually cause no symptoms. The symptoms of EBV infection in children can be indistinguishable from the symptoms of other typical childhood illnesses. Individuals not infected as a child have a risk of being infected during adolescence or young adulthood, which often causes infectious mononucleosis (mono). Symptoms of infectious mononucleosis include fever, sore throat, and swollen lymph glands, less often a swollen spleen or liver involvement may develop. Rarely, heart problems or involvement of the central nervous system occur. Infectious mononucleosis is almost never fatal. The symptoms of infectious mononucleosis usually resolve in 1 or 2 months, but EBV remains dormant or latent in a few cells in the throat and blood for the rest of the infected person's life. Periodically, the virus can reactivate and is commonly found in the saliva of infected persons. Reactivation usually occurs without symptoms of illness.

EBV is thought to be associated with a number of other diseases including Burkitt's lymphoma, nasopharyngeal carcinoma, and Hodgkin's disease. Diseases caused by EBV are particularly common among people with reduced immunity. EBV is associated with a tumor often found in organ transplant patients that is referred to as post-transplant lymphoproliferative disease. The immune systems of such patients are usually artificially suppressed by drug therapy to help prevent the body from rejecting the new organ. Individuals infected with HIV, and have AIDS, also have reduced immunity and commonly suffer from oral hairy leukoplakia, a condition involving considerable replication of EBV in cells along the edge of the tongue. It has also been suggested that the high incidence of malaria in countries where Burkitt's lymphoma is prevalent may also play a role in the disease by suppressing the body's immune system.

Scientists are finding it difficult to explain why the virus causes a relatively mild disease like glandular fever in some people and malignant tumors in others. Genetic factors may play a role. Regardless, treatments are needed to combat EBV.

As used herein, the terms “herpes simplex virus” or HSV refers to any strain of herpes simplex virus, including, but not limited to HSV-1, HSV-2, HSV-3 (Varcella-zoster virus or chicken pox), and HSV-4 (or EBV). Thus, “treating HSV infections” will encompass the treatment of a person who is actively infected with, or carrier of a latent infection of, any of the HSV family of herpes viruses.

As used herein, the term “HSV infection” generally encompasses infection of a human by any strain of herpes simplex virus, and includes both active and latent infections. Thus, “treating HSV infection” means the treatment of a person who is a carrier of any strain of HSV. For example, a person can be identified as an HSV carrier on the basis that the person is anti-HSV antibody positive or has symptoms of an HSV infection. Hence, “treating HSV infection” should be understood as treating a patient who is at any one of the several stages of HSV infection progression. In addition, the term “treating HSV infection” will also encompass treating individuals with a suspected HSV infection after suspected exposure to HSV by, e.g., contact with HSV-contaminated blood, blood transfusion, exchange of body fluids, “unsafe” sex with an infected person, accidental needle stick, receiving a tattoo or acupuncture with contaminated instruments, or transmission of the virus from a mother to a baby during pregnancy, delivery or shortly thereafter. The term “treating HSV infection” will also encompass treating a person who is free of HSV infection but is believed to be at risk of infection by HSV.

In yet another aspect, a method of treating HSV infection in a patient co-infected with HSV and HIV is provided by administering a therapeutically effective amount of a compound according to the present invention to such a patient. Particularly, HIV infection is associated with an increase in active HSV infections, presumably due to the immunocompromised state created by the HIV infection. The compounds according to the present invention are particularly suitable for patients co-infected with HIV and HSV. The presently marketed drug interferon alpha is not effective in treating HBV and HIV co-infection. Lamivudine and some other reverse transcriptase inhibitors are useful in treating such co-infections, but Lamivudine is particularly toxic and can cause hepatic injury which worsens hepatitis B. In addition, such reverse transcriptase inhibitors often must be used in cocktails. In contrast, the compounds according to the present invention are significantly less toxic, and are less likely to result in evolved viral resistance. Thus, in accordance with the present invention, a compound according to the present invention is administered alone, or in combination with another anti-HIV or anti-HSV drug, in a therapeutically effective amount to a mammal, particularly a human co-infected with both HSV and HIV. The method may include a step of identifying a patient co-infected with HSV and HIV by techniques commonly known in the art. For example, PCR tests can be used to detect HSV DNA or RNA and HIV RNA in blood samples obtained from a test subject. Alternatively, virus-specific antibodies or antigens may be also employed for the detection of HSV and HIV infection.

The term “delaying the onset of HSV-associated symptoms” as used herein means preventing in a patient who has an HSV infection, is suspected to have an HSV infection, or is at risk of contracting an HSV infection, from developing oral herpes, genital herpes, chickenpox or shingles, or a chronic EBV infection.

Influenza

Influenza infection is associated with an average of 36,000 deaths and 114,000 hospitalizations per year in the United States alone. Although there are three recognized types of influenza viruses, influenza A, B, and C, types A and B are responsible for annual winter flu epidemics. Influenza A infects many different animal species besides humans, including ducks, chickens, pigs, whales, horses, and seals. Influenza B viruses generally only infect humans.

All three types of influenza virus have genomes composed of eight different RNA helices, which encodes a single gene and are bound by a nucleoprotein that determines the viral type: A, B, or C. In effect, the influenza genome is made up of eight separate pieces of nucleic acid that can come together to form viruses with new combinations of viral genes when cells become co-infected by more than one viral type. Two of these RNA helices encode the important viral surface proteins hemagglutinin and neuramidase, which are embedded in the lipid bilayer of a mature virus particle.

Variations in the viral hemagglutinin and neuramidase determine the viral subtype. Hemagglutinin is responsible for entry of the virus into the host cell, while neuramidase is important in the release of newly formed viruses from the infected cells. Antibodies to hemagglutinin can neutralize the virus and are the major determinant for immunity. Antibodies to neuramidase do not neutralize the virus but may limit viral replication and the course of infection. Host antibodies to specific types of hemagglutinin and neuramidase prevent and generally ameliorate future infection by the same viral strain. However, since the genetic makeup of viral strains is dynamic and ever-changing, immunity gained through successful resistance to one strain gained during an infection one year may be useless in combating a new, recombined, variant strain the next year.

Epidemics of influenza are thought to result when viral strains change over time by the process of antigenic drift. Antigenic drift (caused by mutations in the principal viral antigen genes, especially in the hemagglutinin or neuramidase genes) results in small changes in surface antigens, and occurs essentially continuously over time. When these changes occur in the right places in the genes, they render the new antigens unrecognizable by the antibodies raised against other influenza virus strains during previous infections.

Influenza pandemics (or worldwide epidemics) occur as a result of “antigenic shift.” Antigenic shift is an abrupt, major change in an influenza A virus that results from a new hemagglutinin and/or new hemagglutinin and neuraminidase protein appearing in an influenza A strain. Such shifts are generally thought to occur when a new combination of viral genomic RNAs is created, possibly in a non-human species, and that new combination is passed to humans. When such an antigenic shift occurs, most humans have little or no protection against the virus, and an infection can prove lethal.

Influenza pandemics have resulted in massive loss of life during the history of man. The influenza pandemic of 1918-1919 resulted in the deaths of about 20-40 million people. In support of the antigenic shift hypothesis presented above, molecular analyses recently demonstrated that the influenza virus responsible for the 1918-19 pandemic is related to a swine influenza virus that belongs to the same family of influenza virus that still causes the flu in humans today.

Two categories of treatment/preventative strategies are available for influenza infection: vaccination with “the flu shot” and administration of antiviral drugs. The flu shot involves vaccination with killed or inactivated influenza viruses. The antiviral drugs available for treating influenza infection including amantadine, rimantadine, zanamivir, and osteltamivir. Amantadine and rimantadine are used for treating and preventing influenza A infection, zanamivir is used for treating influenza A and B infection, and osteltamivir is used for treating and preventing influenza A and B infection.

Despite the numerous drugs and vaccinations available, there is a need for improved methods and compositions for both treating and preventing influenza infection.

As used herein, the term “influenza” and “influenza virus” refer to any type or subtype of influenza, including types A, B and C, and all subtypes thereof. Consequently, the term “influenza infection” encompasses infection by any strain of influenza, and the term “treating influenza infection” is understood to mean the treatment of an animal, particularly a human, infected by any strain of influenza. In addition, the term “treating influenza infection” will also encompass treating individuals with a suspected influenza infection after suspected exposure to influenza. The term “treating influenza infection” will also encompass treating a person who is apparently free of an influenza infection but is believed to be at risk of infection by influenza.

Poxviruses

As used herein, the terms “smallpox virus” or “variola virus” refers to any strain of smallpox virus including variola major and variola minor (also referred to as alastrim). Examples of such human variola virus isolates are well known and the complete genomic nucleotide sequence one strain has been determined (See, e.g., Harrison's 15th Edition Principles of Internal Medicine, Braunwald et al. EDS. McGraw-Hill, United States, and Genbank accession no. NC_{—}001611). Skilled artisans are capable of diagnosing individuals infected or suspected of being infected with smallpox. The term “treating smallpox” or “treating variola virus” refers to both treating the symptoms of the disease as well as reducing the viral load, infectivity and/or replication of the virus. The term of “delaying the onset of symptoms associated with smallpox infection” as used herein means treating a patient who is free of smallpox infection, or is believed to be at risk of infection by smallpox, or is infected with smallpox to delay the onset of one or more symptoms associated with smallpox infection by at least 3 months. The term “treating smallpox” also encompasses treating a person who either has smallpox infection, is suspected to have smallpox infection, or is at risk of developing smallpox from a smallpox virus infection (which is characterized by more serious smallpox-defining symptoms like macular rash, fever, vesicular lesions and pustular lesions).

An outbreak of monkeypox occurred for the first time in the United States in June of 2003. The causative agent is the monkeypox virus, which belongs to the group of viruses that includes the smallpox virus (variola), the virus used in the smallpox vaccine (vaccinia), and the cowpox virus. In humans, the signs and symptoms of monkeypox are like those of smallpox, but usually much milder, although monkeypox, unlike smallpox causes the lymph nodes to swell. In Africa, where most cases of monkeypox are known to occur, infections result in deaths of between 1% and 10% of infected individuals. As used herein, the term “treating monkeypox” or “treating monkeypox virus” refers to both treating the symptoms of the disease as well as reducing the viral load, infectivity and/or replication of the virus. The term of “preventing monkeypox infection” as used herein means preventing infection in a patient who is free of monkeypox infection but is believed to be at risk of infection by monkeypox. The term of “delaying the onset of symptoms associated with monkeypox infection” as used herein means treating a patient who is free of monkeypox infection, or is believed to be at risk of infection by monkeypox, or is infected with monkeypox to delay the onset of one or more symptoms associated with monkeypox infection by at least 3 months.

Coronaviruses

As used herein, the terms “SARS-CoV”, “SARS” or “SARS-associated Coronavirus” refers to any strain of coronavirus associated with severe acute respiratory syndrome. Examples of such human coronavirus isolates are known as HCoV-OC43 and HCoV-229E (See, e.g., Marra et al. Science 300:1399 (2003) and Rota et al. Science 300:1394 (2003) (Genbank accession no. AY278741). Skilled artisans are capable of diagnosing individuals infected or suspected of being infected with a SARS associated Coronavirus. The term “treating SARS” or “treating SARS associated Cornoavirus” refers to both treating the symptoms of the disease, as well as reducing the infectivity and/or replication of the SARS-associated Coronavirus. The term “treating SARS” also encompasses treating a person who is free of SARS-CoV infection but is believed to be at risk of infection by SARS-CoV. The term of “preventing SARS” as used herein means preventing in a patient who has SARS-CoV infection or is suspected to have SARS-CoV infection or is at risk of SARS-CoV infection from developing SARS (which is characterized by more serious SARS-CoV-defining symptoms like severe respiratory illness, fever, dry nonproductive cough, shortness of breath, and atypical pneumonia).

West Nile Virus

West Nile (WN) virus has emerged in recent years in temperate regions of Europe and North America, presenting a threat to public, equine, and animal health. The most serious manifestation of WN virus infection is fatal encephalitis (inflammation of the brain) in humans and horses, as well as mortality in certain domestic and wild birds. WN virus infection is a growing problem in North America. During 2002 in the United States alone, there were 4,156 documented cases of WN virus infections of humans and 284 deaths. As used herein, the terms “treating West Nile virus,” “treating West Nile disease” refer to treating the symptoms of the disease in both known and suspected cases of WN virus infection.

In one embodiment, the methods of treatment are generally used to treat an individual experiencing an active viral infection, whether acute or chronic, by any of the aforementioned viruses. In another embodiment, the methods are generally used for treating a carrier of any of the aforementioned viruses who is not experiencing an active viral outbreak. In yet another embodiment, the methods are generally used to treat an individual who is known or suspected to have been exposed to any of the aforementioned viruses. In still another embodiment, the methods are generally used to prophylactically treat an individual who is likely to be exposed to, or is at risk of being exposed to, any of the aforementioned viruses, and thereby prevent infection or lessen its symptoms.

In one particular embodiment, the methods are used for treating an HIV carrier who is not diagnosed as having developed AIDS (which is characterized by more serious AIDS-defining illnesses and/or a decline in the circulating CD4 cell count to below a level that is compatible with effective immune function, i.e., below about 200 μl). For example, the methods can be used in treating a patient at any stages the HIV infection prior to diagnosis of AIDS, including acute HIV syndrome (or acute primary HIV infection syndrome) and asymptomatic infection (which is the long latent period with a gradual decline in the number of circulating CD4 T cells).

In one aspect, the present invention provides methods for treating viral infection—at any stage, and caused by any of the aforementioned viruses, and particularly HIV—in patients who have been, or are being, treated with one or more established antiviral drugs. Examples of such other antiviral compounds include, but are not limited to, protease inhibitors, nucleoside reverse transcriptase inhibitors, non-nucleoside reverse transcriptase inhibitors, integrase inhibitors, fusion inhibitors, and combinations thereof. The compounds of the present invention can be administered to patients who do not respond well to other antiviral drugs (e.g., non-responding, or developing viral resistance) or who experience relapses after treatment with one or more other antiviral drugs or regimens. As used herein, “non-responding patient” or patient “who does not respond well to other antiviral drugs” connote professional observations or judgment by a physician under relevant medical standard or customary practice in the field of antiviral infection therapy. For example, in the case of HIV, a patient may be characterized as non-responding or not responding well if his or her plasma HIV RNA level (or equivalent thereof) does not substantially decrease after treatment with one or more other anti-HIV drugs for a sufficient period of time, or if the reduction of plasma HIV RNA level (or equivalent thereof) is less than a tenfold drop by 4 weeks following the initiation of therapy. Other indications for non-responding patients may include, e.g., persistent decline of CD4 T-cell numbers, adverse drug reaction or toxicity, and clinical deterioration. Thus, the method of the present invention includes a step of identifying such a patient and subsequently administering to the patient a pharmaceutical composition or medicament having a therapeutically effective amount of a compound of the present invention.

In another embodiment, a compound of the present invention is administered to a patient who has undergone a treatment with one or more drugs that target a viral protein such as viral protease, reverse transcriptase, integrase, envelope protein (e.g., gp120 and gp41 for anti-fusion or homolog thereof), and has not responded well to the treatment. The compounds of the present invention belong to a novel class of antiviral drug that is believed to target certain host cell protein(s). Their mode of action is distinct from other antiviral drugs. Thus, they can be especially effective in treating virus-infected patients who do not respond to one or more other antiviral drugs of a different class or who experience relapse after treatment with one or more antiviral drugs of a different class.

In addition, the present invention further provides methods for delaying the onset of acute infection comprising administering a pharmaceutical composition or medicament having a prophylactically effective amount of a compound of the present invention to an individual having an acute viral infection or at risk of viral infection or at risk of developing symptomatic infection. For example, in delaying the onset of symptomatic infection, an individual infected with a virus or at risk of viral infection can be identified, and administered with a prophylactically effective amount of a compound according to the present invention, that is, an amount sufficient to delay the onset of acute viral infection by at least six months. Preferably, an amount is used sufficient to delay the onset of acute viral infection by at least 12 months, 18 months or 24 months.

In addition, the present invention also provides methods for delaying the onset of a symptomatic viral infection comprising identifying an individual who (1) is at risk of infection by a virus, or (2) is suspected of infection by a virus or of exposure to a virus, or (3) has a suspected past exposure to a virus, and administering to the individual a pharmaceutical composition or medicament having a prophylactically effective amount of a compound of the present invention.

For purposes of preventing viral infection, treating asymptomatic viral infection, delaying the onset of symptomatic viral infection, or treating symptomatic viral infection, a compound of the present invention may be used in combination with one or more other antiviral compounds, preferably other antiviral compounds that act through different mechanisms of action. Examples of such other antiviral compounds include, but are not limited to, protease inhibitors, nucleoside reverse transcriptase inhibitors, non-nucleoside reverse transcriptase inhibitors, integrase inhibitors, fusion inhibitors, and a combination thereof. “Co-administration or co-administering” means that the active pharmaceutical agents are administered together as a part of the same therapeutic or treatment regime. The active pharmaceutical agents can be administered separately at different times of the day or at the same time. Additionally, the present invention also provides a pharmaceutical composition having a compound according to Formula I and a compound selected from protease inhibitors, nucleoside reverse transcriptase inhibitors, non-nucleoside reverse transcriptase inhibitors, integrase inhibitors, fusion inhibitors, maturation inhibitors, immunomodulators, vaccines, and combinations thereof. However, it is to be understood that such other antiviral compounds should not interfere with, or adversely affect, the intended effects of the active compounds of this invention. Co-administering to an individual in need of treatment a therapeutically effective amount of a compound of the present invention and a therapeutically effective amount of one or more other antiviral compounds provide a method according to this aspect of the invention.

Accordingly, the present invention also provides pharmaceutical compositions or medicaments useful for the above treatment and prevention purposes and having a therapeutically effective amount of a compound according to Formula I and a therapeutically effective amount of one or more other antiviral compounds. Preferably, such other antiviral compounds have a different mode of action than that of the compounds according to the present invention. More preferably, such other antiviral compounds target a viral protein. Examples of such compounds include, but are not limited to, protease inhibitors, nucleoside reverse transcriptase inhibitors, non-nucleoside reverse transcriptase inhibitors, integrase inhibitors, fusion inhibitors, and combinations thereof.

The present invention also provides methods for treating cancer by administering to a patient (either a human or other animal) in need of such treatment a pharmaceutical composition or medicament having a therapeutically effective amount of a compound of the present invention.

As used herein, “treating cancer” specifically refers to administering therapeutic agents to a subject diagnosed with cancer, i.e., having established cancer in the subject, to inhibit the further growth or spread of the malignant cells in the cancerous tissue, and/or to cause the death of the malignant cells. Treating cancer also encompasses treating a subject having premalignant conditions to stop the progression of, or cause regression of, the premalignant conditions. Examples of premalignant conditions include hyperplasia, dysplasia, and metaplasia.

The present invention further provides an article of manufacture comprising a pharmaceutical composition or medicament having a therapeutically or prophylactically effective amount of a compound according to the present invention. The pharmaceutical composition or medicament can be in a container such as bottle, gel capsule, vial or syringe. The article of manufacture may also include instructions for the use of the pharmaceutical composition or medicament in the various antiviral applications provided above. The instructions can be printed on paper, or in the form of a pamphlet or book. Preferably, the article of manufacture according to the present invention further comprises a therapeutically or prophylactically effective amount of one or more other antiviral compounds as described above.

Typically, compounds according to the present invention can be effective at an amount of from about 0.01 μg/kg to about 100 mg/kg per day based on total body weight. The active ingredient may be administered at once, or may be divided into a number of smaller doses to be administered at predetermined intervals of time. The suitable dosage unit for each administration can be, e.g., from about 1 μg to about 2000 mg, preferably from about 5 μg to about 1000 mg. In the case of combination therapy, a therapeutically effective amount of one or more other antiviral compounds can be administered in a separate pharmaceutical composition, or alternatively included in the pharmaceutical composition according to the present invention which contains a compound according to the present invention. The pharmacology and toxicology of many of such other antiviral compounds are known in the art. See e.g., Physicians Desk Reference, Medical Economics, Montvale, N.J.; and The Merck Index, Merck & Co., Rahway, N.J. The therapeutically effective amounts and suitable unit dosage ranges of such compounds used in art can be equally applicable in the present invention.

It should be understood that the dosage ranges set forth above are exemplary only and are not intended to limit the scope of this invention. The therapeutically effective amount for each active compound can vary with factors including but not limited to the activity of the compound used, stability of the active compound in the patient's body, the severity of the conditions to be alleviated, the total weight of the patient treated, the route of administration, the ease of absorption, distribution, and excretion of the active compound by the body, the age and sensitivity of the patient to be treated, and the like, as will be apparent to a skilled artisan. The amount of administration can be adjusted as the various factors change over time.

In the pharmaceutical compositions, the active agents can be in any pharmaceutically acceptable salt form. As used herein, the term “pharmaceutically acceptable salts” refers to the relatively non-toxic, organic or inorganic salts of the active compounds, including inorganic or organic acid addition salts of the compound. Examples of salts of basic active ingredient compounds include, but are not limited to, hydrochloride salts, hydrobromide salts, sulfate salts, bisulfate salts, nitrate salts, acetate salts, phosphate salts, nitrate salts, oxalate salts, valerate salts, oleate salts, borate salts, benzoate salts, laurate salts, stearate salts, palmitate salts, lactate salts, tosylate salts, citrate salts, maleate, salts, succinate salts, tartrate salts, napththylate salts, fumarate salts, mesylate salts, laurylsuphonate salts, glucoheptonate salts, and the like. See, e.g., Berge, et al. J. Pharm. Sci., 66:1-19 (1977). Examples of salts of acidic active ingredient compounds include, e.g., alkali metal salts, alkaline earth salts, and ammonium salts. Thus, suitable salts may be salts of aluminum, calcium, lithium, magnesium, potassium, sodium and zinc. In addition, organic salts may also be used including, e.g., salts of lysine, N,N′-dibenzylethylenediamine, chloroprocaine, choline, diethanolamine, ethylenediamine, meglumine (N-methylglucamine), procaine and tris.

For oral delivery, the active compounds can be incorporated into a formulation that includes pharmaceutically acceptable carriers such as binders (e.g., gelatin, cellulose, gum tragacanth), excipients (e.g., starch, lactose), lubricants (e.g., magnesium stearate, silicon dioxide), disintegrating agents (e.g., alginate, Primogel, and corn starch), and sweetening or flavoring agents (e.g., glucose, sucrose, saccharin, methyl salicylate, and peppermint). The formulation can be orally delivered in the form of enclosed gelatin capsules or compressed tablets. Capsules and tablets can be prepared in any conventional techniques. The capsules and tablets can also be coated with various coatings known in the art to modify the flavors, tastes, colors, and shapes of the capsules and tablets. In addition, liquid carriers such as fatty oil can also be included in capsules.

Suitable oral formulations can also be in the form of suspension, syrup, chewing gum, wafer, elixir, and the like. If desired, conventional agents for modifying flavors, tastes, colors, and shapes of the special forms can also be included. In addition, for convenient administration by enteral feeding tube in patients unable to swallow, the active compounds can be dissolved in an acceptable lipophilic vegetable oil vehicle such as olive oil, corn oil and safflower oil.

The active compounds can also be administered parenterally in the form of solution or suspension, or in lyophilized form capable of conversion into a solution or suspension form before use. In such formulations, diluents or pharmaceutically acceptable carriers such as sterile water and physiological saline buffer can be used. Other conventional solvents, pH buffers, stabilizers, anti-bacteria agents, surfactants, and antioxidants can all be included. For example, useful components include sodium chloride, acetates, citrates or phosphates buffers, glycerin, dextrose, fixed oils, methyl parabens, polyethylene glycol, propylene glycol, sodium bisulfate, benzyl alcohol, ascorbic acid, and the like. The parenteral formulations can be stored in any conventional containers such as vials and ampoules.

Routes of topical administration include nasal, bucal, mucosal, rectal, or vaginal applications. For topical administration, the active compounds can be formulated into lotions, creams, ointments, gels, powders, pastes, sprays, suspensions, drops and aerosols. Thus, one or more thickening agents, humectants, and stabilizing agents can be included in the formulations. Examples of such agents include, but are not limited to, polyethylene glycol, sorbitol, xanthan gum, petrolatum, beeswax, or mineral oil, lanolin, squalene, and the like. A special form of topical administration is delivery by a transdermal patch. Methods for preparing transdermal patches are disclosed, e.g., in Brown, et al., Annual Review of Medicine, 39:221-229 (1988), which is incorporated herein by reference.

Subcutaneous implantation for sustained release of the active compounds may also be a suitable route of administration. This entails surgical procedures for implanting an active compound in any suitable formulation into a subcutaneous space, e.g., beneath the anterior abdominal wall. See, e.g., Wilson et al., J. Clin. Psych. 45:242-247 (1984). Hydrogels can be used as a carrier for the sustained release of the active compounds. Hydrogels are generally known in the art. They are typically made by crosslinking high molecular weight biocompatible polymers into a network, which swells in water to form a gel like material. Preferably, hydrogels are biodegradable or biosorbable. For purposes of this invention, hydrogels made of polyethylene glycols, collagen, or poly(glycolic-co-L-lactic acid) may be useful. See, e.g., Phillips et al., J. Pharmaceut. Sci., 73:1718-1720 (1984).

The active compounds can also be conjugated, to a water soluble non-immunogenic non-peptidic high molecular weight polymer to form a polymer conjugate. For example, an active compound is covalently linked to polyethylene glycol to form a conjugate. Typically, such a conjugate exhibits improved solubility, stability, and reduced toxicity and immunogenicity. Thus, when administered to a patient, the active compound in the conjugate can have a longer half-life in the body, and exhibit better efficacy. See generally, Burnham, Am. J. Hosp. Pharm., 15:210-218 (1994). PEGylated proteins are currently being used in protein replacement therapies and for other therapeutic uses. For example, PEGylated interferon (PEG-INTRON A®) is clinically used for treating Hepatitis B. PEGylated adenosine deaminase (ADAGEN®) is being used to treat severe combined immunodeficiency disease (SCIDS). PEGylated L-asparaginase (ONCAPSPAR®) is being used to treat acute lymphoblastic leukemia (ALL). It is preferred that the covalent linkage between the polymer and the active compound and/or the polymer itself is hydrolytically degradable under physiological conditions. Such conjugates known as “prodrugs” can readily release the active compound inside the body. Controlled release of an active compound can also be achieved by incorporating the active ingredient into microcapsules, nanocapsules, or hydrogels generally known in the art.

Liposomes can also be used as carriers for the active compounds of the present invention. Liposomes are micelles made of various lipids such as cholesterol, phospholipids, fatty acids, and derivatives thereof. Various modified lipids can also be used. Liposomes can reduce the toxicity of the active compounds, and increase their stability. Methods for preparing liposomal suspensions containing active ingredients therein are generally known in the art. See, e.g., U.S. Pat. No. 4,522,811; Prescott, Ed., Methods in Cell Biology, Volume XIV, Academic Press, New York, N.Y. (1976).

The active compounds can also be administered in combination with another active agent that synergistically treats or prevents the same symptoms or is effective for another disease or symptom in the patient treated so long as the other active agent does not interfere with or adversely affect the effects of the active compounds of this invention. Such other active agents include but are not limited to anti-inflammation agents, antiviral agents, antibiotics, antifungal agents, antithrombotic agents, cardiovascular drugs, cholesterol lowering agents, anti-cancer drugs, hypertension drugs, and the like. In this combination therapy approach, the two different pharmaceutically active compounds can be administered separately or in the same pharmaceutical composition.

Examples of antiviral compounds suitable for use in combination therapy with compounds of the present invention include, but are not limited to, HIV protease inhibitors, nucleoside HIV reverse transcriptase inhibitors, non-nucleoside HIV reverse transcriptase inhibitors, HIV integrase inhibitors, HIV fusion inhibitors, HIV maturation inhibitors, immunomodulators, and vaccines.

Examples of nucleoside HIV reverse transcriptase inhibitors include 3′-Azido-3′-deoxythymidine (Zidovudine, also known as AZT and RETROVIR®), 2′,3′-Didehydro-3′-deoxythymidine (Stavudine, also known as 2′,3′-dihydro-3′-deoxythymidine, d4T, and ZERIT®), (2R-cis)-4-Amino-1-[2-(hydroxymethyl)-1,3-oxathiolan-5-yl]-2(1H)-pyrimidinone (Lamivudine, also known as 3TC, and EPIVIR®), 2′,3′-dideoxyinosine (ddI), and 9-[(R)-2-[[bis[[isopropoxycarbonyl)oxy]methoxy]phosphinyl]methoxy]propyl]adenine fumarate (Tenofovir disoproxil fumarate, also known as Viread™).

Examples of non-nucleoside HIV reverse transcriptase inhibitors include (−)-6-Chloro-4-cyclopropylethynyl-4-trifluoromethyl-1,4-dihydro-2H-3,1-benzoxazin-2-one (efavirenz, also known as DMP-266 or SUSTIVAR®) (see U.S. Pat. No. 5,519,021), 1-[3-[(1-methylethyl)aminol]-2-pyridinyl]-4-[[5-[(methylsulfonyl)amino]-1H-indol-2-yl]carbonyl]piperazine (Delavirdine, see PCT International Patent Application No. WO 91/09849), and (1S,4R)-cis-4-[2-amino-6-(cycloprpoylamino)-9H-purin-9-yl]-2-cyclopentene-1-methanol (Abacavir).

Examples of protease inhibitors include [5S-(5R*,8R*,10R*,11R*)]-10-hydroxy-2-methyl-5-(1-methylethyl)-1-[2-(1-methylethyl)-4-thiazolyl]-3,6-dioxo-8,11-bis(phenylmethyl)-2,4,7,12-tetraazamidecan-13-oic acid 5-thiazolylmethyl ester (Ritonavir, marketed by Abbott as NORVIR®), [3S-[2(2S*,3S*),3a,4ab,8ab]]-N-(1,1-dimethylethyl)decahydro-2-[2-hydroxy-3-[(3-hydroxy-2-methylbenzoyl)amino]-4-(phenylthio)butyl]-3-isoquinolinecarb oxamide monomethanesulfonate (Nelfinavir, marketed by Agouron as VIRACEPT®), N-(2(R)-hydroxy-1(S)-indanyl)-2(R)-phenylmethyl-4-(S)-hydroxy-5-(1-(4-(2-benzo[b]furanylmethyl)-2(S)—N′(t-butylcarboxamido)-piperazinyl))-pentaneamide (See U.S. Pat. No. 5,646,148), N-(2(R)-hydroxy-1(S)-indanyl)2(R)-phenylmethyl-4-(S)-hydroxy-5-(1-(4-(3-pyridylmethyl)-2(S)—N′-(t-butylcarboxamido)-piperazinyl))-pentaneamide (Indinavir, marketed by Merck as CRIXIVAN®), 4-amino-N-((2 syn,3S)-2-hydroxy-4-phenyl-3-((S)-tetrahydrofuran-3-yloxycarbonylamino)-butyl)-N-isobutyl-benzenesulfonamide (amprenavir, see U.S. Pat. No. 5,585,397), and N-tert-butyl-decahydro-2-[2(R)-hydroxy-4-phenyl-3(S)-[[N-(2-quinolylcarbonyl)-L-asparaginyl]amino]butyl]-(4aS,8aS)-isoquinoline-3(S)-carboxamide (Saquinavir, marketed by Roche Laboratories as INVIRASE®).

Examples of suitable HIV integrase inhibitors are disclosed in U.S. Pat. Nos. 6,110,716; 6,124,327; and 6,245,806, which are incorporated herein by reference.

Various other antiviral agents can also be used in a combination therapy with compounds of the present invention, including, but not limited to, 9-(2-hydroxyethoxymethyl)guanine (acyclovir), 2-amino-9-(2-hydroxyethoxymethyl)purine, suramin, ribavirin, antimoniotungstate (HPA-23), interferon, interleukin II, and phosphonoformate (Foscarnet). In addition, other medications such as levamisol or thymosin which would stimulate lymphocyte growth and/or function may also be employed.

Examples of HIV fusion inhibitors include antibodies against HIV envelope proteins (e.g., gp120, gp41) and peptides derived from the HIV envelope proteins. For example, a gp41-derived peptide called T-20 (Trimeris Inc., Durham, N.C.) has been shown to be effective in treating HIV infection in a phase III clinical trial.

Any suitable pharmaceutically acceptable derivatives of the above compounds may also be used including pharmaceutically acceptable salts and esters thereof.

### EXAMPLES

The examples below are intended to exemplify the practice of the present invention but are by no means intended to limit the scope thereof.

### Example 1

### Synthesis

Synthesis of compounds of the present invention can be accomplished according to the following general synthetic route. See Tables 1-7 for representative structures and relevant characterization data.

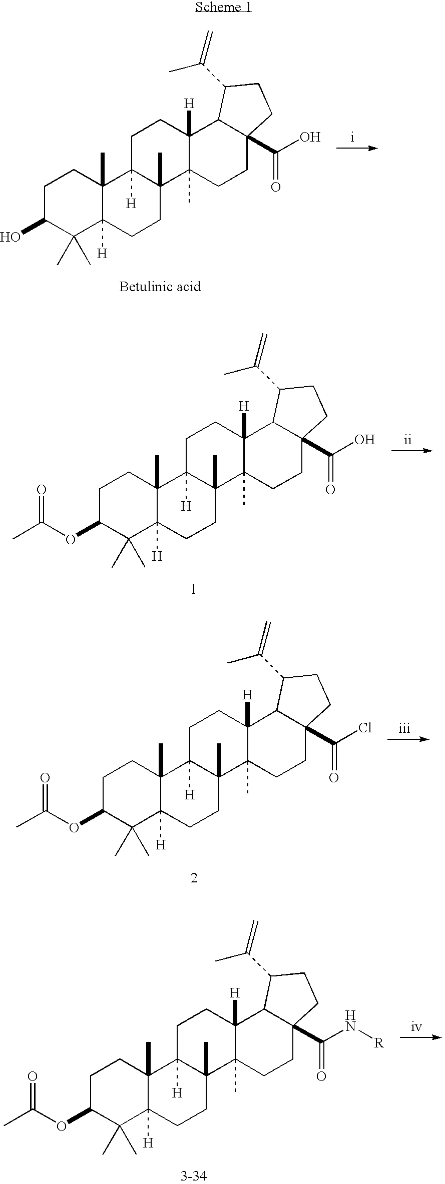

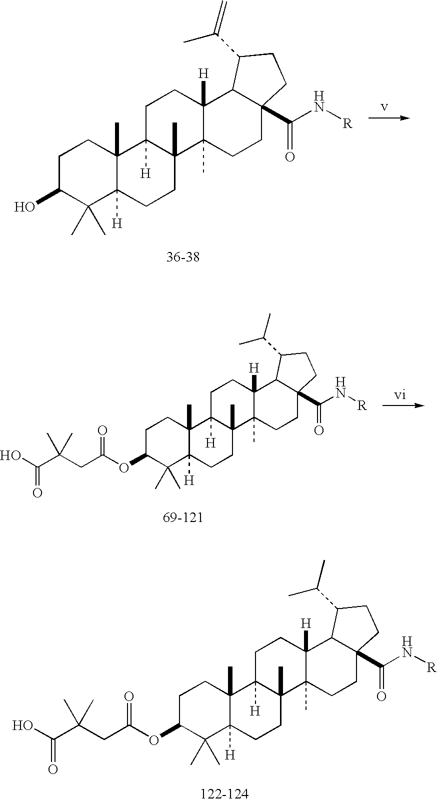

The above scheme summarizes the synthetic routes to the compounds in Tables 1-7 where the reagents/conditions are: i. Ac₂O, DMAP, Py, Δ. ii. Oxalyl Chloride (2M), CH₂Cl₂. iii. NHR₁R₂, TEA, CH₂Cl₂. iv. NaOH (4M), THF/MeOH. v. 2,2-Dimethylsuccinic anhydride, DMAP, Py, Δ. vi. PtO₂, H₂ (15 psi), AcOH.

In general, compounds of the invention can be synthesized by:

(i) adding a protecting group to the chosen position of the starting material (i.e. the C3 position of betulinic acid);

(ii) forming an acyl chloride at any desired position of the compound formed in step (i) (i.e. the C28 position);

(iii) allowing the acyl chloride formed in step (ii) to react with the appropriate desired moiety (such as the NH₂—R group in the scheme above);

(iv) removing the protecting group added in step (i); and optionally

(v) adding any moiety to the deprotected position of the compound formed in step (iv) (i.e. adding the dimethylsuccinyl group to the C3 position as shown in the scheme above).

Optionally, unsaturated bonds can be reduced to form compounds of the invention. Compounds of the invention can also be synthesized by

(i) activating the chosen position of the starting material (i.e. the C28 position of betulinic acid);

(ii) allowing the compound formed in step (i) to react with the appropriate desired moiety (such as the NH₂—R group in the scheme above); and

(iii) adding any moiety to other desired positions of the material formed in step (ii) (i.e. adding the dimethylsuccinyl group to the C3 position as shown in the scheme above).

Protecting groups refer to moieties that protect a chemical group from undesirable reactions. For example, protecting groups include those known to one skilled in the art such as those set forth in Protective Groups in Organic Synthesis, Greene, T., John Wiley & Sons, New York, N.Y., (1st Edition, 1981), which can be added or removed using the procedures set forth therein. Examples of protected hydroxyl groups include, but are not limited to, silyl ethers such as those obtained by reaction of a hydroxyl group with a reagent such as, but not limited to, t-butyldimethyl-chlorosilane, trimethylchlorosilane, triisopropylchlorosilane, triethylchlorosilane; substituted methyl and ethyl ethers such as, but not limited to methoxymethyl ether, methylthiomethyl ether, benzyloxymethyl ether, t-butoxymethyl ether, 2-methoxyethoxymethyl ether, tetrahydropyranyl ethers, 1-ethoxyethyl ether, allyl ether, benzyl ether; esters such as, but not limited to, benzoylformate, formate, acetate, trichloroacetate, and trifluoracetate. Examples of protected amine groups include, but are not limited to, amides such as, formamide, acetamide, trifluoroacetamide, and benzamide; imides, such as phthalimide, and dithiosuccinimide; and others. Examples of protected sulfhydryl groups include, but are not limited to, thioethers such as S-benzyl thioether, and S-4-picolyl thioether; substituted S-methyl derivatives such as hemithio, dithio and aminothio acetals; and others. Examples of protecting groups for protein synthesis, include, but are not limited to, BOC, FMOC and CBZ (i.e., tert-butyloxycarbonyl, 9-fluorenylmethoxycarbonyl and benzyloxycarbonyl, respectively).

Groups can be added and removed during the synthesis process by performing procedures known in the art. For example, protecting groups can be added by adding an activated acid (such as acetic anhydride) and an organic base (such as triethylamine or pyridine) and heating the resultant mixture. Positions of compounds can be activated by reaction with an activating agent known in the art, such as dicyclohexylcarbodiimide, EDCI, HATU, or PyBOP. Acyl chlorides can be formed by allowing the carboxylic acid to react with a chlorination agent, such as thionylchloride, oxalylchloride, phosphorousoxychloride, and cyanuric chloride. Acyl chlorides can reacted with appropriate moieties, such as primary and secondary amines, to form the desired group, such as amide groups.

Protecting groups can be removed by methods known to those of skill in the art. For example, removing an acetate protecting group can be accomplished by contacting the material with a base, such as an aqueous sodium hydroxide solution. Additional moieties can be added at desired positions of the material, such as adding a dimethylsuccinyl group to the C3 position, by reacting the material with dimethylsuccinic anhydride in the presence of a base, such as pyridine.

Compounds of Formulae II and III can also be synthesized according to the general synthetic route above by substituting the appropriate starting material for betulinic acid. For example, compounds of Formula II may be synthesized according to the general synthetic route above by substituting oleanolic acid for betulinic acid; and compounds of Formula III may be synthesized by substituting ursolic acid for betulinic acid.

General procedure for HPLC purification: Samples were dissolved in DMSO (˜50 mg/mL), and purified on a Phenomenex Synergi Hydro-RP (00G-4376-P0) HPLC column (250×21.2 mm, 10μ sphere size, 80 Å pore size), the solvent system is 50-90% acetonitrile in water (0.01% trifluoroacetic acid), run isocratic for up to 25 minutes. Fraction collection is based on absorption at 203λ.

### (3β)-3-(acetyloxy)lup-20(29)-en-28-oic acid (1)

A solution of betulinic acid (0.50 g, 1.1 mmol) in anhydrous pyridine (10 mL) under nitrogen atmosphere was treated with Ac₂O (0.26 ml, 2.8 mmol) and DMAP (0.14 g, 1.1 mmol) and the mixture was refluxed for 1 h. The reaction mixture was diluted with CHCl₃ and washed with water. The organic layer was dried over MgSO₄ and concentrated under reduced pressure to give 1 (0.42 g, 76%).

¹H NMR (DMSO-d₆, 400 MHz) δ 0.79 (s, 6H, CH₃), 0.80 (s, 3H, CH₃), 0.87 (s, 3H, CH₃), 0.94 (s, 3H, CH₃), 1.25-1.62 (m, 18H, CH₂), 1.65 (s, 3H, CH₃), 1.75-1.85 (m, 2H, CH₂), 1.99 (s, 3H, CH₃CO), 2.08-2.14 (m, 1H), 2.18-2.27 (m, 1H), 2.90-3.00 (m, 1H), 4.36 (dd, 1H, J=11.24 Hz, J=4.8 Hz, H-3), 4.56 (m, 1H, CH═), 4.69 (d, 1H, J=2.15 Hz, CH═), 12.10 (bs, 1H, CO₂H).

### Preparation of the Acid Chlorides of 3(3β)-3-(acetyloxy)lup-20(29)-en-28-oic acid (2)

Oxalyl chloride solution (2M in CH₂Cl₂, 4 mL) was added to the 3-O-acetyl-betulinic acid (0.1 g, 0.2 mmol) and stirred for 2 h. The mixture was concentrated to dryness under reduced pressure. The residue was diluted with dry CH₂Cl₂ (3×1 mL), concentrated to dryness under reduced pressure, and used without further purification.

General Procedure for Synthesizing Compounds (3-34)

To a solution of the acid chloride 2 (0.2 mmol) in dry CH₂Cl₂ (5 mL) under nitrogen atmosphere was added the appropriate amine (0.26 mmol) and TEA (0.44 mmol, 0.061 mL). The reaction mixture was stirred at room temperature overnight, diluted with CH₂Cl₂ and then the CH₂Cl₂ layer washed with H₂O. The organic layer was dried over MgSO₄ and concentrated under reduced pressure to give the amide compound. In some cases the products were pure enough to use them directly for the next step, and some products were purified by HPLC.

TABLE 1

| Compound | | NMR (DMSO-d₆, 400 MHz). |
|---|---|---|
| No. | Structure | LC-MS (ESI) |
| 3 | 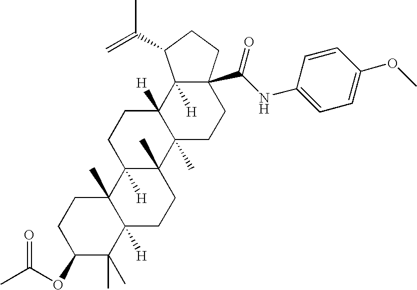 | 0.78 (s, 6 H, CH₃), 0.81 (s, 3 H, CH₃),0.86 (s, 3 H, CH₃), 0.96 (s, 3 H, CH₃),1.00-1.85 (m, 25 H), 1.99 (s, 3 H,CH₃CO), 2.30-2.40 (m, 1 H), 2.70(m, 1 H), 3.20-3.00 (m, 2 H), 3.70 (s,3 H, OCH₃), 4.36 (dd, 1 H, J = 11.6Hz, J = 4.8 Hz, H-3), 4.55 (bs, 1 H,CH═), 4.68 (bs, 1 H, CH═), 6.84 (d,2 H, J = 9.1, CH Arom), 7.45 (d, 2 H,J = 9.1 Hz, CH Arom), 9.29 (s, 1 H,NH).604.54 (M + H)⁺. |
| 4 | 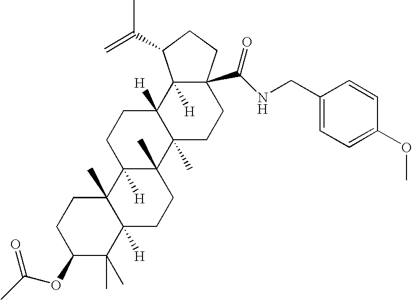 | 0.79 (s, 12 H, CH₃), 0.80 (s, 3 H,CH₃), 0.92 (s, 3 H, CH₃), 1.00-1.85(m, 23 H, CH₂, CH₃), 1.99 (s, 3 H,CH₃CO), 2.10-2.20 (m, 1 H), 2.95-3.05 (m, 1 H), 3.72 (s, 3 H, CH₃O),4.10 (dd, 1 H, J = 15.0 Hz, J = 6.0Hz, CH₂N), 4.22 (dd, 1 H, J = 15.0Hz, J = 6.0 Hz, CH₂N), 4.36 (dd,1 H, J = 10.8 Hz, J = 5.0 Hz, H-3),4.53 (bs, 1 H, CH═), 4.65 (bs, 1 H,CH═), 6.84 (d, 2 H, J = 8.8 Hz, CHArom), 7.15 (d, 2 H, J = 8.8 Hz, CHArom), 8.09 (t, 1 H, J = 6.2 Hz, NH).618.49 (M + H)⁺. |
| 5 | 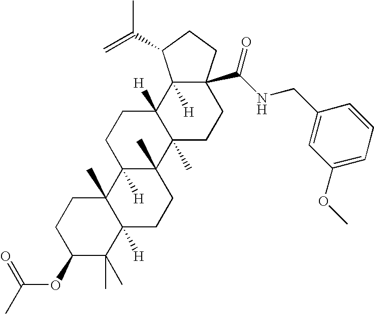 | 0.79 (s, 15 H, CH₃), 0.92 (s, 3 H,CH₃), 1.00-1.90 (m, 23 H), 1.99 (s,3 H, CH₃CO), 2.10-2.25 (m, 1 H),2.95-3.05 (m, 1 H), 3.72 (s, 3 H,CH₃O), 4.17 (dd, 1 H, J = 15.2 Hz, J =5.9 Hz, CH₂N), 4.25 (dd, 1 H, J =15.2 Hz, J = 5.3 Hz, CH₂N), 4.30-4.40 (m, 1 H, H-3), 4.53 (bs, 1 H,CH═), 4.65 (bs, 1 H, CH═), 6.70-6.85(m, 3 H, CH Arom), 7.20 (t, 1 H, J =7.7 Hz, CH Arom), 8.16 (bs, 1 H,NH).618.30 (M + H)⁺. |
| 6 | 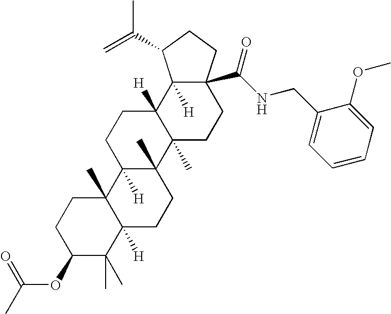 | 0.79 (s, 12 H, CH₃), 0.93 (s, 3 H,CH₃), 1.00-1.95 (m, 26 H), 1.99 (s,3 H, CH₃CO), 2.20-2.30 (m, 1 H),2.955-3.05 (m, 1 H), 3.79 (s, 3 H,CH₃O), 4.20 (d, 2 H, J = 5.8 Hz,CH₂N), 4.36 (dd, 1 H, J = 11.1 Hz, J =4.8 Hz, H-3), 4.53 (bs, 1 H, CH═),4.64 (d, 1 H, J = 2.5 Hz, CH═), 6.87(dt, 1 H, J = 8.2 Hz, J = 0.8 Hz, CHArom), 6.95 (d, 1 H, J = 8.2 Hz, CHArom), 7.11 (d, 1 H, J = 6.1 Hz, CHArom), 7.20 (dt, 1 H, J = 8.6 Hz, J =1.6 Hz, CH Arom), 7.97 (t, 1 H, J =5.8 Hz, NH).618.4576 (M + H)⁺. |
| 7 | 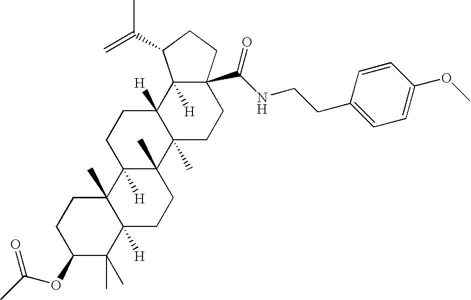 | 0.76 (s, 3 H, CH₃), 0.85 (s, 9 H, CH₃),0.90-1.08(m, 6 H), 1.09-1.75 (m,23 H), 1.99 (s, 3 H, CH₃CO), 2.02-2.08 (m, 1 H), 2.56-2.76 (m, 2 H),2.95-3.05 (m, 1 H, J = 11.0 Hz, J =4.7 Hz), 3.10-3.16 (m, 1 H), 3.28-3.35 (m, 1 H), 3.70 (s, 3 H, CH₃O),4.35 (dd, 1 H, J = 11.1 Hz, J = 4.8Hz, H-3), 4.52 (bs, 1 H, CH═), 4.64(d, 1 H, J = 2.5 Hz, CH═), 6.82 (d,2 H, J = 8.61 Hz, CH Arom), 7.09 (d,2 H, J = 8.6 Hz, CH Arom), 7.57 (t,1 H, J = 5.4 Hz, NH). |
| 8 | 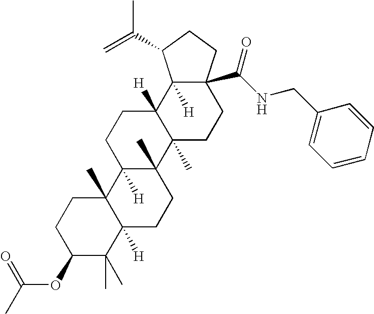 | 0.79 (s, 9 H, CH₃), 0.80 (s, 3 H, CH₃),0.92 (s, 3 H, CH₃), 1.00-1.85 (m,26 H), 1.99 (s, 3 H, CH₃CO), 2.15-2.25 (m, 1 H), 2.97-3.15 (m, 1 H),4.17 (dd, 1 H, J = 15.0 Hz, J = 6.2Hz, CH₂N), 4.25-4.40 (m, 2 H, H-3and CH₂N), 4.53 (bs, 1 H, CH═),4.65 (d, 1 H, J = 2.5 Hz, CH═), 7.18-7.32 (m, 5 H, CH Arom), 8.17 (t, 1 H,J = 6.1 Hz, NH).588.55 (M + H)⁺. |
| 9 | 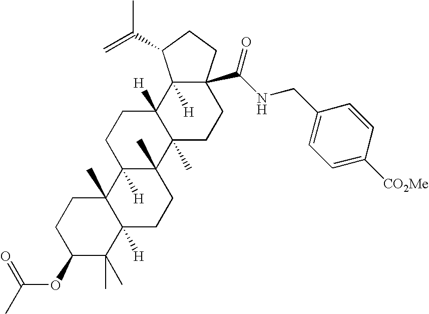 | 0.77 (s, 3 H, CH₃), 0.79 (s, 9 H, CH₃),0.92 (s, 3 H, CH₃), 1.00-1.90 (m,26 H), 1.99 (s, 3 H, CH₃CO), 2.15-2.25 (m, 1 H), 2.95-3.05 (m, 1 H),3.84 (s, 3 H, CO₂Me), 4.24 (dd, 1 H, J =16.05 Hz, J = 6.0 Hz, CH₂N),4.30-4.40 (m, 2 H, H-3 and CH₂N),4.53 (bs, 1 H, CH═), 4.64 (d, 1 H, J =2.1 Hz, CH═), 7.37 (d, 2 H, J = 8.4Hz, CH Arom), 7.89 (d, 2 H, J = 8.4Hz, CH Arom), 8.27 (t, 1 H, J = 6.0Hz, NH).646.54 (M + H)⁺. |
| 10 | 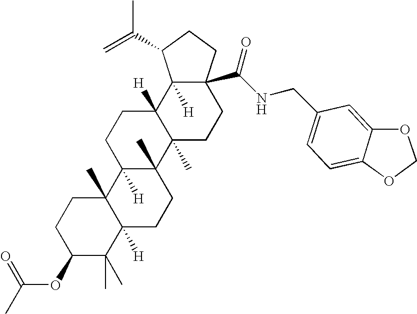 | 0.79 (s, 9 H, CH₃), 0.80 (s, 3 H, CH₃),0.92 (s, 3 H, CH₃), 1.00-1.85 (m,26 H), 1.99 (s, 3 H, CH₃CO), 2.10-2.20 (m, 1 H), 2.95-3.08 (m, 1 H),4.06 (dd, 1 H, J = 14.7 Hz, J = 6.1Hz, CH₂N), 4.20 (dd, 1 H, J = 14.7Hz, J = 6.0 Hz, CH₂N), 4.36 (dd, 1 H,J = 11.1 Hz, J = 4.8 Hz, H-3), 4.53(bs, 1 H, CH═), 4.65 (d, 1 H, J = 2.3Hz, CH═), 5.96 (d, 2 H, J = 2.2 Hz,OCH₂O), 6.70 (dd, 1 H, J = 7.8 Hz, J =1.5 Hz, CH Arom), 6.78 (d, 1 H, J =1.5 Hz, CH Arom), 6.82 (d, 1 H, J =7.8 Hz, CH Arom), 8.10 (t, 1 H, J = 5.7 Hz, NH).632.46 (M + H)⁺. |
| 11 | 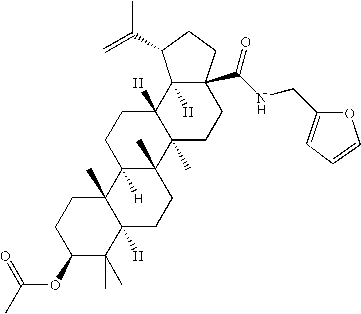 | 0.79 (s, 9 H, CH₃), 0.80 (s, 3 H, CH₃),0.92 (s, 3 H, CH₃), 0.95-1.85 (m,26 H), 1.99 (s, 3 H, CH₃CO), 2.10-2.20 (m, 1 H), 2.95-3.08 (m, 1 H),4.13 (dd, 1 H, J = 16.1 Hz, J = 5.6Hz, CH₂N), 4.29 (dd, 1 H, J = 16.1Hz, J = 5.9 Hz, CH₂N), 4.36 (dd,1 H, J = 11.0 Hz, J = 4.9 Hz, H-3),4.53 (bs, 1 H, CH═), 4.65 (bs, 1 H,CH═), 6.12 (dd, 1 H, J = 3.2 Hz, J =0.9 Hz, CH Arom), 6.37 (dd, 1 H, J =3.2 Hz, J = 1.9 Hz, CH Arom), 7.52(dd, 1 H, J = 1.9 Hz, J = 0.9 Hz, CHArom), 8.08 (t, 1 H, J = 5.9 Hz, NH).578.41 (M + H)⁺. |
| 12 | 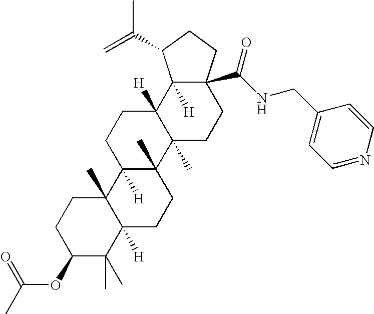 | 0.75 (s, 3 H, CH₃), 0.79 (s, 9 H, CH₃),0.93 (s, 3 H, CH₃), 1.00-1.90 (m,26 H), 1.99 (s, 3 H, CH₃CO), 2.15-2.25 (m, 1 H), 2.90-3.06 (m, 1 H),4.22 (dd, 1 H, J = 14.6 Hz, J = 6.4Hz, CH₂N), 4.35-4.40 (m, 2 H, H-3and CH₂N), 4.53 (bs, 1 H, CH═),4.64 (bs, 1 H, CH═), 7.22 (d, 2 H, J =5.9 Hz, CH Arom), 8.28 (bs, 1 H,NH), 8.47 (d, 2 H, J = 5.9 Hz, CHArom). 589.71 (M + H)⁺. |
| 13 | 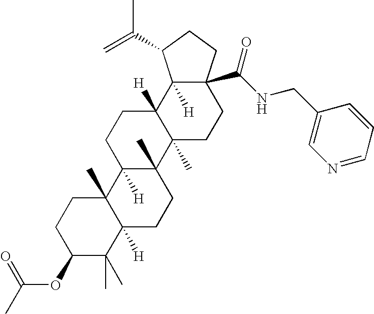 | 0.72 (s, 3 H, CH₃), 0.78 (s, 9 H, CH₃),0.91 (s, 3 H, CH₃), 1.00-1.90 (m,26 H), 1.99 (s, 3 H, CH₃CO), 2.10-2.20 (m, 1 H), 2.95-3.06 (m, 1 H),4.16 (dd, 1 H, J = 15.3 Hz, J = 6.0Hz, CH₂N), 4.30 (dd, 1 H, J = 15.3Hz, J = 5.9 Hz, CH₂N), 4.35 (dd,1 H, J = 11.3 Hz, J = 4.8 Hz, H-3),4.53 (bs, 1 H, CH═), 4.65 (d, 1 H, J =2.5 Hz, CH═), 7.30-7.38 (m, 1 H, CHArom), 7.63 (dt, 1 H, J = 8.0 Hz, J =1.8 Hz, CH Arom), 8.25 (t, 1 H, J =6.1 Hz, NH), 8.42 (dd, 1 H, J = 4.7Hz, J = 1.8 Hz, CH Arom), 8.46 (d,1 H, J = 1.8 Hz, CH Arom).589.4372 (M + H)⁺. |
| 14 | 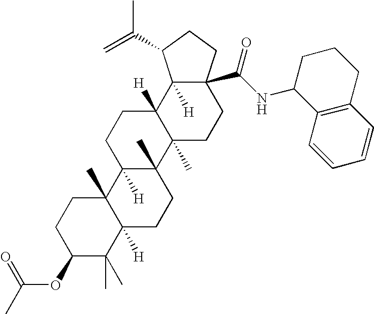 | 0.80 (s, 9 H, CH₃), 0.84 (s, 3 H, CH₃),0.93 (s, 3 H, CH₃), 0.95 (s, 3 H, CH₃),0.98 (s, 3 H, CH₃), 1.00-1.95 (m,28 H), 2.00 (s, 3 H, CH₃CO), 2.15-2.25 (m, 1 H), 2.60-2.80 (m, 4 H),3.00-3.15 (m, 1 H), 4.30-4.40 (m,1 H, H-3), 4.55 (bs, 1 H, CH═), 4.65-4.70 (m, 1 H, CH═), 4.95-5.10 (m,1 H, CHN), 7.00-7.20 (m, 4 H, CHArom), 7.80-7.90 (m, 1 H, NH).628.4716 (M + H)⁺. |
| 15 | 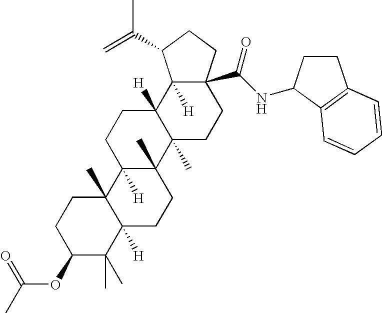 | 0.79 (s, 6 H, CH₃), 0.84 (s, 3 H, CH₃),0.88 (s, 3 H, CH₃), 0.94 (s, 3 H, CH₃),0.95 (s, 3 H, CH₃), 0.97 (s, 3 H, CH₃),1.00-1.95 (m, 20 H), 1.99 (s, 3 H,CH₃CO), 2.25-2.35 (m, 1 H), 2.55-2.80 (m, 4 H), 3.00-3.20 (m, 1 H),4.33-4.42 (m, 1 H, H-3), 4.55 (bs,1 H, CH═), 4.65-4.72 (m, 1 H, CH═),5.25-5.40 (m, 1 H, CHN), 7.05-7.35(m, 4 H, CH Arom), 7.85 (t, 1 H, J =8.02 Hz, NH).614.4557 (M + H)⁺. |
| 16 | 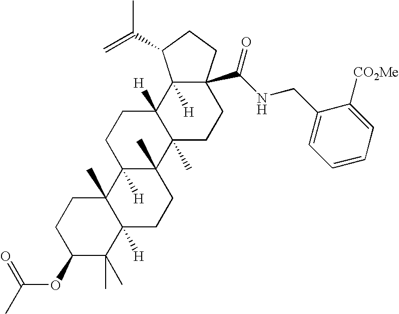 | 0.75 (s, 3 H, CH₃), 0.79 (s, 9 H, CH₃),0.93 (s, 3 H, CH₃), 1.00-1.95 (m,26 H), 1.99 (s, 3 H, CH₃CO), 2.15-2.25 (m, 1 H), 2.90-3.05 (m, 1 H),3.85 (s, 3 H, CH₃O), 4.35-4.40 (m,2 H, H-3 and CH₂N), 4.53 (m, 2 H,CH═ and CH₂N)), 4.64 (bs, 1 H,CH═), 7.30-7.70 (m, 3 H, CH Arom),7.84 (d, 1 H, J = 6.5 Hz, CH Arom),8.08 (t, 1 H, J = 5.3 Hz, NH).668.52 (M + H)⁺. |
| 17 | 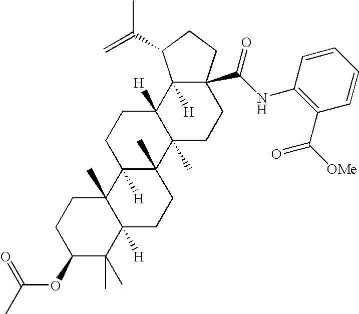 | 0.78 (s, 6 H, CH₃), 0.80 (s, 3 H, CH₃),0.84 (s, 3 H, CH₃), 0.98 (s, 3 H, CH₃),1.10-1.95 (m, 26 H), 1.99 (s, 3 H,CH₃CO), 2.25-2.15 (m, 1 H), 3.00-3.15 (m, 1 H), 3.87 (s, 3 H, OCH₃),4.36 (dd, 1 H, J = 11.0 Hz, J = 5.1Hz, H-3), 4.58 (bs, 1 H, CH═), 4.71(bs, 1 H, CH═), 7.14-7.18 (m, 1 H,CH Arom), 7.59-7.63 (m, 1 H, CHArom), 7.96 (dd, 1 H, J = 8.0 Hz, J =1.6 Hz, CH Arom), 8.42 (d, 1 H, J =7.4 Hz, CH Arom), 10.91 (s, 1 H,NH). 630.41640 (M − H)⁻. |
| 18 | 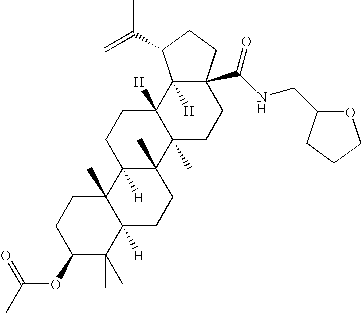 | 0.78 (s, 9 H, CH₃), 0.84 (s, 3 H, CH₃),0.92 (s, 3 H, CH₃), 1.00-1.90 (m,30 H), 1.99 (s, 3 H, CH₃CO), 2.10-2.20 (m, 1 H), 2.90-3.05 (m, 3 H),3.10-3.25 (m, 1 H), 3.54-3.62 (m,1 H, OCH), 3.68-3.76 (m, 1 H, OCH),3.70-3.88 (m, 1 H, OCH), 4.30-4.40(m, 1 H, H-3), 4.53 (bs, 1 H, CH═),4.65 (bs, 1 H, CH═), 7.62 (bs, 1 H,NH). 582.4537 (M + H)⁺. |
| 19 | 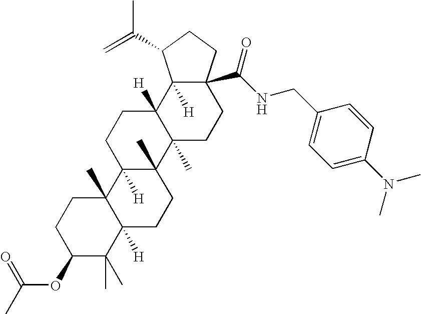 | 0.79 (s, 12 H, CH₃), 0.80 (s, 3 H,CH₃), 0.91 (s, 3 H, CH₃), 1.00-1.85(m, 23 H), 1.99 (s, 3 H, CH₃CO),2.10-2.20 (m, 1 H), 2.95-3.05 (m,1 H), 2.84 (s, 6 H, NCH₃), 4.06 (dd,1 H, J = 14.6 Hz, J = 5.8 Hz, CH₂N),4.17 (dd, 1 H, J = 14.6 Hz, J = 5.8Hz, CH₂N), 4.36 (dd, 1 H, J = 11.1Hz, J = 4.9 Hz, H-3), 4.53 (bs, 1 H,CH═), 4.65 (d, 1 H, J = 2.1 Hz,CH═), 6.64 (d, 2 H, J = 8.8 Hz, CHArom), 7.05 (d, 2 H, J = 8.8 Hz, CHArom), 8.00 (t, 1 H, J = 6.1 Hz, NH).631.4826 (M⁺ + 1). |
| 20 | 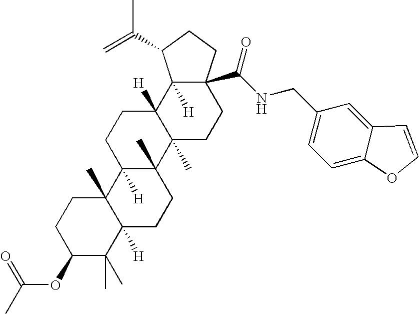 | 0.75 (s, 3 H, CH₃), 0.79 (s, 9 H, CH₃),0.91 (s, 3 H, CH₃), 1.00-1.88 (m,25 H), 1.99 (s, 3 H, CH₃CO), 2.15-2.22 (m, 1 H), 2.95-3.10 (m, 1 H),4.06 (dd, 1 H, J = 14.6 Hz, J = 5.8Hz, CH₂N), 4.22 (dd, 1 H, J = 14.7Hz, J = 5.9 Hz, CH₂N), 4.34-4.43(m, 2 H, H-3 and CH₂N), 4.53 (bs,1 H, CH═), 4.65 (bs, 1 H, CH═), 6.90(d, 1 H, J = 2.1 Hz, J = 1.0 Hz, CHArom), 7.20 (dd, 1 H, J = 8.7 Hz, J =1.7 Hz, CH Arom), 7.53-7.48 (m,2 H, CH Arom), 7.96 (d, 1 H, J = 2.1Hz, CH Arom), 8.20 (t, 1 H, J = 6.0Hz, NH). 628.4370 (M⁺+ 1). |
| 21 | 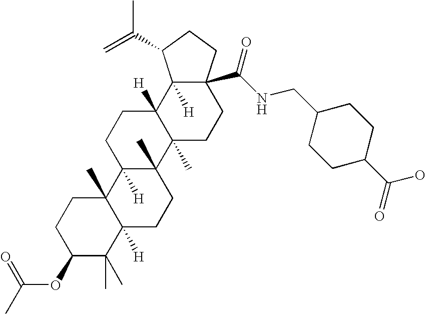 | 0.79 (s, 9 H, CH₃), 0.80 (s, 3 H, CH₃),0.84 (s, 3 H, CH₃), 0.92 (s, 3 H, CH₃),1.00-1.95 (m, 32 H), 1.99 (s, 3 H,CH₃CO), 2.30-2.20 (m, 1 H), 2.15-2.22 (m, 1 H), 2.65-2.80 (m, 1 H),2.95-3.10 (m, 2 H), 3.58 (s, 3 H,CO₂Me), 4.36 (dd, 1 H, J = 10.9 Hz,J = 4.7 Hz, H-3), 4.53 (bs, 1 H,CH═), 4.65 (d, 1 H, J = 2.3 Hz,CH═), 7.61 (t, 1 H, J = 6.1 Hz, NH).652.5003 (M⁺ + 1). |
| 22 | 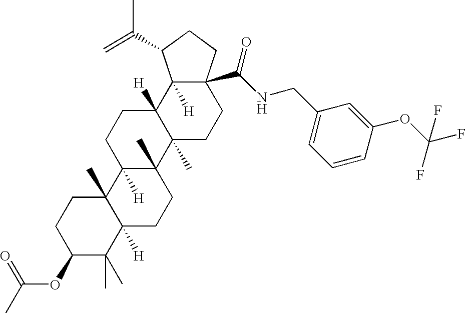 | 0.74 (s, 3 H, CH₃), 0.78 (s, 9 H, CH₃),0.92 (s, 3 H, CH₃), 1.00-1.90 (m,26 H), 1.99 (s, 3 H, CH₃CO), 2.15-2.25 (m, 1 H), 2.95-3.05 (m, 1 H),4.19 (dd, 1 H, J = 15.2 Hz, J = 6.0Hz, CH₂N), 4.30-4.40 (m, 2 H, H-3and CH₂N), 4.53 (bs, 1 H, CH═),4.65 (bs, 1 H, CH═), 7.19-7.22 (m,2 H, CH Arom), 7.28 (d, 1 H, J = 7.8Hz, CH Arom), 7.43 (t, 1 H, J = 7.8Hz, CH Arom), 8.28 (t, 1 H, J = 5.7Hz, NH). 672.49 (M⁺ + 1). |
| 23 | 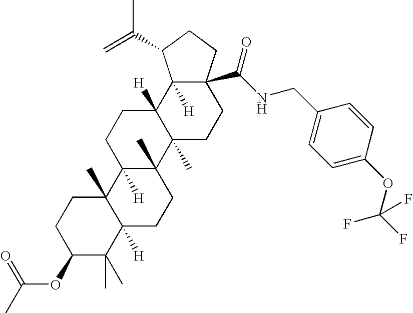 | 0.68 (s, 3 H, CH₃), 0.78 (s, 9 H, CH₃),0.91 (s, 3 H, CH₃), 0.95-1.90 (m,26 H), 1.99 (s, 3 H, CH₃CO), 2.15-2.22 (m, 1 H), 2.95-3.05 (m, 1 H),4.16 (dd, 1 H, J = 15.0 Hz, J = 6.3Hz, CH₂N), 4.29 (dd, 1 H, J = 15.0Hz, J = 6.3 Hz, CH₂N), 4.37 (dd,1 H, J = 11.6 Hz, J = 4.4 Hz, H-3),4.53 (bs, 1 H, CH═), 4.64 (bs, 1 H,CH═), 7.29 (d, 2 H, J = 8.6 Hz, CHArom), 7.35 (d, 2 H, J = 8.6 Hz, CHArom), 8.23 (t, 1 H, J = 5.7 Hz, NH).672.45 (M⁺ + 1). |
| 24 | 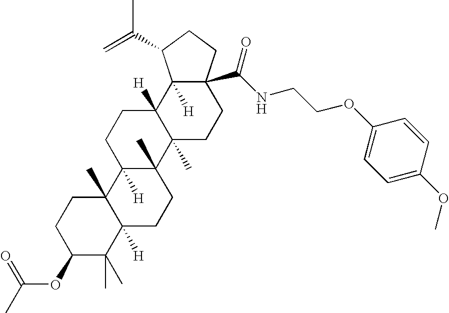 | 0.69 (s, 3 H, CH₃), 0.76 (s, 3 H, CH₃),0.78 (s, 6 H, CH₃), 0.85-1.00 (m, 5 H,CH₃, CH₂), 1.00-1.80 (m, 24 H), 1.99(s, 3 H, CH₃CO), 2.10-2.20 (m, 1 H),2.95-3.05 (m, 1 H), 3.20-3.30 (m,1 H, CH₂O), 3.40-3.50 (m, 1 H,CH₂O), 3.69 (s, 3 H, CH₃O), 3.85-3.90 (m, 2 H, CH₂N), 4.35 (dd, 1 H, J =11.4 Hz, J = 5.0 Hz, H-3), 4.53(bs, 1 H, CH═), 4.64 (d, 1 H, J = 2.3Hz, CH═), 6.84 (s, 4 H, CH Arom),7.79 (t, 1 H, J = 5.7 Hz, NH).648.4636 (M⁺ + 1). |
| 25 | 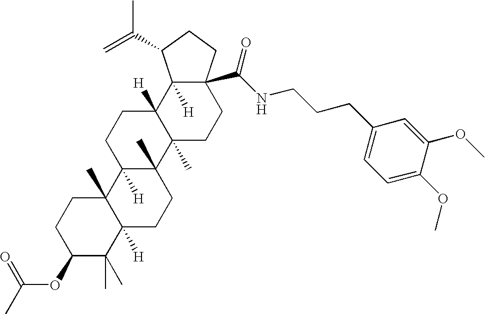 | 0.79 (s, 9 H, CH₃), 0.84 (s, 3 H, CH₃),0.93 (s, 6 H, CH₃), 1.00-1.85 (m,26 H), 1.99 (s, 3 H, CH₃CO), 2.10-2.20 (m, 1 H), 2.55-2.65 (m, 1 H),2.90-3.07 (m, 2 H, CH₂N and CH),3.09-3.20 (m, 1 H, CH₂N), 3.71 (s,3 H, CH₃O), 3.73 (s, 3 H, CH₃O),4.36 (dd, 1 H, J = 11.1 Hz, J = 4.6Hz, H-3), 4.53 (bs, 1 H, CH═), 4.65(bs, 1 H, CH═), 6.68 (d, 1 H, J = 8.2Hz, CH Arom), 6.76 (bs, 1 H, CHArom), 6.84 (d, 1 H, J = 8.2 Hz, CHArom), 7.61 (bs, 1 H, NH).698.49 (M⁺ + 23). |
| 26 | 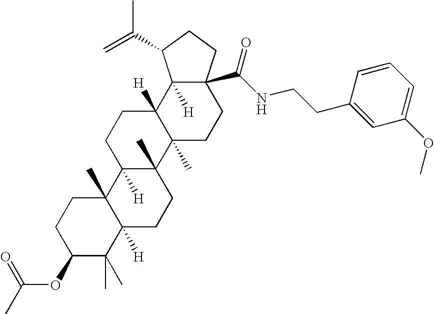 | 0.70-0.80 (s, 9 H, CH₃), 0.85-1.00 (s,6 H), 1.10-1.80 (m, 26 H), 1.99 (s,3 H, CH₃CO), 2.02-2.10 (m, 1 H),2.60-2.75 (m, 2 H), 2.95-3.05 (m,1 H), 3.25-3.45 (m, 1 H, CH₂N), 3.72(s, 3 H, CH₃O), 4.37 (dd, 1 H, J =11.2 Hz, J = 4.6 Hz, H-3), 4.53 (bs,1 H, CH═), 4.64 (bs, 1 H, CH═), 6.70-6.80 (m, 3 H, CH Arom), 7.18 (dd,1 H, J = 8.9 Hz, J = 7.3 Hz, CHArom), 7.60 (t, 1 H, J = 6.1 Hz, NH).632.4674 (M⁺ + 1). |
| 27 | 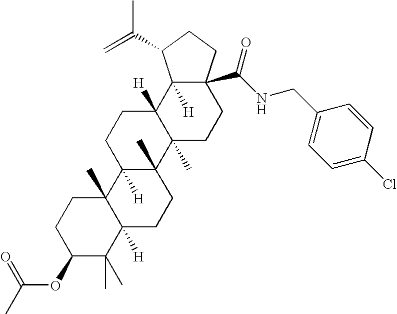 | 0.75 (s, 3 H, CH₃), 0.78 (s, 6 H, CH₃),0.79 (s, 3 H, CH₃), 0.86 (s, 3 H, CH₃),0.92 (s, 3 H, CH₃), 0.98-1.86 (m,23 H), 1.99 (s, 3 H, CH₃CO), 2.12-2.22 (m, 1 H), 2.95-3.06 (m, 1 H),4.15 (dd, 1 H, J = 15.2 Hz, J = 5.9Hz, CH₂N), 4.25 (dd, 1 H, J = 15.2Hz, J = 5.8 Hz, CH₂N), 4.36 (dd,1 H, J = 11.2 Hz, J = 4.8 Hz, H-3),4.53 (bs, 1 H, CH═), 4.65 (d, 1 H, J =2.5 Hz, CH═), 7.25 (d, 2 H, J = 8.4Hz, CH Arom), 7.35 (d, 2 H, J = 8.4Hz, CH Arom), 8.21 (t, 1 H, J = 6.0Hz, NH). 622.54 (M⁺ + 1). |
| 28 | 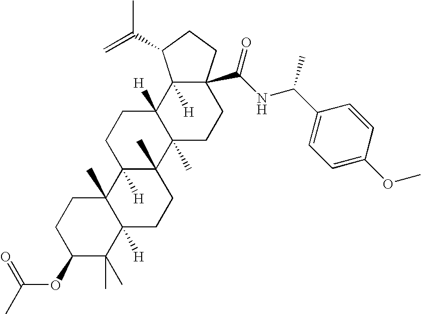 | 0.58 (s, 3 H, CH₃), 0.75 (s, 3 H, CH₃),0.77 (s, 6 H, CH₃), 0.80-0.95 (s, 6 H),1.00-1.90 (m, 26 H), 1.99 (s, 3 H,CH₃CO), 2.20-2.30 (m, 1 H), 2.95-3.07 (m, 1 H), 3.72 (s, 3 H, CH₃O),4.34 (dd, 1 H, J = 10.9 Hz, J = 5.1Hz, H-3), 4.53 (bs, 1 H, CH═), 4.64(bs, 1 H, CH═), 4.84-4.94 (m, 1 H,CHN), 6.83 (d, 2 H, J = 8.7 Hz, CHArom), 7.21 (d, 2 H, J = 8.7 Hz, CHArom), 7.77 (d, 1 H, J = 8.4 Hz,NH). 632.4684 (M⁺ + 1). |
| 29 | 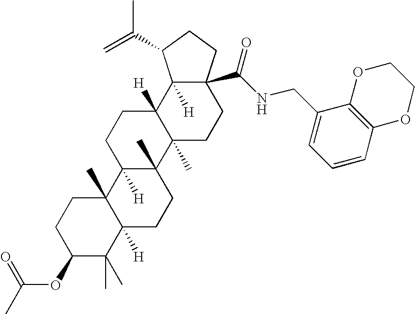 | 0.77 (s, 3 H, CH₃), 0.79 (s, 6 H, CH₃),0.91 (s, 3 H, CH₃), 0.95-1.85 (m,26 H), 1.99 (s, 3 H, CH₃CO), 2.10-2.20 (m, 1 H), 2.95-3.07 (m, 1 H),4.05 (m, 1 H, CH₂N), 4.15 (m, 1 H,CH₂N), 4.19 (s, 4 H, CH₂O), 4.32-4.40 (m, 1 H, H-3), 4.53 (bs, 1 H,CH═), 4.64 (bs, 1 H, CH═), 6.67-6.76(m, 3 H, CH Arom), 8.07 (bs, 1 H,NH). 646.4458 (M⁺ + 1). |
| 30 | 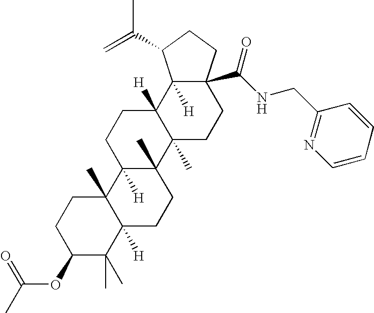 | 0.78 (s, 3 H, CH₃), 0.79 (s, 6 H, CH₃),0.93 (s, 6 H, CH₃), 1.00-1.94 (m,25 H), 1.99 (s, 3 H, CH₃CO), 2.18-2.28 (m, 1 H), 2.95-3.08 (m, 1 H),4.05 (m, 1 H, CH₂N), 4.26 (dd, 1 H, J =15.8 Hz, J = 5.9 Hz, CH₂N), 4.33-4.40 (m, 2 H, H-3 and CH₂N), 4.53(bs, 1 H, CH═), 4.64 (bs, 1 H, CH═),7.20-7.26 (d, 2 H, J = 7.6 Hz, CHArom), 7.73 (dt, 1 H, J = 7.7 Hz, J =1.8 Hz, CH Arom), 8.26 (t, 1 H, NH),8.47 (dd, 1 H, J = 5.9 Hz, J = 1.8 Hz,CH Arom). 589.4433 (M⁺ + 1). |
| 31 | 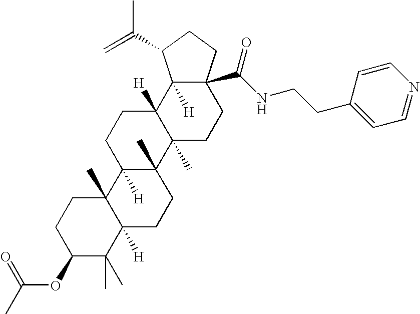 | 0.76 (s, 3 H, CH₃), 0.78 (s, 6 H, CH₃),0.79 (s, 3 H, CH₃), 0.89 (s, 3 H, CH₃),1.00-1.74 (m, 26 H), 1.99 (s, 3 H,CH₃CO), 2.00-2.08 (m, 1 H), 2.68-2.80 (m, 2 H), 2.90-3.00 (m, 1 H),3.20-3.45 (m, 2 H, CH₂N), 4.35 (dd,1 H, J = 11.1 Hz, J = 4.5 Hz, H-3),4.52 (bs, 1 H, CH═), 4.64 (d, 1 H, J =2.3 Hz, CH═), 7.21 (d, 2 H, J = 6.1Hz, CH Arom), 7.65 (t, 1 H, J = 5.5Hz, NH), 8.43 (d, 2 H, J = 6.1 Hz,CH Arom). 603.5701 (M⁺ + 1). |
| 32 | 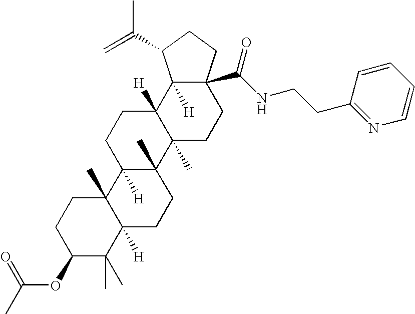 | 0.70-1.00 (m, 9 H), 1.00-1.75 (m,33 H), 1.99 (s, 3 H, CH₃CO), 2.00-2.10 (m, 1 H), 2.75-3.05 (m, 3 H),3.20-3.40 (m, 1 H), 4.36 (dd, 1 H, J =10.9 Hz, J = 4.7 Hz, H-3), 4.53 (bs,1 H, CH═), 4.64 (bs, 1 H, CH═), 7.18-7.24 (m, 2 H, CH Arom), 7.62 (t, 1 H,J = 5.4 Hz, NH), 7.68 (dt, 1 H, J =7.6 Hz, J = 1.8 Hz, CH Arom), 8.47(ddd, 1 H, J = 4.9 Hz, J = 1.8 Hz, J =0.9 Hz, CH Arom). 603.4648 (M⁺ + 1). |
| 33 | 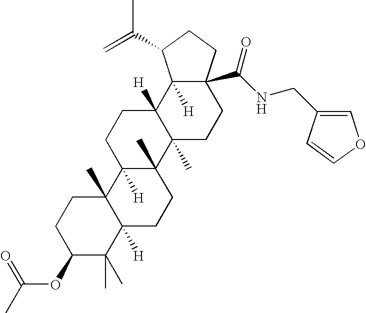 | 0.79 (s, 6 H, CH₃), 0.80 (s, 6 H, CH₃),0.91 (s, 3 H, CH₃), 0.95-1.85 (m,26 H), 1.99 (s, 3 H, CH₃CO), 2.00-2.15 (m, 1 H), 2.95-3.10 (m, 1 H),3.98 (dd, 1 H, J = 14.7 Hz, J = 5.1Hz, CH₂N), 4.12 (dd, 1 H, J = 14.7Hz, J = 6.1 Hz, CH₂N), 4.36 (dd,1 H, J = 10.8 Hz, J = 4.3 Hz, H-3),4.54 (bs, 1 H, CH═), 4.66 (bs, 1 H,CH═), 6.37 (bs, 1 H, CH Arom), 7.46(bs, 1 H, CH Arom), 7.56 (bs, 1 H,CH Arom), 7.98 (t, 1 H, NH).578.47 (M⁺ + 1). |
| 34 | 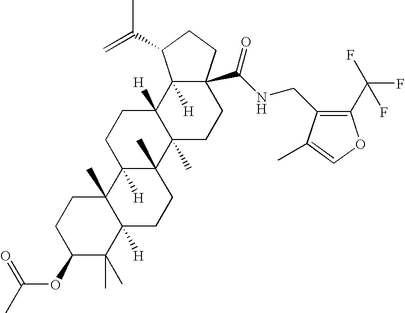 | 0.76 (s, 3 H, CH₃), 0.79 (s, 6 H, CH₃),0.80 (s, 3 H, CH₃), 0.92 (s, 3 H, CH₃),0.95-1.85 (m, 25 H), 1.99 (s, 3 H,CH₃CO), 2.10-2.20 (m, 1 H), 2.28 (s,3 H, CH₃), 2.97-3.05 (m, 1 H), 4.06(dd, 1 H, J = 15.3 Hz, CH₂N), 4.22(dd, 1 H, J = 15.3 Hz, CH₂N), 4.36(dd, 1 H, J = 10.4 Hz, J = 5.1 Hz, H-3), 4.54 (bs, 1 H, CH═), 4.66 (bs, 1 H,CH═), 5.76 (bs, 1 H, CH Arom),6.17 (bs, 1 H, CH Arom), 8.13 (t, 1 H,NH). 660.44 (M⁺ + 1). |

General Procedure for Synthesizing Compounds (35-68)

A solution of the appropriate amide (0.21 mmol) in THF (1.6 mL) and Methanol (1 mL) was treated with NaOH (4M, 0.27 mL). The mixture was stirred at room temperature overnight, and then the solvents were evaporated under reduced pressure. The residue was diluted with CH₂Cl₂ and washed with a HCl solution (0.5 N). The organic layer was dried over MgSO₄ and concentrated under reduced pressure to give the amide compounds 35-68.

TABLE 2

| | | NMR (DMSO-d₆, 400 MHz) |
|---|---|---|
| Compound No. | Structure | LC-MS (ESI) |
| 35 | 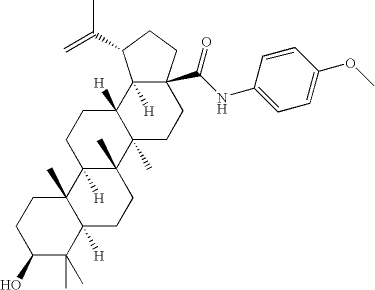 | 0.64 (s, 3 H, CH₃), 0.76 (s, 3 H,CH₃), 0.85 (s, 3 H, CH₃), 0.86 (s,3 H, CH₃),0.94 (s, 3 H, CH₃), 1.00-2.05 (m,24 H), 2.40-2.30 (m, 1 H), 2.65-2.75 (m, 1 H), 2.90-3.10 (m, 2 H,CH₂), 3.55-3.65 (m, 1 H), 3.71 (s,3 H, OCH₃), 4.28 (d, 1 H, J = 5.29Hz, H-3), 4.55 (bs, 1 H, CH═),4.68 (d, 1 H, J = 2.5 Hz, CH═),6.84 (d, 2 H, J = 9.1, CH Arom),7.45 (d, 2 H, J = 9.1 Hz, CHArom), 9.29 (s, 1 H, NH).562.55 (M + H)⁺. |
| 36 | 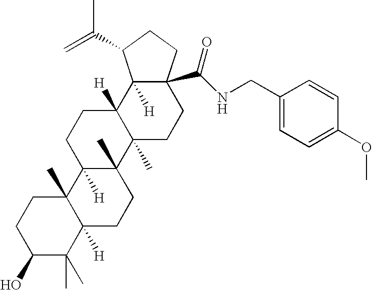 | 0.65 (s, 3 H, CH₃), 0.76 (s, 3 H,CH₃), 0.78 (s, 3 H, CH₃), 0.86 (s,3 H, CH₃), 0.90 (s, 3 H, CH₃),0.95-1.85 (m, 27 H), 2.10-2.20(m, 1 H), 2.90-3.10 (m, 2 H), 3.72(s, 3 H, CH₃O), 4.10 (dd, 1 H, J =14.8 Hz, J = 5.9 Hz, CH₂N), 4.21(dd, 1 H, J = 14.8 Hz, J = 6.0 Hz,CH₂N), 4.27 (d, 1 H, J = 5.1 Hz,H-3), 4.53 (bs, 1 H, CH═), 4.64(bs, 1 H, CH═), 6.84 (d,2 H, J =8.7 Hz, CH Arom), 7.15 (d, 2 H,J = 8.7 Hz, CH Arom), 8.09 (t,1 H, J = 6.1 Hz, NH).576.4390 (M + H)⁺. |
| 37 | 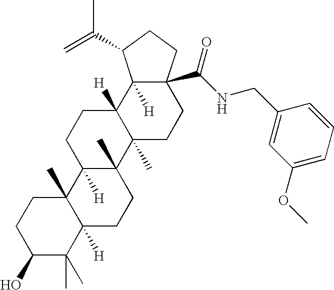 | 0.65 (s, 3 H, CH₃), 0.75 (s, 3 H,CH₃), 0.77 (s, 3 H, CH₃), 0.86 (s,3 H, CH₃), 0.91 (s, 3 H, CH₃),1.00-1.90 (m, 26 H), 2.15-2.25(m, 1 H), 2.92-3.05 (m, 2 H), 3.71(s, 3 H, CH₃O), 4.17 (dd, 1 H, J =15.3 Hz, J = 5.9 Hz, CH₂N), 4.25(dd, 1 H, J = 15.3 Hz, J = 6.1 Hz,CH₂N), 4.27 (d, 1 H, J = 5.1 Hz,H-3), 4.53 (bs, 1 H, CH═), 4.65(d, 1 H, J = 2.3 Hz, CH═), 6.74-6.84 (m, 3 H, CH Arom), 7.19 (t,1 H, J = 7.7 Hz, CH Arom), 8.15(t, 1 H, J = 6.1 Hz, NH).576.53 (M + H)⁺. |
| 38 | 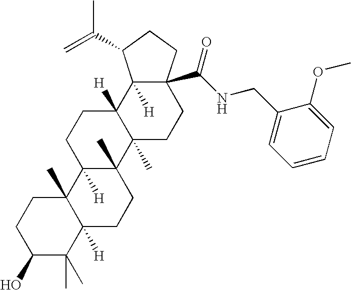 | 0.65 (s, 3 H, CH₃), 0.75 (s, 3 H,CH₃), 0.77 (s, 3 H, CH₃), 0.87 (s,3 H, CH₃), 0.91 (s, 3 H, CH₃),1.00-1.90 (m, 26 H), 2.25-2.35(m, 1 H), 2.90-3.05 (m, 2 H), 3.79(s, 3 H, CH₃O), 4.20 (d, 2 H, J =5.8 Hz, CH₂N), 4.27 (d, 1 H, J =5.1 Hz, H-3), 4.52 (bs, 1 H,CH═),4.64 (d, 1 H, J = 2.5 Hz,CH═), 6.86 (dt, 1 H, J = 7.5 Hz, J =1.0 Hz, CH Arom), 6.95 (d,1 H, J = 7.43 Hz, CH Arom),7.11 (d, 1 H, J = 5.9 Hz, CHArom), 7.19 (dt, 1 H, J = 8.6 Hz,J = 1.6 Hz, CH Arom), 7.97 (t,1 H, J = 5.8 Hz, NH). |
| 39 | 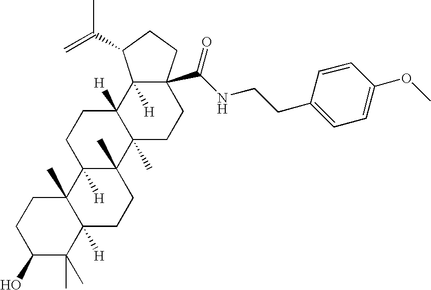 | 0.64 (s, 3 H, CH₃), 0.75 (s, 6 H,CH₃), 0.86 (s, 3 H, CH₃), 0.88 (s,3 H, CH₃), 1.00-1.80 (m, 27 H),2.00-2.10 (m, 1 H), 2.55-2.70 (m,2 H), 2.90-3.05 (m, 2 H), 3.10-3.20 (m, 1 H), 3.70 (s, 3 H,CH₃O), 4.27 (d, 1 H, J = 5.1 Hz,H-3), 4.52 (bs, 1 H, CH═), 4.63(d, 1 H, J = 2.0 Hz, CH═), 6.82(d, 2 H, J = 8.7 Hz, CH Arom),7.10 (d, 2 H, J = 8.7 Hz, CHArom), 7.57 (t, 1 H, J = 5.9 Hz,NH). 590.53 (M + H)⁺. |
| 40 | 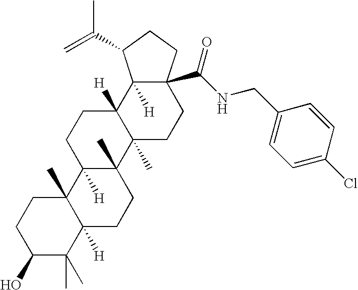 | 0.65 (s, 3 H, CH₃), 0.74 (s, 3 H,CH₃), 0.75 (s, 3 H, CH₃), 0.86 (s,3 H, CH₃), 0.90 (s, 3 H, CH₃),0.95-1.85 (m, 26 H), 2.10-2.25(m, 1 H), 2.90-3.07 (m, 2 H), 4.15(dd, 1 H, J = 15.0 Hz, J = 6.1 Hz,CH₂N), 4.25 (dd, 1 H, J = 15.0Hz, J = 5.7 Hz, CH₂N), 4.27 (d,1 H, J = 5.3 Hz, H-3), 4.53 (bs,1 H, CH═), 4.64 (d, 1 H, J = 2.1Hz, CH═), 7.25 (d, 2 H, J = 8.5Hz, CH Arom), 7.35 (d, 2 H, J =8.5 Hz, CH Arom), 8.20 (t, 1 H, J =5.9 Hz, NH). 580.58 (M + H)⁺. |
| 41 | 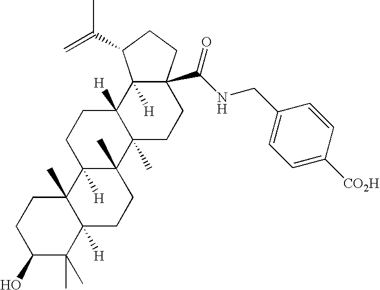 | 0.65 (s, 3 H, CH₃), 0.75 (s, 3 H,CH₃), 0.77 (s, 3 H, CH₃), 0.86 (s,3 H, CH₃), 0.91 (s, 3 H, CH₃),1.00-1.90 (m, 26 H), 2.15-2.25(m, 1 H), 2.90-3.07 (m, 2 H), 4.22(dd, 1 H, J = 15.6 Hz, J = 5.9 Hz,CH₂N), 4.27 (d, 1 H, J = 5.1 Hz,H-3), 4.34 (dd, 1 H, J = 15.6 Hz,J = 6.0 Hz, CH₂N), 4.53 (bs, 1 H,CH═), 4.64 (bs, 1 H, CH═), 7.34(d, 2 H, J = 8.1 Hz, CH Arom),7.86 (d, 2 H, J = 8.1 Hz, CHArom), 8.25 (t, 1 H, J = 6.0 Hz,NH), 12.87 (bs, 1 H, CO₂H).588.4057 (M − H)⁻. |
| 42 | 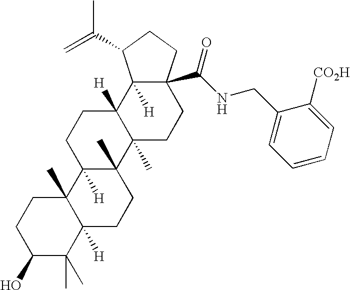 | 0.64 (s, 3 H, CH₃), 0.66 (s, 3 H,CH₃), 0.73 (s, 3 H, CH₃), 0.86 (s,3 H, CH₃), 0.89 (s, 3 H, CH₃),0.90-1.85 (m, 26 H), 2.05-2.15(m, 1 H), 2.85-3.08 (m, 2 H), 4.27(bs, 1 H, H-3), 4.44 (d, 2 H, J =5.5 Hz, CH₂N), 4.52 (bs, 1 H,CH═), 4.64 (bs, 1 H, CH═), 7.21(t, 2 H, J = 6.6 Hz, CH Arom),7.29 (t, 1 H, J = 6.8 Hz, CHArom), 7.71 (d, 1 H, J = 7.2 Hz,CH Arom), 8.48 (bs, 1 H, NH).590.4197 (M + H)⁺. |
| 43 | 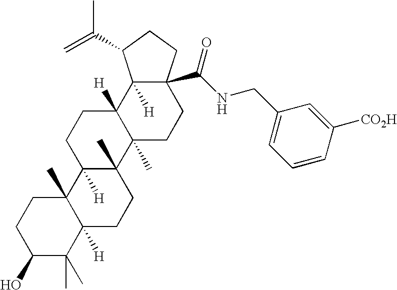 | 0.64 (s, 3 H, CH₃), 0.70 (s, 3 H,CH₃), 0.73 (s, 3 H, CH₃), 0.86 (s,CH₃), 0.90 (s, 3 H, CH₃),1.00-1.80 (m, 26 H), 2.15-2.25(m, 1 H), 2.90-3.08 (m, 2 H), 4.20(dd, 1 H, J = 15.0 Hz, J = 5.6 Hz,CH₂N), 4.27 (d, 1 H, J = 4.9 Hz,H-3), 4.34 (dd, 1 H, J = 15.0 Hz,J = 5.6 Hz, CH₂N), 4.53 (bs, 1 H,CH═), 4.64 (bs, 1 H, CH═), 7.41(t, 1 H, J = 7.6 Hz, CH Arom),7.47 (d, 1 H, J = 7.6 Hz, CHArom), 7.78 (d, 1 H, J = 7.2 Hz,CH Arom), 7.85 (s, 1 H, CHArom), 8.25 (t, 1 H, J = 5.7 Hz,NH), 12.88 (bs, 1 H, CO₂H).590.4207 (M + H)⁺. |
| 44 | 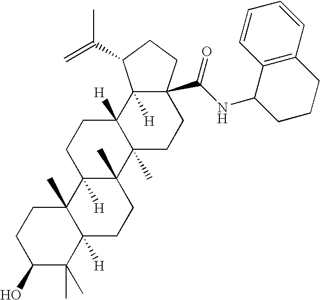 | 0.65 (s, 3 H, CH₃), 0.66 (s, 3 H,CH₃), 0.77 (s, 3 H, CH₃), 0.79 (s,3 H, CH₃), 0.87 (s, 6 H, CH₃),0.92 (s, 3 H, CH₃), 0.93 (s, 3 H,CH₃), 0.97 (s, 3 H, CH₃), 1.00-1.95 (m, 17 H), 2.15-2.27 (m,1 H), 2.55-2.80 (m, 3 H), 2.80-3.17 (m, 2 H), 4.28 (dd, 1 H, J =5.1 Hz, J = 2.3 Hz, H-3), 4.55(bs, 1 H, CH═), 4.67 (d, 1 H, J =6.3 Hz, CH═), 4.95-5.10 (m, 1 H,CNH), 7.00-7.20 (m, 4 H, CHArom), 7.80-7.80 (m, 1 H, NH).589.63 (M + H)⁺. |
| 45 | 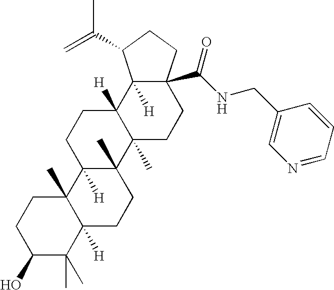 | 0.64 (s, 3 H, CH₃), 0.71 (s, 3 H,CH₃), 0.74 (s, 3 H, CH₃), 0.86 (s,9 H, CH₃), 0.90 (s, 3 H, CH₃),0.95-1.90 (m, 26 H), 2.10-2.20(m, 1 H), 2.90-3.10 (m, 2 H), 4.16(dd, 1 H, J = 15.0 Hz, J = 5.8 Hz,CH₂N), 4.27 (d, 1 H, J = 5.1 Hz,H-3), 4.29 (dd, 1 H, J = 15.0 Hz,J = 5.5 Hz, CH₂N), 4.53 (bs, 1 H,CH═), 4.64 (bs, 1 H, CH═), 7.30-7.36 (m, 1 H, CH Arom), 7.63(dt, 1 H, J = 8.0 Hz, J = 1.6 Hz,CH Arom), 8.25 (t, 1 H, J = 5.9Hz, NH), 8.42 (dd, 1 H, J = 4.7Hz, J = 1.6 Hz, CH Arom), 8.46(d, 1 H, J = 1.6 Hz, CH Arom).547.66 (M + H)⁺. |
| 46 | 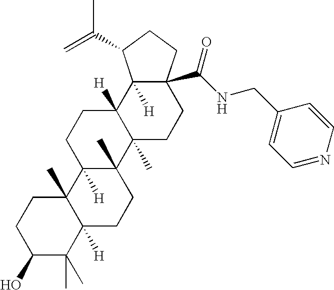 | 0.64 (s, 3 H, CH₃), 0.72 (s, 3 H,CH₃), 0.74 (s, 3 H, CH₃), 0.87 (s,9 H, CH₃), 0.91 (s, 3 H, CH₃),1.00-1.95 (m, 26 H), 2.15-2.25(m, 1 H), 2.90-3.02 (m, 2 H), 4.36(d, 2 H, J = 5.8 Hz CH₂N), 4.53(bs, 1 H, CH═), 4.63 (d, 1 H, J =2.1 Hz, CH═), 7.60 (d, 2 H, J =6.3 Hz, CH Arom), 8.44 (t, 1 H, J =5.8 Hz, NH), 8.71 (d, 2 H, J =6.3 Hz, CH Arom).547.4272 (M + H)⁺. |
| 47 | 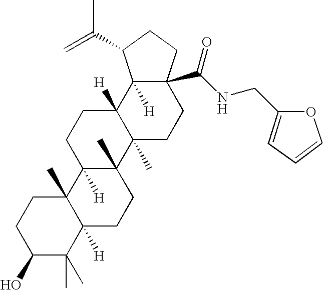 | 0.64 (s, 3 H, CH₃), 0.75 (s, 3 H,CH₃), 0.78 (s, 3 H, CH₃), 0.86 (s,3 H, CH₃), 0.90 (s, 3 H, CH₃),0.95-1.85 (m, 26 H), 2.10-2.20(m, 1 H), 2.90-3.08 (m, 2 H), 4.13(dd, 1 H, J = 15.7 Hz, J = 5.6 Hz,CH₂N), 4.25-4.33 (m, 2 H, H-3and CH₂N), 4.53 (bs, 1 H, CH═),4.65 (d, 1 H, J = 2.3 Hz, CH═),6.11 (dd, 1 H, J = 3.1 Hz, J = 0.8Hz, CH Arom), 6.37 (dd, 1 H, J =3.1 Hz, J = 1.8 Hz, CH Arom),7.52 (dd, 1 H, J = 1.8 Hz, J = 0.8Hz, CH Arom), 8.07 (t, 1 H, J =5.9 Hz, NH). 536.4100 (M + H)⁺. |
| 48 | 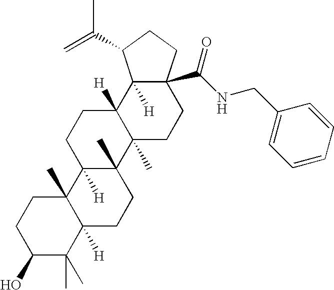 | 0.65 (s, 3 H, CH₃), 0.75 (s, 3 H,CH₃), 0.78 (s, 3 H, CH₃), 0.86 (s,3 H, CH₃), 0.91 (s, 3 H, CH₃),1.00-1.85 (m, 26 H), 2.14-2.25(m, 1 H), 2.90-3.10 (m, 2 H), 4.16(dd, 1 H, J = 15.2 Hz, J = 6.0 Hz,CH₂N), 4.25-4.35 (m, 2 H, H-3and CH₂N), 4.53 (bs, 1 H, CH═),4.65 (d, 1 H, J = 1.8 Hz, CH═),7.16-7.34 (m, 5 H, CH Arom),8.17 (t, 1 H, J = 5.9 Hz, NH).546.57 (M + H)⁺. |
| 49 | 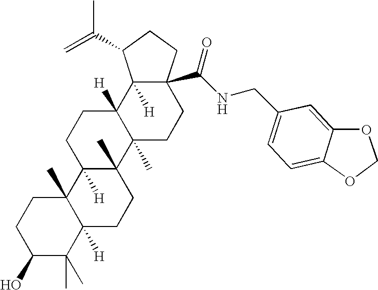 | 0.65 (s, 3 H, CH₃), 0.75 (s, 3 H,CH₃), 0.77 (s, 3 H, CH₃), 0.86 (s,3 H, CH₃), 0.90 (s, 3 H, CH₃),1.00-1.85 (m, 26 H), 2.10-2.20(m, 1 H), 2.90-3.08 (m, 2 H), 4.06(dd, 1 H, J = 15.0 Hz, J = 6.1 Hz,CH₂N), 4.20 (dd, 1 H, J = 15.0Hz, J = 6.0 Hz, CH₂N), 4.27 (d,1 H, J = 5.1 Hz, H-3), 4.53 (bs,1 H, CH═), 4.65 (d, 1 H, J = 2.2Hz, CH═), 5.96 (d, 2 H, J = 2.2Hz, OCH₂O), 6.70 (dd, 1 H, J =8.0 Hz, J = 1.7 Hz, CH Arom),6.78 (d, 1 H, J = 1.7 Hz, CHArom), 6.82 (d, 1 H, J = 8.0 Hz,CH Arom), 8.10 (t, 1 H, J = 5.9Hz, NH). 590.46 (M + H)⁺. |
| 50 | 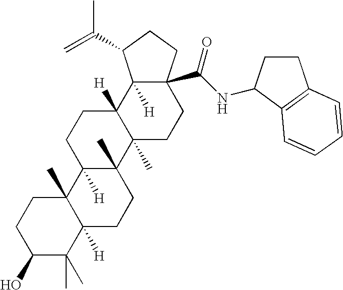 | 0.65 (s, 3 H, CH₃), 0.66 (s, 3 H,CH₃), 0.77 (s, 3 H, CH₃), 0.79 (s,3 H, CH₃), 0.87 (s, 6 H, CH₃),0.92 (s, 3 H, CH₃), 0.93 (s, 3 H,CH₃), 0.96 (s, 3 H, CH₃), 1.00-1.95 (m, 14 H), 2.05-2.35 (m,2 H), 2.55-3.15 (m, 5 H), 4.26-4.30 (m, 1 H, H-3), 4.55 (bs, 1 H,CH═), 4.72-4.65 (m, 1 H, CH═),5.28-5.40 (m, 1 H, CNH), 7.04-7.26 (m, 4 H, CH Arom), 7.84(m, 1 H, NH). 572.4455 (M + H)⁺. |
| 51 | 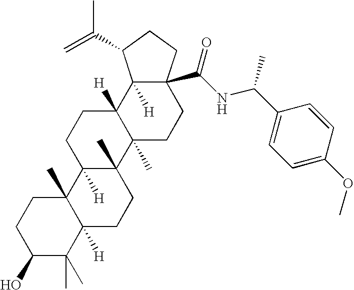 | 0.57 (s, 3 H, CH₃), 0.63 (s, 3 H,CH₃), 0.71 (s, 3 H, CH₃), 0.75-1.00 (m, 10 H, CH₃, CH₂), 1.00-1.90 (m, 25 H), 2.20-2.30 (m,1 H), 2.90-3.05 (m, 2 H), 3.72 (s,3 H, OCH₃), 4.26 (d, 1 H, J = 5.0Hz, H-3), 4.53 (bs, 1 H, CH═),4.64 (d, 1 H, J = 2.3 Hz, CH═),4.85-4.95 (m, 1 H, NCH), 6.83(d, 2 H, J = 8.7 Hz, CH Arom),7.20 (d, 2 H, J = 8.7 Hz, CHArom), 7.76 (d, 1 H, J = 7.6 Hz,NH). 590.4917 (M⁺ + 1). |
| 52 | 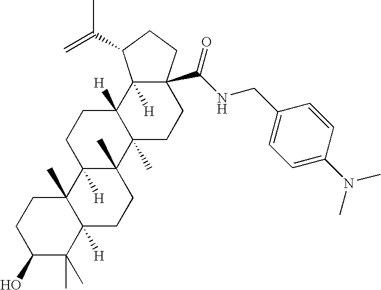 | 0.65 (s, 3 H, CH₃), 0.76 (s, 3 H,CH₃), 0.79 (s, 3 H, CH₃), 0.86 (s,3 H, CH₃), 0.90 (s, 3 H, CH₃),0.95-1.85 (m, 25 H), 2.10-2.20(m, 1 H), 2.84 (s, 6 H, NCH₃),2.92-3.08 (m, 2 H), 4.06 (dd, 1 H,J = 14.5 Hz, J = 6.0 Hz, CH₂N),4.16 (dd, 1 H, J = 5.9 Hz, J =14.5 Hz, CH₂N), 4.28 (d, 1 H, J =5.3 Hz, H-3), 4.53 (bs, 1 H,CH═), 4.65 (d, 1 H, J = 2.3 Hz,CH═), 4.85-4.95 (m, 1 H, NCH),6.64 (d, 2 H, J = 8.8 Hz, CHArom), 7.05 (d, 2 H, J = 8.8 Hz,CH Arom), 7.99 (t, 1 H, J = 6.1Hz, NH). 589.4744 (M⁺ + 1). |
| 53 | 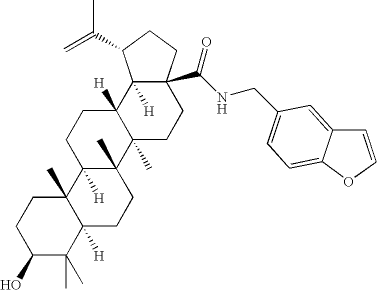 | 0.64 (s, 3 H, CH₃), 0.74 (s, 3 H,CH₃), 0.75 (s, 3 H, CH₃), 0.86 (s,3 H, CH₃), 0.90 (s, 3 H, CH₃),0.95-1.90 (m, 26 H), 2.25-2.32(m, 1 H), 2.92-3.08 (m, 2 H), 4.23(dd, 1 H, J = 6.0 Hz, J = 14.7 Hz,CH₂N), 4.27 (d, 1 H, J = 5.3 Hz,H-3), 4.40 (dd, 1 H, J = 14.7 Hz,J = 6.1 Hz, CH₂N), 4.53 (bs, 1 H,CH═), 4.65 (d, 1 H, J = 2.3 Hz,CH═), 6.89 (dd, 1 H, J = 2.2 Hz,J = 1.0 Hz, CH Arom), 7.20 (dd,1 H, J = 8.5 Hz, J = 1.5 Hz, CHArom), 7.48-7.52 (m, 2 H, CHArom), 7.96 (d, 1 H, J = 2.2 Hz,CH Arom), 8.19 (t, 1 H, J = 5.87Hz, NH). 586.4254 (M⁺ + 1). |
| 54 | 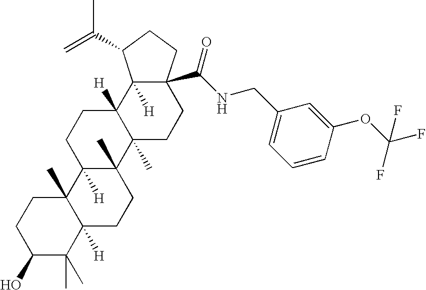 | 0.64 (s, 3 H, CH₃), 0.74 (s, 6 H,CH₃), 0.86 (s, 3 H, CH₃), 0.91 (s,3 H, CH₃), 0.96-1.90 (m, 26 H),2.12-2.22 (m, 1 H), 2.92-3.06 (m,2 H), 4.19 (dd, 1 H, J = 6.0 Hz, J =15.5 Hz, CH₂N), 4.27 (d, 1 H, J =5.1 Hz, H-3), 4.34 (dd, 1 H, J =15.5 Hz, J = 5.5 Hz, CH₂N), 4.53(bs, 1 H, CH═), 4.64 (bs, 1 H,CH═), 6.80-7.24 (m, 2 H, CHArom), 7.27 (d, 1 H, J = 7.4 Hz,CH Arom), 7.43 (t, 1 H, J = 7.6Hz, CH Arom), 8.27 (t, 1 H, J =5.8 Hz, NH). 630.51 (M⁺ + 1). |
| 55 | 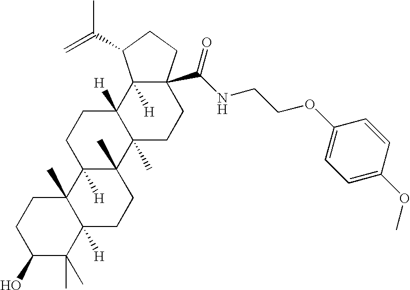 | 0.63 (s, 3 H, CH₃), 0.68 (s, 3 H,CH₃), 0.72 (s, 3 H, CH₃), 0.76-1.00 (m, 9 H), 1.00-1.80 (m,24 H), 2.12-2.20 (m, 1 H), 2.92-3.06 (m, 2 H), 3.20-3.30 1 H),3.40-3.50 (m, 1 H), 3.69 (s, 3 H,OCH₃), 3.84-3.96 (m, 2 H,CH₂N), 4.53 (bs, 1 H, CH═), 4.64(d, 1 H, J = 2.0 Hz, CH═), 6.84(s, 4 H, CH Arom), 7.77 (t, 1 H, J =5.7 Hz, NH). 606.4564 (M⁺ + 1). |
| 56 | 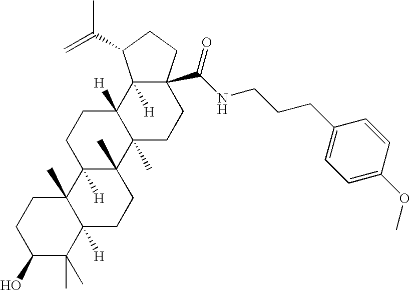 | 0.64 (s, 3 H, CH₃), 0.75 (s, 3 H,CH₃), 0.86 (s, 3 H, CH₃), 0.91 (s,3 H, CH₃), 1.00-1.85 (m, 33 H),2.10-2.20 (m, 1 H), 2.55-2.65 (m,1 H), 2.90-3.15 (m, 3 H), 3.71 (s,3 H, OCH₃), 4.27 (d, 1 H, J = 5.1Hz, H-3), 4.53 (bs, 1 H, CH═),4.64 (d, 1 H, J = 2.7 Hz, CH═),6.83 (d, 2 H, J = 8.7 Hz, CHArom), 7.09 (d, 2 H, J = 8.7 Hz,CH Arom), 7.61 (t, 1 H, J = 5.7Hz, NH). 604.55 (M⁺ + 1). |
| 57 | 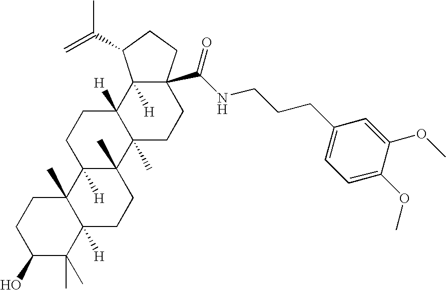 | 0.64 (s, 3 H, CH₃), 0.75 (s, 3 H,CH₃), 0.83 (s, 3 H, CH₃), 0.86 (s,3 H, CH₃), 0.91 (s, 3 H, CH₃),1.00-1.85 (m, 28 H), 2.10-2.20(m, 1 H), 2.55-2.65 (m, 1 H),2.90-3.07 (m, 3 H), 3.09-3.20 (m,1 H), 3.55-3.65 (m, 1 H), 3.71 (s,3 H, OCH₃), 3.71 (s, 3 H, OCH₃),4.27 (d, 1 H, J = 5.1 Hz, H-3),4.53 (bs, 1 H, CH═), 4.65 (bs,1 H, CH═), 6.68 (d, 1 H, J = 8.1Hz, CH Arom), 6.76 (bs, 1 H, CHArom), 6.84 (d, 1 H, J = 8.1 Hz,CH Arom), 7.61 (bs, 1 H, NH).634.60 (M⁺ + 1). |
| 58 | 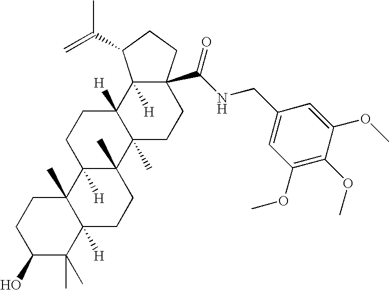 | 0.60-0.95 (m, 15 H), 1.00-1.90(m, 26 H), 2.15-2.25 (m, 1 H),2.55-2.65 (m, 1 H), 2.92-3.07 (m,2 H), 3.61 (s, 3 H, OCH₃), 3.73 (s,6 H, OCH₃), 4.13 (dd, 1 H, J =15.3 Hz, J = 5.8 Hz, CH₂N),4.23 (dd, 1 H, J = 15.3 Hz, J =6.0 Hz, CH₂N), 4.53 (bs, 1 H,CH═), 4.65 (d, 1 H, J = 2.3 Hz,CH═), 6.53 (s, 2 H, CH Arom),8.15 (t, 1 H, J = 6.0 Hz, NH).636.4665 (M⁺ + 1). |
| 59 | 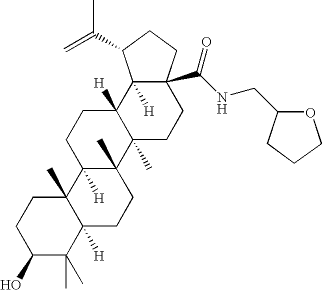 | 0.64-0.89 (m, 20 H), 0.95-1.90(m, 30 H), 2.10-2.24 (m, 2 H),2.90-3.02 (m, 2 H), 3.53-3.63 (m,1 H, CHO), 3.66-3.76 (m, 1 H,CHO), 3.77-3.86 (m, 1 H, CHO),4.28 (d, 1 H, J = 4.9 Hz, H-3),7.54 (bs, 1 H, NH).542.4579 (M⁺ + 1). |
| 60 | 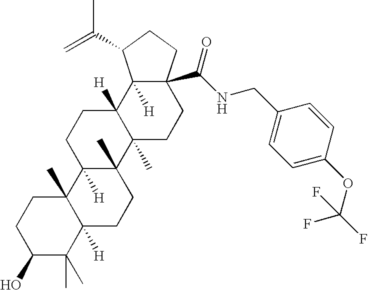 | 0.64 (s, 3 H, CH₃), 0.67 (s, 3 H,CH₃), 0.73 (s, 3 H, CH₃), 0.86 (s,3 H, CH₃), 0.89 (s, 3 H, CH3),0.95-1.87 (m, 26 H), 2.15-2.22(m, 1 H), 2.92-3.07 (m, 2 H), 4.16(dd, 1 H, J = 15.1 Hz, J = 5.7 Hz,CH₂N), 4.25-4.32 (m, 2 H, H-3and CH₂N), 4.53 (bs, 1 H, CH═),4.64 (d, 1 H, J = 1.8 Hz, CH═),7.29 (d, 2 H, J = 8.4 Hz, CHArom), 7.36 (d, 2 H, J = 8.4 Hz,CH Arom), 8.23 (t, 1 H, J = 6.1Hz, NH). 630.4126 (M⁺ + 1). |
| 61 | 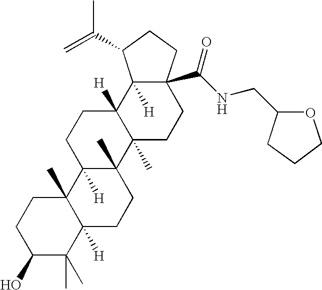 | 0.64 (s, 3 H, CH₃), 0.76 (s, 3 H,CH₃), 0.84 (s, 3 H, CH₃), 0.86 (s,3 H, CH₃), 0.90 (s, 3 H, CH₃),0.95-1.85 (m, 30 H), 2.10-2.20(m, 1 H), 2.90-3.05 (m, 3 H),3.17-3.10 (m, 1 H), 3.55-3.63 (m,1 H, CHO), 3.66-3.76 (m, 1 H,CHO), 3.77-3.86 (m, 1 H, CHO),4.27 (d, 1 H, J = 5.3 Hz, H-3),4.53 (bs, 1 H, CH═), 4.64 (bs,1 H, CH═), 7.63 (bs, 1 H, NH).540.4411 (M⁺ + 1). |
| 62 | 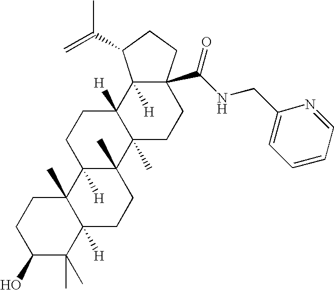 | 0.64 (s, 3 H, CH₃), 0.75 (s, 3 H,CH₃), 0.77 (s, 3 H, CH₃), 0.87 (s,3 H, CH₃), 0.92 (s, 3 H, CH₃),1.00-1.92 (m, 26 H), 2.20-2.25(m, 1 H), 2.90-3.05 (m, 2 H),4.22-4.30 (m, 2 H, H-3 andCH₂N), 4.36 (dd, 1 H, J = 15.8Hz, J = 5.8 Hz, CH₂N), 4.53 (bs,1 H, CH═), 4.64 (d, 1 H, J = 2.3Hz, CH═), 7.20-7.26 (m, 2 H, CHArom), 7.73 (dt, 1 H, J = 7.6 Hz,J = 1.8 Hz, CH Arom), 8.25 (t,1 H, J = 6.0 Hz, NH), 8.46 (ddd,1 H, J = 4.6 Hz, J = 2.0 Hz, J =1.2 Hz, CH Arom).547.4461 (M⁺ + 1). |
| 63 | 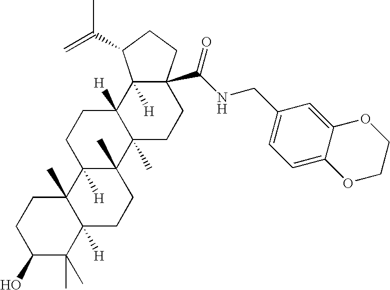 | 0.64 (s, 3 H, CH₃), 0.76 (s, 6 H,CH₃), 0.86 (s, 3 H, CH₃), 0.90 (s,3 H, CH₃), 0.95-1.90 (m, 28 H),2.10-2.22 (m, 1 H), 2.90-3.07 (m,2 H), 4.00-4.30 (m, 5 H, H-3,CH₂O, CH₂N), 4.53 (bs, 1 H,CH═), 4.64 (bs, 1 H, CH═), 6.60-6.80 (m, 3 H, CH Arom), 8.06(bs, 1 H, NH)604.4346 (M⁺ + 1). |
| 64 | 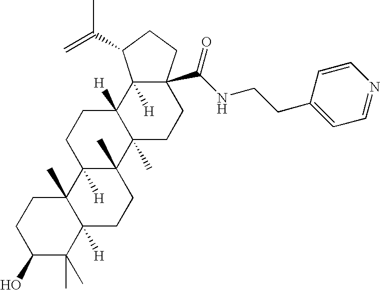 | 0.64 (s, 3 H, CH₃), 0.75 (s, 6 H,CH₃), 0.86 (s, 3 H, CH₃), 0.87 (s,3 H, CH₃), 1.00-1.75 (m, 28 H),1.97-2.08 (m, 1 H), 2.90-3.00 (m,2 H), 3.20-3.45 (m, 2 H, CH₂N),4.27 (d, 1 H, J = 5.3 Hz, H-3),4.52 (bs, 1 H, CH═), 4.63 (d, 1 H,J = 2.1 Hz, CH═), 7.21 (d, 2 H, J =6.0 Hz, CH Arom), 7.65 (t, 1 H,J = 5.8 Hz, NH), 8.43 (d, 2 H, J =6.0 Hz, CH Arom).561.3 (M⁺ + 1). |
| 65 | 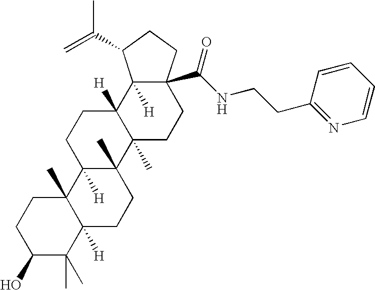 | 0.64 (s, 3 H, CH₃), 0.75 (s, 3 H,CH₃), 0.79 (s, 3 H, CH₃), 0.86 (s,3 H, CH₃), 0.88 (s, 3 H, CH₃),1.00-1.80 (m, 26 H), 2.00-2.10(m, 1 H), 2.80-3.05 (m, 4 H),3.35-3.50 (m, 1 H, CH₂N), 3.55-3.65 (m, 1 H, CH₂N), 4.27 (d,1 H, J = 5.3 Hz, H-3), 4.53 (bs,1 H, CH═), 4.64 (bs, 1 H, CH═),7.17-7.23 (m, 2 H, CH Arom),7.62 (bs, 1 H, NH), 7.68 (dt, 1 H,J = 7.5 Hz, J = 2.0 Hz, CHArom), 8.47 (d, 1 H, J = 3.7 Hz,CH Arom). 561.42 (M⁺ + 1). |
| 66 | 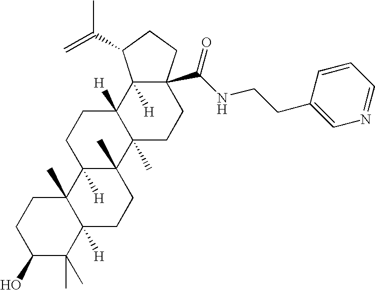 | 0.65 (s, 3 H, CH₃), 0.72 (s, 3 H,CH₃), 0.75 (s, 3 H, CH₃), 0.87 (s,6 H, CH₃), 1.00-1.70 (m, 26 H),1.95-2.05 (m, 1 H), 2.75-3.00 (m,4 H), 3.35-3.50 (m, 1 H, CH₂N),3.30-3.50 (m, 2 H, H-3 andCH₂N), 4.52 (bs, 1 H, CH═), 4.62(bs, 1 H, CH═), 7.65 (m, 2 H, NHand CH Arom), 8.03 (bs, 1 H, CHArom), 8.60 (bs, 2 H, CH Arom).561.49 (M⁺ + 1). |
| 67 | 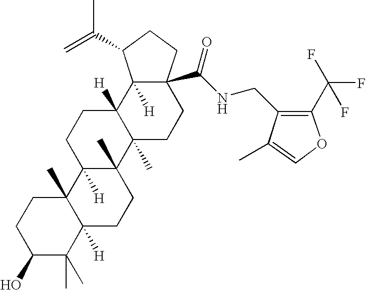 | 0.65 (s, 3 H, CH₃), 0.76 (s, 6 H,CH₃), 0.86 (s, 3 H, CH₃), 0.90 (s,3 H, CH₃), 0.95-1.85 (m, 26 H),2.10-2.18 (m, 1 H), 2.28 (s, 3 H,CH₃), 2.90-3.08 (m, 2 H), 4.06(dd, 1 H, J = 15.0 Hz, J = 5.6 Hz,CH₂N), 4.21 (dd, 1 H, J = 15.0Hz, J = 4.4 Hz, CH₂N), 4.27 (d,1 H, J = 4.7 Hz, H-3), 4.53 (bs,1 H, CH═), 4.66 (bs, 1 H, CH═),6.17 (s, 1 H, CH Arom), 8.13 (bs,1 H, NH). 618.37 (M⁺ + 1). |
| 68 | 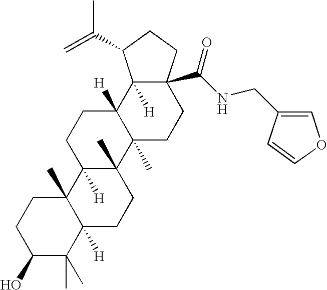 | 0.65 (s, 3 H, CH₃), 0.76 (s, 3 H,CH₃), 0.80 (s, 3 H, CH₃), 0.86 (s,3 H, CH₃), 0.90 (s, 3 H, CH₃),0.94-1.82 (m, 26 H), 2.08-2.18(m, 1 H), 2.90-3.08 (m, 2 H), 3.98(dd, 1 H, J = 15.2 Hz, J = 5.0 Hz,CH₂N), 4.11 (dd, 1 H, J = 15.2Hz, J = 5.4 Hz, CH₂N), 4.28 (d,1 H, J = 5.1 Hz, H-3), 4.53 (bs,1 H, CH═), 4.66 (bs, 1 H, CH═),6.36 (s, 1 H, CH Arom), 7.46 (s,1 H, CH Arom), 7.56 (bs, 1 H, CHArom), 7.97 (bs, 1 H, NH).536.42 (M⁺ + 1). |

General procedure for Synthesizing compounds (69-121)

A solution of the appropriate amide 35-68 (0.17 mmol) in dry Pyridine (4 mL), under nitrogen atmosphere, was treated with 2,2-Dimethylsuccinic anhydride (0.109 g, 0.85 mmol) and DMAP (0.021 g, 0.17 mmol) and the mixture was refluxed overnight. The reaction mixture was diluted with CH₂Cl₂ and washed with H₂O. The organic layer was dried over MgSO₄ and concentrated under reduced pressure to give the carboxylic acid product. The crude material was purified by HPLC.

TABLE 3

| Com- | | |
|---|---|---|
| pound | | NMR (DMSO-d₆,400 MHz) |
| No. | Structure | LC-MS (ESI) |
| 69 | 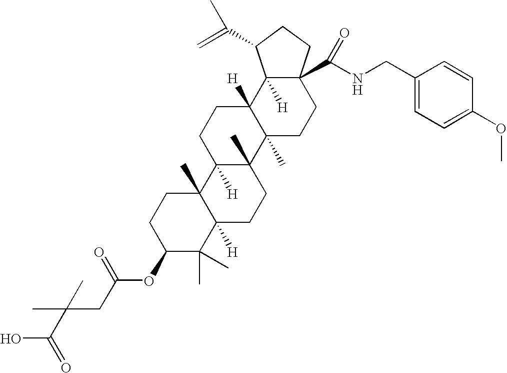 | 0.78 (s, 6 H, CH₃), 0.79 (s, 3 H, CH₃),0.80 (s, 3 H, CH₃), 0.92 (s, 3 H, CH₃),1.15 (s, 3 H, CH₃), 1.16 (s, 3 H, CH₃),1.20-1.85 (m, 32 H), 2.12-2.20 (m,1 H), 2.98-3.08 (m, 1 H), 3.72 (s, 3 H,CH₃O), 4.10 (dd, 1 H, J = 14.8 Hz, J = 6.3 Hz,CH₂N), 4.21 (dd, 1 H, J = 14.8 Hz,J = 5.9 Hz, CH₂N), 4.36 (dd, 1 H,J = 11.4 Hz, J = 5.0 Hz, H-3), 4.53 (bs,1 H, CH═), 4.65 (d, 1 H, J = 2.1 Hz,CH═), 6.84 (d, 2 H, J = 8.6 Hz, CHArom), 7.15 (d, 2 H, J = 8.6 Hz, CHArom), 8.09 (t, 1 H,J = 5.8 Hz, NH).705.50 (M + H)⁺. |
| 70 | 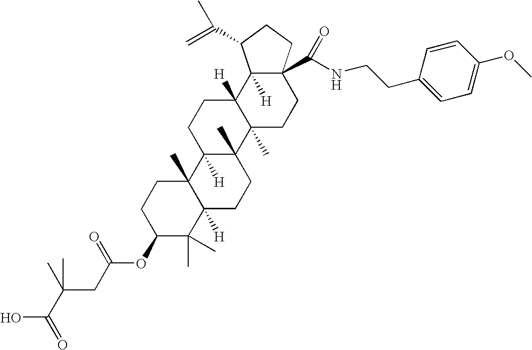 | 0.75 (s, 3 H, CH₃), 0.77 (s, 3 H, CH₃),0.78 (s, 6 H, CH₃), 0.89 (s, 3 H, CH₃),1.15 (s, 3 H, CH₃), 1.16 (s, 3 H, CH₃),1.20-1.80 (m, 29 H), 2.00-2.10 (m,1 H), 2.55-2.70 (m, 2 H), 2.90-3.02 (m,1 H), 3.10-3.20 (m, 1 H), 3.70 (s, 3 H,CH₃O), 4.35 (dd, 1 H, J = 11.3 Hz, J = 4.5 Hz,H-3), 4.52 (bs, 1 H, CH═), 4.63(bs, 1 H, CH═), 6.82 (d, 2 H,J =8.6 Hz, CH Arom), 7.09 (d, 2 H,J = 8.6 Hz, CH Arom), 7.57 (t, 1 H, J = 5.5 Hz, NH), 12.17 (bs, 1 H, CO₂H).718.58 (M + H)⁺. |
| 71 | 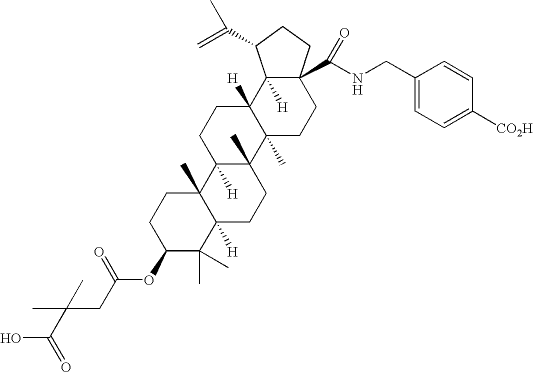 | 0.78 (s, 6 H, CH₃), 0.79 (s, 3 H, CH₃),0.92 (s, 3 H, CH₃), 1.16 (s, 3 H, CH₃),1.17 (s, 3 H, CH₃), 1.20-1.85 (m, 33 H),2.12-2.25 (m, 1 H), 2.95-3.08 (m, 1 H),4.22 (dd, 1 H, J = 15.4 Hz, J = 5.7 Hz,CH₂N), 4.30-4.40 (m, 2 H, H-3 andCH₂N), 4.53(bs, 1 H, CH═), 4.65 (bs,1 H, CH═), 7.34 (d, 2 H, J = 8.3 Hz, CHArom), 7.86 (d, 2 H, J = 8.3 Hz, CHArom), 8.26 (t, 1 H, J = 5.7 Hz, NH).718.55 (M + H)⁺. |
| 72 | 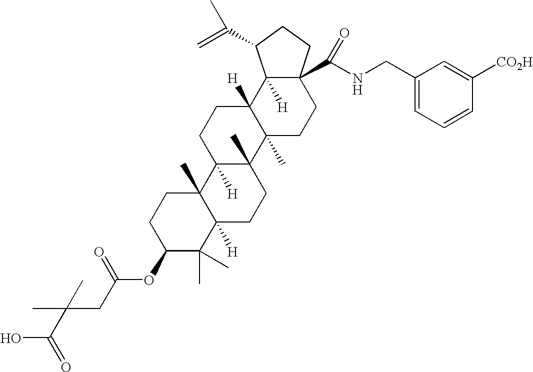 | 0.71 (s, 3 H, CH₃), 0.77 (s, 9 H, CH₃),0.91 (s, 3 H, CH₃), 1.15 (s, 3 H, CH₃),1.16 (s, 3 H, CH₃), 1.20-1.90 (m, 30 H),2.12-2.25 (m, 1 H), 2.95-3.08 (m, 1 H),4.20 (dd, 1 H, J = 15.3 Hz, J = 5.5 Hz,CH₂N), 4.29-4.40 (m, 2 H, H-3 andCH₂N), 4.53 (bs, 1 H, CH═), 4.64 (bs,1 H, CH═), 7.41 (t, 1 H, J = 7.5 Hz, CHArom), 7.48 (d, 1 H, J = 7.6 Hz, CHArom), 7.78 (d, 1 H, J = 7.4 Hz, CHArom), 7.85 (bs, 1 H, CH Arom), 8.26(t, 1 H, J = 5.8 Hz, NH).718.4710 (M + H)⁺. |
| 73 | 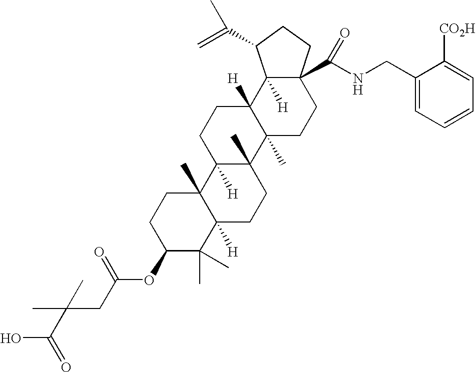 | 0.75 (s, 3 H, CH₃), 0.78 (s, 9 H, CH₃),0.93 (s, 3 H, CH₃), 1.00-1.95 (m, 36 H),2.12-2.25 (m, 1 H), 2.95-3.08 (m, 1 H),4.30-4.40 (m, 1 H, H-3), 4.45-4.60 (m,3 H, CH═ and CH₂N), 4.63 (bs, 1 H,CH═), 7.30-7.40 (m, 2 H, CH Arom),7.50 (m, 1 H,CH Arom), 7.85 (d, 1 H,J = 7.4 Hz, CH Arom), 8.08 (bs, 1 H, NH).716.59 (M − H)⁻. |
| 74 | 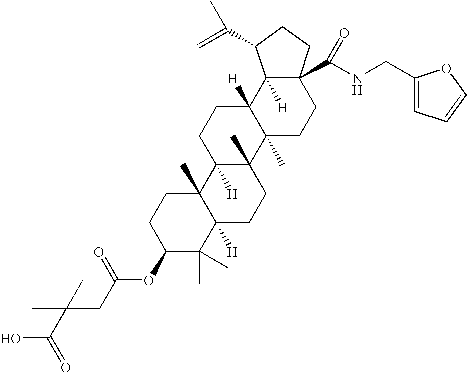 | 0.78 (s, 6 H, CH₃), 0.79 (s, 6 H, CH₃),0.91 (s, 3 H, CH₃), 1.05-1.85 (m, 34 H),2.10-2.20 (m, 1 H), 2.95-3.05 (m, 1 H),4.13 (dd, 1 H, J = 5.8 Hz,CH₂N), 4.25-4.40 (m, 2 H, H-3 andCH₂N), 4.53 (bs, 1 H, CH═), 4.65 (bs,1 H, CH═), 6.11 (dd, 1 H, J = 3.0 Hz,J = 0.7 Hz, CH Arom), 6.37 (dd, 1 H,J = 3.0 Hz, J = 1.9 Hz, CH Arom), 7.52(dd, 1 H, J = 1.9 Hz, J = 0.7 Hz, CHArom), 8.08 (t, 1 H, J = 5.9 Hz, NH),12.1 (bs, 1 H, CO₂H).664.49 (M + H)⁺. |
| 75 | 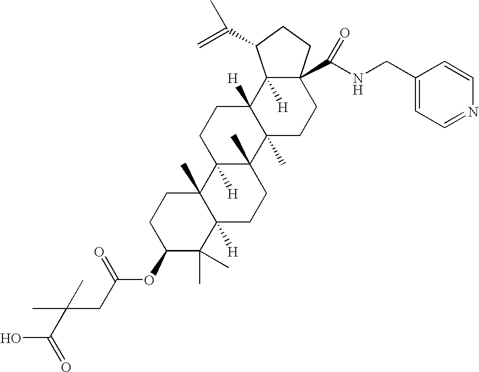 | 0.75 (s, 3 H, CH₃), 0.78 (s, 9 H, CH₃),0.92 (s, 3 H, CH₃), 1.15 (s, 3 H, CH₃),1.16 (s, 3 H, CH₃), 1.20-1.85 (m, 28 H),2.12-2.25 (m, 1 H), 2.95-3.08 (m, 1 H),4.19 (dd, 1 H, J = 16.1 Hz, J = 6.1 Hz,CH₂N), 4.26 (dd, 1 H, J = 16.1 Hz, J = 5.8 Hz,CH₂N), 4.36 (dd, 1 H, J = 11.5 Hz,J = 4.9 Hz, H-3), 4.53 (bs, 1 H,CH═), 4.65 (bs, 1 H, CH═), 7.22 (d,2 H, J = 6.0 Hz, CH Arom), 8.29 (t,1 H, J = 5.5 Hz, NH), 8.47 (d, 2 H,J = 6.0 Hz, CH Arom), 12.17 (bs, 1 H, CO₂H).675.67 (M + H)⁺. |
| 76 | 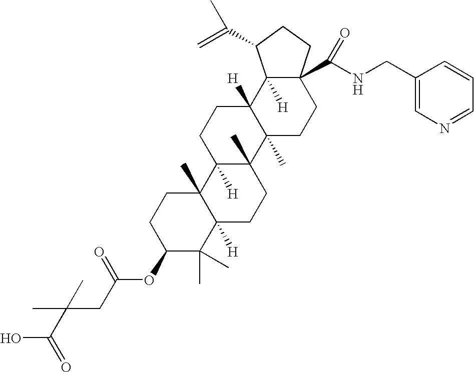 | 0.68 (s, 3 H, CH₃), 0.77 (s, 9 H, CH₃),0.91 (s, 3 H, CH₃), 1.15 (s, 3 H, CH₃),1.16 (s, 3 H, CH₃), 1.20-1.85 (m, 29 H),2.12-2.20 (m, 1 H), 2.95-3.05 (m, 1 H),4.22 (dd, 1 H, J = 15.1 Hz, J = 5.7 Hz,CH₂N), 4.30-4.40 (m, 2 H, H-3 andCH₂N), 4.53 (bs, 1 H, CH═), 4.64 (bs,1 H, CH═), 7.55-7.60 (m, 1 H, CHArom), 7.88-7.94 (m, 1 H, CH Arom),8.32 (bs, 1 H, NH), 8.55-8.60 (m, 2 H,CH Arom).675.4873 (M + H)⁺. |
| 77 | 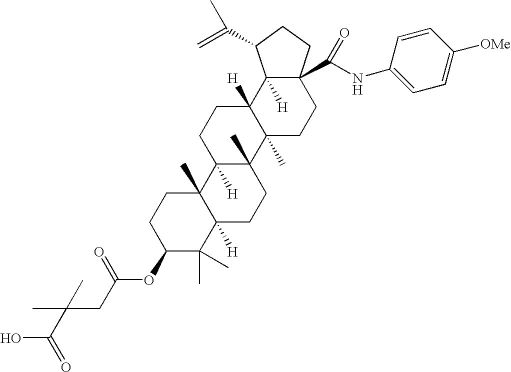 | 0.77 (s, 3 H, CH₃), 0.78 (s, 3 H, CH₃),0.80 (s, 3 H, CH₃), 0.86 (s, 9 H, CH₃),0.96 (s, 3 H, CH₃), 1.15 (s, 3 H, CH₃),1.16 (s, 3 H, CH₃), 1.20-2.05 (m, 28 H),2.30-2.40 (m, 1 H), 2.95-3.08 (m, 1 H),3.71 (s, 3 H, CH₃), 4.36 (dd, 1 H,J = 11.4 Hz, J = 4.6 Hz, H-3), 4.55 (bs,1 H, CH═), 4.68 (d, 1 H, J = 2.2 Hz,CH═), 6.85 (d, 2 H, J = 9.0 Hz, CHArom), 7.45 (d, 2 H, J = 9.0 Hz, CHArom), 9.29 (bs, 1 H, NH).690.4727 (M + H)⁺. |
| 78 | 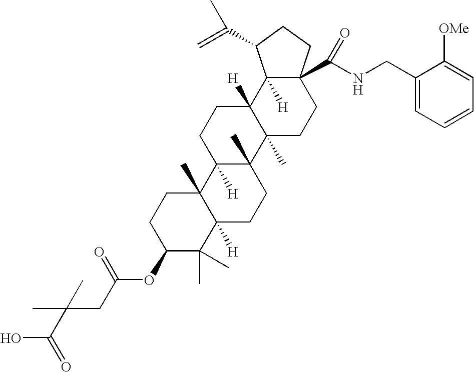 | 0.78 (s, 12 H, CH₃), 0.93 (s, 3 H, CH₃),1.15 (s, 3 H, CH₃), 1.16 (s, 3 H, CH₃),1.20-1.95 (m, 27 H), 2.15-2.25 (m,1 H), 2.95-3.08 (m, 1 H), 3.79 (s, 3 H,CH₃O), 4.20 (d, 2 H, J = 5.9 Hz,CH₂N), 4.36 (dd, 1 H, J = 11.3 Hz,J = 4.7 Hz,H-3), 4.53 (bs, 1 H, CH═), 4.64(d, 1 H, J = 2.5 Hz, CH═), 6.86 (dt,1 H, J = 7.4 Hz, J = 1.0 Hz, CH Arom),6.95 (d, 1 H, J = 7.6 Hz, CH Arom),7.11 (d, 1 H, J = 7.4 Hz, CH Arom),7.20 (dt, 1 H, J = 7.7 Hz, J = 1.7 Hz,CH Arom), 7.97 (t, 1 H, J = 5.9 Hz,NH), 12.18 (bs, 1 H, CO₂H).704.4877 (M + H)⁺. |
| 79 | 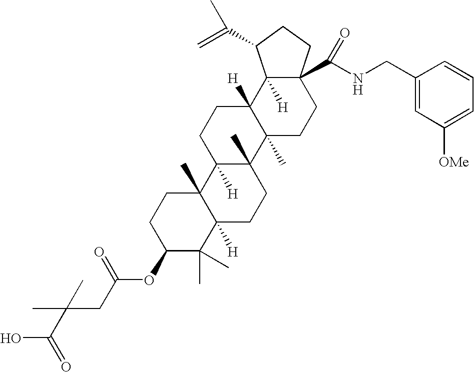 | 0.78 (s, 12 H, CH₃), 0.92 (s, 3 H, CH₃),1.15 (s, 3 H, CH₃), 1.16 (s, 3 H, CH₃),1.20-1.90 (m, 28 H), 2.15-2.25 (m,1 H), 2.95-3.08 (m, 1 H), 3.71 (s, 3 H,CH₃O), 4.17 (dd, 1 H, J = 15.2 Hz,J = 6.0 Hz, CH₂N), 4.25 (dd, 1 H,J = 15.2 Hz, J = 6.1 Hz, CH₂N), 4.36 (dd,1 H, J = 11.3 Hz, J = 4.7 Hz, H-3),4.53 (bs, 1 H, CH═), 4.65 (d, 1 H,J = 1.8 Hz, CH═), 6.72-6.82 (m, 3 H, CHArom), 7.19 (t, 1 H, J = 7.8 Hz, CHArom), 8.16 (t, 1 H, J = 6.0 Hz, NH),12.18 (bs, 1 H, CO₂H).704.4870 (M + H)⁺. |
| 80 | 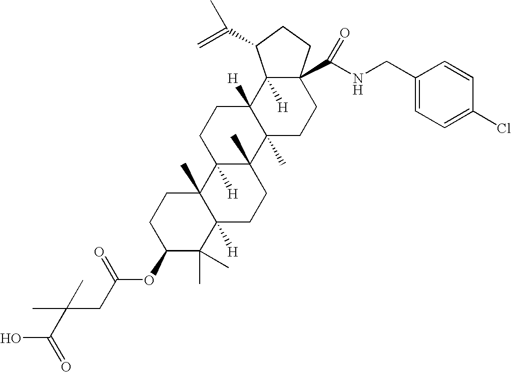 | 0.75 (s, 3 H, CH₃), 0.78 (s, 3 H, CH₃),0.79 (s, 3 H, CH₃), 0.92 (s, 3 H, CH₃),0.98-1.86 (m, 37 H), 2.14-2.22 (m,1 H), 2.96-3.06 (m, 1 H), 4.13 (dd, 1 H,J = 15.1 Hz, J = 6.1 Hz, CH₂N), 4.25(dd, 1 H, J = 15.1 Hz, J = 5.8 Hz,CH₂N), 4.36 (dd, 1 H, J = 11.3 Hz,J = 4.9 Hz, H-3), 4.53 (bs, 1 H, CH═), 4.64(d, 1 H, J = 2.3 Hz, CH═), 7.25 (d, 2 H,J = 8.5 Hz, CH Arom), 7.35 (d, 2 H,J = 8.5 Hz, CH Arom), 8.21 (t, 1 H,J = 6.0 Hz, NH), 12.20 (bs, 1 H, CO₂H).708.54 (M⁺ + 1). |
| 81 | 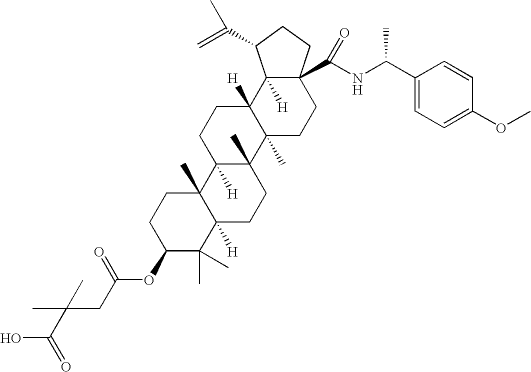 | 0.58 (s, 3 H, CH₃), 0.75 (s, 3 H, CH₃),0.76 (s, 3 H, CH₃), 0.77 (s, 3 H, CH₃),0.88 (s, 3 H, CH₃), 1.00-1.80 (m, 37 H),2.20-2.30 (m, 1 H), 2.95-3.06 (m, 1 H),3.72 (s, 3 H, CH₃O), 4.33 (dd, 1 H,J = 11.3 Hz, J = 4.9 Hz, H-3), 4.53(bs,1 H, CH═), 4.64 (d, 1 H, J = 2.5 Hz,CH═), 4.84-4.94 (1 H, m, CHN), 6.83(d, 2 H, J = 8.8 Hz, CH Arom), 7.21 (d,2 H, J = 8.8 Hz, CH Arom), 7.77 (d,1 H, J = 8.0 Hz, NH), 12.20 (bs, 1 H, CO₂H).718.30 (M⁺ + 1). |
| 82 | 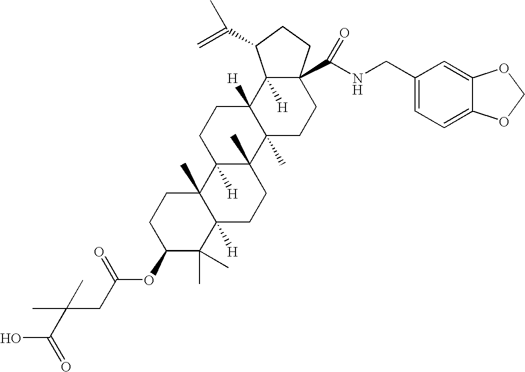 | 0.78 (s, 9 H, CH₃), 0.79 (s, 3 H, CH₃),0.92 (s, 3 H, CH₃), 0.93-1.06 (m, 2 H),1.15 (s, 3 H, CH₃), 1.16 (s, 3 H, CH₃),1.20-1.84 (m, 26 H), 2.12-2.20 (m,1 H), 2.98-3.07 (m, 1 H), 4.05 (dd, 1 H,J = 14.5, J = 6.0 Hz, CH₂N), 4.20 (dd,1 H, J = 14.5, J = 6.1 Hz, CH₂N), 4.36(dd, 1 H, J = 11.5 Hz, J = 4.9 Hz, H-3),4.53 (bs, 1 H, CH═), 4.64 (bs, 1 H,CH═), 5.96 (d, 1 H, J = 0.9 Hz,OCH₂O), 5.97 (d, 1 H, J = 0.9 Hz,OCH₂O), 6.70 (dd, 1 H, J = 7.8 Hz,J = 1.6 Hz, CHArom), 6.78 (d, 1 H,J = 1.6 Hz, CH Arom), 6.81 (d, 1 H,J = 7.8 Hz, CH Arom), 8.11 (d, 1 H,J = 6.3 Hz, NH), 12.18 (bs, 1 H, CO₂H).718.30 (M⁺ + 1). |
| 83 | 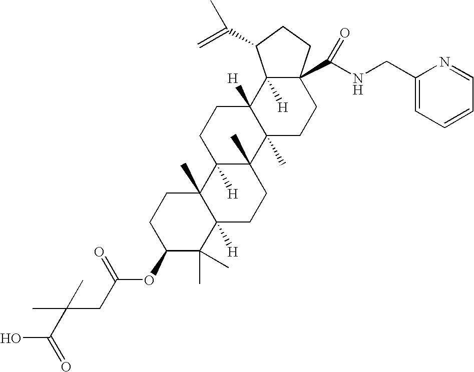 | 0.74 (s, 3 H, CH₃), 0.78 (s, 6 H, CH₃),0.93 (0.93, 3 H, CH₃), 1.00-1.92 (m,37 H), 2.18-2.26 (m, 1 H), 2.94-3.04(m, 1 H), 4.26-4.44 (m, 3 H, H-3 andCH₂N), 4.53 (bs, 1 H, CH═), 4.64 (bs1 H, CH═), 7.32-7.44 (m, 2 H, CHArom), 7.91 (bs, 1 H, CH Arom), 8.34(bs, 1 H, NH), 8.56 (bs, 1 H, CHArom), 12.16 (bs, 1 H, CO₂H).675.4716 (M⁺ + 1). |
| 84 | 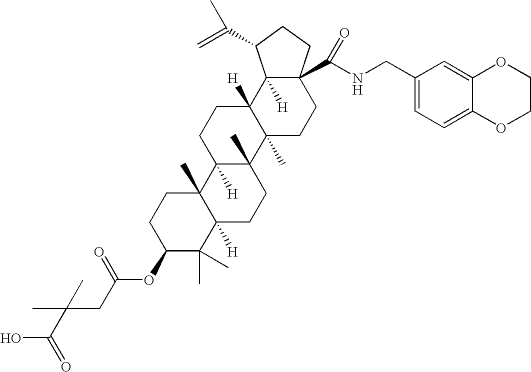 | 0.77 (s, 3 H, CH₃), 0.78 (s, 3 H, CH₃),0.79 (s, 3 H, CH₃), 0.91 (0.93, 3 HCH₃), 0.95-1.85 (m, 37 H), 2.15-2.21(m, 1 H), 2.95-3.05 (m, 1 H), 4.05 (dd,1 H, J = 14.6 Hz, J = 5.8 Hz, CH₂N),4.15 (dd, 1 H, J = 14.6 Hz, J = 5.5 Hz,CH₂N),4.19 (s, 4 H, CH₂O), 4.36 (dd,1 H, J = 10.6 Hz, J = 4.9 Hz, H-3),4.53 (bs, 1 H, CH═), 4.65 (bs, 1 H,CH═), 6.66-6.76 (m, 3 H, CH Arom),8.07 (bs, 1 H, NH), 12.18 (bs, 1 H, CO₂H).732.4823 (M⁺ + 1). |
| 85 | 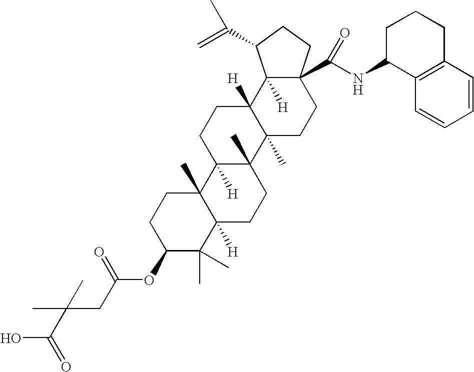 | 0.79 (s, 6 H, CH₃), 0.83 (s, 3 H, CH₃),0.95 (s, 3 H, CH₃), 0.98 (s, 3 H, CH₃),1.00-1.95 (m, 37 H), 2.15-2.21 (m,1 H), 2.60-2.75 (m, 3 H), 3.02-3.12 (m,1 H), 4.37 (dd, 1 H, J = 11.1 Hz, J = 4.7 Hz,H-3), 4.54 (bs, 1 H, CH═), 4.67(bs, 1 H, CH═),5.04-4.96 (bs, 1 H,CHN), 7.06-7.18 (m, 4 H, CH Arom),8.81 (d, 1 H, J = 8.2 Hz, NH), 12.18(bs, 1 H, CO₂H).714.5098 (M⁺ + 1). |
| 86 | 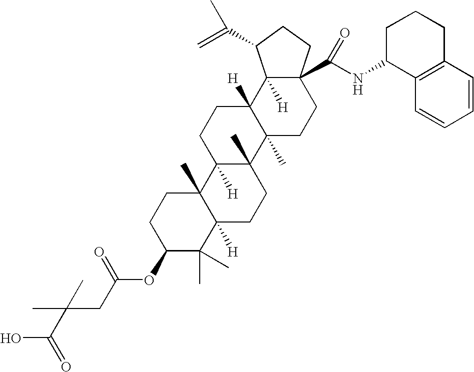 | 0.78 (s, 6 H, CH₃), 0.81 (s, 3 H, CH₃),0.88 (s, 3 H, CH₃), 0.93 (s, 3 H, CH₃),1.00-1.95 (m, 37 H), 2.20-2.30 (m,1 H), 2.60-2.75 (m, 3 H), 3.05-3.15 (m,1 H), 4.37 (dd, 1 H, J = 11.3 Hz, J = 4.7 Hz,H-3), 4.55 (bs, 1 H, CH═), 4.69(bs, 1 H, CH═), 5.07-4.90 (bs, 1 H,CHN), 7.00-7.20 (m, 4 H, CH Arom),7.88 (d, 1 H, J = 8.8 Hz, NH), 12.20(bs, 1 H, CO₂H).714.5095 (M⁺ + 1). |
| 87 | 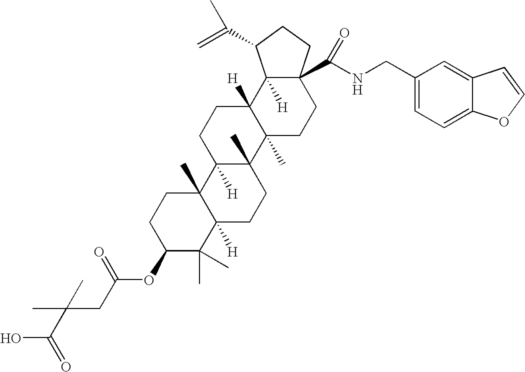 | 0.74 (s, 3 H, CH₃), 0.78 (s, 6 H, CH₃),0.79 (s, 3 H, CH₃), 0.91 (s, 3 H, CH₃),0.95-1.85 (m, 34 H), 2.15-2.23 (m,1 H), 3.00-3.10 (m, 1 H), 4.22 (dd, 1 H,J = 14.6 Hz, J = 5.8 Hz, CH₂N), 4.32-4.44 (m, 2 H, H-3 and CH₂N), 4.53 (bs,1 H, CH═), 4.65 (d, 1 H, J = 2.3 Hz,CH═), 6.90 (dd, 1 H, J = 2.3 Hz,J = 0.9 Hz, CH Arom), 7.20 (dd, 1 H,J = 8.8 Hz, J = 1.4 Hz, CH Arom), 7.48-7.52 (m, 2 H, CH Arom), 7.96 (d, 1 H,J = 2.1 Hz, CH Arom), 8.20 (t, 1 H,J = 6.1 Hz, NH), 12.18 (bs, 1 H, CO₂H).714.40 (M⁺ + 1). |
| 88 | 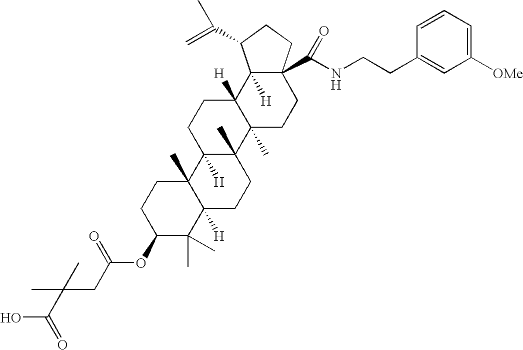 | 0.78 (s, 6 H, CH₃), 0.79 (s, 3 H, CH₃),0.85-0.99 (m, 4 H), 1.00-1.80 (m,36 H), 2.02-2.10 (m, 1 H), 2.60-2.75(m, 2 H, CH₂Ar), 2.95-3.05 (m, 1 H),3.15-3.25 (m, 1 H, CH₂N), 3.28-3.40(m, 1 H, CH₂N), 3.72 (s, 3 H, CH₃O),4.35 (dd, 1 H, J = 11.6 Hz, J = 5.0 Hz,H-3), 4.52 (bs, 1 H, CH═), 4.64 (d, 1 H,J = 2.1 Hz, CH═), 6.72-6.78 (m, 3 H,CH Arom), 7.18 (dd, 1 H, J = 8.9 Hz,J = 7.3 Hz, CH Arom), 7.60 (t, 1 H,J = 6.1 Hz, NH), 12.11 (bs, 1 H, CO₂H).718.5064 (M⁺ + 1). |
| 89 | 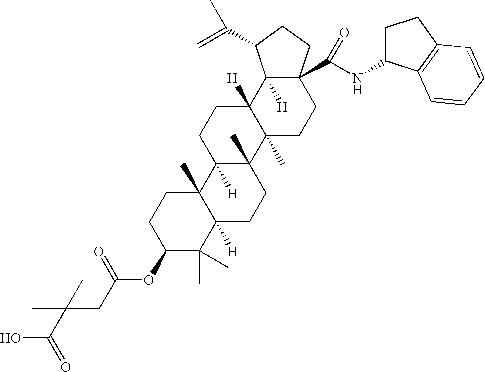 | 0.79 (s, 6 H, CH₃), 0.83 (s, 3 H, CH₃),0.94 (s, 3 H, CH₃), 0.97 (s, 3 H, CH₃),1.00-1.95 (m, 34 H), 2.15-2.25 (m,1 H), 2.25-2.35 (m, 1 H), 2.60-2.85 (m,2 H), 2.95-2.85 (m, 1 H), 3.05-3.15 (m,1 H), 4.37 (dd, 1 H, J = 11.5 Hz, J = 4.7 Hz, H-3), 4.55 (bs, 1 H, CH═), 4.67 (d,1 H, J = 2.0 Hz, CH═), 5.28-38 (m, 1 H,CHN), 7.12-7.26 (m, 4 H, CH Arom),7.84 (d, 1 H, J = 8.0 Hz, NH), 12.18(bs, 1 H, CO₂H).700.4925 (M⁺ + 1). |
| 90 | 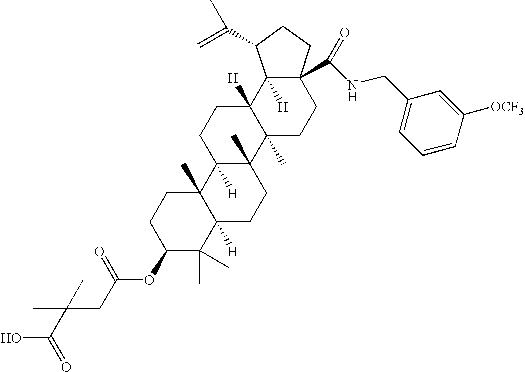 | 0.74 (s, 3 H, CH₃), 0.78 (s, 9 H, CH₃),0.84-1.90 (m, 37 H), 2.14-2.22 (m,1 H), 2.95-3.06 (m, 1 H), 4.19 (dd, 1 H,J = 15.4 Hz, J = 5.7 Hz, CH₂N), 4.30-4.40 (m, 2 H, H-3 and CH₂N), 4.53 (bs,1 H, CH═), 4.65 (bs, 1 H, CH═), 7.19-7.22 (m, 2 H,CH Arom), 7.28 (d, 1 H,J = 7.7 Hz, CH Arrom), 7.43 (t, 1 H,J = 7.7 Hz, CH Arom), 8.27 (t, 1 H,J = 5.7 Hz, NH).758.4656 (M⁺ + 1). |
| 91 | 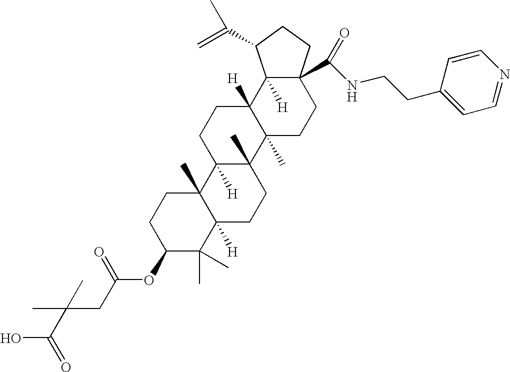 | 0.70 (s, 3 H, CH₃), 0.78 (s, 6 H, CH₃),0.85-1.75 (m, 42 H), 1.95-2.05 (m,1 H), 2.87-3.00 (m, 2 H), 3.30-3.52 (m,2 H, CH₂N), 4.35 (dd, 1 H, J = 11.3 Hz,J = 4.9 Hz, H-3), 4.52 (bs, 1 H, CH═),4.63 (d, 1 H, J = 2.3 Hz, CH═), 7.64-7.72 (m, 2 H, CH Arom), 8.70 (d, 2 H,J = 5.9 Hz, CH Arom), 12.11 (bs, 1 H, CO₂H).689.4997 (M⁺ + 1). |
| 92 | 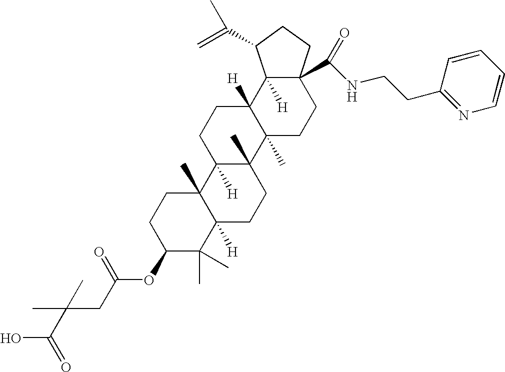 | 0.70 (s, 3 H, CH₃), 0.79 (s, 6 H, CH₃),0.88-1.70 (m, 42 H), 1.95-2.05 (m,1 H), 2.87-3.00 (m, 2 H), 3.30-3.52 (m,2 H, CH₂N), 4.35 (dd, 1 H, J = 11.3 Hz,J = 4.9 Hz, H-3), 4.52 (bs, 1 H, CH═),4.63 (d, 1 H, J = 2.3 Hz, CH═), 7.55(m, 2 H, CH Arom), 7.68 (m, 1 H), 8.64(m, 1 H, CH Arom), 12.20 (bs, 1 H, CO₂H).689.50 (M⁺ + 1). |
| 93 | 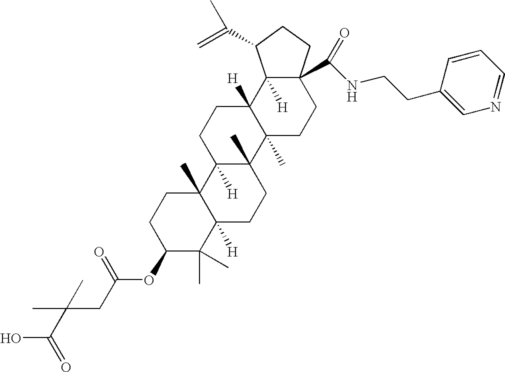 | 0.70 (s, 3 H, CH₃), 0.79 (s, 6 H, CH₃),0.88-1.70 (m, 42 H), 1.95-2.05 (m,1 H), 2.87-3.00 (m, 2 H), 3.34-3.41 (m,2 H, CH₂N), 4.35 (dd, 1 H, J = 11.3 Hz,J = 4.9 Hz, H-3), 4.52 (bs, 1 H, CH═),4.62 (d, 1 H, J = 2.3 Hz, CH═), 7.68(m, 2 H, CH Arom), 8.08 (m, 2 H, CH Arom),8.08 (m, 2 H, CH Arom, amide NH),8.64 (m, 1 H, CH Arom), 12.20(bs, 1 H, CO₂H).689.52 (M⁺ + 1). |
| 94 | 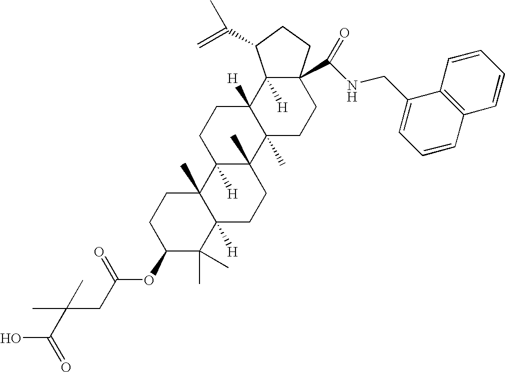 | 0.78 (s, 6 H, CH₃), 0.79 (s, 3 H, CH₃),0.88-1.70 (m, 42 H), 1.95-2.05 (m,1 H), 2.87-3.00 (m, 2 H), 3.34-3.41 (m,2 H, CH₂N), 4.36 (dd, 1 H, J = 11.9 Hz,J = 4.69 Hz, H-3), 4.54 (bs, 1 H, CH═),4.64 (m, 2 H), 4.75 (d, 1 H, J = 5.3 Hz,CH═), 7.44 (m, 2 H, CH Arom), 7.52(m, 2 H, CH Arom), 7.82 (d, 1 H, J = 7.6 Hz,CH Arom), 7.92 (m, 1 H, Arom),8.12 (m, 1 H, Arom), 8.18 (t, 1 H,J = 7.6 Hz, amide NH), 12.18 (s, 1 H, CO₂H).724.49 (M⁺ + 1). |
| 95 | 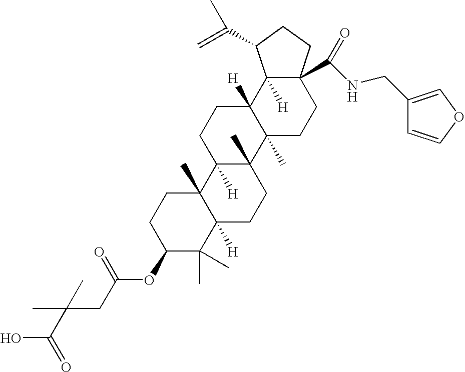 | 0.78 (s, 6 H, CH₃), 0.80 (s, 3 H, CH₃),0.88-1.70 (m, 42 H), 1.95-2.55 (m,2 H), 3.05 (m, 1 H), 3.99 (m, 1 H,CH₂N), 4.11 (m, 1 H, CH₂N), 4.35 (dd,1 H, J = 12.1 Hz, J = 5.2 Hz, H-3),4.54 (bs, 1 H, CH═), 4.66 (s, 1 H,CH═), 6.36 (s, 1 H), 7.46 (s, 1 H), 7.56(s, 1 H), 7.98 (t, 1 H, J = 6.1 Hz, amideNH), 12.18 (s, 1 H, CO₂H).664.47 (M⁺ + 1). |
| 96 | 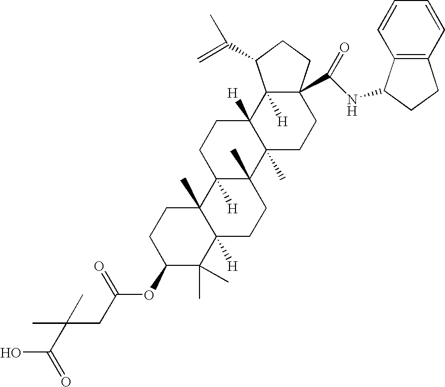 | 0.79 (s, 6 H, CH₃), 0.83 (s, 3 H, CH₃),0.94 (s, 3 H, CH₃), 0.97 (s, 3 H, CH₃),1.00-1.95 (m, 34 H), 2.15-2.25 (m,1 H), 2.25-2.35 (m, 1 H), 2.60-2.85 (m,2 H), 2.95-2.85 (m, 1 H), 3.05-3.15 (m,1 H), 4.37 (dd, 1 H, J = 11.5 Hz, J = 4.7 Hz,H-3), 4.55 (bs, 1 H, CH═), 4.67 (d,1 H, J = 2.0 Hz, CH═), 5.35 (m, 1 H,CHN), 7.12-7.26 (m, 4 H, CH Arom),7.84 (d, 1 H, J = 8.0 Hz, NH), 12.18(bs, 1 H, CO₂H).700.4925 (M⁺ + 1). |
| 97 | 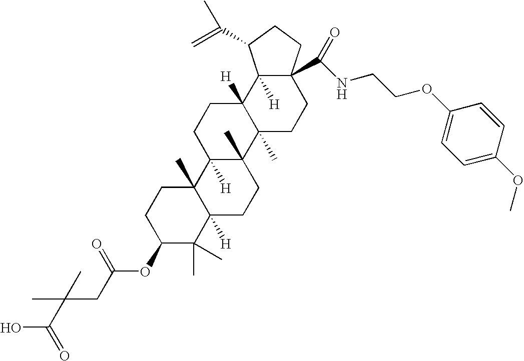 | 0.79 (s, 6 H, CH₃), 0.83 (s, 3 H, CH₃),0.94 (s, 3 H, CH₃), 0.97 (s, 3 H, CH₃),1.00-1.95 (m, 34 H), 2.15-2.25 (m,1 H), 2.25-2.35 (m, 1 H), 2.60-2.85 (m,2 H), 2.95-2.85 (m, 1 H), 3.05 (m, 1 H),3.25 (m, 1 H), 3.49 (m, 1 H), 3.67 (s,3 H), 3.91 (m, 2 H, CHN), 4.35 (dd, 1 H,J = 11.5 Hz, J = 4.7 Hz, H-3), 4.53 (s,1 H, CH═), 4.65 (s, 1 H, CH═), 4.65(s, 1 H, CH═), 6.84 (m, 4 H, CH Arom), 7.78(d, 1 H, J = 8.0 Hz, NH).734.50 (M⁺ + 1). |
| 98 | 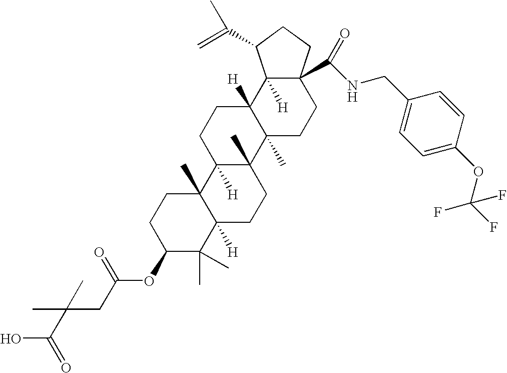 | 0.68 (s, 3 H, CH₃), 0.77 (s, 6 H, CH₃),0.92 (s, 3 H, CH₃), 0.98-1.86 (m, 37 H),2.14-2.22 (m, 1 H), 3.06 (m, 1 H), 4.16(dd, 1 H, J = 15.1 Hz, J = 6.1 Hz,CH₂N), 4.28 (dd, 1 H, J = 15.1 Hz,J = 5.9 Hz, CH₂N), 4.35 (dd, 1 H, J = 11.4 Hz,J = 4.9 Hz, H-3), 4.53 (bs, 1 H,CH═), 4.64 (d, 1 H, J = 2.4 Hz, CH═),7.29 (d, 2 H, J = 7.8 Hz, CH Arom),7.36 (d, 2 H, J = 8.8 Hz, CH Arom),8.24 (t, 1 H, J = 6.0 Hz, NH), 12.20(bs, 1 H, CO₂).758.51 (M⁺ + 1). |
| 99 | 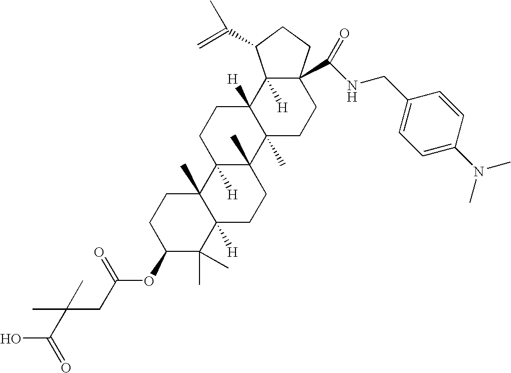 | 0.78 (s, 12 H, CH₃), 0.92 (s, 6 H, CH₃),1.16 (s, 3 H), 1.17 (s, 3 H), 1.2-1.86 (m,19 H), 2.14-2.22 (m, 1 H), 2.90 (s, 6 H),3.03 (m, 1 H), 4.09 (dd, 1 H, J = 14.5 Hz,J = 6.3 Hz, CH₂N), 4.20 (dd, 1 H,J = 14.5 Hz, J = 5.7 Hz, CH₂N), 4.36(dd, 1 H,J = 11.3 Hz, J = 5.1 Hz, H-3),4.53 (bs, 1 H, CH═), 4.65 (bs, 1 H,CH═), 6.81 (d, 2 H, CH Arom), 7.11(bd, 2 H, CH Arom), 8.05 (bs, 1 H,amide NH).717.53 (M⁺ + 1). |
| 100 | 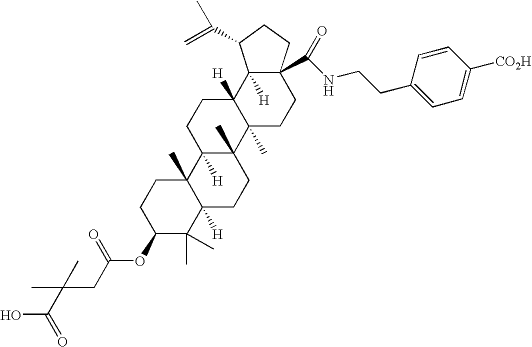 | 0.70 (s, 3 H, CH₃), 0.78 (s, 6 H, CH₃),0.85-1.75 (m, 42 H), 1.95-2.05 (m,1 H), 2.87-3.00 (m, 2 H), 3.30-3.52 (m,2 H, CH₂N), 4.35 (m, 1 H, H-3), 4.51(bs, 1 H, CH═), 4.63 (bd, 1 H, CH═),7.31 (d, 2 H, J = 8.2 Hz, CH Arom),7.60 (bs, 1 H, amide NH), 7.83 (d, 2 H,J = 8.2 Hz, CH Arom).732.49 (M⁺ + 1). |
| 101 | 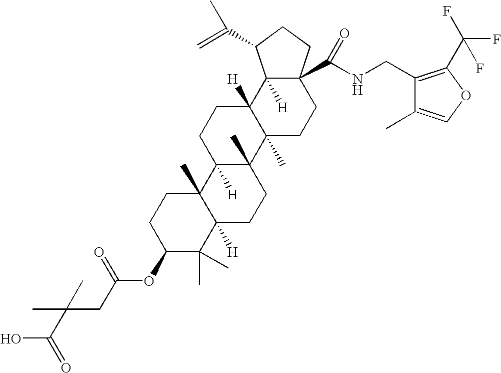 | 0.76 (s, 6 H, CH₃), 0.80 (s, 3 H, CH₃),0.88-1.70 (m, 42 H), 2.15 (m, 1 H),2.28 (s, 3 H), 3.05 (m, 1 H), 4.06 (m,1 H, CH₂N), 4.21 (m, 1 H, CH₂N), 4.36(dd, 1 H, J = 11.5 Hz, J = 4.9 Hz, H-3),4.54 (bs, 1 H, CH═), 4.66 (s, 1 H,CH═), 6.17 (s, 1 H), 8.13 (bt, 1 H,amide NH), 12.19 (s, 1 H, CO₂H).746.46 (M⁺ + 1). |
| 102 | 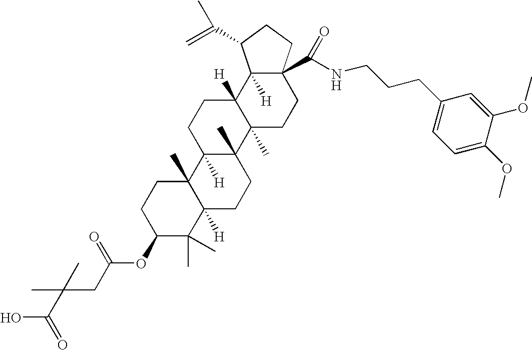 | 0.78 (m, 9 H, CH₃), 0.84 (s, 3 H, CH₃),0.92 (s, 3 H), 0.88-1.70 (m, 42 H), 2.18(m, 1 H), 2.53 (m, 3 H), 3.01 (m, 3 H),3.71 (s, 3 H), 3.73 (s, 3 H), 4.36 (dd,1 H, J = 11.5 Hz, J = 4.9 Hz, H-3),4.53 (bs, 1 H, CH═), 4.65 (d, 1 H,J = 2.4 Hz, CH═), 6.68 (dd, 1 H, J = 8.2,2.0 Hz), 6.76 (d, 1 H, J = 2.0 Hz), 6.84(d, 1 H, J = 8.2 Hz), 7.61 (t, 1 H,J = 5.4 Hz, amide NH), 12.18 (s, 1 H,CO₂H).762.53 (M⁺ + 1). |
| 103 | 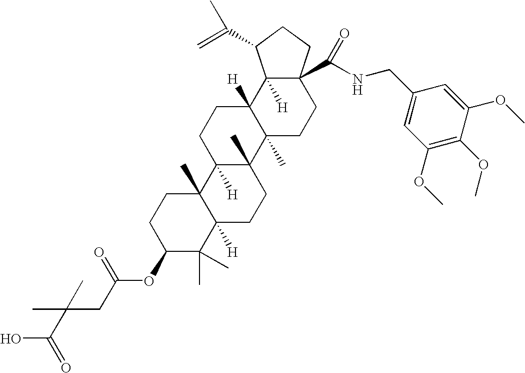 | 0.76 (m, 12 H, CH₃), 0.92 (s, 6 H),0.88-1.90 (m, 42 H), 2.20 (m, 1 H),2.53 (m, 3 H), 3.00 (m, 1 H), 3.62 (s,3 H), 3.73 (s, 6 H), 4.18 (dd, 1 H,J = 15.0 Hz, J = 5.7 Hz, CH₂N), 4.36 (dd,1 H, J = 11.3 Hz, J = 5.1 Hz, H-3),4.53 (bs, 1 H, CH═), 4.65 (d, 1 H,J = 2.4 Hz, CH═), 6.53 (s, 2 H), 8.15 (t,1 H, J = 6.1 Hz, amide NH), 12.18 (s,1 H, CO₂H).764.51 (M⁺ + 1). |
| 104 | 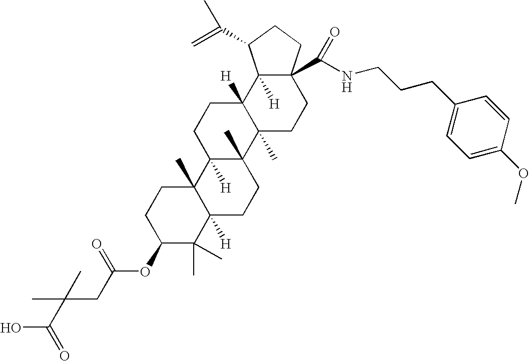 | 0.78 (m, 9 H, CH₃), 0.84 (s, 3 H, CH₃),0.92 (s, 6 H), 0.88-1.70 (m, 42 H), 2.18(m, 1 H), 2.53 (m, 3 H), 3.01 (m, 3 H),3.72 (s, 3 H), 4.37 (bm, 1 H, H-3), 4.53(bs, 1 H, CH═), 4.65 (bs, 1 H, CH═),6.83 (d, 2 H, J = 8.6 Hz), 7.09 (d, 2 H,J = 8.2 Hz), 7.61 (bt, 1 H, amide NH).732.53 (M⁺ + 1). |
| 105 | 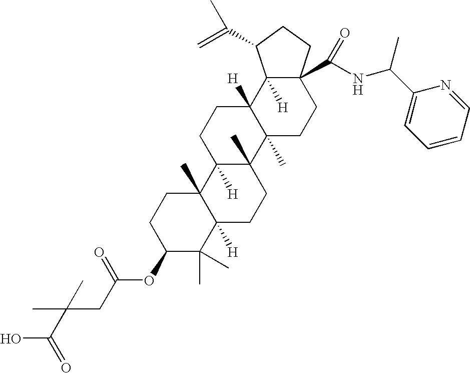 | δ 12.16 (bs, 1 H), 8.48 (bt, 1 H), 7.90 (q,1 H, J = 7.63 Hz), 7.71 (p, 1 H, J = 9.6 Hz),7.30 (t, 1 H, J = 8.8 Hz), 7.23(bm, 1 H), 5.77 (s, 1 H), 4.95 (q, 1 H,J = 6.8 Hz), 4.62 (d, 1 H, J = 8.8 Hz),4.53 (s, 1 H), 4.36 (m, 1 H), 3.24 (m,2 H), 3.00 (m, 1 H), 2.80 (m, 2 H), 2.48-2.05 (m, 5 H), 1.65-1.17 (m, 35 H),1.16 (s, 6 H), 0.86 (s, 3 H), 0.78 (s,9 H), 0.70 (s, 3 H).689.50 (M⁺ + 1). |
| 106 | 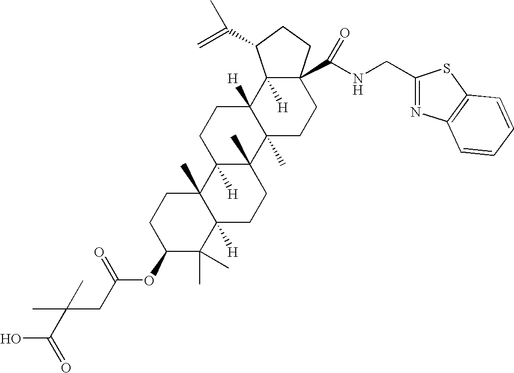 | 0.78 (s, 9 H, 3 × CH3), 0.83 (s, 3 H,CH3), 0.94 (s, 6 H, 2 × CH3), 1.16 (s,3 H, CH3), 1.25-1.62 (m, 18 H, CH₂),1.64 (s, 3 H, CH3), 1.75-1.85 (m, 2 H,CH₂), 2.18-2.27 (m, 2 H, CH2), 2.97(bs, 1 H), 4.35 (dd, 1 H, J = 11.25 Hz,J = 5.08 Hz,), 4.54 (s, 1 H, CH═), 4.62(d, 2 H, J = 5.67, CH2), 4.66 (s, 1 H,CH═), 7.41 (dd, 1 H, J = 7.63 Hz, Ar),7.50 (dd, 1 H, J = 9.0 Hz, Ar), 7.92 (d,1 H, J = 8.02 Hz, Ar), 8.06 (dd, 1 H,J = 8.02 Hz, Ar), 8.64 (bs, 1 H, NH),(M + 1 = 731.47). |
| 107 | 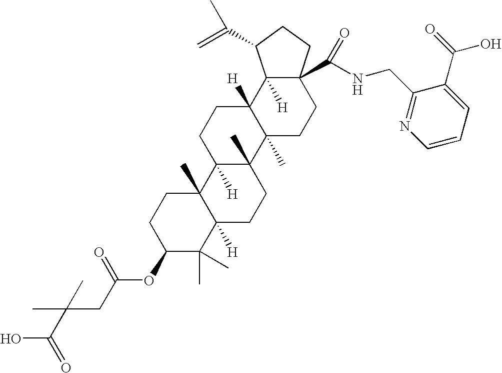 | δ 8.63 (d, 1 H, J = 4.8 Hz), 8.20 (d,1 H, J = 8.0 Hz), 7.96 (m, 1 H), 7.39(m, 1 H), 4.65 (m, 3 H), 4.52 (s, 1 H),4.36 (dd, 1 H, J = 11.2 Hz, 4.4 Hz),2.98 (m, 1 H), 2.43 (m, 1 H), 2.23 (d,1 H, J = 11.6 Hz), 1.93 (m, 1 H), 1.80-1.00 (m, 32 H), 0.92 (s, 3 H), 0.77(s, 9 H), 0.73 (s, 3 H).TOF-MS m/z 719 (M + H)⁺ |
| 108 | 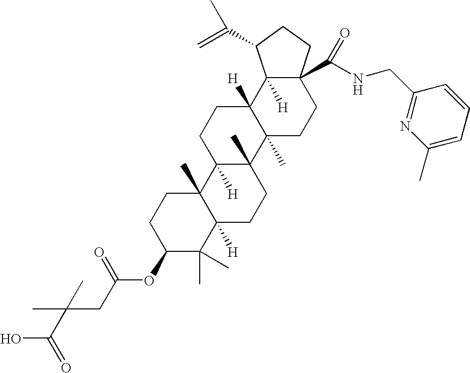 | δ 8.35 (t, 1 H, J = 5.6 Hz), 7.82 (br s,1 H), 7.29 (d, 1 H, J = 5.6 Hz), 7.20 (d,1 H, J = 5.6 Hz), 4.64 (s, 1 H), 4.52 (s,1 H), 4.35 (m, 3 H), 2.98 (m, 1 H), 2.41(s, 3 H), 2.39 (m, 1 H), 2.23 (d, 1 H,J = 11.6 Hz), 1.89 (m, 1 H), 1.80-1.00 (m,32 H), 0.92 (s, 3 H), 0.78 (s, 9 H), 0.74 (s, 3 H).TOF-MS m/z 689 (M + H)⁺ |
| 109 | 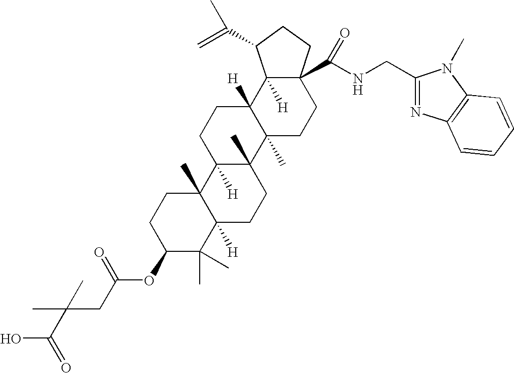 | δ 0.60 (s, 3 H, CH3), 0.73 (s, 3 H,CH3), 0.7 (s, 6 H, 2 × CH3), 0.91 (s,3 H, CH3), 1.16 (s, 6 H, 2 × CH3),1.25-1.62 (m, 18 H, CH₂), 1.62 (s, 3 H,CH3), 1.75-1.85 (m, 2 H, CH₂), 2.18-2.27 (m, 2 H, CH2), 2.97 (bs, 1 H), 3.87(s, 3 H, CH3N), 4.35 (dd, 1 H,J = 11.25 Hz, J = 5.08 Hz, J = 4.89 Hz),4.53 (s, 1 H, CH═), 5.53-4.61 (bs, 2 H,CH2), 4.64 (bs, 1 H, CH═), 7.35 (bs,2 H, Ar), 7.65 (bs, 2 H, Ar), 8.41 (bs,1 H, NH), 12.18 (bs, 1 H, CO₂H),(M + 1 = 728.50). |
| 110 | 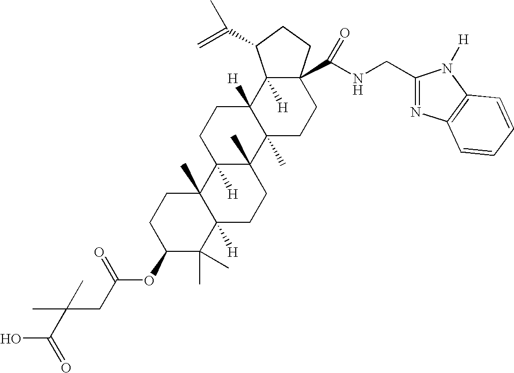 | δ 0.70 (s, 3 H, CH3), 0.75 (s, 3 H,CH3), 0.76 (s, 3 H, CH3), 0.77 (s,3 H, CH3), 0.91 (s, 3 H, CH3), 1.155 (s,3 H, CH3), 1.163 (s, 3 H, CH3), 1.25-1.62 (m, 18 H, CH₂), 1.62 (s, 3 H, CH3),1.75-1.85 (m, 2 H, CH₂), 2.18-2.27 (m,2 H CH2), 3.00-3.071 (m, 1 H), 4.35(dd, 1 H, J = 11.25 Hz, J = 5.08 Hz,J = 4.89 Hz), 4.4 (d, 1 H, J = 5.09 Hz),4.53 (s, 1 H, CH═), 4.64 (d, J = 2.85 HzCH═), 7.13 (dd, J = 5.97 Hz,J = 3.13 Hz, J = 3.32 Hz, Ar), 7.48 (dd,J = 5.87 Hz, J = 3.13 Hz, Ar), 8.25 (t,J = 11.15 Hz, J = 5.87 Hz, J = 5.28 Hz,NH) 12.12 (bs, 1 H, CO₂H), (M − 1 = 712.6). |
| 111 | 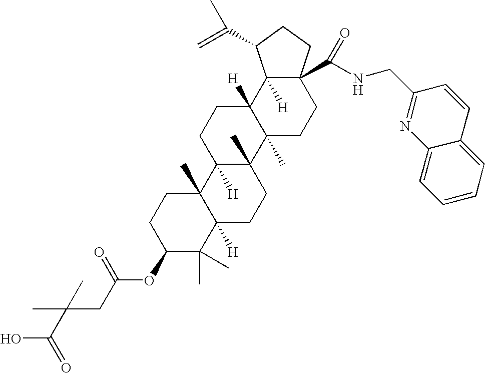 | δ 0.745 (s, 3 H, CH3), 0.75 (s, 3 H,CH3), 0.77 (s, 3 H, CH3), 0.78 (s, 3 H,CH3), 0.94 (s, 3 H, CH3), 1.154 (s, 3 H,CH3), 1.162 (s, 3 H, CH3), 1.25-1.58(m, 18 H, CH₂), 1.64 (s, 3 H, CH3),1.75-1.85 (m, 2 H, CH₂), 2.18-2.27 (m,2 H, CH2), 2.97 (bs, 1 H), 4.34 (bs, 1 H,CH2), 4.4 (dd, 1 H, J = 10.96 Hz,J = 4.69 Hz), 4.53 (bs, 2 H CH═, 1 H,CH2), 4.64 (bs, 1 H, CH═), 7.4 (d, 1 H,J = 8.41, Ar), 7.57 (dd, 1 H, J = 14.48,J = 7.63, J = 6.85, Ar), 7.74 (dd, 1 H,J = 14.48, J = 7.63, J = 6.85, Ar), 7.94(dd, 2 H, J = 14.48,J = 7.63, J = 6.85,Ar), 8.3 (d, 1 H, J = 7.83, Ar), 8.40(bs, 1 H, NH), 12.09 (bs, 1 H, CO₂H),M + 1 = 725.49). |
| 112 | 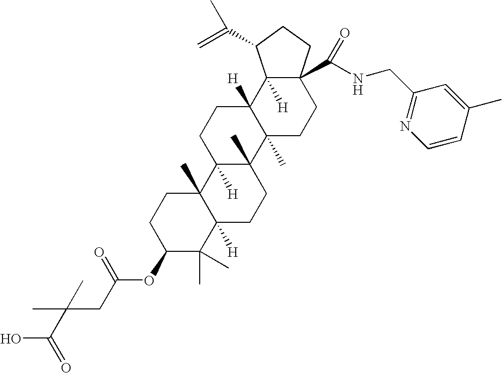 | δ 8.56 (d, 1 H, J = 5.6 Hz), 8.46 (t, 1 H,J = 5.6 Hz), 7.50 (d, 1 H, J = 5.6 Hz),7.39 (s, 1 H), 4.64 (s, 1 H), 4.52 (s,1 H), 4.35 (m, 3 H), 2.98 (m, 1 H), 2.41(s, 3 H), 2.39 (m, 1 H), 2.23 (d, 1 H,J = 11.6 Hz), 1.89 (m, 1 H), 1.80-1.00 (m,32 H), 0.92 (s, 3 H), 0.77 (s, 9 H), 0.68(s, 3 H).TOF-MS m/z 689 (M + H)⁺ |
| 113 | 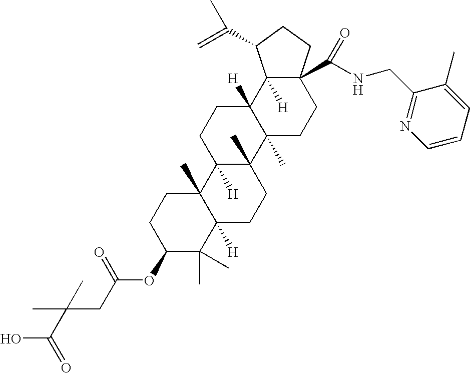 | δ 8.57 (d, 1 H, J = 5.6 Hz), 8.38 (br s,1 H), 8.09 (d, 1 H, J = 5.6 Hz), 7.64 (br s,1 H), 4.63 (s, 1 H), 4.53 (s, 1 H), 4.40(m, 3 H), 2.98 (m, 1 H), 2.41 (s, 3 H),2.37 (m, 1 H), 2.17 (d, 1 H, J = 11.6 Hz),1.83 (m, 1 H), 1.70-0.98 (m,32 H), 0.90 (s, 3 H), 0.76 (s, 9 H), 0.55(s, 3 H).TOF-MS m/z 689 (M + H)⁺ |
| 114 | 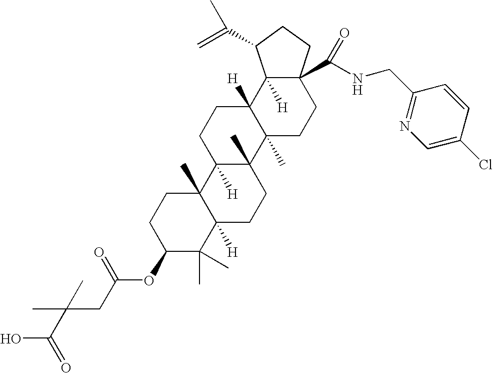 | δ 8.52 (s, 1 H), 8.30 (t, 1 H, J = 6.0 Hz),7.89 (d, 1 H, J = 8.4 Hz), 7.26 (d,1 H, J = 8.4 Hz), 4.64 (s, 1 H), 4.52 (s,1 H), 4.30 (m, 3 H), 2.98 (m, 1 H), 2.47(m, 1 H), 2.21 (d, 1 H, J = 11.6 Hz),1.87 (m, 1 H), 1.80-1.00 (m, 32 H),0.92 (s, 3 H), 0.78 (s, 9 H), 0.75 (s, 3 H).TOF-MS m/z 709 (M + H)⁺ |
| 115 | 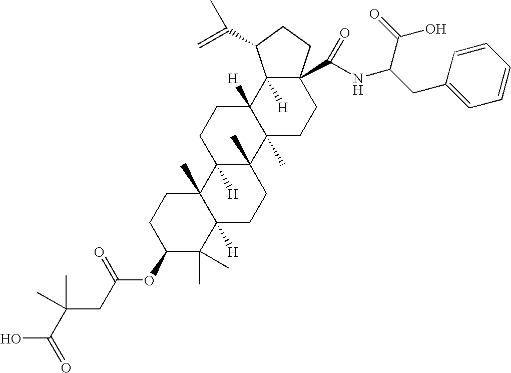 | 12.30 (bs, 1 H, COOH), 7.20 (m, 5 H,CH Arom), 7.70 (dd, 1 H, amide NH),4.60 (bd, 1 H, CH═), 4.51 (bs, 1 H,CH═), 4.35 (m, 1 H, H-3), 3.30-3.52(m, 2 H, CH₂N), 3.10 (m, 1 H), 2.87-3.00 (m, 2 H), 1.95-2.05 (m, 1 H), 0.85-1.75 (m, 42 H), 0.78 (s, 6 H, CH₃), 0.70(s,3 H, CH₃).732.48 (M⁺ + 1). |
| 116 | 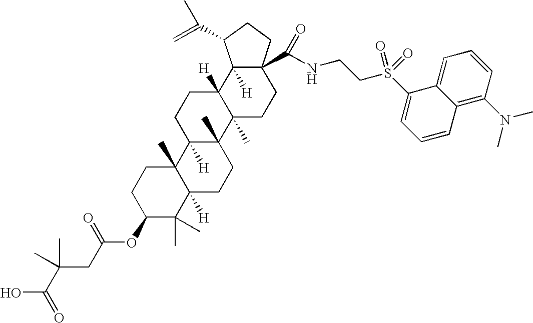 | δ 0.612 (s, 3 H, CH3), 0.765 (s, 3 H,CH3), 0.784 (s, 3 H, CH3), 0.793 (s,3 H, CH3), 0.860 (s, 3 H, CH3), 1.158(s, 3 H, CH3), 1.167 (s, 3 H, CH3),1.25-1.58 (m, 18 H, CH₂), 1.59 (s, 3 H,CH3), 2.83 (s, 6 H, N(CH3)2), 4.35(dd, 1 H, J = 10.96 Hz, J = 4.69 Hz),4.50 (s, 1 H, CH2═), 4.61 (s, 2 H, CH═),7.26 (d, 1 H, J = 7.05, Ar), 7.51 (bs,1 H, NH), 7.57-7.64 (m, 2 H, Ar), 7.93(bs, 1 H, NH), 8.1 (dd, 1 H, J = 7.23,J = 1.18, Ar), 8.27 (d, 1 H, J = 8.81,Ar), 8.46 (d, 1 H, J = 8.41, Ar), 12.1(bs, 1 H, CO₂H), (M + 1 = 860.52). |
| 117 | 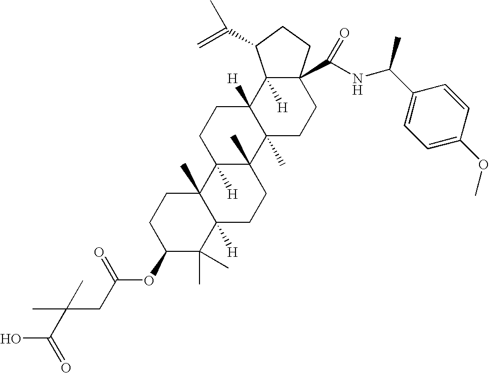 | δ 12.20 (bs, 1 H, CO₂H), 7.77 (d, 1 H,J = 8.0 Hz, NH), 7.21 (d, 2 H, J = 8.8 Hz,CH Arom), 6.83 (d, 2 H, J = 8.8 Hz,CH Arom), 4.84-4.94 (1 H, m,CHN), 4.64 (d, 1 H, J = 2.5 Hz, CH═),4.53 (bs, 1 H, CH═), 4.32 (dd, 1 H,J = 11.3 Hz, J = 4.9 Hz, H-3), 3.77 (s, 3 H,CH₃O), 2.95-3.06 (m, 1 H), 2.20-2.30(m, 1 H), 1.00-1.80 (m, 37 H), 0.88 (s,3 H, CH₃), 0.77 (s, 3 H, CH₃), 0.76 (s,3 H, CH₃), 0.75 (s, 3 H, CH₃), 0.58 (s,3 H, CH₃)718.51 (M⁺ + 1). |
| 118 | 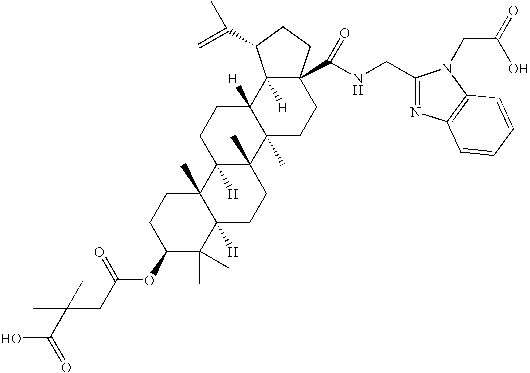 | δ 0.65 (s, 3 H, CH3), 0.742 (s, 3 H,CH3), 0.765 (s, 3 H, CH3), 0.772 (s,3 H, CH3), 0.94 (s, 3 H, CH3), 1.154 (s,3 H, CH3), 1.162 (s, 3 H, CH3), 1.25-1.58 (m, 18 H, CH₂), 1.62 (s, 3 H,CH3), 1.75-1.85 (m, 2 H, CH₂), 2.18-2.27 (m, 2 H, CH2), 2.97 (bs, 1 H), 4.35(dd, 1 H,J = 10.96 Hz, J = 4.69 Hz),4.53 (bs, 1 H, CH═, 2 H, CH2), 4.64 (bs,1 H, CH═), 5.3 (dd, 2 H, J = 36.59,J = 18.78, NCH2CO2H), 7.36 (bs, 2 H,Ar), 7.66 (bs, 2 H, Ar), 8.40 (s, 1 H,NH), (M + 1 = 772.50). |
| 119 | 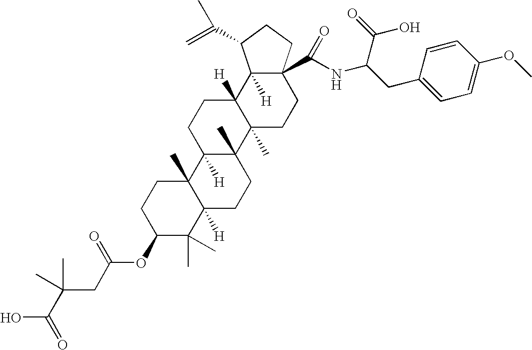 | ¹H NMR (DMSO-d₆, 400 MHz)δ 0.738 (s, 3 H, CH3), 0.776 (s,3 H, CH3), 0.809 (s, 3 H, CH3), 0.860(s, 3 H, CH3), 0.877 (s, 3 H, CH3),1.154 (s, 3 H, CH3), 1.163 (s, 3 H,CH3), 1.25-1.58 (m, 18 H, CH₂),1.58 (s, 3 H, CH3), 1.75-1.85 (m, 2 H,CH₂), 2.18-2.27 (m,2 H, CH2), 2.97(bs, 1 H), 3.688 (s, 3 H, OCH3) 4.35(dd, 1 H, J = 11.25 Hz, J = 4.7 Hz),4.48 (s, 1 H CH═,), 4.59 (s, 1 H, CH═),6.7 (d, 2 H, J = 8.65, Ar), 7.1 (d, 2 H,J = 8.8, Ar), 12.1 (bs, 1 H, CO₂H),(M + 1 = 762.49). |
| 120 | 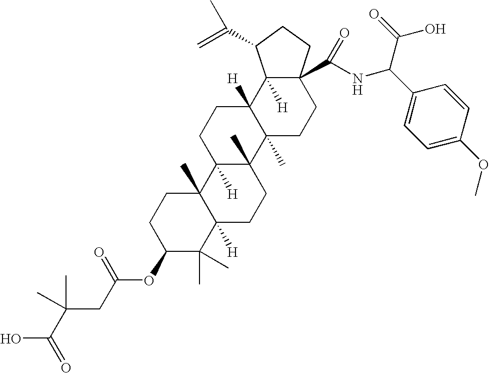 | δ 0.720 (s, 3 H, CH3), 0.769 (s, 6 H,2 × CH3), 0.776 (s, 3 H, CH3), 0.897 (s,3 H, CH3), 1.153 (s, 3 H, CH3), 1.162(s, 3 H, CH3), 1.25-1.58 (m, 18 H,CH₂), 1.63 (s, 3 H, CH3), 1.75-1.85(m, 2 H, CH₂), 2.18-2.27 (m, 2 H,CH2), 2.97 (bs, 1 H), 3.742 (s, 3 H,OCH3), 4.35 (dd,1 H, J = 11.25 Hz,J = 4.7 Hz), 4.53 (s, 1 H CH═,), 4.65 (s,1 H, CH═), 5.24 (d, 1 H, J = 7.24), 6.9(d, 2 H, J = 8.8, Ar), 7.3 (d, 2 H,J = 8.8, Ar), 7.9 (d, 1 H, J = 7.04, NH),12.1 (bs, 1 H, CO₂H), (M + 1 = 748.47). |
| 121 | 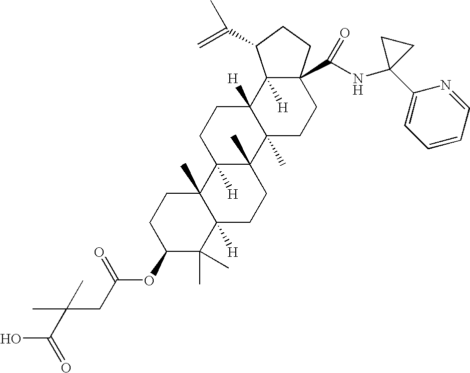 | (CDCl₃), δ 0.70-0.90 (m, 4 H), 0.77 (s,3 H, CH₃), 0.792 (s, 3 H, CH₃), 0.80 (s,3 H, CH₃), 0.82 (s, 3 H, CH₃), 0.93 (s,3 H, CH₃), 1.65 (s, 3 H, CH₃)1.10-2.05(m, 31 H), 2.32-2.43 (m, 1 H), 3.00-3.10 (m, 1 H), 4.50-4.40 (m, 1 H, H-3),4.56 (br s, 1 H, CH═), 4.69 (bs, 1 H,CH═), 7.092 (br s, 1 H, NH), 7.12 (d oft, 1 H, J = 8 and 1.6 Hz), 7,46 (br d,1 H, J = 8 Hz, CH Arom), 7.64 (d of t,1 H, J = 8 and 1,6 Hz, CH Arom),8.44. (br d, 1 H, J = 8 Hz, CH Arom).701.4904. (M + H)⁺. |

Synthesis of Compound 81

The starting material 3-acetoxy betulinic acid was prepared as follows; a solution of betulinic acid (0.50 g, 1.1 mmol) in anhydrous pyridine (10 mL) under nitrogen atmosphere was treated with Ac₂O (0.26 ml, 2.8 mmol) and DMAP (0.14 g, 1.1 mmol) and the mixture was heated at reflux for 3 to 18 h. The reaction mixture was diluted with CHCl₃ and washed with water. The organic layer was dried over MgSO₄ and concentrated under reduced pressure to give the desired compound (0.42 g, 76%). ¹H-NMR (400 MHz, d₆-DMSO) δ 0.79 (s, 6H, CH₃), 0.80 (s, 3H, CH₃), 0.87 (s, 3H, CH₃), 0.94 (s, 3H, CH₃), 1.25-1.62 (m, 18H, CH₂), 1.65 (s, 3H, CH₃), 1.75-1.85 (m, 2H, CH₂), 1.99 (s, 3H, CH₃CO), 2.08-2.14 (m, 1H), 2.18-2.27 (m, 1H), 2.90-3.00 (m, 1H), 4.36 (dd, 1H, J=11.2 Hz, J=4.8 Hz, H-3), 4.56 (m, 1H, CH═), 4.69 (d, 1H, J=2.15 Hz, CH═), 12.10 (bs, 1H, CO₂H).

A solution of oxalyl chloride (2M in CH₂Cl₂, 4 mL) was added to a solution of 3-acetoxy betulinic acid (0.175 g, 0.35 mmol) in dry CH₂Cl₂ (5 mL) and catalytic DMF (1 drop) was added. After stirring at ambient temperatures for 2 h, the mixture was concentrated under reduced pressure, diluted with dry CH₂Cl₂ (3×1 mL) and again concentrated to dryness under reduced pressure. This material was used without further purification. To a solution of the acid chloride (0.175 mmol) in dry CH₂Cl₂ (5 mL) under nitrogen atmosphere was added commercially available (R)-1-(4-methoxy-phenyl)-ethylamine (0.07 g, 0.45 mmol) and Et₃N (0.107 mL, 0.77 mmol). After stirring at ambient temperatures for 18 h, the mixture was diluted with CH₂Cl₂ (10 mL) and washed with H₂O. The organic layer was dried (MgSO₄) and concentrated under reduced pressure to give compound 28 (213 g, 96% yield). See Table 1 for appropriate analytical data.

A solution of 28 (0.201 g, 0.32 mmol) in THF (1.6 mL) and MeOH (1 mL) was treated with NaOH (4M, 0.41 mL). After stirring at ambient temperatures for 18 h, the mixture was concentrated under reduced pressure, diluted with CH₂Cl₂ and washed with aqueous HCl (0.5 N). The organic layer was dried (MgSO₄), filtered, and concentrated under reduced pressure to give amide 51 (175 mg, 93% yield). See Table 2 for appropriate analytical data.

Under nitrogen atmosphere a solution of 51 (0.169 g, 0.28 mmol) in dry pyridine (4 mL) was treated with commercially available 2,2-dimethylsuccinic anhydride (0.183 g, 1.43 mmol) and DMAP (0.035 g, 0.28 mmol) and the mixture was heated at reflux overnight. The reaction mixture was diluted with CH₂Cl₂ and washed with H₂O. The organic layer was dried (MgSO₄), filtered, and concentrated under reduced pressure to give crude 81 that was purified by RP-HPLC according to the conditions outlined herein. See Table 3 for appropriate analytical data.

Synthesis of Compound 105

To a solution of betulinic acid (0.17 g, 0.3722 mmol) in dry DMF (1.5 mL) was added commercially available (R,S)-pyridine-2-yl-ethylamine (0.068 g, 0.5583 mmol), EDCl—HCl (0.11 g, 0.5583 mmol) and HOAt (0.025 g, 0.1861 mmol). To this was then added iPr₂NEt (0.25 mL, 1.3958 mmol). After stirring for 18 h at ambient temperatures, the mixture was transferred onto rapidly stirring H₂O (5 mL) and the resultant precipitate was collected by filtration. Material was used as is without additional purification. Analytical data; LC-MS (ESI): 561.937 (M+H)⁺. To a solution of the aforementioned amide (0.107 g, 0.191 mmol) in dry pyridine (2 mL) was added commercially available 2,2-dimethylsuccinic anhydride (0.122 g, 0.954 mmol) and 4-DMAP (0.023 g, 0.191 mmol). The mixture was then heated at reflux under an inert atmosphere. After heating overnight (18 h), the mixture was concentrated under reduced pressure and purified by medium pressure liquid chromatography (SiO₂, 0-5% MeOH—CH₂Cl₂) providing 105 (75 mg, 60% yield). See Table 3 for appropriate analytical data.

Synthesis of Compound 117

To a solution of betulinic acid (0.18 g, 0.394 mmol) in dry DMF (2 mL) was added EDCl—HCl (0.113 g, 0.5912 mmol), HOAt (0.054 g, 0.3941 mmol) and iPr₂NEt (0.21 mL, 1.182 mmol) at ambient temperatures. After stirring for 10 minutes, commercially available (S)-1-(4-methoxy-phenyl)-ethylamine (0.09 g, 0.59 mmol) was introduced and the resulting mixture was allowed to stir for 18 h at ambient temperatures. After this time the mixture was transferred onto aqueous 1% HCl, the solid collected by filtration, and then purified by medium pressure liquid chromatography (SiO₂, 0-50% EtOAc-hexane) to give intermediate amide (168 mg, 72% yield). Analytical data; 1H-NMR (400 MHz, d₆-DMSO) δ 7.76 (d, J=7.6 Hz, 1H), 7.20 (d, J=8.7 Hz, 2H), 6.83 (d, J=8.7 Hz, 2H), 4.95-4.85 (m, 1H), 4.64 (d, J=2.3 Hz, 1H), 4.53 (bs, 1H), 4.26 (d, J=5.0 Hz, 1H), 3.72 (s, 3H), 2.90-3.05 (m, 2H), 2.30-2.20 (m, 1H), 1.90-1.00 (m, 25H), 1.00-0.75 (m, 10H), 0.71 (s, 3H), 0.63 (s, 3H), 0.57 (s, 3H); LC-MS (ESI): 590.4917 (M+H)⁺. To a solution of the aforementioned amide (0.153 g, 0.259 mmol) in dry pyridine (1 mL) was added commercially available 2,2-dimethylsuccinic anhydride (0.166 g, 1.297 mmol) and 4-DMAP (0.032 g, 0.259 mmol). The mixture was then heated at reflux under an inert atmosphere. After heating overnight (18 h) the mixture was diluted with toluene, concentrated under reduced pressure, recovered in CH₂Cl₂ and washed with 10% HCl. The organic layer was dried (Na₂SO₄), filtered, and concentrated under reduced pressure. Purification by medium pressure liquid chromatography (SiO₂, 0-20% MeOH—CH₂Cl₂) provided 117 (143 mg, 77% yield). See Table 3 for appropriate analytical data.

Synthesis of Compound 121

To a stirred solution of 3-acetoxy betulinic acid chloride (0.50 g, 0.967 mmol, derived from 3-acetoxybetulinic acid and oxalyl chloride as described above) in CH₂Cl₂ (15 mL) was added 1-pyridin-2-yl-cyclopropylamine (0.27 g, 2 mmol, see J. Org. Chem., 2002, 67, 3865) and Et₃N (0.57 mL, 4 mmol). After stirring at ambient temperatures 24 h, the mixture was diluted with CH₂Cl₂, washed with 1 N HCl and brine, dried (Na₂SO₄), filtered and concentrated. A solution of the crude amide in MeOH (6 mL) and THF (12 mL) was treated with 4 M NaOH (5 mL) and stirred at ambient temperatures for 18 h. After this time, the mixture was acidified, concentrated to a white paste, diluted with CH₂Cl₂ and washed with H₂O, brine and dried (Na₂SO₄). Removal of drying agent, concentration and purification by medium pressure liquid chromatography (SiO₂, 0-20% MeOH—CH₂Cl₂) provided intermediate amide. Analytical data; ¹H-NMR (400 MHz, d₆-DMSO): δ 8.45 (br s, 1H), 8.47 (d, J=4.4 Hz, 1H), 7.90-7.80 (br m, 1H), 7.43-7.40 (br m, 1H), 7.32-7.29 (br m, 1H), 4.62 (s, 1H), 4.53 (s, 1H), 3.16-2.67 (m, 2H), 2.33-2.26 (m, 1H), 1.93-1.99 (m, 2H), 1.8-0.6 (m, 44H); LCMS (m/z): 573 (M+1). To a solution of the aforementioned amide (0.118 g, 0.20 mmol) in dry pyridine (5 mL) was added commercially available 2,2-dimethylsuccinic anhydride (0.128 g, 1.0 mmol) and 4-DMAP (0.125 g, 1.0 mmol). The mixture was then heated at reflux under an inert atmosphere. After heating for 18 h the mixture was concentrated under reduced pressure and the residue was purified by RP-HPLC providing 121 (25 mg, 18% yield). See Table 3 for appropriate analytical data.

Synthesis of Compound 126

To a solution of 3-acetoxybetulinic acid (10 g, 20.1 mmol) in of CH₂Cl₂ (100 mL) was added SOCl₂ (29.2 mL, 20 equiv.) slowly at room temperature. The mixture was heated at reflux for 1.5 h, concentrated in vacuo, and dissolved in CH₂Cl₂ (100 mL). To this was then added 1-methyl-1-pyridin-2-yl-ethylamine (5.0 g, 1.5 equiv., see synthesis of compound 217, Scheme 8) and Et₃N (9.9 mL, 3.5 equiv.) and the mixture was stirred at room temperature overnight. The mixture was treated H₂O (100 mL), the organic layer separated, dried (Na₂SO₄) and concentrated to give oil. The resulting intermediate amide was dissolved in THF (100 mL) and MeOH (50 mL) and treated with 4 N NaOH (50 mL). After stirring for 4 h at room temperature the mixture was extracted with Et₂O (100 mL), the organic layer was separated and dried (MgSO₄) and concentrated. The residue was dissolved in pyridine (90 mL) and treated with commercially available 2,2-dimethylsuccinic acid (6.6 g, 5 equiv.) and DMAP (3.2 g, 1.5 equiv.) and heated at 125° C. for 18 h. The solution was concentrated, treated with aqueous 2 N HCl (200 mL) which gave a white precipitate which was collected by filtration. This material was dissolved in MeOH (30 mL) and EtOAc (120 mL) then 0.5 M NaOMe in MeOH (34 mL, 1 equiv.) was added slowly at 0° C. (ice bath). After stirring for 5 minutes, the solution was diluted with n-hexane (600 mL) providing a white precipitate that was collected by filtration and dried to give the title compound (7.13 g, 49%). Analytical data: ¹H NMR (400 MHz, d6-DMSO) δ 8.45 (d, J=6.4 Hz, 1H), 7.86 (s, 1H), 7.69 (t, J=7.6 Hz, 1H), 7.36 (d, J=7.6 Hz, 1H), 7.18 (t, J=6.4 Hz, 1H), 4.60 (s, 1H), 4.50 (s, 1H), 4.31 (m, 1H), 2.87 (m, 1H), 2.45 (m, 1H), 2.34 (m, 2H), 1.99 (m, 1H), 1.76-1.20 (m, 30H), 1.09 (m, 2H), 1.04 (s, 6H), 0.93 (s, 3H), 0.80 (s, 3H), 0.78 (s, 6H), 0.77 (s, 3H); Mass Spec (m/z): 703 (M+1).

General Procedure for Synthesizing Compound (122)

Compound 73 (0.112 g, 0.15 mmol) was suspended in glacial acetic acid (10 mL) and flushed with nitrogen. A catalytic amount of platinum (IV) oxide (0.012 g) was added. The reaction was placed under 15 psi of hydrogen gas overnight. The mixture was filtered through a pad of Celite and the solvent was evaporated under reduced pressure. The crude compound 122 obtained was purified by HPLC.

Compounds 123 and 124 were synthesized similar to compound 122.

| Com- | | |
|---|---|---|
| pound | | NMR (DMSO-d₆, 400 MHz) |
| No. | Structure | LC-MS (ESI) |
| 122 | 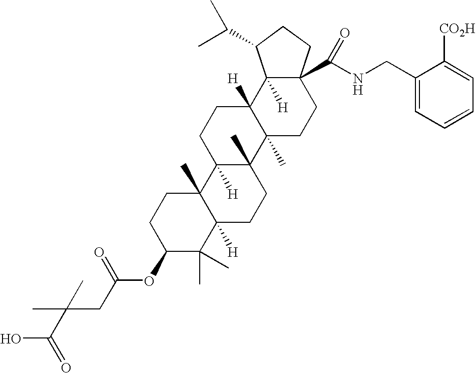 | δ 0.72 (s, 6 H, CH₃), 0.785 (s, 12 H, CH₃),0.91 (s, 3 H, CH₃), 1.16 (s, 3 H, CH₃), 1.17 (s, 3 H,CH₃), 1.20-1.85 (m, 26 H), 2.10-2.25 (m, 2 H), 4.37(dd, 1 H, J = 11.3 Hz, J = 5.0 Hz, H-3), 4.53 (d, 2 H,J = 5.9 Hz, CH₂N), 7.30-7.38 (m, 2 H),CH Arom), 7.49 (t, 1 H, J = 6.9 Hz, CH Arom),7.85 (dd, 1 H, J = 7.6 Hz, J = 1.4 Hz, CH Arom),7.99 (t, 1 H, J = 5.9 Hz, NH), 12.25 (bs, 1 H,CO₂H), 13.00 (bs, 1 H, CO₂H).LC-MS (ESI): 720.4837 (M + H)⁺. |
| 123 | 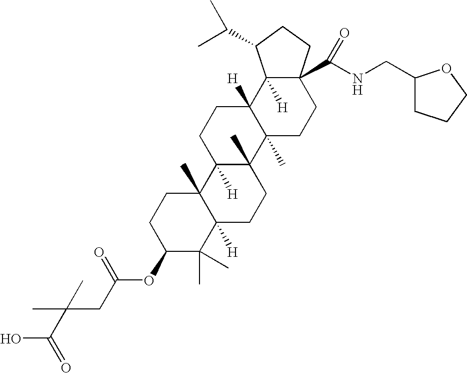 | 0.70 (d, 3 H, CH₃), 0.78, 0.79, 0.80 (s, 12 H, CH₃),0.84 (s, 3 H, CH₃), 0.90 (s, 3 H, CH₃), 0.92-1.86 (m,37 H), 2.10-2.24 (m, 1 H), 2.88-3.02 (m, 1 H),3.10-3.26 (m, 1 H, CH₂N), 3.54-3.62 (m, 1 H, CH₂N),3.68-3.76 (m, 1 H, CH₂O), 3.77-3.86 (m, 1 H,CH₂O), 4.37 (dd, 1 H, J = 11.3 Hz,J = 4.7 Hz, H-3), 7.55 (t, 1 H, J = 6.7 Hz, NH).670.5086 (M⁺ + 1). |
| 124 | 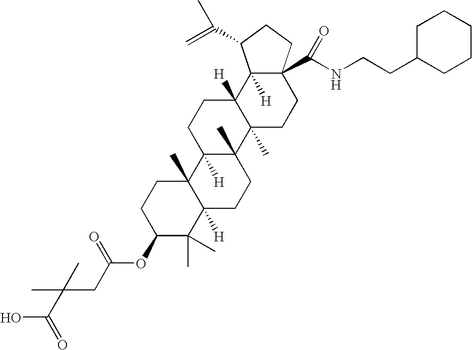 | 0.70 (d, 3 H, CH₃), 0.78, 0.79, 0.81 (s, 12 H, CH₃),0.85 (s, 3 H, CH₃), 0.90 (s, 3 H, CH₃), 0.92-1.75 (m,43 H), 2.10-2.24 (m, 1 H), 2.55-2.65 (m, 1 H),2.90-3.00 (m, 1 H), 3.10-3.26 (m, 1 H, CH₂N),3.05-3.16 (m, 1 H, CH₂N), 4.37 (dd, 1 H, J = 11.3 Hz,J = 4.9 Hz, H-3), 7.44 (t, 1 H, J = 5.8 Hz, NH),12.19 (bs, 1 H, CO₂H).696.5809 (M⁺ + 1). |

General Procedure for Synthesizing Compound (125)

Compound 125 was synthesized similar to the synthesis scheme above for compounds in Tables 1-3 provided the starting material of betulinic acid was replaced with ursolic acid.

| Com- | | |
|---|---|---|
| pound | | NMR (DMSO-d₆, 400 MHz) |
| No. | Structure | LC-MS (ESI) |
| 125 | 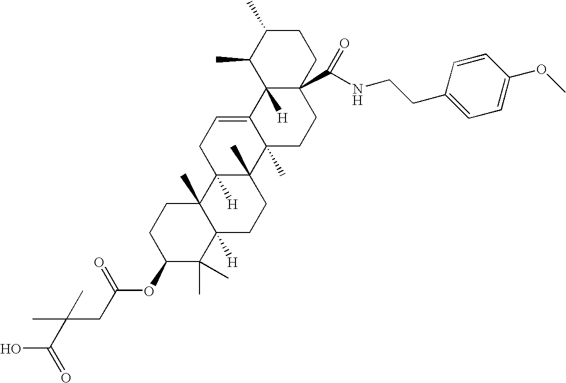 | 0.57 (s, 3 H, CH₃), 0.79-0.91 (s,9 H, CH₃), 1.02 (s, 6 H), 1.16-1.90(m, 26 H), 2.10 (m, 1 H), 2.60 (m,2 H), 3.15 (m, 2 H), 3.72 (s, 3 H),4.38 (dd, 1 H, J = 11.2 Hz, J = 4.9 Hz,H-3), 4.53 (bs, 1 H, CH═), 5.10(bt, 1 H, CH═), 6.84 (d, 2 H, J = 8.6 Hz),7.09 (bt, 1 H, amide NH), 7.10(d, 2 H, J = 8.6 Hz). 718.52 (M⁺ + 1). |

General Procedure for Synthesizing Compounds (83, 121 & 126), Scheme 1

### (3β)-3-(acetyloxy)lup-20(29)-en-28-oic acid (202)

A solution of betulinic acid (0.50 g, 1.1 mmol) in anhydrous pyridine (10 mL) under nitrogen atmosphere was treated with Ac₂O (0.26 ml, 2.8 mmol) and DMAP (0.14 g, 1.1 mmol) and the mixture was refluxed for 1 h. The reaction mixture was diluted with CHCl₃ and washed with water. The organic layer was dried over MgSO₄ and concentrated under reduced pressure to give 202 (0.42 g, 76%). ¹H NMR (DMSO-d₆, 400 MHz) δ 0.79 (s, 6H, CH₃), 0.80 (s, 3H, CH₃), 0.87 (s, 3H, CH₃), 0.94 (s, 3H, CH₃), 1.25-1.62 (m, 18H, CH₂), 1.65 (s, 3H, CH₃), 1.75-1.85 (m, 2H, CH₂), 1.99 (s, 3H, CH₃CO), 2.08-2.14 (m, 1H), 2.18-2.27 (m, 1H), 2.90-3.00 (m, 1H), 4.36 (dd, 1H, J=11.24 Hz, J=4.8 Hz, H-3), 4.56 (m, 1H, CH═), 4.69 (d, 1H, J=2.15 Hz, CH═), 12.10 (bs, 1H, CO₂H).

Oxalyl chloride solution (2M in CH₂Cl₂, 4 mL) was added to 202 (0.1 g, 0.2 mmol) and stirred for 2 h. The mixture was concentrated to dryness under reduced pressure. The residue was diluted with dry CH₂Cl₂ (3×1 mL), concentrated to dryness under reduced pressure, and used without further purification. To a solution of the acid chloride (0.2 mmol) in dry CH₂Cl₂ (5 mL) under nitrogen atmosphere was added the appropriate amine (0.26 mmol) and TEA (0.44 mmol, 0.061 mL). The reaction mixture was stirred at room temperature overnight, diluted with CH₂Cl₂ and then the CH₂Cl₂ layer washed with H₂O. The organic layer was dried over MgSO₄ and concentrated under reduced pressure to give the amide compound. In most cases the products were pure enough to use them directly for the next step, and some products were purified by chromatography.

A solution of the appropriate amide (0.21 mmol) in THF (1.6 mL) and Methanol (1 mL) and THF (1 mL) was treated with NaOH (4M, 0.27 mL). The mixture was stirred at room temperature overnight, and then the solvents were evaporated under reduced pressure. The residue was diluted with CH₂Cl₂ and washed with aqueous HCl (0.5 N). The organic layer was dried over MgSO₄ and concentrated under reduced pressure to give amide derivatives 204.

A solution of the appropriate amide 204 (0.17 mmol) in dry Pyridine (4 mL), under nitrogen atmosphere, was treated with 2,2-dimethylsuccinic anhydride (0.109 g, 0.85 mmol) and DMAP (0.021 g, 0.17 mmol) and the mixture was heated at reflux overnight. The reaction mixture was diluted with CH₂Cl₂ and washed with H₂O. The organic layer was dried over MgSO₄ and concentrated under reduced pressure to give the carboxylic acid product. The crude material was purified by HPLC.

General Procedure for Synthesizing Compounds (105a-105b), Scheme 5

To a suspension of 1 (50 g, 109.6 mmol) and anhydrous K₂CO₃ (22.85 g, 164.4 mmol) in a dry acetone (1 L) was added benzylbromide (20.6 g, 120.6 mmol) and the mixture stirred at room temp for 24 h. Solvent was evaporated and the residue was suspended in 2 N HCl (1 L) and stirred at room temp for 2 hr and filtered. The solid was washed with mixture of AcCN and H₂O (20:80) (200 mL) and dried. Yield 56 g (93%). To a stirred solution of alcohol 205 (56 g, 102.5 mmol) in pyridine (500 mL) was added DMAP (19 g, 122.17 mmol) and dimethylsuccinic anhydride (65.3 g, 510 mmol) and heated at reflux for 24 h. The solvent was evaporated and the residue was suspended in cold 1 N HCl (500 mL) and stirred for 2 h at 0 C. The solid was filtered and washed with 1 N HCl (200 mL) and dried providing 68 g (98%). To an ice-cold solution of acid (68.4 g, 102 mmol) in MeOH (200 mL) and THF (200 mL) was added SOCl₂ (29.4 mL, 408 mmol) slowly over a 2 h period and then was allowed to stir overnight at room temperature. The solvent was evaporated and the residue was suspended in 1 N HCl (500 mL) and stirred at room temp for 2 h and then filtered. The filtered residue was suspended in MeOH (200 mL) and heated for 20 min at 60 C and filtered hot and dried overnight. This provided 69 g (96%) of compound 206.

To the benzyl ester 206 (50 g, 72.5 mmol) dissolved in mixture of THF (200 mL) and MeOH (150 mL) was added 10% Palladium/Charcoal (5 g) and the mixture stirred while ammonium formate (5 g, 79.8 mmol) was added slowly. The mixture was then stirred at room temp for 2 h. Upon completion of the reaction, catalyst was filtered and washed with THF (100 mL). The residue thus obtained after evaporation of the washings was suspended in hot AcCN and stirred for 30 min, filtered hot and dried overnight. Yield 41 g (95%). To an ice-cold solution of acid (46 gm, 76.8 mmol) in dry CH₂Cl₂ (350 mL) was added SOCl₂ (19.4 mL, 269 mmol) and a few drops of DMF and stirred for 30 min. The cooling bath was removed and the solution was heated at reflux for 2 h. The solvent was evaporated and the residual SOCl₂ was removed by adding CHCl₃ and evaporating. The crude acid chloride thus obtained was dissolved in dry CH₂Cl₂ (300 mL) and stirred at 0 C. At this time, either (S)-1-pyridin-2-yl-ethylamine (S-211) (11.5 g, 92.16 mmol) or (R)-1-pyridin-2-yl-ethylamine (R-211) (11.5 g, 92.16 mmol) was added slowly followed by Et₃N (33 mL, 230 mmol) and stirred at room temperature overnight. The solvent was evaporated, and the residue thus obtained was suspended in 1 N HCl (500 mL) and stirred at room temp for 2 h and then filtered to provide compound 208.

Ester 208, obtained in the previous step, was hydrolyzed as a solution in THF (300 mL), MeOH (400 mL) and 4 M NaOH (130 mL) room temperature for 8 h. The solvent was evaporated the solid residue was filtered and washed repeatedly with cold H₂O. The mono-sodium salt thus obtained was precipitated with a mixture EtOAc and hexane to yield 44 g (83% yield for three steps) of 105a or 105b as white powders.

### Synthesis of (R)- and (S)-1-pyridin-2-yl-ethylamine (211), Scheme 6

Please see; Brunner, H.; Niemetz, N. Monatshefte fur Chimie 2002, 133, 115-126.

### Synthesis of 1-pyridin-2-yl-cyclopropylamine (215), Scheme 7

A 1 N solution of LiHMDS in THF (1277 mL, 1277 mmol), under nitrogen atmosphere, was treated with compound 213 (63 g, 336 mmol). After 10 minutes, tBuOH (94 mL, 1007 mmol) was added. After an additional 10 minutes, 1,2-dibromoethane (87 mL, 1007 mmol) was added. The mixture was then heated at 60° C. for 16 h after which it was cooled to room temperature and stripped of solvent in vacuo to give oil which was taken up in MeOH (1000 mL). A 4 N solution of NaOH (500 mL) was then added and the solution was stirred at room temperature for 4 h. The solution was then acidified to a pH<1 by the addition of concentrated HCl after which the solvent was removed in vacuo providing the product along with inorganic salts. The product was removed from the salts by trituration with MeOH (500 mL) then isolation of the solids by filtration. The methanolic solution was then treated with Et₂O (2000 mL) causing the product to precipitate out of solution. The product was then isolated by filtration and dried giving compound 214 as a light brown solid in 77% yield.

Under a nitrogen atmosphere, compound 214 (51.5 g) was suspended in toluene (1000 mL), and Et₃N was added followed by diphenylphosphorylazide. The reaction was then heated at 90° C. overnight. The solids were filtered off and discarded and the solvent was removed in vacuo. The residue was dissolved in a 1:1 solution of MeOH and 4 N NaOH (1000 mL) and heated to 70° C. for 3 h. The product was extracted into EtOAc, then acidified and extracted into H₂O, then re-basified and once again extracted into EtOAc. The solvent was removed in vacuo and the residue was taken up in methanolic HCl (300 mL). The solvent was stripped in vacuo to give compound 215 as a grey/green solid in 20% yield.

### Synthesis of 1-methyl-1-pyridin-2-yl-ethylamine (217), Scheme 8

To a solution of 2-cyanopyridine (33.0 g, 0.32 mol) in 800 mL of toluene was added MeMgBr (566 mL, 2.5 equiv) slowly at 0° C. The mixture was heated at 100° C. overnight, and then quenched with 2 N HCl in an ice bath. The aqueous layer was collected and basified with 4 N NaOH, and then extracted with ether (500 mL×3). The combined organic layer was dried and concentrated to give the title compound (35.0 g, 81%).

TABLE 6

| | | | | Metabolic | |
|---|---|---|---|---|---|
| | | | | Stability in | |
| | | | | Human liver | |
| | | | | Microsomes | |
| | | | | % | |
| | | | | compound | |
| | | | PBMC | remaining | |
| | | Analytical | IC₅₀ | at 40 | T_{1/2} |
| No. | Structure | data | (μM) | minutes | minutes |
| 83 | 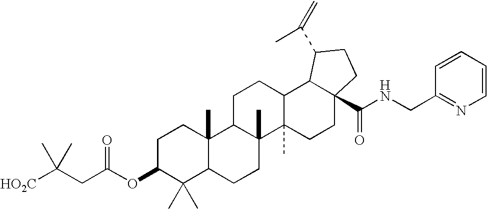 | δ 0.74 (s, 3 H, CH₃), 0.78 (s,6 H, CH₃), 0.93 (0.93, 3 H,CH₃), 1.00-1.92 (m, 37 H),2.18-2.26 (m, 1 H), 2.94-3.04 (m, 1 H), 4.26-4.44 (m,3 H, H-3 and CH₂N), 4.53(bs, 1 H, CH═), 4.64 (bs,1 H, CH═), 7.32-7.44 (m,2 H, CH Arom), 7.91 (bs,1 H, CH Arom), 8.34(bs, 1 H, NH), 8.56 (bs,1 H, CH Arom), 12.16 (bs,1 H, CO₂H).675.4716 (M⁺ + 1). | 0.0046(MT4assay) | 11 | <10 |
| 105a | 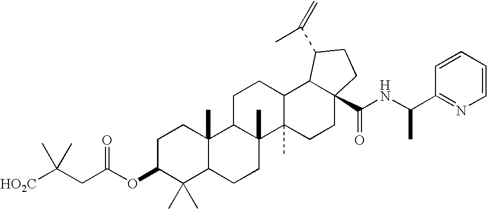 | δ 8.474 (1 H, d, J = 4.6 Hz),7.90 (1 H, d, J =7.4 Hz),7.71 (1 H, d, J = 7.4 Hz),7.31 (1 H, d, J = 7.8 Hz),7.22 (1 H, m), 4.93 (1 H,quintet, J = 7.2 Hz), 4.63(1 H, s), 4.53 (1 H, s), 4.30(1 H, m), 3.0 (1 H, m), 0.57-2.5 (54 H, m).689.50(M⁺ + 1). | 0.0026 | 44 | 36 |
| 105b | 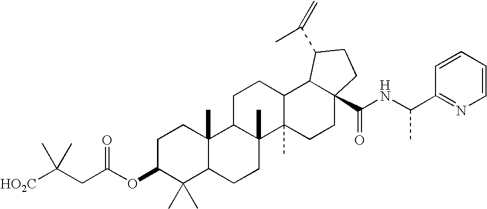 | δ 8.50-8.47 (1 H, m), 7.94(1 H, d, J = Hz), 7.74 (1 H,d of t, J =8 and 2 Hz), 7.28(1 H, d, J = 8.4 Hz), 7.24-7.20 (1 H, m), 4.95 (1 H,quintet, J = 7.6 Hz), 4.61(1 H, s), 4.51 (1 H, s), 4.34-4.30 (1 H, m), 2.96-2.80 (1 H,m), 0.57-2.6 (54 H, m).689.50(M⁺ + 1). | 0.0031 | 32 | 27 |
| 121 | 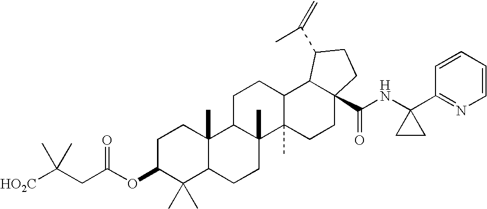 | δ 0.70-0.90 (m, 4 H), 0.77(s, 3 H, CH₃), 0.792 (s, 3 H,CH₃), 0.80 (s, 3 H, CH₃),0.82 (s, 3 H, CH₃), 0.93 (s,3 H, CH₃), 1.65 (s, 3 H,CH₃)1.10-2.05 (m, 31 H),2.32-2.43 (m, 1 H), 3.00-3.10 (m, 1 H), 4.50-4.40 (m,1 H, H-3), 4.56 (br s, 1 H,CH═),4.69 (bs, 1 H,CH═), 7.092 (br s, 1 H, NH),7.12 (d of t, 1 H, J = 8 and1.6 Hz), 7,46 (br d, 1 H,J = 8 Hz, CH Arom), 7.64(d of t, 1 H, J = 8 and1,6 Hz, CH Arom), 8.44.(br d, 1 H, J = 8 Hz,CH Arom). 701.4904.(M + H)⁺. | 0.018 | 42 | 30 |
| 126 | 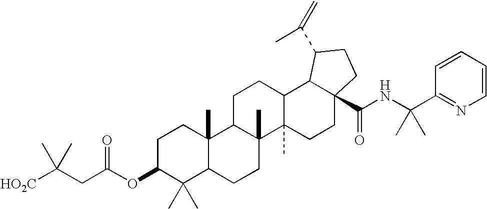 | ¹H NMR (DMSO-d₆,400 MHz) δ 8.45 (d, 1 H,J = 6.4 Hz), 7.86 (s,1 H), 7.69 (t, 1 H,J = 7.6 Hz), 7.36 (d,1 H, J = 7.6 Hz), 7.18 (t, 1 H,J = 6.4 Hz), 4.60 (s, 1 H),4.50 (s, 1 H), 4.31 (m, 1 H),2.87 (m,1 H), 2.45 (m, 1 H),2.34 (m, 2 H), 1.99 (m, 1 H),1.76-1.20 (m, 30 H), 1.09(m, 2 H), 1.04 (s, 6 H), 0.93(s, 3 H), 0.80 (s, 3 H), 0.78(s, 6 H), 0.77 (s, 3 H).TOF-MS m/z 703 (M + H)⁺ | 0.0475 | 47 | 38 |

### Synthesis of Aryl Ethyl and Aryl Methyl Amines

Non-commercially available starting materials for synthesis of compounds of the present invention can be synthesized according to the following general synthetic routes.

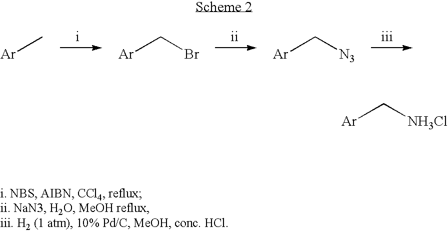

### Representative Bromination Procedure

### Scheme 1, Step i; 2-Bromomethyl-5-fluoro-benzoic Acid Methyl Ester

To a 0.5 M solution of methyl 5-fluoro-2-methylbenzoic acid (1 equiv.) in CCl₄ was added N-bromo succinimide (1.2 equiv.) followed by AIBN (0.1 equiv.) at room temperature. The mixture was then heated at reflux temperature for 18 h, cooled and filtered. The filtrate was concentrated, recovered in Et₂O (75 mL) and washed with H₂O (3×20 mL), saturated NaCl solution (2×20 mL), and then dried over MgSO₄. Filtration and removal of solvent under reduced pressure gave colorless oil (85% yield) that was used without further purification.

The following benzyl and aryl methyl halides were prepared according to this procedure:
5-Bromomethyl-thiophene-2-carboxylic acid methyl ester
3-Bromomethyl-pyridine-2-carboxylic acid methyl ester

### Representative Azide Displacement and Catalytic Hydrogenation Procedures

### Scheme 1, Steps ii and iii

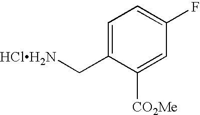

### Methyl 2-aminomethyl-5-fluoro-benzoate Hydrochloride for Preparation of Compound 128

To a 0.4 M solution of 2-bromomethyl-5-fluoro-benzoic acid methyl ester (1 equiv.) in MeOH was added a 7 M solution of NaN₃ (1.5 equiv.) in H₂O at room temperature. The mixture was then heated at reflux temperature for 2 h. The reaction mixture was concentrated then recovered in Et₂O and H₂O. The organic layer was separated, washed with saturated NaCl solution, and then dried over MgSO₄. Filtration and removal of solvent under reduced pressure gave colorless oil. The crude material was dissolved in a mixture of MeOH (0.4 M) and concentrated HCl (1.2 equiv.) and 10% Pd/C (10% by weight) was added at room temperature. The mixture was placed under hydrogen atmosphere (1 atm) for 2.5 h. Catalyst was removed by filtration through Celite; the pad was washed with MeOH, and the pale yellow solution was concentrated under reduced pressure to give a yellow solid. Trituration with diethyl ether and drying under vacuum gave the desired product that was used without further purification.

The hydrochloride salts listed below were prepared according to this procedure:
Methyl 5-aminomethyl-thiophene-2-carboxylate (for Compd 129)
Methyl 3-aminomethyl-pyridine-2-carboxylic acid methyl ester (Cmpd 130)

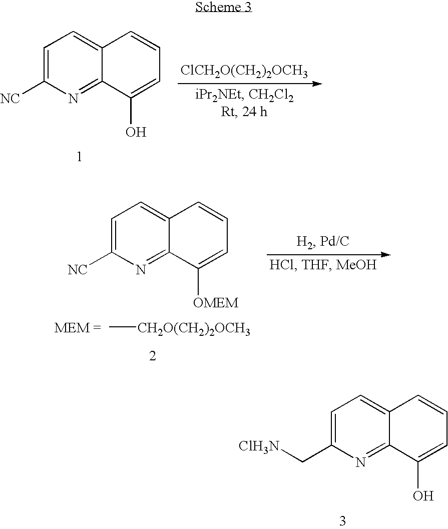

### Synthesis of 2-aminomethyl-quinolin-8-ol Hydrochloride (3) for Preparation of Cmpd 131

A solution of 8-hydroxyquinoline-2-carbonitrile (1) (2.9 mmol, 1 eq) in CH₂Cl₂ (20 mL) was treated with 2-methoxyethoxymethyl chloride (4.4 mmol, 1.5 eq) followed by iPr₂NEt (5.8 mmol, 2 eq). After stirring at room temperature for 18 h, the mixture was quenched with H₂O, extracted with CH₂Cl₂, dried and concentrated to provide 2 (83% yield).

Without further purification compound 2 was added to a mixture of MeOH (20 mL) and THF (10 mL) containing 10% Pd/C (70 mg). The resultant mixture was stirred under H₂ gas (1 atmosphere) at room temperature for 18 h. The flask was thoroughly evacuated, back-filled with N₂, and the catalyst removed by filtration over Celite. Removal of solvent the desired product which was used as is in subsequent steps.

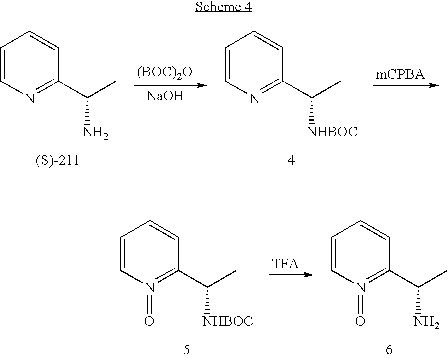

### Synthesis of (S)-1-(1-oxy-pyridin-2-yl)-ethylamine (6) for Preparation Cmpd 132

To a solution of (S)-1-Pyridin-2-yl-ethylamine (519 mg, 4.25 mmol) ((S)-211) in THF (25 mL) was added di-tert-butyl-dicarbonate (976 μL, 4.25 mmol) and aqueous NaOH solution (1 N NaOH, 8.56 mL, 8.5 mmol) and the mixture was stirred at room temperature for 5 h. The mixture was diluted with EtOAc (25 mL), the organic layer was washed with H₂O, brine and dried over anhydrous Na₂SO₄. The residue obtained after evaporation of the solvent was dissolved in CH₂Cl₂ (25 mL) and cooled to ice-cold temperature. To this solution was slowly added mCPBA (1.7 g, 10 mmol) and the solution was stirred at room temperature for overnight. The mixture was diluted with CH₂Cl₂ (25 mL), washed with saturated Na₂CO₃ (15 mL×2), H₂O and dried over anhydrous Na₂SO₄. The residue obtained after evaporation of the solvent was purified (silica gel column using EtOAc—hexanes as eluent). To a stirred solution of [1-(1-oxy-pyridin-2-yl)-ethyl]-carbamic acid tert-butyl ester (5) (500 mg, 2.25 mmol) in CH₂Cl₂ (25 mL) was added 10 mL of trifluoroacetic acid (TFA) and the mixture was stirred at room temp for 5 h. Evaporation of solvent and TFA provided 250 mg of (S)-1-(1-oxy-pyridin-2-yl)-ethylamine (6) that was used as such in the standard amide coupling reaction.

For synthesis of Compound 133, see general procedure for synthesizing compound 122.

TABLE 7

| | | NMR (DMSO-d₆, | MT4 | PBMC |
|---|---|---|---|---|
| Cmpnd | | 400 MHz) | EC₅₀ | IC₅₀ |
| No. | Structure | LC-MS (ESI) | (μM) | (μM) |
| 127 | 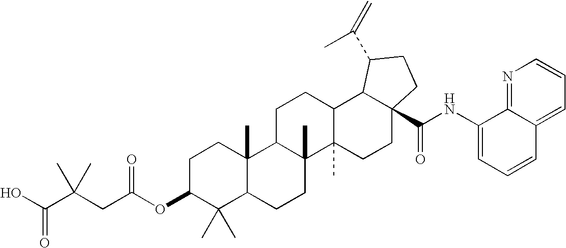 | δ 12.19 (bs, 1 H), 10.09(s 1 H), 8.92 (m, 1 H),8.60 (dd, 1 H, J = 1.36,7.56 Hz), 8.43 (dd, 1 H,J = 1.61, 8.24 Hz), 7.66(m, 2 H), 7.59 (t, 1 H,J = 8.46 Hz), 4.75 (m,1 H), 4.60 (m, 1 H), 4.36(m, 1 H), 3.16 (m, 1 H),3.00 (m, 1 H), 2.80 (m,2 H), 2.48-2.05(m, 5 H),2.03-0.70 (m, 42 H).MS (m/z): 715.01(M⁺ + 1). | 3.1 | NT |
| 128 | 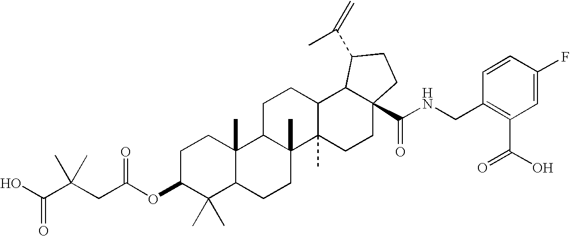 | δ 8.08 (1 H, t,J = 5.6 Hz), 7.58 (1 H, dd,J = 2.8 Hz and 9.6 Hz),7.42-7.34 (2 H, m),4.74 (1 H, br s), 4.53-4.5 (3 H, m), 4.35 (1 H,m), 3.2-2.8 (1 H, m),2.5-0.6 (52 H, m).MS (m/z): 736.458(M + 1). | 0.028 | 0.0036 |
| 129 | 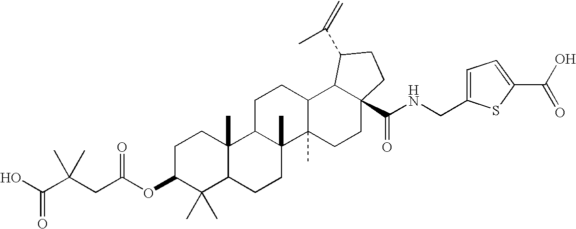 | δ 8.37 (1 H, t, J = 6 Hz),7.54 (1 H, d, J = 3.6 Hz),7.97 (1 H, d, J = 4 Hz),4.66 (1 H, br s),4.54 (1 H, br s), 4.45-4.32 (2 H, m), 3.1-2.95(1 H, m), 2.6-0.8 (53 H, m).MS (m/z): 724.424(M + 1). | 0.371 | NT |
| 130 | 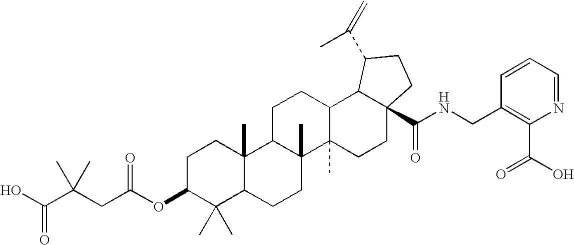 | δ 8.52 (dd, 1 H, J = 1.6 Hz,4.4 Hz), 7.75 (d, 1 H,J = 8.4 Hz), 7.55 (m, 1 H,)4.64 (bs, 1 H), 4.52-4.48(m, 3 H), 4.77-4.31 (m,1 H), 2.95-2.90 (m, 1 H),2.6-0.8 (m, 52 H).MS (m/z): 719 (M + 1). | 0.024 | 0.044 |
| 131 | 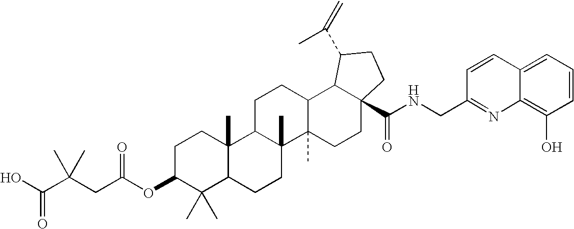 | δ 8.43 (bs, 1 H, Ar),8.367 (bs, 1 H, NH),7.488-7.428 (m, 3 H,Ar), 7.150 (bs, 1 H, Ar),4.641-4.530 (m, 5 H,CH2), 4.369 (bs, 1 H,CH), 2.988-0.767(m, 52 H, CH)MS (m/z): 741.490(M + 1). | 0.003 | 0.0035 |
| 132 | 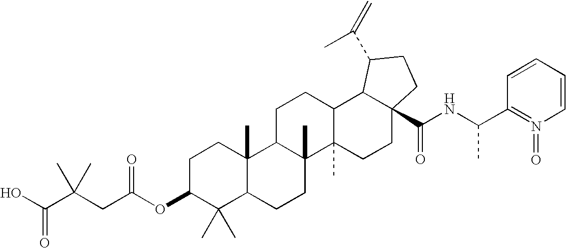 | δ 8.25 (d, J = 6.4 Hz, 1 H),8.19 (1 H, d, J = 8 Hz),7.35-7.28 (m, 3 H),5.3 (m, 1 H), 4.59 (bs,1 H), 4.5 (bs, 1 H), 4.39-4.30 (m, 1 H), 2.9-2.8(m, 1 H), 2.6-0.8 (m,54 H).MS (m/z): 705.47 (M + 1). | 0.036 | 0.044 |
| 133 | 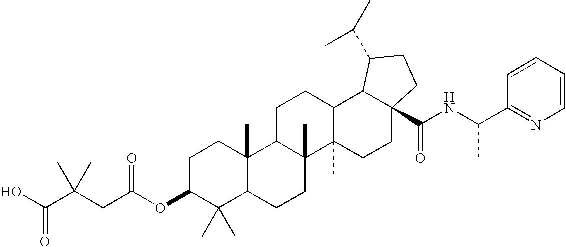 | δ 8.58 (1 H, m), 7.92 (1 H,d, J = 7.6 Hz), 7.69(1 H, d of t, J = 7.8 and2 Hz), 7.28 (1 H, d,J = 8.7 Hz), 7.25-7.19(1 H, m), 4.95 (1 H,quintet, J = 7.6 Hz),4.34-4.30 (1 H, m), 2.8-0.56 (59 H, m).MS (m/z): 689.70(M⁺ − 1). | NT | NT |

### Example 2

### Determination of Antiviral Activity

The compounds of the invention can be tested in the following assays to detect antiviral activity and general toxicity.

MT-4 Cytoprotection Assay

The HTLV-1 transformed T cell line, MT-4, is highly susceptible to HIV-1 infection. Anti-HIV-1 agents were evaluated in this target cell line by protection from the HIV-induced cytopathic effect. In this assay, viability of both HIV-1 and mock-infected cells was assessed in a calorimetric assay that monitors the ability of metabolically-active cells to reduce the tetrazolium salt WST-1. Cytoprotection by antiviral compounds is indicated by the positive readout of increased WST-1 cleavage.

Briefly, exponentially growing MT-4 cells were mock-infected or batch-infected with the HIV-1 laboratory strain, NL4-3, at a multiplicity of infection of 0.0005. Following a two hour infection, the cells were washed to remove unbound virus and plated in the presence of increasing concentrations of compound. After four days incubation, cytoprotection in the infected cells and compound toxicity in mock-infected cells were analyzed using the WST-1 assay.

PBMC Drug Susceptibility Assay

Human peripheral blood mononuclear cells (PBMCS) were used to test compound antiviral activity as an indicator for clinical efficacy. PBMCs were isolated from two donors using a Ficoll-Hypaque density gradient, pooled and stimulated with PHA-L for three days. After stimulation, the cells were washed and maintained in culture medium containing IL-2. The stimulated cells were then mock-infected or batch-infected with the strain HIV-1_{IIIB} at MOI 0.01 for one hour. Cells (unwashed) were then plated in the presence of increasing concentrations of compound and incubated for seven days. The readout for virus replication in these cultures is the concentration of HIV-1 p24 in the supernatant because PBMCs generally do not succumb to HIV-induced cytopathic effects. Compound toxicity in mock-infected cells was analyzed using the WST-1 assay.

It was found that compounds of the invention have antiviral activity according to these assays. Compound 71 has an EC50 (concentration of compound that reduces the virus induced cytopathic effect by 50% (MT-4) (antiviral activity measure)) of about 126 nanomolar and a TC50 (TC50 is the concentration of compound that results in death of 50% of the host cells (toxicity measure)) of about 7.7 micromolar. Compound 73 has an EC50 of about 8.1 nanomolar and a TC50 of about 6.3 micromolar. Compound 70 has an EC50 of about 2.9 nanomolar and a TC50 of greater than 10 micromolar. Compound 76 has an EC50 of about 11 nanomolar and a TC50 of greater than 10 micromolar. Compound 46 has an EC50 of about 8.6 micromolar and a TC50 of greater than 10 micromolar. Representative compounds of the invention include those with an EC50 of less than about 100 nm, such as compounds 69, 70, 73-84, 87, 88, 91-95, 97, 99-106, 108-117, 119-124.

### Example 3

### Metabolic Stability of Specific Compounds

Compounds 105a and 105b of the present invention may be synthesized as follows:

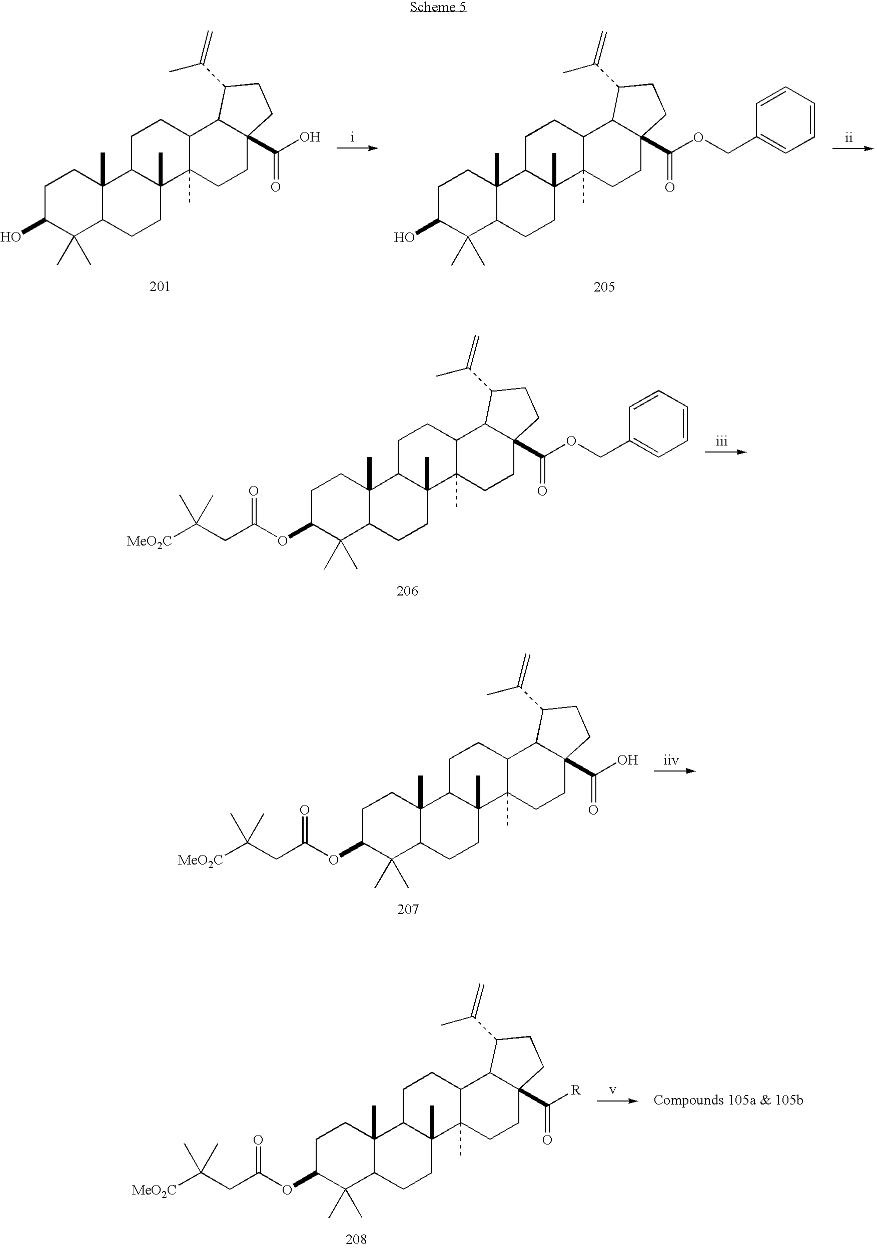

i. Benzyl bromide, K₂CO₃, DMF; ii. 2,2-dimethylsuccinic anhydride, DMAP, Py, Δ, then MeOH, SOCl₂, reflux; iii. Pd/C, ammonium formate; iv. SOCl₂, CH₂Cl₂, pyridine then H₂NR, TEA, CH₂Cl₂; v. NaOH (4M), THF/MeOH.

According to this scheme, diastereomeric compounds 105, having the structure

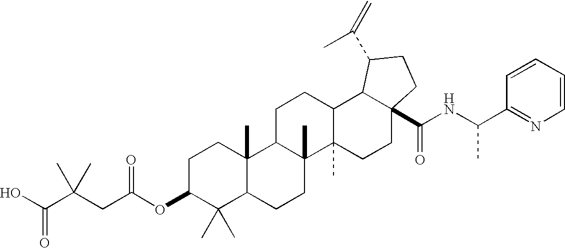

are prepared by providing a compound 208 according to Scheme 5 above and converting compound 208 to compound 105.

Compound 208 is provided by converting compound 207 according to Scheme 5 to compound 208.

Compound 207 is provided by converting compound 206 of Scheme 5 to compound 207.

Compound 206 is provided by converting compound 205 of Scheme 5 to compound 206.

Compound 205 is provided by converting compound 201 of Scheme to compound 205.

Alternatively, compound 207 of Scheme 5 may be provided by converting compound 201 of Scheme 5 to compound 207.

Scheme 6 below illustrates a method of synthesizing the pyridine-containing side chain of compound 105:

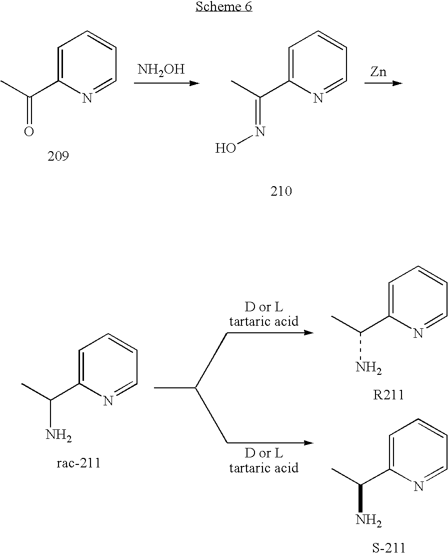

executed according to procedures contained in Brunner, H.; Niemetz, N. Monatshefte fur Chimie 2002, 133, 115-126.

Scheme 7 below illustrates a method of synthesizing the pyridine-containing side chain of e.g., compound 121.

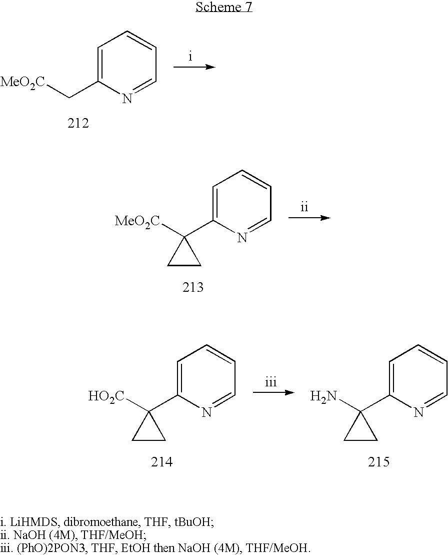

Scheme 8 below illustrates a method of synthesizing a pyridine-containing side chain of a compound containing two methyl substituents.

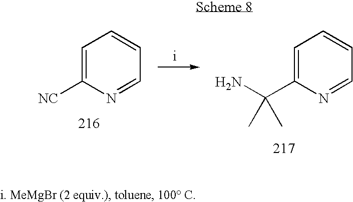

### Example 4

### Scheme and Procedures for Preparation of Betulinic Acid Oxetane Derivatives

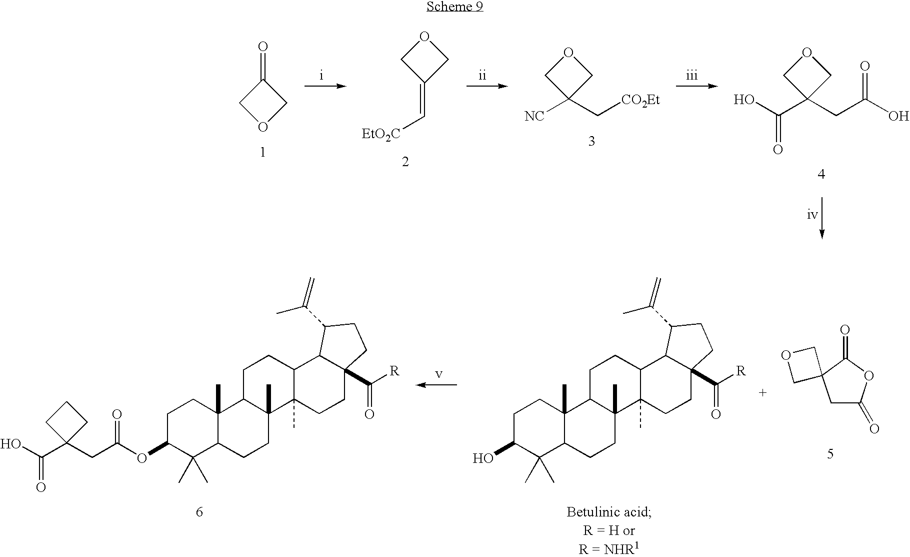

The above scheme summarizes the synthetic routes to the compounds in Tables 1-3 where the reagents/conditions are: i. See Wuitschik, G., Rogers-Evans, M.; Muller K., Fischer, H.; Wagner, B.; Schuler, F.; Polonchuk, L.; Carreira, E. M. Angew. Chem. Int. Ed. Engl. 2006, 45, 7736-9. ii. NaCN, EtOH, H₂O, reflux. iii. MeOH, THF, NaOH, 30% H₂O₂. iv. Ac₂O (0.9 equiv.), reflux. v. DMAP, pyridine, 95° C.

A solution (0.2 M) of oxetan-3-ylidene-acetic acid ethyl ester (2) (1 equiv.) in 20% aqueous EtOH is treated with NaCN (2 equiv.) and heated at reflux temperature for 6-12 hours. The reaction mixture is diluted with CH₂Cl₂ and washed with water and the organic layer is dried over Na₂SO₄. Removal of solvent provides desired product 3.

To a solution (0.2 M) of (3-cyano-oxetan-3-yl)-acetic acid ethyl ester (3) in a mixture of MeOH and THF (1:1) is added 2M NaOH (2 equiv.) and 30% H₂O₂ (2 equiv.). The mixture is then stirred at reflux temperature for 12-24 hours, acidified with concentrated HCl and the aqueous solution extracted with CH₂Cl₂. The combined organic extracts are dried over anhydrous Na₂SO₄ and then concentrated providing compound 4.

A mixture of 3-carboxymethyl-oxetane-3-carboxylic acid (4) (1 equiv.) and Ac2O (0.9 equiv.) is heated at reflux for 10-12 hours. The round bottomed flask is fitted with a short-path distillation head and the desired product 5 is collected in a cooled receiver flask.

Betulinic acid derivatives 6 are prepared using 5 according to the general procedure.

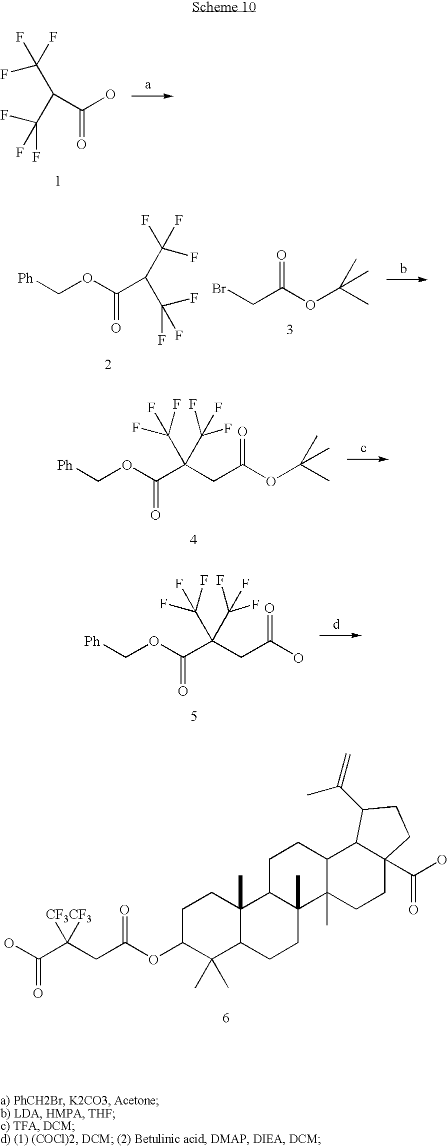

Step A

To a stirred solution of the 3,3,3-Trifluoro-2-trifluoromethyl-propionic acid 1 (2 mmols, 392 mg) in acetone (25 mL) is added anhydrous potassium carbonate (3 mmols, 414 mg) and benzyl bromide (2.1 mmols, 359 mg) and the mixture was allowed to stir overnight at room temp. Evaporated the solvent and redissolved in ethylether and washed with water. Ether layer is dried over sodium sulfate and evaporated. The residue thus obtained purified with silica gel column chromatography employing ethyl ether and hexane as eluent.

Step B

The succinic acid diester 4 is prepared according to the similar procedure reported in N. Petraganani, M. Yonashiro, Synthesis page 710, 1980. To a stirred solution of LDA (1 mmol) in THF at −78 C was added benzyl 3,3,3-Trifluoro-2-trifluoromethyl-propionate 2 (1 Mmols) in THF and t-butyl bromomethylacetate 3 (1 mmols) followed by HMPA (1 equivalent) and stirred at that temp for 3 hr. 1N HCl saturated with sodium chloride is added and extracted with ether. Ether layer was dried over anhydrous sodium sulfate and rotovaped to get the residue. The residue was purified over silica gel chromatography using ethyl ether and hexane as eluents.

Step C

To a stirred solution of t-butyl ester 4 (2 mmols) in DCM is added TFA (5 mmols) and stirred at room temp overnight. Evaporated the solvent and the benzyl ester residue is used as such in the next step. The residue acid 5 thus obtained is redissolved in dichloromethane (25 mL), added oxalyl chloride (5 mmols) and a catalytic amount of DMF stirred at room temp 2 hrs. Evaporated the solvent and the residue is used as such in the next step.

Step D

To a stirred solution of betulinic acid (1 mmols) in dichloromethane (10 mL) is added was added DMAP (5 mmols), diispropylethylamine (2 mmols) and acid chloride (derived from acid 5) (5 mmols) dissolved in DCM. The reaction mixture is refluxed over 24 hrs. Evaporated the solvent and the residue is purified by reverse phase HPLC to obtain compound 6.

### Example 5

### Human Liver Microsome Assay

The microsomal fraction of liver homogenates contains endoplasmic reticulum derived cytochrome P450 enzymes that constitute the major phase I drug metabolizing enzymes. Briefly, to aid prediction of the in vivo rate of clearance of a dosed test article, an in vitro incubation containing human liver microsomes (0.5 mg/mL final protein concentration) and test article (1 μM incubation concentration) in the presence and absence of the necessary cofactor NADPH (1 mM incubation concentration) is conducted. The incubation is performed in a buffered aqueous system (100 mM potassium phosphate, pH 7.4). The concentration of the parent compound is determined using liquid chromatography coupled with tandem mass spectrometry and reported as percent remaining from a zero minute concentrations.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference. The mere mentioning of the publications and patent applications does not necessarily constitute an admission that they are prior art to the instant application.

Although the foregoing invention has been described in some detail by way of illustration and example for purposes of clarity of understanding, it will be obvious that certain changes and modifications may be practiced within the scope of the appended claims.

What is claimed:

1. A compound having a structure 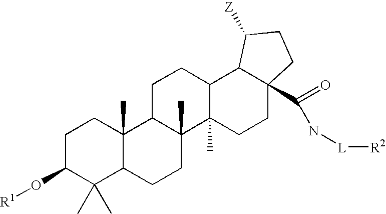
and pharmaceutically acceptable salts and stereoisomers thereof, wherein
L is —(CH₂)ₙ—, wherein n is an integer chosen from 0, 1, 2, and 3, and L can be substituted with alkyl;
R¹ is R¹¹—C(O)—, wherein R¹¹ is C₁₋₂₀ alkyl or C₁₋₂₀ alkenyl, each being optionally substituted with one or more substituents independently chosen from the group consisting of: C₁₋₆ alkyl; —CN; hydroxyl; heteroaryl; heterocycle; —C(O)R¹², wherein R¹² is C₁₋₆ alkoxy, C₁₋₆ alkenyloxy, or heterocycle; —C(O)—N(R¹³)(R¹⁴), wherein R¹³ and R¹⁴ are independently H, C₁₋₆ alkyl, heteroaryl, —P(O)(OH)₂, (C₁₋₆ alkyl)phosphono, or —SO₃R¹⁵, wherein R¹⁵ is H, C₁₋₆ alkyl or aryl, or R¹³ and R¹⁴ together with the nitrogen atom they are linked to form a 3 to 6-membered heterocycle; —N(R¹³)(R¹⁴), wherein R¹³ and R¹⁴ are independently H, C₁₋₆ alkyl, aryl, heteroaryl, or R¹³ and R¹⁴ together with the nitrogen atom they are linked to form a 3 to 6-membered heterocycle; —SO₃R¹⁵, wherein R¹⁵ is C₁₋₆ alkyl, or heteroaryl; —NHSO₃R¹⁶, wherein R¹⁶ is C₁₋₆ alkyl, or heteroaryl; and —P(O)(OR¹⁷)₂, wherein R¹⁷ is H or C₁₋₆ alkyl; wherein optionally two substituents at one carbon atom of R¹¹ may, together with the one carbon atom they are attached to, form a 3 to 6-membered heterocycle;
R² is chosen from cycloalkyl, aryl, arylalkyl, and heterocycle optionally substituted with 1-6 substituents independently chosen from:
(1) halo;
(2) hydroxyl;
(3) C₁₋₁₀ alkyl or C₃₋₆ cycloalkyl, optionally substituted with 1-3 moieties independently chosen from: hydroxyl; halo; C₁₋₆ alkoxy; C₁₋₆ haloalkoxy; C₃₋₁₀ cycloalkyl; heterocycle; aryl; heteroaryl; —C(O)R⁴, wherein R^{4a} is —OH, C₁₋₆ alkoxy, C₁₋₆ alkenyloxy, C₁₋₆ alkynyloxy, C₃₋₆ cycloalkoxy or heterocycle; —C(O)—N(R^{4c})(R^{4d}), wherein R^{4c} and R^{4d} are independently H, C₁₋₆ alkyl, aryl, heteroaryl, C₃₋₆ cycloalkyl, or —SO₃R^{4e}, wherein R^{4e} is H, C₁₋₆ alkyl or aryl, or R^{4c} and R^{4d} together with the nitrogen atom they are linked to form a 3 to 6-membered heterocycle; —N(R^{4c})(R^{4d}), wherein R^{4c} and R^{4d} are independently H, C₁₋₆ alkyl, C₁₋₆ hydroxyalkyl, aryl, heteroaryl, C₃₋₆ cycloalkyl, or —SO₃R^{4e}, wherein R^{4e} is H, C₁₋₆ alkyl or aryl, or R^{4c} and R^{4d} together with the nitrogen atom they are linked to form a 3 to 6-membered heterocycle; —SO₃R⁴, wherein R^{4f} is C₁₋₆ alkyl, aryl or heteroaryl; —NHSO₃R^{4g}, wherein R^{4g} is C₁₋₆ alkyl, aryl, or heteroaryl; —N(R^{4b})—C(O)R^{4h}, wherein R^{4b} is H or methyl or ethyl, R^{4h} is C₁₋₆ alkyl; and —N(R^{4b})—C(O)—N(R^{4c})(R^{4d}), wherein R^{4b} is H or methyl or ethyl, R^{4c} and R^{4d} are independently H, C₁₋₆ alkyl, aryl, heteroaryl, C₃₋₆ cycloalkyl, or —SO₃R^{4e}, wherein R^{4e} is H, C₁₋₆ alkyl or aryl, or R^{4c} and R^{4d} together with the nitrogen atom they are linked to form a 3 to 6-membered heterocycle;
(4) —CO₂R⁴ⁱ or —O(C═O)R⁴ⁱ, wherein R⁴ⁱ is H or C₁₋₆ alkyl;
(5) —N(R^{4c})(R^{4d}) or —SO₂N(R^{4c})(R^{4d}), wherein R^{4c} and R^{4d} are independently H, OH(R^{4c} and R^{4d} are not both OH), C₁₋₆ hydroxyalkyl, or C₁₋₆ alkyl, aryl or heteroaryl, or R^{4c} and R^{4d} taken together with the nitrogen they are attached to form a 3 to 6-membered heterocycle;
(6) —SO₃R^{4e}, wherein R^{4e} is C₁₋₆ alkyl, aryl or heteroaryl;
(7) —NHSO₃R^{4f}, wherein R^{4f} is C₁₋₆ alkyl, aryl, or heteroaryl;
(8) —N(R^{4b})C(═O)R^{4h}, —N(R^{4b})C(═O)N(R^{4c})(R^{4d}), or —OC(═O)N(R^{4c})(R^{4d}), wherein R^{4b} is H or methyl or ethyl; R^{4h}, R^{4c} and R^{4d} are independently H, OH(R^{4c} and R^{4d} are not both OH), C₁₋₁₀ alkyl, C₂₋₁₀ alkenyl, C₂₋₁₀ alkynyl, C₁₋₁₀ alkoxy, C₁₋₁₀ alkylthiol, C₂₋₁₀ alkenyloxy, C₂₋₁₀ alkynyloxy, C₁₋₁₀ haloalkyl, C₂₋₆ hydroxyalkyl, C₁₋₆ alkyl-O—C₁₋₆ alkyl-, cycloalkyl, heterocycle, aryl, heteroaryl, or R^{4c} and R^{4d} together with the nitrogen atom to which they are both linked form a 3 to 6-membered heterocycle;
(9) C₁₋₆ alkoxy optionally substituted with 1-3 substituents each being independently chosen from the group consisting of: hydroxyl; halo; —CO₂R⁴ⁱ, wherein R⁴ⁱ is H or C₁₋₆ alkyl; heterocycle optionally substituted with 1-3 substituents each being independently halo, C₁₋₆ alkyl, or C₁₋₃ haloalkyl; heteroaryl optionally substituted with 1-3 substituents each being independent halo, hydroxyl, C₁₋₆ alkyl, C₁₋₆ alkoxy, carboxyl, C₁₋₃ alkoxycarbonyl, C₁₋₃ hydroxyalkyl, C₁₋₃ haloalkyl, or —N(R^{4c})(R^{4d}) or —SO₂N(R^{4c})(R^{4d}), wherein R^{4c} and R^{4d} are independently H, OH(R^{4c} and R^{4d} are not both OH), C₁₋₃ alkyl, C₁₋₆ hydroxyalkyl, or C₁₋₆ alkyl, or R^{ae} and R^{af} taken together with the nitrogen they are attached to form a 3 to 6-membered heterocycle; and —N(R^{4c})(R^{4d}), wherein R^{4c} and R^{4d} are independently H, hydroxyl, C₁₋₆ alkyl, C₁₋₆ hydroxyalkyl, or —N(R^{4m})(R⁴ⁿ), wherein R^{4m} and R⁴ⁿ are independently H or C₁₋₃ alkyl, or R^{4c} and R^{4d} can be taken together with the nitrogen they are attached to form a 3 to 6-membered heterocycle, and/or R^{4m} and R⁴ⁿ can be taken together with the nitrogen they are attached to form a 3 to 6-membered heterocycle;
(10) —CON(R^{4p})(R^{4q}), wherein R^{4p} and R^{4q} are independently H, or C₁₋₁₀ alkyl that is optionally substituted with 1-3 substituents each being independently hydroxyl; halo; —N(R^{4r})(R^{4t}), wherein R^{4r} and R^{4t} are independently H, C₁₋₃ alkyl, hydroxyl, or C₁₋₃ hydroxylalkyl; heterocycle optionally substituted with 1-3 substituents each being independently halo, C₁₋₆ alkyl, or C₁₋₃ haloalkyl; C₁₀ alkoxy, C₁₀ alkylthiol, C₂₋₁₀ alkenyloxy, C₂₋₁₀ alkynyloxy; and aryl or heteroaryl, optionally substituted with 1-3 substituents each being independently halo, hydroxyl, C₁₋₆ alkyl, C₁₋₃ haloalkyl, carboxyl, C₁₋₃ alkoxycarbonyl, —N(R^{4c})(R^{4d}) or —SO₂N(R^{4c})(R^{4d}), wherein R^{4c} and R^{4d} are independently H, OH(R^{4c} and R^{4d} are not both OH), C₁₋₃ alkyl, C₁₋₆ hydroxyalkyl, or C₁₋₆ alkyl, or R^{4c} and R^{4d} taken together with the nitrogen they are attached to form a 3 to 6-membered heterocycle; and
(11) cycloalkyl, heterocycle, aryl, or heteroaryl, optionally substituted with 1-3 substituents each being independently halo; hydroxyl; C₁₋₆ alkyl; C₁₋₃ haloalkyl; —CO₂R⁴ⁱ or —O(C═O)R⁴, wherein R⁴ⁱ is H or C₁₋₃ alkyl; —N(R^{4c})(R^{4d}) or —SO₂N(R^{4c})(R^{4d}), wherein R^{4c} and R^{4d} are independently H, OH (R^{4c} and R^{4d} are not both OH), C₁₋₃ alkyl, C₁₋₆ hydroxyalkyl, or C₁₋₆ alkyl, or R^{4c} and R^{4d} taken together with the nitrogen they are attached to form a 3 to 6-membered heterocycle; and
Z is chosen from isopropyl and isopropenyl optionally substituted with one or two substituents independently selected from hydroxyl, halo, amino, pyrrolidinyl, and piperidinyl.

2. The compound according to claim 1, wherein
R¹ is succinyl, glutaryl, 3′-methylglutaryl, 3′-methylsuccinyl, 3′,3′-dimethylsuccinyl or 3′,3′-dimethylglutaryl, or an alkyl ester thereof, and
Z is isopropenyl.

3. A pharmaceutical composition comprising a compound according to claim 1, and a pharmaceutically acceptable carrier.

4. The pharmaceutical composition according to claim 3, further comprising an antiviral agent.

5. The pharmaceutical composition according to claim 4, wherein said antiviral agent is amantadine.

6. A method for inhibiting a retroviral infection in cells or tissue of an animal comprising administering an effective retroviral inhibiting amount of a compound according to claim 1.

7. The method according to claim 6, wherein said retroviral infection does not respond to other therapies.

8. A method for inhibiting a retroviral infection in cells or tissue of an animal comprising administering an effective retroviral inhibiting amount of a pharmaceutical composition according to claim 5.

9. The method according to claim 8, wherein the retroviral infection does not respond to other therapies.

10. The method according to claim 6, wherein said composition is administered to provide said compound in an amount ranging from about 0.01 μg/kg to about 100 mg/kg body weight.

11. The method according to claim 6, wherein said animal is a human.

12. A method of delaying the onset of HIV infection from a mother to a baby, comprising administering to said mother and/or said baby a therapeutically effective amount of a compound of claim 1, during pregnancy, delivery, or shortly thereafter.

13. A method of delaying the onset of HIV infection in an individual who has sex with an infected person, comprising administering a therapeutically effective amount of a compound of claim 1 to vaginal or other mucosa prior to having sex.
